(19) United States
(12) Patent Application Publication
(10) Pub. No.: US 2022/0017898 A1
(43) Pub. Date: Jan. 20, 2022

(54) OLIGONUCLEOTIDES CONJUGATES COMPRISING 7'-5'-ALPHA-ANOMERIC-BICYCLIC SUGAR NUCLEOSIDES

(71) Applicant: ALPHA ANOMERIC SAS, Paris (FR)

(72) Inventor: Wolfgang RENNER, Bözberg (CH)

(21) Appl. No.: 17/054,724

(22) PCT Filed: May 10, 2019
(86) PCT No.: PCT/EP2019/062064

§ 371 Date: Nov. 12, 2020

Related U.S. Application Data
(60) Provisional application No. 62/670,319, filed on May 11, 2018.

(51) Int. Cl. C12N 15/113

(52) U.S. Cl. CPC C12N 15/113; C12N 2310/11; C12N 2310/315; C12N 2310/3515; C12N 2320/33; C12N 2310/337

(57) ABSTRACT

The invention provides for an oligonucleotide lipid group conjugate, wherein the oligonucleotide comprises at least two alpha anomeric bicyclo-DNA residues connected by a phosphodiester bond, and wherein the lipid group is attached to the oligonucleotide via a linker. The invention also provides for methods of modulating gene expression using an oligonucleotide lipid group conjugate.

### TECHNICAL FIELD

The invention is directed to oligonucleotide conjugates, and their use to modulate gene expression.

### BACKGROUND OF THE INVENTION

Antisense oligonucleotides influence RNA processing and modulate protein expression. In certain instances, antisense compounds result in altered transcription or translation of a target. Such modulation of expression can be achieved by, for example, target mRNA degradation or occupancy-based inhibition. Oligonucleotide analogs that exhibit strong, sequence specific binding to single-stranded or a double-stranded target, and are resistant to chemical degradation are potentially useful as therapeutic agents. Chemically modified oligonucleotides have been designed for therapeutic uses.

### SUMMARY OF THE INVENTION

The invention provides for oligonucleotides comprising abc-DNA nucleosides and conjugated to a lipid group. The abc-DNA nucleosides are preferably connected via a phosphodiester bond.

The invention provides for an oligonucleotide-lipid group conjugate wherein the oligonucleotide comprises at least two alpha anomeric bicyclo-DNA (abc-DNA) residues connected by a phosphodiester bond, and wherein the lipid group is covalently attached to the oligonucleotide.

In one embodiment, the lipid group is covalently attached to the oligonucleotide via a linker.

In another embodiment, the oligonucleotide comprises 12 to 24 residues. In another embodiment, the oligonucleotide comprises 14 to 20 residues. In another embodiment, the oligonucleotide comprises 14 to 19 residues. In another embodiment, the oligonucleotide comprises 15 to 19 residues. In another embodiment, the oligonucleotide comprises 15 residues. In another embodiment, the oligonucleotide comprises 16 residues. In another embodiment, the oligonucleotide comprises 17 residues. In another embodiment, the oligonucleotide comprises 18 residues. In another embodiment, the oligonucleotide comprises 19 residues.

In another embodiment, the abc-DNA residue has the formula (V)

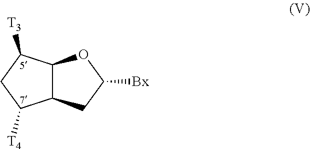

wherein independently for each of the at least two abc-DNA residue of formula (IV) one of T₃ or T₄ is a nucleosidic linkage group; the other of T₃ and T₄ is OR₁, OR₂, a 5′ terminal group, a 7′ terminal group or a nucleosidic linkage group, and wherein

R₁ is H or a hydroxyl protecting group, and

R₂ is a phosphorus moiety; and

Bx is a nucleobase, wherein preferably Bx is selected from a purine base or pyrimidine base, and wherein further preferably Bx is selected from uracil, thymine, cytosine, 5-methylcytosine, adenine or guanine.

Thus, in another embodiment, the abc-DNA residue has the formula (V)

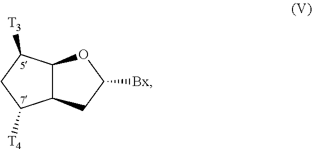

wherein
(i) T₃ is a nucleosidic linkage group, and T₄ is a 7′ terminal group, OR₁, or OR₂, preferably T₄ is a 7′ terminal group or OR₁; or
(ii) T₃ is a 5′ terminal group, OR₁, or OR₂, preferably T₃ is a 5′ terminal group or OR₂; and

T₄ is a nucleosidic linkage group; or
(iii) T₃ and T₄ are independently of each other a nucleosidic linkage group;

and wherein

R₁ is H or a hydroxyl protecting group, and

R₂ is a phosphorus moiety; and

Bx is a nucleobase, wherein preferably Bx is selected from a purine base or pyrimidine base, and wherein further preferably Bx is selected from uracil, thymine, cytosine, 5-methylcytosine, adenine or guanine.

In another embodiment, all of the residues are abc-DNA residues.

In another embodiment, the at least two abc-DNA residues are connected via phosphodiester bonds to adjacent residues. In another embodiment, the at least two abc-DNA residues are connected via phosphodiester bonds to adjacent residues and each further nucleosidic linkage group is independently of each other selected from a phosphodiester linkage group, a phosphotriester linkage group, a phosphorothioate linkage group, a phosphorodithioate linkage group, a phosphonate linkage group, a phosphonothioate linkage group, a phosphinate linkage group, a phosphorthioamidate linkage or a phosphoramidate linkage group

In another embodiment, all of the residues are abc-DNA residues and are connected via phosphodiester bonds. Thus, in another embodiment, each nucleosidic linkage group is a phosphodiester linkage group.

In another embodiment, the lipid group is covalently attached to a terminal residue of the oligonucleotide.

In another embodiment, the oligonucleotide comprises residues connected via a phosphorous containing nucleosidic linkage group selected from the group consisting of: a phosphodiester linkage group, a phosphotriester linkage group, a phosphorothioate linkage group, a phosphorodithioate linkage group, a phosphonate linkage group, a phosphonothioate linkage group, a phosphinate linkage group, a phosphorthioamidate linkage and a phosphoramidate linkage group.

In another embodiment, the linker is a hydrocarbon linker or a polyethylene glycol (PEG) linker.

In another embodiment, the linker is selected from the group consisting of: an amino-alkyl-phosphorothioate linker, an amino-PEG-phosphorothioate linker, an alpha-carboxylate-amino-alkyl phosphorothioate linker, and an alpha-carboxylate-amino-PEG-phosphorothioate linker.

In another embodiment, the linker comprises a cleavable group.

In another embodiment, the lipid group is a fatty acid derived group.

In one embodiment, the fatty acid is saturated or unsaturated.

In another embodiment, the fatty acid has a length from 4 to 28 carbon atoms.

In another embodiment, the fatty acid derived group comprises a carboxylic acid group.

In another embodiment, the fatty acid derived group is derived from a dicarboxylic acid.

In another embodiment, the fatty acid is selected from the fatty acids presented in Table 1 or Table 2.

In another embodiment, the fatty acid is hexadecanoic acid.

In one embodiment, the lipid group is attached to the linker via a thiophosphate group.

In one embodiment, the lipid group is attached to the oligonucleotide via a thiophosphate group.

In another embodiment, the oligonucleotide conjugate binds to the pre-mRNA corresponding to a portion of exon 51 of the Duchenne Muscular Dystrophy (DMD) gene.

In another embodiment, the oligonucleotide conjugate comprises a sequence selected from the group consisting of SEQ ID NOs: 4, 5, 22 to 24, 36 to 39, 51 to 55, 404 and 414 to 425. In another embodiment, the oligonucleotide conjugate comprises the sequence of SEQ ID NO: 417 or SEQ ID NO: 418.

In another embodiment, the oligonucleotide comprises any one of the sequences provided in Table 3.

In one embodiment, the oligonucleotide conjugate binds to the pre-mRNA corresponding to a portion of exon 53 of the DMD gene.

In one embodiment, the oligonucleotide comprises any one of the sequences provided in Table 4.

In another embodiment, the oligonucleotide conjugate binds to the pre-mRNA corresponding to a portion of exon 45 of the DMD gene.

In one embodiment, the oligonucleotide comprises any one of the sequences provided in Table 5.

The invention also provides for a pharmaceutical composition comprising an oligonucleotide-lipid group conjugate, wherein the oligonucleotide comprises at least two alpha anomeric bicyclo-DNA (abc-DNA) residues connected by a phosphodiester bond, and wherein the lipid group is covalently attached to the oligonucleotide, in combination with a suitable carrier.

The invention also provides a method for altering expression of a gene by permitting hybridization of an oligonucleotide-lipid group conjugate, wherein the oligonucleotide comprises at least two alpha anomeric bicyclo-DNA (abc-DNA) residues connected by a phosphodiester bond, and wherein the lipid group is covalently attached to the oligonucleotide, to an RNA expressed from the gene, the oligonucleotide comprising a sequence that is complementary to a portion of the RNA.

The invention also provides for a method for inducing the skipping of exon 51 of the human dystrophin pre-mRNA in a subject with Duchenne Muscular Dystrophy (DMD) or Becker Muscular Dystrophy (BMD), or in a cell derived from the subject, the method comprising providing an oligonucleotide-lipid group conjugate wherein the oligonucleotide comprises at least two alpha anomeric bicyclo-DNA (abc-DNA) residues connected by a phosphodiester bond, and wherein the lipid group is covalently attached to the oligonucleotide, which comprises a sequence selected from the group consisting of SEQ ID NOs: 4, 5, 22 to 24, 36 to 39, 51 to 55, 404 and 414 to 425, preferably of SEQ ID NO: 417 or SEQ ID NO: 418, wherein the oligonucleotide conjugate induces skipping of the exon in the subject or the cell, and wherein mRNA produced from skipping exon 51 of the dystrophin pre-mRNA encodes a functional dystrophin protein or a dystrophin protein of a Becker subject. y

The invention also provides for a method of treating Duchenne Muscular Dystrophy (DMD) or Becker Muscular Dystrophy (BMD) in a subject or in a cell derived from the subject by inducing the skipping of exon 51 of the human dystrophin pre-mRNA, the method comprising providing to the subject or the cell a composition comprising an oligonucleotide-lipid group conjugate wherein the oligonucleotide comprises at least two alpha anomeric bicyclo-DNA (abc-DNA) residues connected by a phosphodiester bond, and wherein the lipid group is covalently attached to the oligonucleotide, comprising a sequence selected from the group consisting of SEQ ID NOs: 4, 5, 22 to 24, 36 to 39, 51 to 55, 404 and 414 to 425, preferably of SEQ ID NO: 417 or SEQ ID NO: 418, wherein the oligonucleotide conjugate induces skipping of the exon in the subject or the cell, and wherein mRNA produced from skipping exon 51 of the dystrophin pre-mRNA encodes a functional dystrophin protein or a dystrophin protein of a Becker subject.

The invention also provides for a method for inducing the skipping of exon 51 of the human dystrophin pre-mRNA in a subject with Duchenne Muscular Dystrophy (DMD) or Becker Muscular Dystrophy (BMD), or in a cell derived from the subject, the method comprising providing an oligonucleotide-lipid group conjugate wherein the oligonucleotide comprises at least two alpha anomeric bicyclo-DNA (abc-DNA) residues connected by a phosphodiester bond, and wherein the lipid group is covalently attached to the oligonucleotide, which comprises any one of the sequences presented in Table 3, wherein preferably all of the residues are abc-DNA residues, wherein the oligonucleotide conjugate induces skipping of the exon in the subject or the cell, and wherein mRNA produced from skipping exon 51 of the dystrophin pre-mRNA encodes a functional dystrophin protein or a dystrophin protein of a Becker subject.

The invention also provides for a method of treating Duchenne Muscular Dystrophy (DMD) or Becker Muscular Dystrophy (BMD) in a subject or in a cell derived from the subject by inducing the skipping of exon 51 of the human dystrophin pre-mRNA, the method comprising providing to the subject or the cell a composition comprising an oligonucleotide-lipid group conjugate wherein the oligonucleotide comprises at least two alpha anomeric bicyclo-DNA (abc-DNA) residues connected by a phosphodiester bond, and wherein the lipid group is covalently attached to the oligonucleotide, comprising any one of the presented in Table 3, wherein preferably all of the residues are abc-DNA residues, wherein the oligonucleotide conjugate induces skipping of the exon in the subject or the cell, and wherein mRNA produced from skipping exon 51 of the dystrophin pre-mRNA encodes a functional dystrophin protein or a dystrophin protein of a Becker subject.

### BRIEF DESCRIPTION OF THE FIGURES

FIG. 1A: Assessment of acidic stability of alpha anomeric oligonucleotides by liquid chromatography-mass spectrometry (LS-MS). LS-MS chromatogram of untreated ON1.

FIG. 1B: LS-MS fragmentation pattern of untreated ON1.

FIG. 1C: LS-MS chromatogram of ON1 treated for 24 hours in acidic conditions.

FIG. 1D: LS-MS fragmentation pattern of ON1 treated for 24 hours in acidic conditions.

FIG. 2A: Assessment of thermal stability of alpha anomeric oligonucleotides by LS-MS. LS-MS chromatogram of untreated ON1.

FIG. 2B: LS-MS fragmentation pattern of untreated ON1.

FIG. 2C: LS-MS chromatogram of ON1 heated at 95° C. for 60 min.

FIG. 2D: LS-MS fragmentation pattern of ON1 heated at 95° C. for 60 min.

FIG. 3: Assessment of biostability stability of alpha anomeric oligonucleotides by 20% denaturing PAGE. PAGE of ON1 and its corresponding natural oligonucleotide incubated in mice serum.

FIG. 4: Mobility shift assay of ON1 incubated at different albumin equivalents.

FIG. 5: Comparison of uncomplexed ON1 incubated at different albumin equivalents. The values were obtained by ultrafiltration experiments.

FIG. 6: Mobility shift assay of ON1 incubated at different mice serum volumes.

FIG. 7: Intensity of nanoparticles present in ON1 solutions.

FIG. 8: Agarose gel for mouse exon 23 skipping efficiency into C2C12 cells detected by nesting RT-PCR.

FIG. 9A: Agarose gel for human exon 51 skipping efficiency in KM155 cells detected by nesting RT-PCR.

FIG. 9B: Agarose gel for human exon 51 skipping efficiency in KM155 cells detected by nesting RT-PCR.

### DETAILED DESCRIPTION

The invention provides for oligonucleotide conjugates comprising at least one (one or more) alpha anomeric bicyclo-DNA (abc-DNA) nucleosides, a phosphodiester group linking the nucleosides of the oligonucleotide, and a lipid group connected to the oligonucleotide via a linker. The invention provides for oligonucleotides comprising abc-DNA nucleosides, connected via phosphodiester internucleosidic bonds, and conjugated to a ligand group.

The oligonucleotides of the invention modulate gene expression by interfering with transcription, translation, splicing and/or degradation and/or by inhibiting the function of a non-coding RNA.

### Definitions

As used herein “alpha anomeric bicyclo-DNA (abc-DNA) nucleoside” means a nucleoside analog containing a bicyclic sugar moiety and having the general structure shown in below.

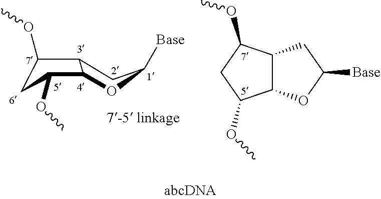

The structure of 7′-5′-alpha-anomeric-bicyclo-DNA is shown below.

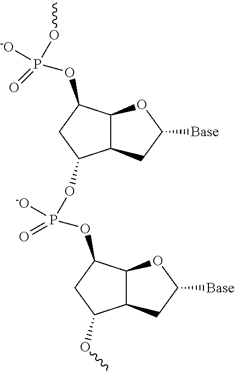

As used herein, a “bicyclic sugar moiety” comprises two interconnected ring systems, e.g. bicyclic nucleosides wherein the sugar moiety has a 2′-O—CH(alkyl)-4′ or 2′-O—CH₂-4′ group, locked nucleic acid (LNA), xylo-LNA, alpha-L-LNA, beta-D-LNA, cEt (2′-0,4′-C constrained ethyl) LNA, cMOEt (2′-0,4′-C constrained methoxyethyl) LNA, or ethylene-bridged nucleic acid.

As used herein, “nucleoside” refers to a nucleobase covalently linked to a sugar.

“Ribonucleoside” refers to a base linked to ribose; “deoxyribonucleoside” refers to a base linked to a 2′-deoxyribose.

As used herein, “nucleotide” means a nucleoside further comprising a phosphorus moiety covalently linked to the sugar of the nucleoside.

As used herein, the term “residue” refers to the nucleoside or nucleotide monomers which form the units of an oligomer—an oligonucleotide polymer.

As used herein, an “oligonucleotide” is an oligomer that may be single-stranded or double-stranded, but binds as a single stranded nucleic acid molecule to a complementary nucleic acid in a cell or organism. An oligonucleotide comprises at least two nucleosides connected to each other each by a nucleosidic linkage group as defined herein. An oligonucleotide may comprise ribonucleotides, deoxyribonucleotides, modified nucleotides (e.g., nucleotides with 2′ modifications, synthetic base analogs, etc.) or combinations thereof. Such modified oligonucleotides can be preferred over native forms because of properties such as, for example, enhanced cellular uptake and increased stability in the presence of nucleases. An oligonucleotide includes compounds comprising naturally occurring nucleotides, modified nucleotides or nucleotide mimetics, and oligonucleotides with modifications made to the sugar and/or nucleobase and/or nucleosidic linkage group as known in the art and described herein.

In certain embodiments, an oligonucleotide of the invention has a length of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides or more, for example 12-50 nucleotides, or 12-40 or 12-24 nucleotides, for example, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 nucleotides.

The term “oligomer”, for example an oligonucleotide, as used herein, refers to a compound comprising two or more monomer subunits linked by nucleosidic linkage groups. An oligomer of the invention has a length of up to 50 monomer subunits, for example, up to 40 monomer subunits, for example, up to 30 monomer subunits, up to 20 monomer subunits, or up to 15 monomer subunits. An oligomer can comprise from 5 to 40 monomeric subunits, from 8 to 30 monomer subunits, from 8 to 25 monomer subunits, or from 8 to 20 monomer subunits.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which, in certain cases, are metabolized in a manner similar to the reference nucleotides. Examples of such analogs include, without limitation, phosphorothioates, phosphoramidates, phosphorodiamidates, methylphosphonates, chiral-methyl phosphonates, 2′-O-methyl ribonucleotides, and peptide-nucleic acids (PNAs).

The invention provides for oligonucleotides that are conjugated via a covalent linkage to a lipid group. As used herein, a “lipid group” is any fatty acid group or fatty acid derived group, any steroid derived group and any lipid soluble vitamin group. An abc-DNA oligonucleotide-lipid group conjugate can exhibit a long half-life in vivo. A lipid group can also increase the binding of an abc-DNA oligonucleotide to albumin and/or other fatty acid receptors or transporters. The structure of an oligonucleotide of the invention conjugated to a lipid group is such that the lipid group is exposed to facilitate binding to albumin and/or other transporters. In another embodiment of the invention, a lipid group further contains one or two carboxylic acid groups, further increasing the interaction with albumin and/or other fatty acid receptors or transporters. In one embodiment, a lipid group is a fatty acid derived group. In another embodiment, a lipid group is a fatty acid derived group from a dicarboxylic acid. Fatty acids include any saturated or unsaturated fatty acid having a hydrocarbon chain of 2 to 28 carbon atoms, and can contain one or two carboxylic groups. One or two fatty acid ligands can be attached to the oligonucleotide via linkers on the 5′ and/or 7′ ends of an abc-DNA oligonucleotide as described herein. Lipid groups useful according to the invention are provided in Tables 1 and 2.

A lipid group of the invention can include cholesterol, vitamin E (tocopherol) or bile acid.

As used herein, a “linker” means a moiety connecting an oligonucleotide of the invention to a lipid group. Linkers useful according to the invention include but are not limited to hydrocarbon and PEG linkers, for example: amino-alkyl-phophorothioate linkers, alpha-carboxylate-amino-alkyl-phosphorothiate linkers, amino-PEG-phosphorothioate linkers and alpha-carboxylate-amino-PEG-phosphorothioate linkers. A linker according to the invention typically and preferably does not decrease or prevent the binding of the oligonucleotide to its target. A linker can include a cleavable group.

As used herein, a “nucleoside linkage group” means a linking group connecting abc-DNA nucleosides of an oligonucleotide. The nucleoside linkage groups of the invention are predominantly phosphodiester internucleosidic linkages or bonds. The term “nucleosidic linkage group” includes phosphorus linkage groups that are not phosphodiester bonds, as well as non-phosphorus linkage groups.

The invention provides for an oligonucleotide conjugated to a lipid group where all of the internucleoside linkages are phosphodiester bonds. In certain embodiments, the internucleoside linkage groups of the lipid group-conjugated oligonucleotide are predominantly phosphodiester bonds. As used herein, “predominantly” means 50% or more, for example, 55%, 60%, 65%, 70%, 75%, 80%, 85%, 90%, 95%, 99% and 100% of the internucleoside linkage groups are phosphodiester bonds. For example, an oligonucleotide can include 1 or more, and up to 50%, phosphorothioate linkages. The nucleosides of the oligonucleotides of the invention are predominantly abc-DNA nucleosides. Predominantly, as it refers to abc-DNA nucleosides, means 50% or more, for example, 55%, 60%, 65%, 70%, 75%, 80%, 85%, 90%, 95%, 99% and 100% of the nucleosides are abc-DNA nucleosides. For example, an oligonucleotide of the invention can include lor more, and up to 50%, nucleosides having a sugar that is not an abc-DNA nucleoside.

The invention provides for nucleosides connected via a phosphorus containing internucleosidic bond, or a phosphodiester bond. The invention also provides for nucleosides connected via predominantly phosphodiester bonds but including, a “phosphorus containing nucleoside linkage group” selected from a phosphotriester linkage group, a phosphorothioate linkage group, a phosphorodithioate linkage group, a phosphonate linkage group, for example, a H-phosphonate linkage group or a methylphosphonate linkage group, a phosphonothioate linkage group, for example, a H-phosphonothioate linkage group, a methyl phosphonothioate linkage group, a phosphinate linkage group, a phosphorthioamidate linkage group, or a phosphoramidate linkage group.

As used herein, a “nucleoside” or “nucleotide” encompasses naturally occurring or modified nucleosides or nucleoside mimetics, or naturally occurring or modified nucleotides or nucleotide mimetics, respectively, that can be incorporated into an oligomer of the invention via chemical or non-chemical methods for oligomer synthesis. As used herein, “natural” or “naturally occurring”, means of natural origin.

The term “modified nucleosides” includes nucleosides having modifications to the sugar and/or nucleobase of a nucleoside as known in the art and described herein. The term “modified nucleotides” includes nucleotides having modifications to the sugar and/or nucleobase and/or nucleosidic linkage group or phosphorus moiety of a nucleotide as known in the art and described herein.

As used herein, “nucleoside mimetic” includes structures used to replace the sugar and the nucleobase. The term “nucleotide mimetic” includes nucleotides used to replace the sugar and the nucleosidic linkage group. Examples of nucleotide mimetics include peptide nucleic acids (PNA) or morpholinos.

A “nucleoside” or “nucleotide” of the invention can include a combination of modifications, for example, more than one nucleobase modification, more than one sugar modification or at least one nucleobase and at least one sugar modification.

The oligonucleotides of the invention comprise predominantly nucleosides having a bicyclo sugar.

However, the oligonucleotides may include a nucleoside having a sugar that is a monocyclic, or tricyclic ring system, a tricyclic or bicyclic system or a monocyclic ribose or de(s)oxyribose. Modifications of the sugar further include but are not limited to modified stereochemical configurations, at least one substitution of a group or at least one deletion of a group. A modified sugar includes a modified version of the ribosyl moiety as naturally occurring in RNA and DNA (i.e. the furanosyl moiety), tetrahydropyrans, 2′-modified sugars, 3′-modified sugars, 4′-modified sugars, 5′-modified sugars, or 4′-subsituted sugars. Examples of suitable sugar modifications are known to the skilled person and include, but are not limited to 2′, 3′ and/or 4′ substituted nucleosides (e.g. 4′-S-modified nucleosides); 2′-O-modified RNA nucleotide residues, such as 2′-O-alkyl or 2′-O-(substituted) alkyl e.g. 2′-O-methyl, 2′-O-(2-cyanoethyl), 2′-O-(2-methoxy)ethyl (2′-MOE), 2′-O-(2-thiomethyl)ethyl, 2′-O-(haloalkoxy) methyl e.g. 2′-O-(2-chloroethoxy)methyl (MCEM), 2′-O-(2,2-dichloroethoxy)methyl (DCEM), 2′-O-alkoxycarbonyl e.g. 2′-O-[2-(methoxycarbonyl) ethyl] (MOCE), 2′-O-[2-(N-methylcarbamoyl)ethyl] (MCE), 2′-O-[2-(N,N-dimethylcarbamoyl)ethyl] (DMCE), for example, a 2′-O-methyl modification or a 2′-O-methoxyethyl (2′-O-MOE), or other modified sugar moieties, such as morpholino (PMO), cationic morpholino (PMOPlus) or a modified morpholino group, such as PMO-X. The term “PMO-X” refers to a modified morpholino group comprising at least one 3′ or 5′ terminal modification, such 3′-fluorescent tag, 3′ quencher (e.g. 3′-carboxyfluorescein, 3′-Gene Tools Blue, 3′-lissamine, 3′-dabcyl), 3′-affinity tag and functional groups for chemical linkage (e.g. 3′-biotin, 3′-primary amine, 3′-disulfide amide, 3′-pyridyl dithio), 5′-end modifications (5′-primary amine, 5′-dabcyl), 3′-azide, 3′-alkyne, 5′-azide, 5′-alkyne, or as disclosed in WO2011/150408 and US2012/0065169.

As used herein, the term “ribonucleotide” encompasses natural and synthetic, unmodified and modified ribonucleotides. Modifications include changes to the sugar moiety, to the base moiety and/or to the linkages between ribonucleotides in the oligonucleotide. As used herein, the term “ribonucleotide” specifically excludes a deoxyribonucleotide, which is a nucleotide possessing a single proton group at the 2′ ribose ring position.

As used herein, the term “deoxyribonucleotide” encompasses natural and synthetic, unmodified and modified deoxyribonucleotides. Modifications include changes to the sugar moiety, to the base moiety and/or to the linkages between deoxyribonucleotide in the oligonucleotide. As used herein, the term “deoxyribonucleotide” also includes a modified ribonucleotide, e.g., a 2′-O-methyl ribonucleotide, a phosphorothioate-modified ribonucleotide residue, etc. . . .

As used herein, the term “PS-NA” refers to a phosphorothioate-modified nucleotide residue. The term “PS-NA” therefore encompasses both phosphorothioate-modified ribonucleotides (“PS-RNAs”) and phosphorothioate-modified deoxyribonucleotides (“PS-DNAs”).

As used herein, “antisense strand” refers to a single stranded nucleic acid molecule which has a sequence complementary to that of a target RNA.

As used herein, “sense strand” refers to a single stranded nucleic acid molecule which has a sequence complementary to that of an antisense strand.

The invention also provides for oligonucleotides coupled to a non-nucleoside compound.

The invention provides for oligonucleotides coupled to a solid support. A solid support includes but is not limited to beads, polymers or resin.

In certain embodiments, the oligonucleotide is modified by covalent attachment of one or more groups, in addition to the lipid group, to the 5′ or 7′ terminus of the oligomer, or at any position of the oligomer. A group that can be conjugated to the 5′ terminal group or 7′ terminal group includes but is not limited to a capping group, diphosphate, triphosphate, label, such as a fluorescent label (e.g. fluorescein or rhodamine), dye, reporter group suitable for tracking the oligomer, solid support, nanoparticle, non-nucleosidic group, antibody or conjugate group. In general, conjugate groups modify one or more properties of the compound they are attached to. Such properties include without limitation, nuclease stability, binding affinity, pharmacodynamics, pharmacokinetics, binding, absorption, cellular distribution, cellular uptake, delivery, charge and clearance. Conjugate groups are routinely used in the chemical arts and are linked directly or via an optional linkage group to a parent compound such as an oligomer. The term “conjugate group” includes without limitation, a lipid group, intercalators, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, lipophilic moieties, coumarins, peptides, antibodies, nanobodies, and oligosaccharides, for example N-acetylgalactosamine.

As used herein, “terminus” refers to the end or terminus of the oligomer, nucleic acid sequence or any one of the compounds described herein, wherein the integer (3′, 5′ or 7′ etc.) designates the carbon atom of the sugar included in the nucleotide of the oligomer, nucleic acid sequence or the compound. As used herein, “5′ terminal group” or “7′ terminal group”, refers to a group located at the 5′ terminus or 7′ terminus, respectively, of the sugar included in any one of the compounds provided herein.

In certain embodiments, the oligomer comprises at least one monomer subunit that is a compound of the formula (IV), formula (V) or a compound of the formula (VI), as described herein. In one embodiment, the oligomer comprises at least one compound of formula (IV), (V) or (VI) and at least one ribonucleotide or deoxyribonucleotide. In another embodiment, the oligomer comprises at least one compound of formula (IV), (V) or (VI) and at least one deoxyribonucleotide.

By “complementary” or “complementarity” is meant that a nucleic acid can form hydrogen bonds with another nucleic acid sequence by either traditional Watson-Crick or Hoogsteen base pairing. In reference to the nucleic acid molecules of the present disclosure, the binding free energy for a nucleic acid molecule with its complementary sequence is sufficient to allow the relevant function of the nucleic acid to proceed, e.g., exon skipping. Determination of binding free energies for nucleic acid molecules is well known in the art (see, e.g., Turner, et al., CSH Symp. Quant. Biol. LII, pp. 123-133, 1987; Frier, et al., Proc. Nat. Acad. Sci. USA 83:9373-9377, 1986; Turner, et al., J. Am. Chem. Soc. 109:3783-3785, 1987). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary, respectively). To determine that a percent complementarity is of at least a certain percentage, the percentage of contiguous residues in a nucleic acid molecule that can form hydrogen bonds (e.g., Watson-Crick base pairing) with a second nucleic acid sequence is calculated and rounded to the nearest whole number (e.g., 12, 13, 14, 15, 16, or 17 nucleotides out of a total of 23 nucleotides in the first oligonucleotide being based paired to a second nucleic acid sequence having 23 nucleotides represents 52%, 57%, 61%, 65%, 70%, and 74%, respectively; and has at least 50%, 50%, 60%, 60%, 70%, and 70% complementarity, respectively). As used herein, “substantially complementary” refers to complementarity between the strands such that they are capable of hybridizing under biological conditions. Substantially complementary sequences have 60%, 70%, 80%, 90%, 95%, or even 100% complementarity. Additionally, techniques to determine if two strands are capable of hybridizing under biological conditions by examining their nucleotide sequences are well known in the art.

The invention also provides for wobble base pairing between two nucleotides in RNA molecules that does not follow Watson-Crick base pair rules. The four main wobble base pairs are guanine-uracil (G-U), hypoxanthine-uracil (I-U), hypoxanthine-adenine (I-A), and hypoxanthine-cytosine (I-C). The thermodynamic stability of a wobble base pair is comparable to that of a Watson-Crick base pair.

Single-stranded nucleic acids that base pair over a number of bases are said to “hybridize.” Hybridization is typically determined under physiological or biologically relevant conditions (e.g., intracellular: pH 7.2, 140 mM potassium ion; extracellular pH 7.4, 145 mM sodium ion). Hybridization conditions generally contain a monovalent cation and biologically acceptable buffer and may or may not contain a divalent cation, complex anions, e.g. gluconate from potassium gluconate, uncharged species such as sucrose, and inert polymers to reduce the activity of water in the sample, e.g. PEG. Such conditions include conditions under which base pairs can form.

Hybridization is measured by the temperature at which 50% of a nucleic acid is single stranded and 50% is double stranded, i.e., (the melting temperature; Tm). The Tₘ is often used as a measure of duplex stability of an antisense compound toward a complementary nucleic acid.

Hybridization conditions are also conditions under which base pairs can form. Various conditions of stringency can be used to determine hybridization (see, e.g., Wahl, G. M. and S. L. Berger (1987) Methods Enzymol. 152:399; Kimmel, A. R. (1987) Methods Enzymol. 152:507). Stringent temperature conditions will ordinarily include temperatures of at least about 30° C., more preferably of at least about 37° C., and most preferably of at least about 42° C. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm° C.)=2 (# of A+T bases)+4 (# of G+C bases). For hybrids between 18 and 49 base pairs in length, Tm (° C.)=81.5+16.6 (log 10[Na+])+0.41 (% G+C)−(600/N), where N is the number of bases in the hybrid, and [Na+] is the concentration of sodium ions in the hybridization buffer ([Na+] for 1×SSC=0.165 M).

Useful variations on hybridization conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Antisense to Molecular Cloning Techniques, 1987, Academic Press, New York); and Sambrook et al., Molecular Cloning: A Laboratory Manual, Cold Spring Harbor Laboratory Press, New York.

As used herein, “alter” means increase or decrease expression, for example gene expression. A decrease in expression means a decrease of 10% or more, for example, 10%, 20%, 25%, 30%, 35%, 40%, 45%, 50%, 55%, 60%, 65%, 70%, 75%, 80%, 85%, 90%, 95% and 100%. A decrease also means a decrease of 2-fold or more, for example, 2-fold, 5-fold, 10-fold, 20-fold, 30-fold, 40-fold, 50-fold, 60-fold, 70-fold, 80-fold, 90-fold, 100-fold, 500-fold or more.

An increase in expression means an increase of 10% or more, for example, 10%, 20%, 25%, 30%, 35%, 40%, 45%, 50%, 55%, 60%, 65%, 70%, 75%, 80%, 85%, 90%, 95% and 100%. An increase also means an increase of 2-fold or more, for example, 2-fold, 5-fold, 10-fold, 20-fold, 30-fold, 40-fold, 50-fold, 60-fold, 70-fold, 80-fold, 90-fold, 100-fold, 500-fold or more.

An increase or decrease in the expression of a gene is relative to the level of expression of a control or reference level, for example, the level of gene expression in the absence of an oligonucleotide lipid group conjugate of the invention.

As used herein, “target RNA” refers to an RNA that would be subject to modulation by an oligonucleotide of the invention.

As used herein, “target” refers to any nucleic acid sequence whose expression or activity is to be modulated by an oligonucleotide of the invention.

As used herein, “reference” is meant a standard or control. As is apparent to one skilled in the art, an appropriate reference is where only one element is changed in order to determine the effect of the one element.

As used herein, a “portion of an RNA” means a length that is equivalent to the oligonucleotide to which it binds, and having a sequence that is complementary to that of the oligonucleotide to which it binds.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, “increase” or “enhance” is meant to alter positively by at least 5% compared to a reference in an assay. An alteration may be by 5%, 10%, 25%, 30%, 50%, 75%, or even by 100% compared to a reference in an assay. By “enhance exon skipping,” it is meant increases the amount of a particular product that is the result of exon skipping.

As used herein “reduce” is meant to alter negatively by at least 5% compared to a reference in an assay. An alteration may be by 5%, 10%, 25%, 30%, 50%, 75%, or even by 100% compared to a reference in an assay.

As used herein, “cell” is meant to include both prokaryotic (e.g., bacterial) and eukaryotic (e.g., mammalian or plant) cells. Cells may be of somatic or germ line origin, may be totipotent or pluripotent, and may be dividing or non-dividing. Cells can also be derived from or can comprise a gamete or an embryo, a stem cell, or a fully differentiated cell. Thus, the term “cell” is meant to retain its usual biological meaning and can be present in any organism such as, for example, a bird, a plant, and a mammal, including, for example, a human, a cow, a sheep, an ape, a monkey, a pig, a dog, and a cat. Within certain aspects, the term “cell” refers specifically to mammalian cells, such as human cells.

As used herein, “animal” is meant a multicellular, eukaryotic organism, including a mammal, particularly a human. The methods of the invention in general comprise administration of an effective amount of the oligonucleotide herein to a subject (e.g., animal, human) in need thereof, including a mammal, particularly a human. Such treatment will be suitably administered to subjects, particularly humans, suffering from, having, susceptible to, or at risk for a disease, or a symptom thereof.

By “pharmaceutically acceptable carrier” is meant, a composition or formulation that allows for the effective distribution of the nucleic acid molecules of the instant disclosure in the physical location most suitable for their desired activity.

The oligonucleotide agents of the instant invention can enhance the following attributes of such agents relative to oligonucleotide agents lacking abc-DNA nucleosides, or oligonucleotides comprising abc-DNA nucleosides but lacking the combination of phosphate internucleosidic linkages and a lipid group: in vitro efficacy (e.g., potency and duration of effect), in vivo efficacy (e.g., potency, duration of effect, pharmacokinetics, pharmacodynamics, intracellular uptake, reduced toxicity).

As used herein, the term “pharmacokinetics” refers to the process by which a drug is absorbed, distributed, metabolized, and eliminated by the body.

As used herein, the term “pharmacodynamics” refers to the action or effect of a drug on a living organism.

As used herein, the term “stabilization” refers to a state of enhanced persistence of an agent in a selected environment (e.g., in a cell or organism). Enhanced stability can be achieved via enhanced resistance of such agents to degrading enzymes (e.g., nucleases) or other agents.

As used herein, “modified nucleotide” refers to a nucleotide that has one or more modifications to the nucleoside, the nucleobase, furanose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2′-methoxyethoxy, 2′-fluoro, 2′-allyl, 2′-O-[2-(methylamino)-2-oxoethyl], 4′-thio, 4′-CH₂—O-2′-bridge, 4′-(CH₂)₂—O-2′-bridge, 2′-LNA, and 2′-O—(N-methylcarbamate) or those comprising base analogs. In connection with 2′-modified nucleotides as described for the present disclosure, by “amino” is meant 2′-NH₂ or 2′-O—NH₂, which can be modified or unmodified. Such modified groups are described, e.g., in Eckstein et al., U.S. Pat. No. 5,672,695 and Matulic-Adamic et al., U.S. Pat. No. 6,248,878.

As used herein, “base analog” refers to a heterocyclic moiety which is located at the 1′ position of a nucleotide sugar moiety in a modified nucleotide that can be incorporated into a nucleic acid duplex (or the equivalent position in a nucleotide sugar moiety substitution that can be incorporated into a nucleic acid duplex). A base analog is generally either a purine or pyrimidine base excluding the common bases guanine (G), cytosine (C), adenine (A), thymine (T), and uracil (U). Base analogs can duplex with other bases or base analogs in dsRNAs. Base analogs include those useful in the compounds and methods of the invention, e.g., those disclosed in U.S. Pat. Nos. 5,432,272 and 6,001,983 to Benner and US Patent Publication No. 20080213891 to Manoharan, which are herein incorporated by reference. Non-limiting examples of bases include 2,6-diaminopurine, hypoxanthine (I), xanthine (X), 3-β-D-ribofuranosyl-(2,6-diaminopyrimidine) (K), 3-β-D-ribofuranosyl-(1-methyl-pyrazolo[4,3-d]pyrimidine-5,7(4H,6H)-d-ione) (P), iso-cytosine (iso-C), iso-guanine (iso-G), 1-β-D-ribofuranosyl-(5-nitroindole), 1-β-D-ribofuranosyl-(3-nitropyrrole), 5-bromouracil, 2-aminopurine, 4-thio-dT, 7-(2-thienyl)-imidazo[4,5-b]pyridine (Ds) and pyrrole-2-carbaldehyde (Pa), 2-amino-6-(2-thienyl)purine (S),2-oxopyridine (Y), difluorotolyl, 4-fluoro-6-methylbenzimidazole, 4-methylbenzimidazole, 3-methyl isocarbostyrilyl, 5-methyl isocarbostyrilyl, and 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenzyl, tetracenyl, pentacenyl, and structural derivatives thereof (Schweitzer et al., J. Org. Chem., 59:7238-7242 (1994); Berger et al., Nucleic Acids Research, 28(15):2911-2914 (2000); Moran et al., J. Am. Chem. Soc., 119:2056-2057 (1997); Morales et al., J. Am. Chem. Soc., 121:2323-2324 (1999); Guckian et al., J. Am. Chem. Soc., 118:8182-8183 (1996); Morales et al., J. Am. Chem. Soc., 122(6):1001-1007 (2000); McMinn et al., J. Am. Chem. Soc., 121:11585-11586 (1999); Guckian et al., J. Org. Chem., 63:9652-9656 (1998); Moran et al., Proc. Natl. Acad. Sci., 94:10506-10511 (1997); Das et al., J. Chem. Soc., Perkin Trans., 1:197-206 (2002); Shibata et al., J. Chem. Soc., Perkin Trans., 1: 1605-1611 (2001); Wu et al., J. Am. Chem. Soc., 122(32):7621-7632 (2000); O'Neill et al., J. Org. Chem., 67:5869-5875 (2002); Chaudhuri et al., J. Am. Chem. Soc., 117:10434-10442 (1995); and U.S. Pat. No. 6,218,108). Base analogs may also be a universal base.

As used herein, “universal base” refers to a heterocyclic moiety located at the 1′ position of a nucleotide sugar moiety in a modified nucleotide, or the equivalent position in a nucleotide sugar moiety substitution, that, when present in a nucleic acid duplex, can be positioned opposite more than one type of base without altering the double helical structure (e.g., the structure of the phosphate backbone). Additionally, the universal base does not destroy the ability of the oligonucleotide in which it resides to duplex to a target nucleic acid. The ability of a single stranded nucleic acid containing a universal base to duplex a target nucleic acid can be assayed by methods apparent to one in the art (e.g., UV absorbance, circular dichroism, gel shift, single stranded nuclease sensitivity, etc.). Additionally, conditions under which duplex formation is observed may be varied to determine duplex stability or formation, e.g., temperature, as melting temperature (Tm) correlates with the stability of nucleic acid duplexes. Compared to a reference single stranded nucleic acid that is exactly complementary to a target nucleic acid, the single stranded nucleic acid containing a universal base forms a duplex with the target nucleic acid that has a lower Tm than a duplex formed with the complementary nucleic acid. However, compared to a reference single stranded nucleic acid in which the universal base has been replaced with a base to generate a single mismatch, the single stranded nucleic acid containing the universal base forms a duplex with the target nucleic acid that has a higher Tm than a duplex formed with the nucleic acid having the mismatched base.

Some universal bases are capable of base pairing by forming hydrogen bonds between the universal base and all of the bases guanine (G), cytosine (C), adenine (A), thymine (T), and uracil (U) under base pair forming conditions. A universal base is not a base that forms a base pair with only one single complementary base. In a duplex, a universal base may form no hydrogen bonds, one hydrogen bond, or more than one hydrogen bond with each of G, C, A, T, and U opposite to it on the opposite strand of a duplex. Preferably, the universal bases do not interact with the base opposite to it on the opposite strand of a duplex. In a duplex, base pairing between a universal base occurs without altering the double helical structure of the phosphate backbone. A universal base may also interact with bases in adjacent nucleotides on the same nucleic acid strand by stacking interactions. Such stacking interactions stabilize the duplex, especially in situations where the universal base does not form any hydrogen bonds with the base positioned opposite to it on the opposite strand of the duplex. Non-limiting examples of universal-binding nucleotides include inosine, 1-beta-D-ribofuranosyl-5-nitroindole, and/or 1-beta-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside. Nucleic Acids Res. 1995 Nov. 11; 23(21):4363-70; Loakes et al., 3-Nitropyrrole and 5-nitroindole as universal bases in primers for DNA sequencing and PCR. Nucleic Acids Res. 1995 Jul. 11; 23(13):2361-6; Loakes and Brown, 5-Nitroindole as a universal base analogue. Nucleic Acids Res. 1994 Oct. 11; 22(20):4039-43).

The term “stereoisomers” refers to compounds, which have identical chemical constitution, but differ with regard to the arrangement of the atoms or groups in space.

“Diastereomer” refers to a stereoisomer with two or more centers of chirality in which the compounds are not mirror images of one another. Diastereomers have different physical properties, e.g. melting points, boiling points, spectral properties, and chemical and biological reactivities. Mixtures of diastereomers may be separated under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McRaw-Hiff Dictionary of Chemical Terms (1984), McGraw-Hill Book Company, New York; and Eliel, E. and Wilen, S., “Stereochemistry of Organic Compounds”, John Wiley & Sons, Inc., New York, 1994.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The present invention can be understood more readily by reference to the following detailed description of the invention and the Examples included therein.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

Alpha Anomeric Bicyclo-DNA (abc-DNA) Nucleosides

Alpha-bicyclic (“abc”) DNA is a nucleoside analog containing a bicyclic sugar moiety, useful in antisense oligonucleotides (AONs), for example to treat disease by causing exon skipping. abc-DNA nucleosides have the general structure shown below.

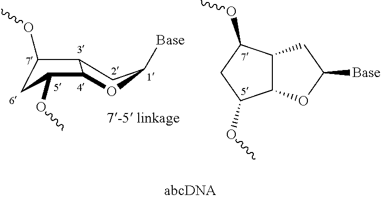

The structure of 7′-5′-alpha-anomeric-bicyclo-DNA is shown below.

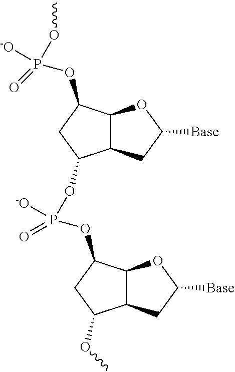

Structure of 7′-5′-alpha-anomeric-bicyclo-DNA

In addition to having high selectivity for RNA, the 7′, 5′-abc-DNA modification has improved mismatch discrimination as compared to DNA, is compatible with phosphorothioate modifications, confers a complete biostability, induces low complement activation, and exhibits high in vitro exon skipping.

The invention provides for oligonucleotides comprising any one of the abc-DNA nucleosides and having any of the substituents disclosed herein.

The invention provides for an oligonucleotide comprising at least one compound of formula (I):

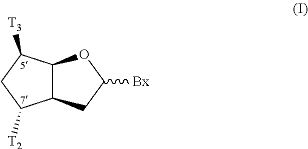

wherein one of T₁ and T₂ is OR₁ or OR₂;
and the other of T₁ and T₂ is OR₁ or OR₂; wherein
R₁ is H or a hydroxyl protecting group, and
R₂ is a phosphorus moiety; and wherein
Bx is a nucleobase.

In one embodiment, the compound of formula (I) of the invention is a compound of formula (II)

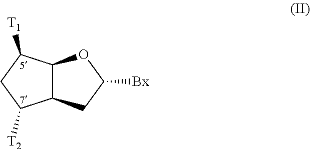

wherein
(i) T₁ is OR₁, and T₂ is OR₁ or OR₂; or
(ii) T₁ is OR₁ or OR₂, T₂ is OR₁:
wherein T₁ is OR₁ or OR₂, T₂ is OR₁.

The compound of formula (II) is an alpha anomer or an alpha anomeric monomer that differs from the beta anomer in the spatial configuration of Bx at the chiral center of the first carbon at the 1′ terminus.

In another embodiment, the compound of formula (I) is a compound of formula (III)

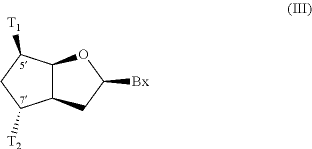

wherein
(i) T₁ is OR₁, and T₂ is OR₁ or OR₂; or
(ii) T₁ is OR₁ or OR₂, T₂ is OR₁:
wherein T₁ is OR₁, and T₂ is OR₁ or OR₂.

The compound of formula (III) is a beta anomer or a beta anomeric monomer that differs from the alpha anomer in the spatial configuration of Bx at the chiral center of the first carbon at the 1′ terminus.

In another embodiment, in the compound of formula (I) or (II), Bx is selected from a purine base or pyrimidine base, wherein Bx is selected from (i) adenine (A), (ii) cytosine (C), (iii) 5-methylcytosine (MeC), (iv) guanine (G), (v) uracil (U), (vi) thymine or (vii) 2,6-diaminopurine or a derivative of (i), (ii), (iii), (iv), (v), (vi) or (vii). In another embodiment, in the compound of formula (I), (II) or (III), Bx is selected from thymine, 5-methylcytosine, uracil, adenine or guanine. In another embodiment, in the compound of formula (I), (II) or (III), Bx is selected from thymine, 5-methylcytosine, adenine or guanine.

The term “nucleobase”, as used herein, and abbreviated as Bₓ, refers to unmodified or naturally occurring nucleobases as well as modified or non-naturally occurring nucleobases and synthetic mimetics thereof A nucleobase is any heterocyclic base that contains one or more atoms or groups of atoms capable of hydrogen bonding to a heterocyclic base of a nucleic acid.

In one embodiment, the nucleobase is a purine base or a pyrimidine base, wherein preferably said purine base is purine or substituted purine, and said pyrimidine base is pyrimidine or substituted pyrimidine. More preferably, the nucleobase is (i) adenine (A), (ii) cytosine (C), (iii) 5-methylcytosine (MeC), (iv) guanine (G), (v) uracil (U), or (vi) 5-methyluracil (MeU), or to a derivative of (i), (ii), (iii), (iv), (v) or (vi). The terms “derivative of (i), (ii), (iii), (iv), (v) or (vi), and “nucleobase derivative” are used herein interchangeably. Derivatives of (i), (ii), (iii), (iv), (v) or (vi), and nucleobase derivatives, respectively, are known to the skilled person in the art and are described, for example, in Sharma V. K. et al., Med. Chem. Commun., 2014, 5, 1454-1471, and include without limitation 5-hydroxymethyl cytosine, xanthine, hypoxanthine, 2-aminoadenine, alkyl adenine, such as 6-methyl adenine, 2-propyl adenine, alkyl guanine, such as 6-methyl guanine, 2-propyl guanine, 2-thiouracil, 2-thiothymine and 2-thiocytosine, 5-halo uracil, 5-halo cytosine, alkynyl pyrimidine bases, such as 5-propynyl (—C═C—CH₃) uracil, 5-propynyl (—C═C—CH₃) cytosine, 6-azo uracil, 6-azo cytosine, 6-azo thymine, pseudo-uracil, 4-thiouracil; 8-substituted purine bases, such as 8-halo-, 8-amino-, 8-thiol-, 8-thioalkyl-, 8-hydroxyl-adenine or guanine, 5-substituted pyrimidine bases, such as 5-halo-, particularly 5-bromo-, 5-trifluoromethyl-uracil or -cytosine; 7-methylguanine, 7-methyladenine, 2-F-adenine, 2-amino-adenine, 8-azaguanine and 8-azaadenine, 7-deazaguanine, 7-deazaadenine, 3-deazaguanine, 3-deazaadenine, hydrophobic bases, promiscuous bases, size-expanded bases, or fluorinated bases. In certain embodiments, the nucleobase includes without limitation tricyclic pyrimidines, such as 1,3-diazaphenoxazine-2-one, 1,3-diazaphenothiazine-2-one or 9-(2-aminoethoxy)-1,3-diazaphenoxazine-2-one (G-clamp). The term “nucleobase derivative” also includes those in which the purine or pyrimidine base is replaced by other heterocycles, for example 7-deazaadenine, 7-deazaguanosine, 2-aminopyridine or 2-pyridone. Further nucleobases of the invention include without limitation those known to skilled artisan (e.g. U.S. Pat. No. 3,687,808; Swayze et al., The Medicinal Chemistry of Oligonucleotides, in Antisense a Drug Technology, Chapter 6, pp. 143-182 (Crooke, S. T., ed., 2008); The Concise Encyclopedia Of Polymer Science And Engineering, Kroschwitz, J. I., Ed., John Wiley & Sons, 1990, pp. 858-859; Englisch et al., Angewandte Chemie, International Edition, 1991, Vol. 30 (6), pp. 613-623; Sanghvi, Y. S., Antisense Research and Applications, Crooke, S. T. and Lebleu, B., Eds., CRC Press, 1993, pp. 273-302).

Preferred nucleobase derivatives include methylated adenine, guanine, uracil and cytosine and nucleobase derivatives, preferably of (i), (ii), (iii) or (iv), wherein the respective amino groups, preferably the exocyclic amino groups, are protected by acyl protecting groups or dialkylformamidino, preferably dimethylformamidino (DMF), and further include nucleobase derivatives such as 2-fluorouracil, 2-fluorocytosine, 5-bromouracil, 5-iodouracil, 2,6-diaminopurine, azacytosine and pyrimidine analogs such as pseudoisocytosine and pseudouracil.

In a further preferred embodiment, said nucleobase derivative is selected from methylated adenine, methylated guanine, methylated uracil and methylated cytosine, and from a nucleobase derivative of (i), (ii), (iii) or (iv), wherein the respective amino groups, preferably the exocyclic amino groups, are protected by a protecting group.

In a further preferred embodiment, said nucleobase derivative is selected from methylated adenine, methylated guanine, methylated uracil and methylated cytosine, and from a nucleobase derivative of (i), (ii), (iii) or (iv), wherein the respective amino groups, preferably the exocyclic amino groups, are protected by acyl protecting groups or dialkylformamidino, preferably dimethylformamidino (DMF).

In a further preferred embodiment, said nucleobase derivative is selected from a nucleobase derivative of (i), (ii), (iii) or (iv), wherein the respective amino groups, preferably the exocyclic amino groups, are protected by a protecting group.

In a further preferred embodiment, said nucleobase derivative is a nucleobase derivative of (i), (ii), (iii) or (iv), wherein the exocyclic amino groups, are protected by acyl protecting groups or dialkylformamidino, preferably dimethylformamidino (DMF).

In a further very preferred embodiment, said acyl protecting group of said exocyclic amino group of said nucleobase derivative of (i), (ii), (iii) or (iv) is —C(O)—R₁₁, wherein independently of each other R₁₁ is selected from C₁-C₁₀alkyl, C₆-C₁₀aryl, C₆-C₁₀arylC₁-C₁₀alkylene, or C₆-C₁₀aryloxyC₁-C₁₀alkylene and wherein said dialkylformamidino protecting group is ═C(H)—NR₁₂R₁₃, wherein R₁₂ and R₁₃ are independently of each other selected from C₁-C₄alkyl.

In a further very preferred embodiment, said acyl protecting group of said exocyclic amino group of said nucleobase derivative of (i), (ii), (iii) or (iv) is —C(O)—R₁₄, wherein independently of each other R₁₄ is selected from C₁-C₄alkyl; phenyl; phenyl substituted with halogen, C₁-C₆alkyl, C₃-C₆cycloalkyl, C₁-C₄alkoxy; benzyl; benzyl substituted with halogen, C₁-C₆alkyl, C₃-C₆cycloalkyl, C₁-C₄alkoxy; or phenyloxyC₁-C₂alkylene optionally substituted with halogen, C₁-C₆alkyl, C₁-C₄alkoxy; and wherein said dialkylformamidino protecting group is ═C(H)—NR₁₂R₁₃, wherein R₁₂ and R₁₃ are independently of each other selected from C₁-C₄alkyl.

In a further very preferred embodiment, said acyl protecting group of said exocyclic amino group of said nucleobase derivative of (i), (ii), (iii) or (iv) is —C(O)—R₁₅, wherein independently of each other R₁₅ is selected from C₁-C₄alkyl; phenyl; phenyl substituted with halogen, C₁-C₄alkyl, C₅-C₆cycloalkyl, C₁-C₄alkoxy; benzyl; benzyl substituted with halogen, C₁-C₄alkyl, C₁-C₄alkoxy; or phenyloxymethylene (CH₂-oC₆H₅) wherein the phenyl is optionally substituted with halogen, C₁-C₄alkyl, C₅-C₆cycloalkyl, C₁-C₄alkoxy; and wherein said dialkylformamidino protecting group is ═C(H)—NR₁₂R₁₃, wherein R₁₂ and R₁₃ are independently of each other selected from C₁-C₄alkyl.

In a further very preferred embodiment, said acyl protecting group of said exocyclic amino group of said nucleobase derivative of (i), (ii), (iii) or (iv) is —C(O)—R₁₆, wherein independently of each other R₁₆ is selected from C₁-C₃alkyl; phenyl; phenyl substituted with C₁-C₃alkyl, methoxy; benzyl; benzyl substituted with C₁-C₃alkyl, methoxy; or phenyloxymethylene (CH₂—OC₆H₅) wherein the C₆H₅ is optionally substituted with C₁-C₃alkyl, methoxy; and wherein said dialkylformamidino protecting group is ═C(H)—NR₁₂R₁₃, wherein R₁₂ and R₁₃ are independently of each other selected from C₁-C₄alkyl.

In a further very preferred embodiment, said acyl protecting group of said exocyclic amino group of said nucleobase derivative of (i), (ii), (iii) or (iv) is —C(O)—R₁₇, wherein independently of each other R₁₇ is selected from C₁-C₃alkyl; phenyl; phenyl substituted with C₁-C₃alkyl, methoxy; benzyl; benzyl substituted with C₁-C₃alkyl, methoxy; or phenyloxymethylene (CH₂—OC₆H₅) wherein the C₆H₅ is optionally substituted with C₁-C₃alkyl, methoxy; and wherein said dialkylformamidino protecting group is dimethylformamidino (DMF).

In a further very preferred embodiment, said acyl protecting group of said exocyclic amino group of said nucleobase derivative of (i), (ii), (iii) or (iv) is —C(O)—R₁₈, wherein independently of each other R₁₈ is selected from methyl, iso-propyl, phenyl, benzyl, or phenyloxymethylene (CH₂—OC₆H₅) wherein the C₆H₅ is optionally substituted with C₁-C₃alkyl, methoxy; and wherein said dialkylformamidino protecting group is dimethylformamidino (DMF).

In a further very preferred embodiment, said acyl protecting group of said exocyclic amino group of said nucleobase derivative of (i), (ii), (iii) or (iv) is —C(O)—R₁₉, wherein independently of each other R₁₉ is selected from methyl, iso-propyl, phenyl, benzyl, or phenyloxymethylene (CH₂—OC₆H₅) wherein the C₆H₅ is optionally substituted with methyl, iso-propyl; and wherein said dialkylformamidino protecting group is dimethylformamidino (DMF).

The term “dialkylformamidino”, as used herein refers to ═C(H)—NR₁₂R₁₃, wherein R₁₂ and R₁₃ are independently of each other selected from C₁-C₄alkyl. In preferred embodiments, said dialkylformamidino is a protecting group of said exocyclic amino group of said nucleobase derivative of (i), (ii), (iii) or (iv). The resulting compounds may be of either the (E)- or (Z)-configuration and both forms, and mixtures thereof in any ratio, should be included within the scope of the present invention. In a preferred embodiment the inventive compounds comprise the dialkylformamidino, preferably dimethylformamidino (DMF), in the (Z) configuration.

According to one embodiment, Bx is selected from uracil, thymine, cytosine, 5-methylcytosine, adenine and guanine. Preferably, Bx is selected from thymine, 5-methylcytosine, adenine and guanine. According to one embodiment, Bx is an aromatic heterocyclic moiety capable of forming base pairs when incorporated into DNA or RNA oligomers in lieu of the bases uracil, thymine, cytosine, 5-methylcytosine, adenine and guanine.

The term “phosphorus moiety”, as used herein, is independently at each occurrence selected from a moiety derived from phosphonates, phosphite triester, monophosphate, diphosphate, triphosphate, phosphate triester, phosphate diester, thiophosphate ester, di-thiophosphate ester or phosphoramidites.

In another embodiment, in the compound of formula (I), the phosphorus moiety R₂ is selected from a phosphate moiety, a phosphoramidate moiety and a phosphoramidite moiety. In another embodiment, in the compound of formula (II) the phosphorus moiety R₂ is selected from a phosphate moiety, a phosphoramidate moiety and a phosphoramidite moiety. In another embodiment, in the compound of formula (III) the phosphorus moiety R₂ is selected from a phosphate moiety, a phosphoramidate moiety and a phosphoramidite moiety.

The term “phosphorus moiety”, as used herein, refers to a moiety comprising a phosphorus atom in the P^{III} or P^{V} valence state and which is represented by formula (VII)

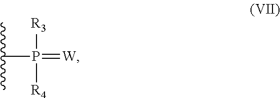

wherein

W represents O, S or Se or W represents an electron pair or W represents BH₂;

R₃ and R₄ are independently of each other H, halogen, OH, OR₅, NR₆R₇, SH, SR₈, C₁-C₆alkyl, C₁-C₆haloalkyl, C₁-C₆alkoxy, C₁-C₆haloalkoxy, C₁-C₆aminoalkyl; wherein R₅ is C₁-C₉alkyl, C₁-C₆alkoxy, each independently of each other optionally substituted with cyano, nitro, halogen, —NHC(O)C₁-C₃ alkyl, —NHC(O)C₁-C₃ halo alkyl, C₁-C₃ alkylsulfonyl; aryl, C₁-C₆alkylenearyl, C₁-C₆alkylenediaryl, each independently of each other optionally substituted with cyano, nitro, halogen, C₁-C₄alkoxy, C₁-C₄haloalkyl, C₁-C₄haloalkoxy, NHC(O)C₁-C₃alkyl, NHC(O)C₁-C₃haloalkyl, C₁-C₃alkylsulfonyl; acetyl; a hydroxyl protecting group; wherein R₆ and R₇ are independently of each other hydrogen, C₁-C₉alkyl optionally substituted with cyano, nitro, halogen, C₂-C₆alkenyl, C₃-C₆cycloalkyl, C₁-C₃alkoxy; aryl optionally substituted with cyano, nitro, halogen, C₁-C₃alkyl, C₁-C₃alkoxy; an amino protecting group; or together with the nitrogen atom to which they are attached form a heterocyclic ring, wherein the heterocyclic ring is selected from pyrollidinyl, piperidinyl, morpholinyl, piperazinyl and homopiperazine, wherein the heterocyclic ring is optionally substituted with C₁-C₃ alkyl; and wherein R₈ is a thiol protecting group; and wherein the wavy line indicates the attachment to the oxygen of the OR₂ group. When W represents O, S or Se then the P atom within the phosphorus moiety is in its P^{V} valence state. When W represents an electron pair then the P atom within the phosphorus moiety is in its P^{III} valence. The moiety of formula (VII) includes any possible stereoisomer. Further included in the moieties represented by formula (VII) are salts thereof, wherein the salts can be formed upon treatment with inorganic bases or amines, and can be salts derived from reaction with the OH or SH groups being (independently of each other) the R₃ and R₄. Inorganic bases or amines leading to the salt formation with the OH or SH groups are well known in the art and include trimethylamine, diethylamine, methylamine or ammonium hydroxide. These phosphorus moieties included in the present invention are, if appropriate, also abbreviated as “O⁻HB⁺”, wherein the HB⁺ refers to the counter cation formed.

In one embodiment, in the “phosphorus moiety”, R₃ and R₄ are independently of each other H, OH, OR₅, NR₆R₇, C₁-C₆alkyl, C₁-C₆alkyl, C₁-C₆haloalkyl, C₁-C₆alkoxy, C₁-C₆haloalkoxy, C₁-C₆aminoalkyl; wherein R₅ is C₁-C₉alkyl optionally substituted with cyano, nitro, halogen; aryl, C₁-C₆alkylenearyl, each independently of each other optionally substituted with cyano, nitro, halogen; acetyl; a hydroxyl protecting group; wherein R₆ and R₇ are independently of each other hydrogen, C₁-C₉alkyl optionally substituted with cyano, nitro, halogen; aryl optionally substituted with cyano, nitro, halogen, C₁-C₃ alkyl, C₁-C₃alkoxy; an amino protecting group; and wherein R₈ is a thiol protecting group; and wherein the wavy line indicates the attachment to the oxygen of the OR₂ group.

The term “phosphorus moiety”, as used herein, includes a moiety derived from phosphonates, phosphite triester, monophosphate, diphosphate, triphosphate, phosphate triester, phosphate diester, thiophosphate ester, di-thiophosphate ester or phosphoramidites.

Thus, in one embodiment, the OR₂ is independently at each occurrence selected from phosphonates, phosphite triester, monophosphate, diphosphate, triphosphate, phosphate triester, phosphate diester, thiophosphate ester, di-thiophosphate ester or phosphoramidites, and wherein the OR₂ is a phosphoramidite or a phosphate triester.

In one embodiment, the phosphorus moiety is derived from a phosphonate represented by formula (VII), wherein W is O, R₃ is selected from C₁-C₆alkyl, C₁-C₆haloalkyl, C₁-C₆alkoxy, C₁-C₆haloalkoxy, C₁-C₆aminoalkyl, and R₄ is OH or O⁻HB⁺; and wherein the wavy line indicates the attachment to the oxygen of the OR₂ group. In another embodiment, the phosphorus moiety of formula (VII) is an H-phosphonate, wherein W is O, R₃ is hydrogen and R₄ is OH or O⁻HB⁺; and wherein the O⁻HB⁺ is HNEt₃⁺. In a further embodiment, the phosphorus moiety of formula (VII) is an alkyl-phosphonate, wherein W is O, R₃ is alkyl, and R₄ is OH or O⁻HB⁺; and wherein the O⁻HB⁺ is HNEt₃⁺. In one embodiment, the phosphorus moiety of formula (VII) is methyl-phosphonate, wherein W is O, R₃ is hydrogen and R₄ is OH or O⁻HB⁺; and wherein the O⁻HB⁺ is HNEt₃⁺). In another embodiment, the phosphorus moiety of formula (VII) is a phosphonocarboxylate, wherein R₃ or R₄ are independently of each other a carboxylic acid. The phosphonocarboxylate can be phosphonoacetic acid or phosphonoformic acid. In a further embodiment, the phosphorus moiety of formula (VII) is a 2-aminoethyl-phosphonate.

In another embodiment, R₃ and R₄ of the phosphorus moiety of formula (VII) are independently of each other H, OH, halogen, OR₅, NR₆R₇, SH, SR₈, C₁-C₄alkyl, for example, C₁-C₂alkyl, C₁-C₄halo alkyl, C₁-C₂halo alkyl, C₁-C₄alkoxy, C₁-C₂alkoxy, C₁-C₄haloalkoxy, C₁-C₂haloalkoxy, C₁-C₄aminoalkyl, C₁-C₂aminoalkyl; and wherein R₅ is C₁-C₆alkyl, for example, C₁-C₃alkyl, each independently of each other optionally substituted with cyano, nitro, halogen, NHC(O)C₁-C₃alkyl, NHC(O)C₁-C₃halo alkyl, C₁-C₃alkylsulfonyl; aryl, C₁-C₃alkylenearyl, C₁-C₃alkylenediaryl, each independently of each other optionally substituted with cyano, nitro, halogen, C₁-C₄alkoxy, C₁-C₄halo alkyl, C₁-C₄haloalkoxy, NHC(O)C₁-C₃alkyl, NHC(O)C₁-C₃haloalkyl, C₁-C₃alkylsulfonyl; acetyl; a hydroxyl protecting group; and wherein R₆ and R₇ are independently of each other hydrogen, C₁-C₆alkyl, for example, C₁-C₄alkyl, each independently of each other optionally substituted with cyano, nitro, halogen, C₂-C₄alkenyl, C₃-C₆cycloalkyl, C₁-C₃alkoxy; aryl optionally substituted with cyano, nitro, halogen, C₁-C₃ alkyl, C₁-C₃alkoxy; an amino protecting group; or together with the nitrogen atom to which they are attached form a heterocyclic ring, wherein the heterocyclic ring is selected from pyrollidinyl, piperidinyl, morpholinyl, piperazinyl and homopiperazine, wherein the heterocyclic ring is optionally substituted with C₁-C₃ alkyl; and wherein R₈ is a thiol protecting group; and wherein the wavy line indicates the attachment to the oxygen of the OR₂ group.

In another embodiment, R₃ or R₄ of the phosphorus moiety of formula (VII) is independently at each occurrence and of each other halogen, for example, chlorine, or OR₅, wherein R₅ is a hydroxyl protecting group. Additional phosphorus moieties used in the invention are disclosed in Tetrahedron Report Number 309 (Beaucage and Iyer, Tetrahedron, 1992, 48, 2223-2311).

The term “phosphorus moiety”, as used herein, includes a group R₂ comprising a phosphorus atom in the P″ or P^{V} valence state and which is represented independently at each occurrence either by formula (VIII), formula (IX) or formula (X),

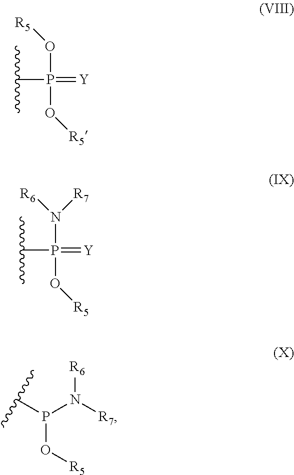

wherein Y is O, S or Se; and wherein R₅ and R_{5′} are independently at each occurrence and of each other hydrogen, C₁-C₉alkyl, C₁-C₆alkoxy, each independently of each other optionally substituted with cyano, nitro, halogen, —NHC(O)C₁-C₃alkyl, —NHC(O)C₁-C₃ halo alkyl, C₁-C₃ alkylsulfonyl; aryl, C₁-C₆alkylenearyl, C₁-C₆alkylenediaryl each independently of each other optionally substituted with cyano, nitro, halogen, C₁-C₄alkoxy, C₁-C₄haloalkyl, C₁-C₄haloalkoxy, —NHC(O)C₁-C₃ alkyl, NHC(O)C₁-C₃haloalkyl, C₁-C₃alkylsulfonyl; a hydroxyl protecting group; wherein R₆ and R₇ are independently of each other hydrogen, C₁-C₉alkyl optionally substituted with cyano, nitro, halogen, C₂-C₆alkenyl, C₃-C₆cycloalkyl, C₁-C₃alkoxy; aryl, for example, phenyl, optionally substituted with cyano, nitro, halogen, C₁-C₃ alkyl, C₁-C₃alkoxy; an amino protecting group; or together with the nitrogen atom to which they are attached form a heterocyclic ring, wherein the heterocyclic ring is selected from pyrollidinyl, piperidinyl, morpholinyl, piperazinyl and homopiperazine, wherein the heterocyclic ring is optionally substituted with C₁-C₃ alkyl; and wherein R₈ is a thiol protecting group; and wherein the wavy line indicates the attachment to the oxygen of the OR₂ group.

In one embodiment, the phosphorus moiety R₂ is represented by formula (VIII)

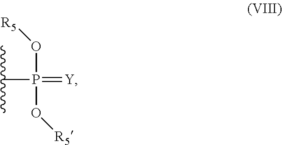

wherein Y is O, S or Se, wherein Y is O or S, or Y is O; and wherein R₅ and R_{5′} are independently at each occurrence and of each other hydrogen, C₁-C₉alkyl, C₁-C₆alkoxy, each independently of each other optionally substituted with cyano, nitro, halogen, —NHC(O)C₁-C₃alkyl, —NHC(O)C₁-C₃ haloalkyl, C₁-C₃ alkylsulfonyl; aryl, C₁-C₆alkylenearyl, C₁-C₆alkylenediaryl each independently of each other optionally substituted with cyano, nitro, halogen, C₁-C₄alkoxy, C₁-C₄haloalkyl, C₁-C₄haloalkoxy, —NHC(O)C₁-C₃alkyl, NHC(O)C₁-C₃haloalkyl, C₁-C₃alkylsulfonyl; a hydroxyl protecting group; P(O)(OR₉)(OR_{9′}), P(O)OP(O)(OR₉)(OR_{9′}); wherein R₉ and R_{9′} are independently at each occurrence and of each other hydrogen, C₁-C₉alkyl optionally substituted with cyano, nitro, halogen, —NHC(O)C₁-C₃ alkyl, —NHC(O)C₁-C₃ halo alkyl, C₁-C₃ alkylsulfonyl; aryl, C₁-C₆alkylene aryl, C₁-C₆alkylenediaryl each independently of each other optionally substituted with cyano, nitro, halogen, C₁-C₄alkoxy, C₁-C₄halo alkyl, C₁-C₄halo alkoxy, —NHC(O)C₁-C₃ alkyl, NHC(O)C₁-C₃haloalkyl, C₁-C₃alkylsulfonyl; a hydroxyl protecting group; and wherein the wavy line indicates the attachment to the oxygen of the OR₂ group.

In another embodiment, R₅ and R_{5′} of formula (VIII) are independently at each occurrence and of each other hydrogen, C₁-C₆alkyl, C₁-C₃alkyl, C₁-C₄alkoxy, C₁-C₂alkoxy, each independently of each other optionally substituted with cyano, nitro, halogen, —NHC(O)C₁-C₃ alkyl, —NHC(O)C₁-C₃ halo alkyl, C₁-C₃ alkylsulfonyl; aryl, for example phenyl, C₁-C₄alkylenearyl, C₁-C₄alkylenediaryl each independently of each other optionally substituted with cyano, nitro, halogen, C₁-C₄alkoxy, C₁-C₄haloalkyl, C₁-C₄haloalkoxy, —NHC(O)C₁-C₃ alkyl, NHC(O)C₁-C₃ halo alkyl, C₁-C₃ alkylsulfonyl; a hydroxyl protecting group.

In another embodiment, R₅ and R_{5′} of formula (VIII) are independently of each other C₁-C₄alkyl or aryl, for example, phenyl. In another embodiment, R₅ and R_{5′} of formula (VIII) are independently of each other methyl or ethyl. In another embodiment, R₅ and R_{5′} of formula (VIII) are independently of each other phenyl or benzyl. In another embodiment, R₅ and R_{5′} are independently at each occurrence and of each other hydrogen or a hydroxyl protecting group. In another embodiment, in formula (VIII), R₅ and R_{5′} are independently at each occurrence and of each other hydrogen, C₁-C₉alkyl, C₁-C₆alkoxy, each independently of each other optionally substituted with cyano, nitro, halogen; aryl, C₁-C₆alkylenearyl, each independently of each other optionally substituted with cyano, nitro, halogen; or a hydroxyl protecting group. In one embodiment, the phosphorus moiety R₂ represented by formula (VIII) is herein referred as “phosphate moiety”.

In one embodiment, the phosphorus moiety R₂ is represented by formula (IX)

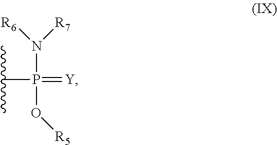

wherein

wherein

Y is O, S or Se, and wherein Y is O or S; and wherein

R₅ is independently at each occurrence hydrogen, C₁-C₉alkyl, C₁-C₆alkoxy, each independently of each other optionally substituted with cyano, nitro, halogen, —NHC(O)C₁-C₃ alkyl, —NHC(O)C₁-C₃ halo alkyl, C₁-C₃ alkylsulfonyl; aryl, C₁-C₆alkylenearyl, C₁-C₆alkylenediaryl each independently of each other optionally substituted with cyano, nitro, halogen, C₁-C₄alkoxy, C₁-C₄haloalkyl, C₁-C₄haloalkoxy, —NHC(O)C₁-C₃alkyl, NHC(O)C₁-C₃haloalkyl, C₁-C₃alkylsulfonyl; a hydroxyl protecting group; wherein

R₆ and R₇ are independently of each other hydrogen, C₁-C₉alkyl optionally substituted with cyano, nitro, halogen, C₂-C₆alkenyl, C₃-C₆cycloalkyl, C₁-C₃alkoxy; aryl, for example, phenyl, optionally substituted with cyano, nitro, halogen, C₁-C₃ alkyl, C₁-C₃alkoxy; an amino protecting group; or together with the nitrogen atom to which they are attached form a heterocyclic ring, wherein the heterocyclic ring is selected from pyrollidinyl, piperidinyl, morpholinyl, piperazinyl and homopiperazine, wherein the heterocyclic ring is optionally substituted with C₁-C₃ alkyl; and wherein the wavy line indicates the attachment to the oxygen of the OR₂ group. In one embodiment, the phosphorus moiety R₂ represented by formula (IX) is referred herein as “phosphoramidate moiety” or, interchangeably used, “phosphoroamidate moiety”.

In another embodiment, the phosphorus moiety R₂ is represented by formula (X)

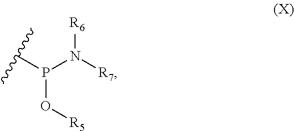

wherein

R₅ is hydrogen, C₁-C₉alkyl, C₁-C₆alkoxy, each independently of each other optionally substituted with cyano, nitro, halogen, —NHC(O)C₁-C₃ alkyl, —NHC(O)C₁-C₃ halo alkyl, C₁-C₃alkylsulfonyl; aryl, C₁-C₆alkylenearyl, C₁-C₆alkylenediaryl independently of each other optionally substituted with cyano, nitro, halogen, C₁-C₄alkoxy, C₁-C₄haloalkyl, C₁-C₄haloalkoxy, —NHC(O)C₁-C₃ alkyl, NHC(O)C₁-C₃ halo alkyl, C₁-C₃ alkylsulfonyl, a hydroxyl protecting group; and wherein

R₆ and R₇ are independently of each other hydrogen, C₁-C₉alkyl optionally substituted with cyano, nitro, halogen, C₂-C₆alkenyl, C₃-C₆cycloalkyl, C₁-C₃alkoxy, aryl, for example phenyl, optionally substituted with cyano, nitro, halogen, C₁-C₃ alkyl, C₁-C₃alkoxy; or together with the nitrogen atom to which they are attached form a heterocyclic ring, wherein the heterocyclic ring is selected from pyrollidinyl, piperidinyl, morpholinyl, piperazinyl and homopiperazine, wherein the heterocyclic ring is optionally substituted with C₁-C₃ alkyl, and wherein the wavy line indicates the attachment to the oxygen of the OR₂ group. Typically and wherein, the phosphorus moiety R₂ represented by formula (X) is referred herein as “phosphoramidite moiety” or, interchangeably used, “phosphoroamidite moiety”.

In another embodiment, in formula (IX) the Y is O; the R₅ is independently at each occurrence hydrogen, C₁-C₉alkyl, C₁-C₆alkoxy, each independently of each other optionally substituted with cyano, nitro, halogen; aryl, C₁-C₆alkylenearyl, each independently of each other optionally substituted with cyano, nitro, halogen; a hydroxyl protecting group; wherein R₆ and R₇ are independently of each other hydrogen, C₁-C₉alkyl optionally substituted with cyano, nitro, halogen, C₂-C₆alkenyl; aryl optionally substituted with cyano, nitro, halogen, C₁-C₃ alkyl, C₁-C₃alkoxy; an amino protecting group; and wherein the wavy line indicates the attachment to the oxygen of the OR₂ group.

In one embodiment, in formula (X) the R₅ is independently at each occurrence hydrogen, C₁-C₉alkyl, C₁-C₆alkoxy, each independently of each other optionally substituted with cyano, nitro, halogen; aryl, C₁-C₆alkylenearyl, each independently of each other optionally substituted with cyano, nitro, halogen; a hydroxyl protecting group; wherein R₆ and R₇ are independently of each other hydrogen, C₁-C₉alkyl optionally substituted with cyano, nitro, halogen, C₂-C₆alkenyl; aryl optionally substituted with cyano, nitro, halogen, C₁-C₃ alkyl, C₁-C₃alkoxy; an amino protecting group; and wherein the wavy line indicates the attachment to the oxygen of the OR₂ group.

In another embodiment, the phosphorus moiety R₂ is independently at each occurrence selected from a phosphate moiety, phosphoramidate moiety and phosphoramidite moiety.

In another embodiment, the R₅ is independently at each occurrence hydrogen, C₁-C₆alkyl, C₁-C₄alkyl, C₁-C₄alkoxy, each independently of each other optionally substituted with cyano, nitro, halogen, —NHC(O)C₁-C₃ alkyl, —NHC(O)C₁-C₃ halo alkyl, C₁-C₃alkylsulfonyl; aryl, C₁-C₄alkylenearyl, C₁-C₄alkylenediaryl each independently of each other optionally substituted with cyano, nitro, halogen, C₁-C₄alkoxy, C₁-C₄haloalkyl, C₁-C₄haloalkoxy, —NHC(O)C₁-C₃ alkyl, NHC(O)C₁-C₃ haloalkyl, C₁-C₃ alkylsulfonyl; a hydroxyl protecting group; wherein R₆ and R₇ are independently of each other hydrogen, C₁-C₆alkyl optionally substituted with cyano, nitro, halogen, C₂-C₄alkenyl, C₃-C₆cycloalkyl, C₁-C₃alkoxy; aryl optionally substituted with cyano, nitro, halogen, C₁-C₃ alkyl, C₁-C₃alkoxy; an amino protecting group; or together with the nitrogen atom to which they are attached form a heterocyclic ring, wherein the heterocyclic ring is selected from pyrollidinyl, piperidinyl, morpholinyl, piperazinyl and homopiperazine, wherein the heterocyclic ring is optionally substituted with C₁-C₃ alkyl; and wherein the wavy line indicates the attachment to the oxygen of the OR₂ group.

In another embodiment, the R₅ is C₁-C₃alkyl optionally substituted with cyano, chlorine, fluorine or bromine; aryl, C₁-C₃alkylenearyl, C₁-C₃alkylenediaryl, each independently of each other optionally substituted with cyano, nitro, chlorine, fluorine, bromine, C₁-C₂alkoxy, C₁haloalkyl. In another embodiment, the R₅ is a C₁-C₃alkyl optionally substituted with cyano, chlorine, fluorine or bromine. In another embodiment, the R₅ is a cyano substituted C₂alkyl, for example, —CH₂CH₂—CN.

In another embodiment, the R₅ is C₁-C₄alkyl, for example, methyl or ethyl; aryl, for example, phenyl or benzyl; chloride or a hydroxyl protecting group. In another embodiment, the R₅ is methyl or a hydroxyl protecting group.

In another embodiment, the R₅ is C₁-C₆alkoxy optionally substituted with cyano, chlorine, fluorine or bromine.

In another embodiment, the R₆ and R₇ are independently of each other H or C₁-C₃alkyl; or together with the nitrogen atom to which they are attached form a heterocyclic ring, wherein the heterocyclic ring is selected from pyrollidinyl, piperidinyl, morpholinyl, piperazinyl wherein the heterocyclic ring is optionally substituted with methyl. In one embodiment, the R₆ and R₇ are independently of each other C₁-C₃alkyl, alkoxy or aryl, wherein the aryl is phenyl or benzyl, optionally substituted with cyano, nitro, chlorine, fluorine, bromine. In another embodiment, the R₆ is hydrogen, and R₇ is (i) C₁-C₉alkyl or (ii) aryl, (i) or (ii) optionally substituted with cyano, nitro, halogen, aryl, wherein R₇ is C₁-C₃alkyl, phenyl or benzyl.

In another embodiment, the R₆ and R₇ are independently of each other selected from methyl, ethyl, isopropyl or isobutyl. In another embodiment, the R₆ and R₇ are independently of each other isopropyl.

In another embodiment, the phosphorus moiety R₂ is represented by formula (X), wherein the R₅ is (i) C₁-C₉alkyl; (ii) aryl, for example, phenyl; or (iii) the (i) or the (ii) optionally substituted with cyano, nitro, halogen, aryl; and wherein the R₆ and R₇ are independently of each other C₁-C₉alkyl, for example, isopropyl.

In another embodiment, the phosphorus moiety R₂ is represented by formula (X), wherein R₅ is C₁-C₉alkyl optionally substituted with cyano, nitro, halogen, —NHC(O)C₁—C₃ alkyl, —NHC(O)C₁-C₃ haloalkyl, C₁-C₃ alkylsulfonyl; aryl, C₁-C₆alkylenearyl, C₁-C₆alkylenediaryl independently of each other optionally substituted with cyano, nitro, halogen, C₁-C₄alkoxy, C₁-C₄halo alkyl, C₁-C₄halo alkoxy, —NHC(O)C₁-C₃ alkyl, —NHC(O)C₁-C₃haloalkyl, C₁-C₃alkylsulfonyl; and R₆ and R₇ are independently of each other C₁-C₉alkyl optionally substituted with cyano, nitro, halogen, C₂-C₆alkenyl, C₃-C₆cycloalkyl, C₁-C₃alkoxy, phenyl optionally substituted with cyano, nitro, halogen, C₁-C₃ alkyl, C₁-C₃alkoxy; or together with the nitrogen atom to which they are attached form a heterocyclic ring, wherein the heterocyclic ring is selected from pyrollidinyl, piperidinyl, morpholinyl, piperazinyl and homopiperazine, wherein the heterocyclic ring is optionally substituted with C₁-C₃ alkyl; and wherein the wavy line indicates the attachment to the oxygen of the OR₂ group.

In another embodiment, the phosphorus moiety R₂ is represented by formula (X), wherein the R₅ is C₁-C₉alkyl optionally substituted with cyano, nitro, chlorine, fluorine, bromine, —NHC(O)C₁-C₃ alkyl, —NHC(O)C₁-C₃ haloalkyl; aryl, C₁-C₆alkylenearyl, C₁-C₆alkylenediaryl independently of each other optionally substituted with cyano, nitro, chlorine, fluorine, bromine, C₁-C₄alkoxy, C₁-C₄haloalkyl.

In another embodiment, the phosphorus moiety R₂ is represented by formula (X), wherein the R₅ is C₁-C₃alkyl optionally substituted with cyano, chlorine, fluorine and bromine; aryl, C₁-C₃alkylenearyl, C₁-C₃alkylenediaryl, independently of each other optionally substituted with cyano, nitro, chlorine, fluorine, bromine, C₁-C₂alkoxy, C₂haloalkyl.

In another embodiment, the phosphorus moiety R₂ is represented by formula (X), wherein the R₅ is C₁-C₃alkyl, 2-cyanoethyl, 2,2,2-trichloroethyl, 2,2,2-tribromoethyl, —(CH₂)ₙNHC(O)CF₃ wherein n=3-6; phenyl, C₁-C₃alkylenephenyl, benzhydryl, independently of each other optionally substituted with cyano, nitro, chlorine, fluorine, bromine, C₁-C₂alkoxy, —CF₃.

In another embodiment, the phosphorus moiety R₂ is represented by formula (X), wherein the R₅ is methyl, ethyl, 2-cyanoethyl, for example, 2-cyanoethyl (CH₂)₂CN).

In another embodiment, the phosphorus moiety R₂ is represented by formula (X), wherein the R₆ and R₇ are independently of each other C₁-C₃alkyl or together with the nitrogen atom to which they are attached form a heterocyclic ring, wherein the heterocyclic ring is selected from pyrollidine, piperidine, morpholine, wherein the heterocyclic ring is optionally substituted with C₁-C₃ alkyl, and wherein the heterocyclic ring is optionally substituted with methyl.

In another embodiment, the phosphorus moiety R₂ is represented by formula (X), wherein R₆ is equal to R₇ and R₆ and R₇ are iso-propyl or methyl.

In another embodiment, the phosphorus moiety R₂ is represented by formula (X), wherein the R₅ is methyl, ethyl, 2-cyanoethyl, and wherein R₆ is equal to R₇ and R₆ and R₇ are iso-propyl or methyl.

Each alkyl moiety either alone or as part of a larger group such as alkoxy or alkylene is a straight or branched chain and can be ═C₁-C₆alkyl, for example, C₁-C₃alkyl. Examples include methyl, ethyl, n-propyl, prop-2-yl (iso-propyl; interchangeably abbreviated herein as iPr or Pri, in particular in the drawn chemical formula), n-butyl, but-2-yl, 2-methyl-prop-1-yl or 2-methyl-prop-2-yl. Examples of an alkoxy include methoxy, ethoxy, propoxy, iso-propoxy, n-butoxy, sec-butoxy, tert-butoxy, n-pentoxy, neo-pentoxy, n-hexoxy. As described herein, alkoxy may include further substituents such as halogen atoms leading to haloalkoxy moieties.

Each alkylene moiety is a straight or branched chain and is, for example, —CH₂—, —CH₂—CH₂—, —CH(CH₃)—, —CH₂—CH₂—CH₂—, —CH(CH₃)—CH₂—, or —CH(CH₂CH₃)—.

Each alkenyl moiety either alone or as part of a larger group such as alkenyloxy or alkenylene is a straight or branched chain and is C₂-C₆alkenyl, for example, C₂-C₄alkenyl. Each moiety can be of either the (E)- or (Z)-configuration. Examples include vinyl and allyl. A compound of the present invention comprising an alkenyl moiety thus may include, if applicable, either the compound with the alkenyl moiety in its (E)-configuration, the compound with the alkenyl moiety in its (Z)-configuration and mixtures thereof in any ratio.

Each alkynyl moiety either alone or as part of a larger group such as alkynyloxy is a straight or branched chain, for example, C₂-C₆alkynyl, or C₂-C₄alkynyl. Examples are ethynyl and propargyl.

Halogen is fluorine, chlorine, bromine, or iodine.

Each haloalkyl moiety either alone or as part of a larger group such as haloalkoxy is an alkyl group substituted by one or more of the same or different halogen atoms. Examples include difluoromethyl, trifluoromethyl, chlorodifluoromethyl and 2,2,2-trifluoroethyl.

In another embodiment, the compound of formula (I) or (II) is linked to a non-nucleosidic compound, for example, a solid-phase.

In another embodiment, the compound of formula (I) is selected from:

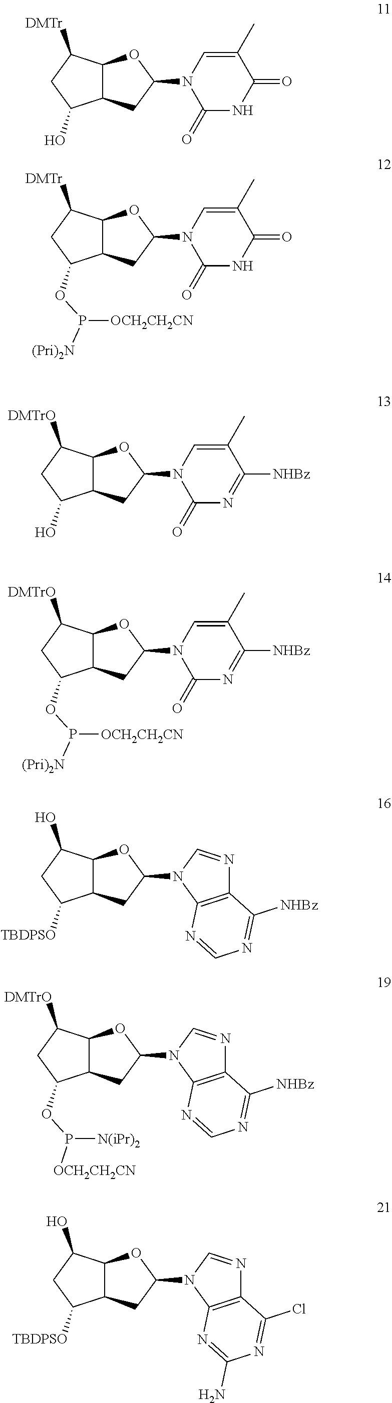

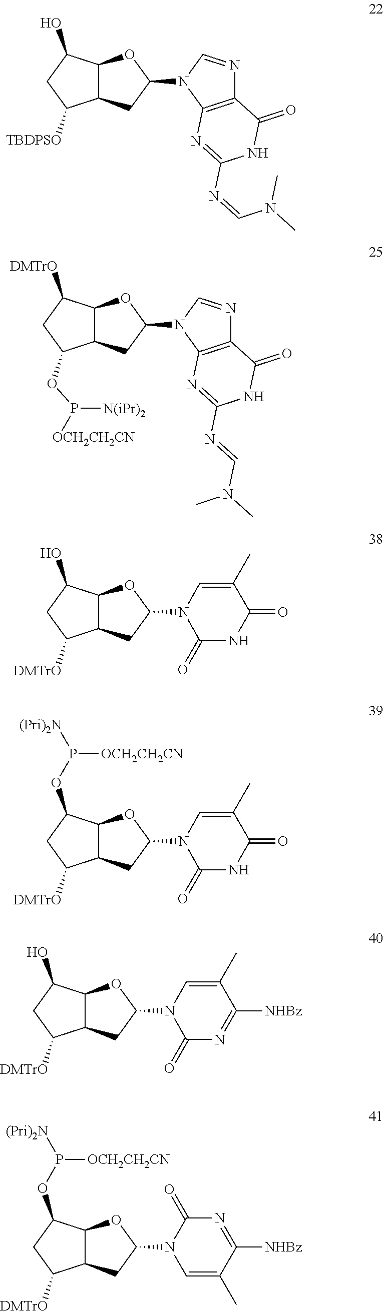

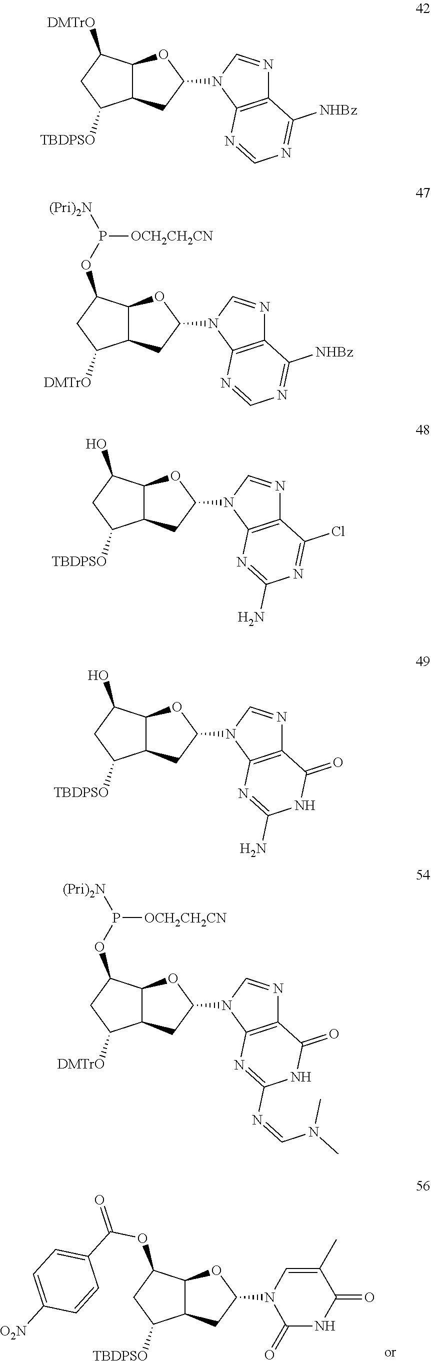

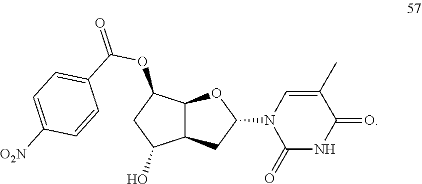 [SMILES: Cc1cn([C@@H]2C[C@@H]3[C@H](O2)[C@H](OC(=O)c2ccc([N+](=O)[O-])cc2)C[C@H]3O)c(=O)[nH]c1=O]

The invention provides for an oligonucleotide comprising at least one compound of formula (IV)

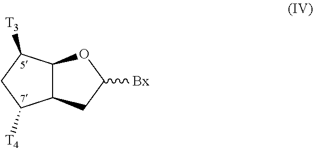

wherein independently for each of the at least one compound of formula (IV) one of T₃ or T₄ is a nucleosidic linkage group;
the other of T₃ and T₄ is OR₁, OR₂, a 5′ terminal group, a 7′ terminal group or a nucleosidic linkage group, wherein R₁ is H or a hydroxyl protecting group, and R₂ is a phosphorus moiety; and Bx is a nucleobase.

In another embodiment, the oligonucleotide of the invention comprises at least one compound of formula (IV), wherein the compound of formula (IV) is a compound of formula (V):

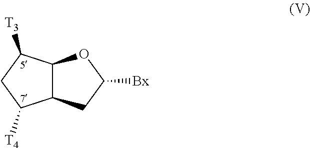

wherein
(i) T₃ is a nucleosidic linkage group, and T₄ is a 7′ terminal group, OR₁, or OR₂, preferably T₄ is a 7′ terminal group or OR₁; or
(ii) T₃ is a 5′ terminal group, OR₁, or OR₂, preferably T₃ is a 5′ terminal group or OR₂; and T₄ is a nucleosidic linkage group; or
(iii) T₃ and T₄ are independently of each other a nucleosidic linkage group.

In another embodiment, the oligonucleotide of the invention comprises at least one compound of formula (IV), wherein said compound of formula (IV) is a compound of formula (VI):

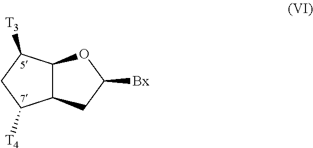

wherein
(i) T₃ is a nucleosidic linkage group, and T₄ is a 7′ terminal group, OR₁, or OR₂, preferably T₄ is a 7′ terminal group or OR₂; or
(ii) T₃ is a 5′ terminal group, OR₁, or OR₂, preferably T₃ is a 5′ terminal group or OR₁; and T₄ is a nucleosidic linkage group; or
(iii) T₃ and T₄ are independently of each other a nucleosidic linkage group.

In another embodiment, the oligonucleotide, comprises a compound of formula (V). In another embodiment, the oligonucleotide, comprises a compound of formula (VI). In another embodiment, the oligonucleotide comprising at least one compound of formula (IV), (V) or (VI) is a DNA or an RNA.

The wavy line within formulaes (I) and (IV) symbolizing the bond between the Bx and the bicyclic core of the inventive compounds indicates that any spatial orientation of the nucleobase Bx are covered by formula (I) or (IV). That means that formulas (I) and (IV) cover either the alpha or the beta conformation or any mixture of alpha and beta anomers of the inventive compounds.

The term “aryl”, as used herein, refers to a monovalent aromatic hydrocarbon radical of 6-14 carbon atoms (C₆-C₁₄) derived by the removal of one hydrogen atom from a single carbon atom of a parent aromatic ring system as well as said aryl optionally substituted independently with one or more substituents, typically and preferably with one or two substituents as described below. Aryl includes bicyclic radicals comprising an aromatic ring fused to a saturated, partially unsaturated ring, or aromatic carbocyclic or heterocyclic ring. Aryl groups are optionally substituted independently with one or more substituents, typically, for example, with one or two substituents, wherein said substituents are independently at each occurrence selected from C₁-C₄alkyl, halogen, CF₃, OH, C₁-C₃alkoxy, NR₂₀R₂₁, C₆H₅, C₆H₅ substituted with halogen, C₁-C₃alkyl, C₁-C₃alkoxy, NR₂₀R₂₁, wherein R₂₀, R₂₁ are independently at each occurrence H, C₁-C₃alkyl. Typical aryl groups include, but are not limited to, radicals derived from benzene (phenyl), substituted phenyls, naphthalene, anthracene, biphenyl, indenyl, indanyl, 1,2-dihydronapthalene, 1,2,3,4-tetrahydronaphthyl and the like. The term “aryl”, as used herein, preferably refers to phenyl optionally substituted with 1 to 3 R₂₂, wherein R₂₂ is independently at each occurrence halogen, —OH, C₁-C₃alkyl optionally substituted with one or two OH, C₁-C₂fluoroalkyl, C₁-C₂alkoxy, C₁-C₂alkoxyC₁-C₃alkyl, C₃-C₆cycloalkyl, —NH₂, NHCH₃ or N(CH₃)₂.

The terms “protecting group for an amino”, “protecting group for an amino group”, or “amino protecting group” as interchangeably used herein, are well known in the art and include those described in detail in Protecting Groups in Organic Synthesis, T. W. Greene and P. G. M. Wuts, 3^{rd} edition, John Wiley & Sons, 1999, Greene's Protective Groups in Organic Synthesis, P. G. M. Wuts, 5^{th} edition, John Wiley & Sons, 2014, and in Current Protocols in Nucleic Acid Chemistry, edited by S. L. Beaucage et al. June 2012, and hereby in particular in Chapter 2. Suitable “amino protecting groups” for the present invention include and are typically and preferably independently at each occurrence selected from methyl carbamate, ethyl carbamate, 9-fluorenylmethyl carbamate (Fmoc), 9-(2-sulfo)fluorenylmethyl carbamate, 2,7-di-t-butyl[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1,1-dimethyl-2,2-dibromoethyl carbamate (DB-t-BOC), 1,1-dimethyl-2,2,2-trichloroethyl carbamate (TCBOC), benzyl carbamate (Cbz), p-methoxybenzyl carbamate (Moz) and 2,4,6-trimethylbenzyl carbamate; as well as formamide, acetamide, benzamide.

The terms “protecting group for a hydroxyl”, “protecting group for a hydroxyl group”, or “hydroxyl protecting group” as interchangeably used herein, are well known in the art and includes those described in detail in Protecting Groups in Organic Synthesis, T. W. Greene and P. G. M. Wuts, 3^{rd} edition, John Wiley & Sons, 1999; Greene's Protective Groups in Organic Synthesis, P. G. M. Wuts, 5^{th} edition, John Wiley & Sons, 2014, and in Current Protocols in Nucleic Acid Chemistry, edited by S. L. Beaucage et al. June 2012, and hereby in particular in Chapter 2. In a certain embodiment, the “hydroxyl protecting groups” of the present invention are independently at each occurrence selected from, acetyl, benzoyl, benzyl, β-methoxyethoxymethyl ether (MEM), dimethoxytrityl, [bis-(4-methoxyphenyl)phenylmethyl] (DMTr), methoxymethyl ether (MOM), methoxytrityl [(4-methoxyphenyl)diphenylmethyl] (MMT), p-methoxybenzyl ether (PMB), methylthiomethyl ether, pivaloyl (Piv), tetrahydropyranyl (THP), tetrahydrofuran (THF), trityl (triphenylmethyl, Tr), silyl ether, such as t-butyldiphenylsilyl (TBDPS), trimethylsilyl (TMS), tert-butyldimethylsilyl (TBDMS), tri-iso-propylsilyloxymethyl (TOM), and triisopropylsilyl (TIPS) ethers; methyl ethers, ethoxyethyl ethers (EE).

In one embodiment, the “hydroxyl protecting groups” of the present invention are independently at each occurrence selected from, acetyl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl (TBDMS), t-butyldiphenylsilyl (TBDPS), triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, 4-monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-y-1 (MOX).

In some embodiments, the hydroxyl protecting group is independently at each occurrence selected from acetyl, benzyl, t-butyldimethylsilyl, t-butyldiphenylsilyl, trityl, 4-monomethoxytrityl, 4,4′-dimethoxytrityl (DMTr), 4,4′,4-trimethoxytrityl (TMTr), 9-phenylxanthin-9-yl (Pixyl) and 9-(p-methoxyphenyl)xanthin-9-yl (MOX).

In some embodiments, the hydroxyl protecting group is independently at each occurrence selected from triphenylmethyl (trityl), 4-monomethoxytrityl, 4,4′-dimethoxytrityl (DMTr), 4,4′,4″-trimethoxytrityl (TMTr), 9-phenylxanthin-9-yl (Pixyl) and 9-(p-methoxyphenyl)xanthin-9-yl (MOX).

In further embodiments, the hydroxyl protecting group is independently at each occurrence selected from trityl, 4-monomethoxytrityl and 4,4′-dimethoxytrityl group.

In another embodiment, the hydroxyl protecting group is independently at each occurrence selected from triphenylmethyl (trityl), 4-monomethoxytrityl, 4,4′-dimethoxytrityl (DMTr), 4,4′,4″-trimethoxytrityl (TMTr), 9-phenylxanthin-9-yl (Pixyl) and 9-(p-methoxyphenyl)xanthin-9-yl (MOX).

In another embodiment, the hydroxyl protecting groups of the present invention is acetyl, dimethoxytrityl (DMTr), tert-butyldimethylsilyl (TBDMS), tri-iso-propylsilyloxymethyl (TOM), or t-butyldiphenylsilyl ether (TBDPS). In another embodiment, the hydroxyl protecting group is independently at each occurrence selected from 4, 4′-dimethoxytrityl (DMTr) or 4-monomethoxytrityl. In another embodiment, the hydroxyl protecting group is 4, 4′-dimethoxytrityl (DMTr).

Where a group is said to be optionally substituted, there can be 1-5 substituents, 1-3 substituents, or 1 or 2 substituents. Where a group is said to be optionally substituted, and where there are more than one substituent, the more than one substituent can either be the same or different.

Internucleoside Phosphorous Containing Linkage Groups

The oligonucleotide of the invention comprises predominantly phosphodiester internucleoside linkages, for example, 50% or more, for example, 55%, 60%, 65%, 70%, 75%, 80%, 85%, 90%, 95%, 99% and 100% of the internucleoside linkage groups are phosphodiester linkage groups.

The oligonucleotide of the invention, can also include, in addition to the predominantly phosphodiester internucleoside linkages, a nucleosidic linkage group selected from a phosphotriester linkage group, a phosphorothioate linkage group, a phosphorodithioate linkage group, a phosphonate linkage group, a phosphonothioate linkage group, a phosphinate linkage group, a phosphorthioamidate linkage or a phosphoramidate linkage group.

The term “nucleosidic linkage group” includes phosphorus linkage groups and non-phosphorus linkage groups.

In one embodiment, the nucleosidic linkage group is a phosphorus linkage group, and the phosphorus linkage group is selected from a phosphodiester linkage group, a phosphotriester linkage group, a phosphorothioate linkage group, a phosphorodithioate linkage group, a phosphonate linkage group, for example, a H-phosphonate linkage group or a methylphosphonate linkage group; a phosphonothioate linkage group, for example, a H-phosphonothioate linkage group, a methyl phosphonothioate linkage group; a phosphinate linkage group, a phosphorthioamidate linkage a phosphoramidate linkage group or a phosphorodiamidate linkage group. In another embodiment, the nucleosidic linkage group is a phosphorus linkage group, and wherein the phosphorus linkage group is selected from a phosphodiester linkage group, a phosphotriester linkage group, a phosphorothioate linkage group, or a phosphonate linkage group, wherein the phosphonate is a H-phosphonate linkage group or methylphosphonate linkage group.

In another embodiment, the nucleosidic linkage group is a phosphorus linkage group, and the phosphorus linkage group is a phosphodiester linkage group. In another embodiment, the nucleosidic linkage group is a phosphorus linkage group, and the phosphorus linkage group is a phosphorothioate linkage group.

The phosphorus linkage group can be selected from an alkyl phosphodiester linkage group, an alkylene phosphodiester linkage group, a thionoalkyl phosphodiester linkage group or an aminoalkyl phosphodiester linkage group, an alkyl phosphotriester linkage group, an alkylene phosphotriester linkage group, a thionoalkyl phosphotriester linkage group or an aminoalkyl phosphotriester linkage group, an alkyl phosphonate linkage group, an alkylene phosphonate linkage group, an aminoalkyl phosphonate linkage group, a thionoalkyl phosphonate linkage group or a chiral phosphonate linkage group. A nucleosidic linkage group according to the invention includes a phosphorus linkage group, and wherein the phosphorus linkage group is a phosphodiester linkage group —O—P(═O)(OH)O— or —O—P(═O)(O⁻)O— with [HB⁺] as counterion, a phosphorothioate —O—P(═S)(OH)O— or —O—P(═S)(O⁻) O— with [HB⁺] as counterion, a methylphosphonate —O—P(═O)(CH₃)O—. Various salts, mixed salts and free acid forms of the phosphorus linkage group are included.

The nucleosidic linkage group can link a nucleoside, nucleotide or oligonucleotide with a further nucleoside, nucleotide or oligonucleotide.

Non-phosphorus linkage groups do not contain a phosphorus atom and examples of non-phosphorus linkage groups include, alkyl, aryl, preferably, phenyl, benzyl, or benzoyl, cycloalkyl, alkylenearyl, alkylenediaryl, alkoxy, alkoxyalkylene, alkylsulfonyl, alkyne, ether, each independently of each other optionally substituted with cyano, nitro, halogen, carboxyl, amide, amine, amino, imine, thiol, sulfide, sulfoxide, sulfone, sulfamate, sulfonate, sulfonamide, siloxane or mixtures thereof. A non-phosphorus linkage group includes amino propyl, long chain alkyl amine group, vinyl, acetylamide, aminomethyl, formacetal, thioformacetal, thioformacetyl, riboacetyl, methyleneimino, methylenehydrazino or a neutral non-ionic nucleoside linkage group, such as amide-3 (3′-CH₂—C(═O)—N(H)-5′) or amide-4 (3′-CH₂—N(H)—C(═O)-5′). A non-phosphorus linkage group includes a compound selected from alkyl, aryl, preferably phenyl, benzyl, or benzoyl, cycloalkyl, alkylenearyl, alkylenediaryl, alkoxy, alkoxyalkylene, alkylsulfonyl, alkyne, or ether, wherein the compound includes C₁-C₉, C₁-C₆, or C₁-C₄.

Lipid Groups

The invention provides for oligonucleotides comprising an abc-DNA nucleoside and a lipid group attached via a linker. The structure of the lipid group conjugated oligonucleotide is such that the hydrocarbon chain of the lipid group, for example a fatty acid, is exposed, thereby allowing the interaction of the hydrocarbon chain with albumin and/or fatty acid receptors or transporters, thereby providing for an oligonucleotide having a long half-life in vivo. The lipid group is conjugated via a linker to the hydroxyl group at the 5′ or 7′ end of the oligonucleotide.

In certain embodiments the lipid group is a fatty acid derived group. In certain embodiments, the fatty acid derived group comprises a carboxy group. Fatty acids include any saturated or unsaturated fatty acid having a hydrocarbon chain of 4 to 28 carbon atoms, and can contain one or two carboxylic acid groups. A fatty acid that contains two carboxylic acid groups is a dicarboxylic acid. One or two fatty acid ligands can be attached to the oligonucleotide via linkers on the 5′ and/or 7′ ends of an abc-DNA oligonucleotide as described herein.

In certain embodiments, the lipid group is a fatty acid derived group, wherein the fatty acid is any one of the fatty acids presented in Tables 1 and 2.

TABLE 1
Saturated Fatty Acids

| | | | |
|---|---|---|---|
| Butyric acid | Butanoic acid | CH₃(CH₂)₂COOH | C4:0 |
| Valeric acid | Pentanoic acid | CH₃(CH₂)₃COOH | C5:0 |
| Caproic acid | Hexanoic acid | CH₃(CH₂)₄COOH | C6:0 |
| Enanthic acid | Heptanoic acid | CH₃(CH₂)₅COOH | C7:0 |
| Caprylic acid | Octanoic acid | CH₃(CH₂)₆COOH | C8:0 |
| Pelargonic acid | Nonanoic acid | CH₃(CH₂)₇COOH | C9:0 |
| Capric acid | Decanoic acid | CH₃(CH₂)₈COOH | C10:0 |
| Undecylic acid | Undecanoic acid | CH₃(CH₂)₉COOH | C11:0 |
| Lauric acid | Dodecanoic acid | CH₃(CH₂)₁₀COOH | C12:0 |
| Tridecylic acid | Tridecanoic acid | CH₃(CH₂)₁₁COOH | C13:0 |
| Myristic acid | Tetradecanoic acid | CH₃(CH₂)₁₂COOH | C14:0 |
| Pentadecylic acid | Pentadecanoic acid | CH₃(CH₂)₁₃COOH | C15:0 |
| Palmitic acid | Hexadecanoic acid | CH₃(CH₂)₁₄COOH | C16:0 |
| Margaric acid | Heptadecanoic acid | CH₃(CH₂)₁₅COOH | C17:0 |
| Stearic acid | Octadecanoic acid | CH₃(CH₂)₁₆COOH | C18:0 |
| Nonadecylic acid | Nonadecanoic acid | CH₃(CH₂)₁₇COOH | C19:0 |
| Arachidic acid | Eicosanoic acid | CH₃(CH₂)₁₈COOH | C20:0 |
| Heneicosylic acid | Heneicosanoic acid | CH₃(CH₂)₁₉COOH | C21:0 |
| Behenic acid | Docosanoic acid | CH₃(CH₂)₂₀COOH | C22:0 |
| Tricosylic acid | Tricosanoic acid | CH₃(CH₂)₂₁COOH | C23:0 |
| Lignoceric acid | Tetracosanoic acid | CH₃(CH₂)₂₂COOH | C24:0 |
| Pentacosylic acid | Pentacosanoic acid | CH₃(CH₂)₂₃COOH | C25:0 |
| Cerotic acid | Hexacosanoic acid | CH₃(CH₂)₂₄COOH | C26:0 |
| Heptacosylic acid | Heptacosanoic acid | CH₃(CH₂)₂₅COOH | C27:0 |
| Montanic acid | Octacosanoic acid | CH₃(CH₂)₂₆COOH | C28:0 |

TABLE 2
Unsaturated fatty acids

| | | | | | |
|---|---|---|---|---|---|
| ω-3 | α-Linolenic | C18:3 | Δ^{9, 12, 15} | CH₃CH₂CH═CHCH₂CH═CHCH₂CH═CH(CH₂)₇COOH | cis |
| | acid | | | | |
| ω-3 | Stearidonic | C18:4 | Δ^{6, 9, 12, 15} | CH₃CH₂CH═CHCH₂CH═CHCH₂CH═CHCH₂CH═CH(CH₂)₄COOH | cis |
| | acid | | | | |
| ω-3 | Eicosapentaenoic | C20:5 | Δ^{5, 8, 11, 14, 17} | CH₃CH₂CH═CHCH₂CH═CHCH₂CH═CHCH₂CH═CHCH₂CH═CH(CH₂)₃COOH | cis |
| | acid | | | | |
| ω-3 | Docosahexacnoic | C22:6 | Δ^{4, 7, 10, 13, 16, 19} | CH₃CH₂CH═CHCH₂CH═CHCH₂CH═CHCH₂CH═CHCH₂CH═CHCH₂CH═CH | cis |
| | acid | | | (CH₂)₂COOH | |
| ω-6 | Linoleic | C18:2 | Δ^{9, 12} | CH₃(CH₂)₄CH═CHCH₂CH═CH(CH₂)₇COOH | cis |
| | acid | | | | |
| ω-6 | Linolelaidic | C18:2 | | CH₃(CH₂)₄CH═CHCH₂CH═CH(CH₂)₇COOH | trans |
| | acid | | | | |
| ω-6 | γ-Linolenic | C18:3 | Δ^{6, 9, 12} | CH₃(CH₂)₄CH═CHCH₂CH═CHCH₂CH═CH(CH₂)₄COOH | cis |
| | acid | | | | |
| ω-6 | Dihomo-γ- | C20:3 | Δ^{8, 11, 14} | CH₃(CH₂)₄CH═CHCH₂CH═CHCH₂CH═CH(CH₂)₆COOH | cis |
| | linolenic | | | | |
| | acid | | | | |
| ω-6 | Arachidonic | C20:4 | Δ^{5, 8, 11, 14} | CH₃(CH₂)₄CH═CHCH₂CH═CHCH₂CH═CHCH₂CH═CH(CH₂)₃COOH | cis |
| | acid | | | | |
| ω-6 | Docosatetraenoic | C22:4 | Δ^{7, 10, 13, 16} | CH₃(CH₂)₄CH═CHCH₂CH═CHCH₂CH═CHCH₂CH═CH(CH₂)₅COOH | cis |
| | acid | | | | |
| ω-7 | Palmitoleic | C16:1 | Δ⁹ | CH₃(CH₂)₅CH═CH(CH₂)₇COOH | cis |
| | acid | | | | |
| ω-7 | Vaccenic | C18:1 | Δ¹¹ | CH₃(CH₂)₅CH═CH(CH₂)₉COOH | trans |
| | acid | | | | |
| ω-7 | Paullinic | C20:1 | Δ¹³ | CH₃(CH₂)₅CH═CH(CH₂)₁₁COOH | cis |
| | acid | | | | |
| ω-9 | Oleic | C18:1 | Δ⁹ | CH₃(CH₂)₇CH═CH(CH₂)₇COOH | cis |
| | acid | | | | |
| ω-9 | Elaidic | C18:1 | Δ⁹ | CH₃(CH₂)₇CH═CH(CH₂)₇COOH | trans |
| | acid | | | | |
| ω-9 | Gondoic | C20:1 | Δ¹¹ | CH₃(CH₂)₇CH═CH(CH₂)₉COOH | cis |
| | acid | | | | |
| ω-9 | Erucic | C22:1 | Δ¹³ | CH₃(CH₂)₇CH═CH(CH₂)₁₁COOH | cis |
| | acid | | | | |
| ω-9 | Nervonic | C24:1 | Δ¹⁵ | CH₃(CH₂)₇CH═CH(CH₂)₁₃COOH | cis |
| | acid | | | | |
| ω-9 | Mead | C20:3 | Δ^{5, 8, 11} | CH₃(CH₂)₇CH═CHCH₂CH═CHCH₂CH═CH(CH₂)₃COOH | cis |
| | acid | | | | |

Additional lipid groups useful according to the invention include cholesterol, Vitamin E (tocopherol) and bile acid.

In one embodiment, said lipid group is a saturated fatty acid derived group having a hydrocarbon chain of 8 to 24 carbon atoms. In certain embodiments, said lipid group is a saturated fatty acid derived group, wherein said fatty acid is selected from the group consisting of octanoic acid, decanoic acid, dodecanoic acid, tetradecanoic acid, hexadecanoic acid, octadecanoic acid, eicosanoic acid, docosanoic acid and tetracosanoic acid. In one embodiment, said lipid group is a saturated fatty acid derived group, wherein said fatty acid is hexadecanoic acid. In one embodiment, said fatty acid derived group is attached to the oligonucleotide via the linker on the 5′ end of the abc-DNA oligonucleotide. In one embodiment, said fatty acid derived group is attached to the oligonucleotide via the linker on the 7′ end of the abc-DNA oligonucleotide.

In one embodiment, said lipid group is an unsaturated fatty acid derived group having a hydrocarbon chain of 8 to 24 carbon atoms. In certain embodiments, said lipid group is an unsaturated fatty acid derived group, wherein said fatty acid is selected from the group consisting of myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid.

Linker

The oligonucleotides of the invention are connected to a lipid group via a linker. In some embodiments, the linker is connected to the lipid group via an amide bond. For hydrocarbon linkers, the linker comprises 2-20 carbons, for example, 2, 3, 4, 5, 6, 7, 8 9 or 10 carbons. For polyethylene glycol (PEG) linkers, the linker comprises 1-20 ethylene glycol subunits, for example, 1, 2, 3, 4, 5, 6, 7, 8 9 or 10 ethylene glycol repeats. A linker can be a hydrocarbon linker or a polyethylene glycol (PEG) linker. A linker according to the invention, wherein the abcDNA is attached to the phosphorous moiety of the linker, and the lipid group, for example a fatty acid derived group, is attached to Y, can have, for example, the general structure shown below:

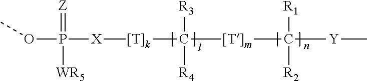

wherein:
If Y ═NH then the fatty acid-derived group is connected via an amide bond;
If n=1, R₁ can be, for example, CO₂H and R₂ can be, for example, H;
T′ can be —CH₂—CH₂—O with m being the number of ethylene glycol repeats;
T can be a biocleavable entity such as a disulfide group, and k is equal to 1,
wherein, in certain embodiments,
X can be oxygen or NH;
Z can be O or S; and
WR₅ can be OH or SH.

Linkers useful according to the invention include but are not limited to the following: amino-alkyl-phosphorothioate linker:

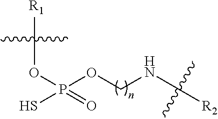

R₁=oligonucleotide

R₂=conjugated lipid group

wherein n is preferably an integer of 2 to 12, preferably of 4 to 10. In one embodiment, n is an integer of 2, 3, 4, 5, 6, 7, 8, 9, 10, 11 or 12. In one embodiment, n is 6.

alpha-carboxylate-amino-alkyl-phosphorothioate linker:

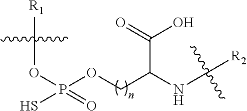

R₁=oligonucleotide

R₂=conjugated lipid group

wherein n is preferably an integer of 2 to 12, preferably of 4 to 10. In one embodiment, n is an integer of 2, 3, 4, 5, 6, 7, 8, 9, 10, 11 or 12. In one embodiment, n is 6.

amino-PEG-phosphorothioate linker:

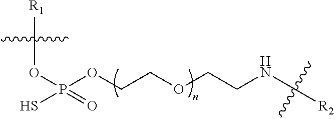

R₁=oligonucleotide

R₂=conjugated lipid group

wherein n is preferably an integer of 1 to 8. In one embodiment, n is an integer of 1, 2, 3, 4, 5, 6, 7 or 8.

and

alpha-carboxylate-amino-PEG-phosphorothioate linker:

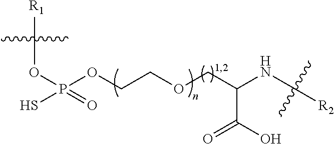

R₁=oligonucleotide

R₂=conjugated lipid group wherein n is preferably an integer of 1 to 8. In one embodiment, n is an integer of 1, 2, 3, 4, 5, 6, 7 or 8.

Thus, in one embodiment said linker is selected from the group consisting of an amino-alkyl-phosphorothioate linker;
(ii) an alpha-carboxylate-amino-alkyl-phosphorothioate linker;
(iii) an amino-PEG-phosphorothioate linker, and
(iv) alpha-carboxylate-amino-PEG-phosphorothioate linker all as defined above in provided formula.

In one embodiment said linker is an amino-alkyl-phosphorothioate linker of the formula

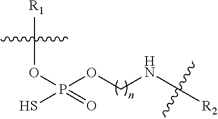

R₁=oligonucleotide

R₂=conjugated lipid group

wherein n is an integer of 2 to 12, preferably of 4 to 10. In one embodiment, n is an integer of 2, 3, 4, 5, 6, 7, 8, 9, 10, 11 or 12. In one embodiment, n is 6.

Thus, the invention provides for an amino alkyl phosphorothioate linker having the structure presented below.

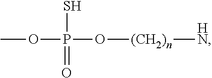

wherein n is an integer of 2 to 12, preferably of 4 to 10. In one embodiment, n is an integer of 2, 3, 4, 5, 6, 7, 8, 9, 10, 11 or 12. In one embodiment, n is 6.

An example of an oligonucleotide of the invention comprising SEQ ID NO: 10 connected to a lipid group via an amino alkyl phosphorothioate linker has the structure:

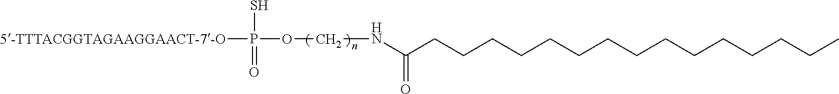

wherein n is an integer of 2 to 12, preferably of 4 to 10, more preferably n is 6. Preferably all of the residues of SEQ ID NO:10 are abc-DNA residues corresponding to SEQ ID NO: 418.

Another example of an oligonucleotide of the invention (SEQ ID NO: 412) connected to a lipid group via an amino alkyl phosphorothioate linker has the structure:

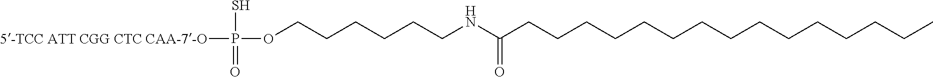

The invention also provides for an amino-PEG-phosphorothioate linker having the structure provided below.

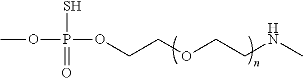

wherein n is preferably an integer of 1 to 8. In one embodiment, n is an integer of 1, 2, 3, 4, 5, 6, 7 or 8.

An example of an oligonucleotide of the invention comprising SEQ ID NO: 10 connected to a lipid group via an amino-PEG-phosphorothioate linker has the structure:

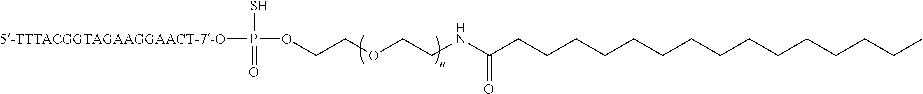

wherein n is preferably an integer of 1 to 8. In one embodiment, n is an integer of 2, 3, 4, 5, 6, 7 or 8. Preferably all of the residues of SEQ ID NO:10 are abc-DNA residues corresponding to SEQ ID NO: 418.

The invention also provides for an alpha-carboxylate-amino-alkyl-phosphorothioate linker having the structure provided below.

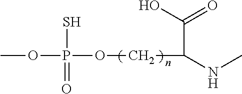

wherein n is preferably an integer of 2 to 12, preferably of 4 to 10. In one embodiment, n is an integer of 2, 3, 4, 5, 6, 7, 8, 9, 10, 11 or 12. In one embodiment, n is 6.

An example of an oligonucleotide of the invention comprising SEQ ID NO: 10 connected to a lipid group via an alpha-carboxylate-amino-alkyl-phosphorothioate linker has the structure:

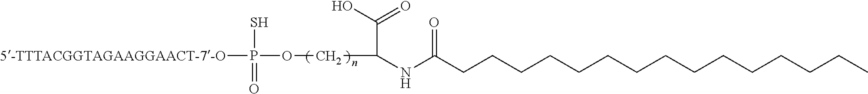

wherein n is an integer of 2 to 12, preferably of 4 to 10, more preferably n is 6. Preferably all of the residues of SEQ ID NO:10 are abc-DNA residues corresponding to SEQ ID NO: 418.

The invention also provides for an alpha-carboxylate-amino-PEG-phosphorothioate linker having the structure provided below.

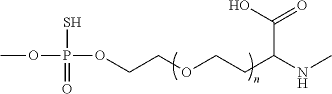

wherein n is preferably an integer of 1 to 8. In one embodiment, n is an integer of 2, 3, 4, 5, 6, 7 or 8.

An example of an oligonucleotide of the invention comprising SEQ ID NO: 10 connected to a lipid group via an alpha-carboxylate-amino-PEG-phosphorothioate linker has the structure:

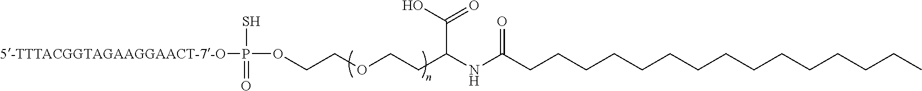

wherein n is preferably an integer of 1 to 8. In one embodiment, n is an integer of 2, 3, 4, 5, 6, 7 or 8. Preferably all of the residues of SEQ ID NO:10 are abc-DNA residues corresponding to SEQ ID NO: 418;

or the structure:

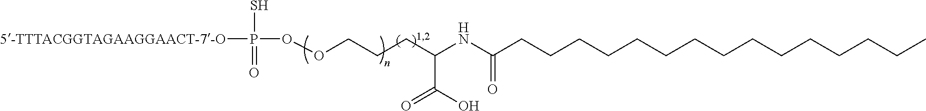

wherein n is preferably an integer of 1 to 8. In one embodiment, n is an integer of 2, 3, 4, 5, 6, 7 or 8. Preferably all of the residues of SEQ ID NO:10 are abc-DNA residues corresponding to SEQ ID NO: 418.

In one embodiment, the invention provides for a linker that is conformationally restrained, for example, based on hydroxyproline, for example,

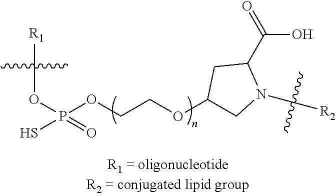

wherein n is preferably an integer of 1 to 8. In one embodiment, n is an integer of 2, 3, 4, 5, 6, 7 or 8.

The linker can be attached to the 5′ and/or 7′ terminal OH group of the oligonucleotide via, for example, a thiophosphate group. In one embodiment, the linker is attached to the 5′ terminal OH group of the oligonucleotide via, for example, a thiophosphate group. In one embodiment, the linker is attached to the 7′ terminal OH group of the oligonucleotide via, for example, a thiophosphate group. Additional groups that can be used to connect a linker to an oligonucleotide include a phosphate group.

In some embodiments, a fatty acid conjugated phosphoramidite may be used for the coupling of a fatty acid to the abc-DNA at either the 5′ terminus, the 7′ terminus, or both the 5′ and 7′ termini. An example of a phosphoramidite which may be used for the coupling of a fatty acid to the abc-DNA has the structure:

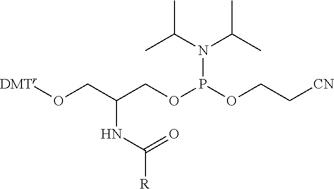

wherein R—CO is a fatty acid moiety.

In other embodiments, the linker is an alpha-carboxylate-amino linker having, for example, the structure:

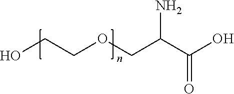

wherein n is preferably an integer of 1 to 8. In one embodiment, n is an integer of 2, 3, 4, 5, 6, 7 or 8.

In its simplest form, the linker is a 2-amino-6-hydroxy-4-oxahexanoic acid linker wherein n=1. Alternatively, a linker having the structure above wherein the stereochemistry at C₂ matches that of serine, is an O-(2-hydroxyethyl)-L-serine linker. In the context of an abcDNA fatty acid conjugate, the hydroxyl function of the linker is connected via a phosphorothioate linkage to the abcDNA and the amino group is connected to the carboxyl group of the fatty acid entity via an amide bond.

In another embodiment, fatty acid conjugated alpha-carboxylate-amino-PEG phosphoramidite reagent has the structure below, whereby R is a suitable protecting group, such as 2-chlorotrityl, used in the final step of solid-phase synthesis of an abcDNA-linker-fatty acid conjugate:

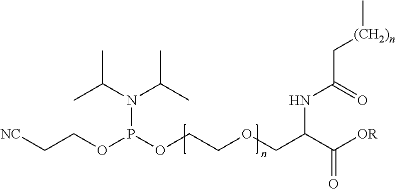

wherein n is preferably an integer of 1 to 8. In one embodiment, n is an integer of 2, 3, 4, 5, 6, 7 or 8.

In other embodiments, a phosphoramidite which may be used for the coupling of a fatty acid to the abc-DNA has the structure (AM Chemicals, LLC, Oceanside, Calif.):

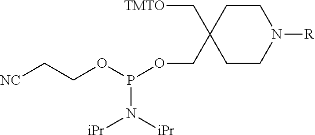

wherein R is a fatty acid moiety.

In some embodiments, a fatty acid conjugated solid phase support may be used for the coupling of a fatty acid to the abc-DNA at the 5′ terminus. An example of a solid phase support which may be used for the coupling of a fatty acid to the abc-DNA has the structure:

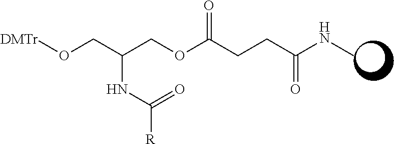

wherein R—CO is a fatty acid moiety and the shaded circle is the solid phase support.

In other embodiments, a solid phase support which may be used for the coupling of a fatty acid to the abc-DNA has the structure (AM Chemicals, LLC, Oceanside, Calif.):

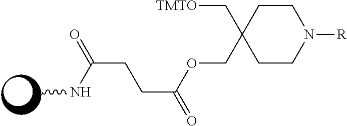

wherein R is a fatty acid moiety and the shaded circle is the solid phase support.

In certain embodiments, the linker contains a cleavable bond, for example, a disulfide bond, an acid cleavable hydrazone bond, or a protease cleavable moiety.

### Methods of Synthesis

Methods of synthesis well known in the art are used to synthesize abc-DNA nucleosides and oligonucleotides comprising abc-DNA nucleosides. In some embodiments, for oligonucleotides conjugated to a lipid group at the 5′ end, the linker of the invention is attached to a solid support prior to synthesis of the oligonucleotide and attachment. In certain embodiments, for oligonucleotides wherein the lipid group is conjugated to the 7′ end of the oligonucleotide, conjugation occurs during solid phase synthesis. In other embodiments, for oligonucleotides wherein the lipid group is conjugated to the 7′ end of the oligonucleotide, conjugation occurs after synthesis is completed.

General Procedures

All reactions are performed in dried glassware and under an inert atmosphere of Argon. Anhydrous solvents for reactions are obtained by filtration through activated alumina or by storage over molecular sieves (4 Å). Column chromatography (CC) is performed on silica gel (SiliaFlash P60, 40-63 μm, 60 Å). Methanol used for CC is of HPLC grade, all other solvents used for CC are of technical grade and distilled prior to use. Thin-layer chromatography is performed on silica gel plates (Macherey-Nagel, pre-coated TLC-plates sil G-25 UV254). Compounds are visualized under UV-light or by dipping in a p-anisaldehyde staining solution [p-anisaldehyde (3.7 mL), glacial acetic acid (3.7 mL), concentrated sulfuric acid (5 mL), ethanol (135 mL)] followed by heating with a heat gun. NMR spectra are recorded at 300 or 400 MHz (¹H), at 75 or 101 MHz (¹³C) and at 122 MHz (³¹P) in either CDCl₃, CD₃OD or CD₃CN. Chemical shifts (6) are reported relative to the residual undertreated solvent peak [CDCl₃: 7.26 ppm (¹H), 77.16 ppm (¹³C); CD₃OD: 3.31 ppm (¹H), 49.00 ppm (¹³C)]. Signal assignments are based APT and DEPT and on ¹H, ¹H and ¹H, ¹³C correlation experiments (COSY, HSQC, HMBC). High resolution mass data are obtained by electrospray ionization in the positive mode (ion trap, ESI⁺).

### Temperature of Melting

UV-melting experiments are recorded on a Varian Cary Bio 100 UV/vis spectrophotometer. Experiments are performed at 2 μM duplex concentration, 10 mM NaH₂PO₄, between 0 M and 150 mM NaCl (alpha anomer) or between 0.05 M and 1.00 M NaCl (beta anomer) and pH adjusted to 7.0. Samples are protected from evaporation by a covering layer of dimethylpolysiloxane. Absorbance is monitored at 260 nm. For every experiment, three cooling-heating cycles are performed with a temperature gradient of 0.5° C./min. The maxima of the curves first derivative are extracted with Varian WinUV software and Tm values are reported as the average of the six ramps.

### Circular Dichroism Spectroscopy

CD-spectra are recorded on a Jasco J-715 spectropolarimeter equipped with a Jasco PFO-3505 temperature controller. Sample conditions are the same as for UV-melting experiments. Spectra are recorded between 210 and 320 nm at a 50 nm/min rate and the temperature is measured directly from the sample. For each experiment, a blank containing the same salt concentrations as the sample are recorded. The reported spectra are obtained by taking a smoothed average of three scans and subtracting the corresponding blank spectrum.

### Syntheses of abcDNA Nucleosides

The bicyclic scaffolds 7 and 10 envisaged for subsequent nucleoside synthesis are constructed from the previously described intermediate 1 (Tarköy, M.; Bolli, M.; Schweizer, B.; Leumann, C. Helv. Chim. Acta 1993, 76, 481) (Scheme 1). The epoxide ring in 1 is efficiently opened by LiHMDS mediated intramolecular elimination at −78° C., yielding the unsaturated ester 2 in good yield. Subsequent nickel-catalyzed NaBH₄ reduction of 2 proceeds stereospecifically from the convex side of the bicyclic core structure, resulting in ester 3 as the only identifiable diastereoisomer. The hydroxyl function in 3 is then TBDPS protected, giving 4 in quantitative yield. Intermediate 4 is consequently reduced with DIBAL at −78° C., leading to aldehyde 5. The acetonide protecting group in 5 is then hydrolyzed under mild conditions with In(OTf)₃ as catalyst (Golden, K. C.; Gregg, B. T.; Quinn, J. F. Tetrahedron Lett. 2010, 51, 4010), in a mixture of MeCN and H₂O, and the resulting bicyclic hemiacetal converted into the methyl glycoside 6 by simply changing the solvent to MeOH. Compound 6 is then acetylated to afford the protected precursor 7 that is used for the synthesis of the corresponding purine nucleosides via Vorbrüggen chemistry.

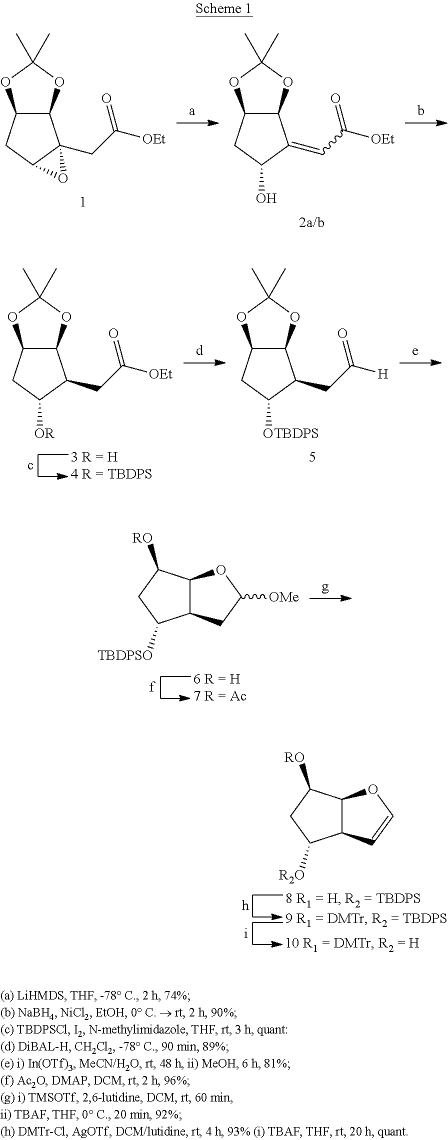

Synthesis of pyrimidine nucleosides of the present invention consists in the well-established application of the β-stereoselective NIS induced addition of the nucleobases to a corresponding bicyclic glycal (Medvecky, M.; Istrate, A.; Leumann, C. J. J. Org. Chem. 2015, 80, 3556; Dugovic, B.; Leumann, C. J. Journal of Organic Chemistry 2014, 79, 1271; Lietard, J.; Leumann, C. J. J. Org. Chem. 2012, 77, 4566). First, to introduce the thymine nucleobase, the N-iodosuccinimide (NIS) induced nucleosidation is performed on the direct precursor of glycal 8, where R₁=TMS, that is easily obtained from 6 by treatment with TMSOTf only. This approach results in the stereoselective formation of the corresponding β-nucleoside, however, with a significant contamination of 7% of the α-anomer that remained inseparable by standard chromatography techniques. It is reasoned that the β-selectivity could be enhanced by increasing steric bulk at R₁ and decreasing it at R₂, as in glycal 10. This would favor initial α-attack of the electrophilic iodine at C(4). To this end compound 6 is converted to glycal 8 with TMSOTf followed by a short treatment with TBAF to remove the newly introduced TMS group selectively. Intermediate 8 is then elaborated into the dimethoxytrityl compound 9 which is finally subjected to removal of the TBDPS protecting group with TBAF to give the desired sugar component 10.

NIS-nucleosidation on the in situ TMS protected glycal 10, followed by radical reduction of the iodide intermediate with Bu₃SnH, yields the DMTr-protected thymidine derivative 11 in good yield containing only trace amounts (<2% by ¹H-NMR) of the α-anomer (Scheme 2). Final phosphitylation with 2-cyanoethyl N,N,N′,N′-tetraisopropylphosphordiamidite leads to the thymidine phosphoramidite building block 12. The synthesis of the 5-methylcytosine nucleoside is achieved by conversion of the base thymine. To this end, nucleoside 11 is TMS protected and converted to the corresponding triazolide by treatment with 1,2,4-triazole and POCl₃. Subsequent treatment of this triazolide in a mixture of ammonia and 1,4-dioxane yields the corresponding 5-methylcytosine nucleoside, which is directly protected with Bz₂O to give 13 in 88% yield over three steps. The phosphoramidite 14 is obtained by a phosphitylation as described above.

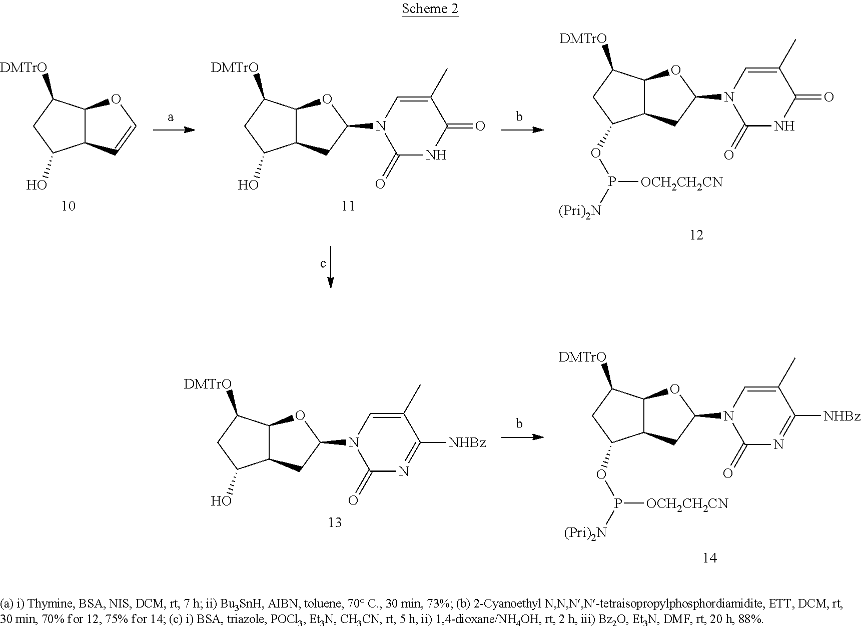

Classical Vorbrüggen nucleosidation is applied for introducing the purine nucleobases resulting generally in the prevalence of the α-nucleosides. The conversion of precursor 7 with either N⁶-benzoyladenine or 2-amino-6-chloropurine leads to the inseparable anomeric mixtures 15 and 20, resp. in α/β ratios of 4:1 and 7:3 (Scheme 3). Separation of anomers is possible after deacetylation, leading to the pure β-anomers 16 and 21. From here, the adenine building block 19 is obtained by standard dimethoxytritylation (→17) followed TBAF mediated cleavage of the silyl protecting group (→19) and phosphitylation. The synthesis of the guanine building block requires the conversion of the 2-amino-6-chloropurine nucleobase. This is achieved by treatment of 21 with 3-hydroxypropionitrile and TBD and subsequent protection of the 2-amino group with DMF, yielding the protected guanosine derivatives 22. Following the same chemical pathway as above, the synthesis of the guanine building block 25 is achieved by dimethoxytritylation (→23) followed by removal of silyl protecting group (→24) and phosphitylation.

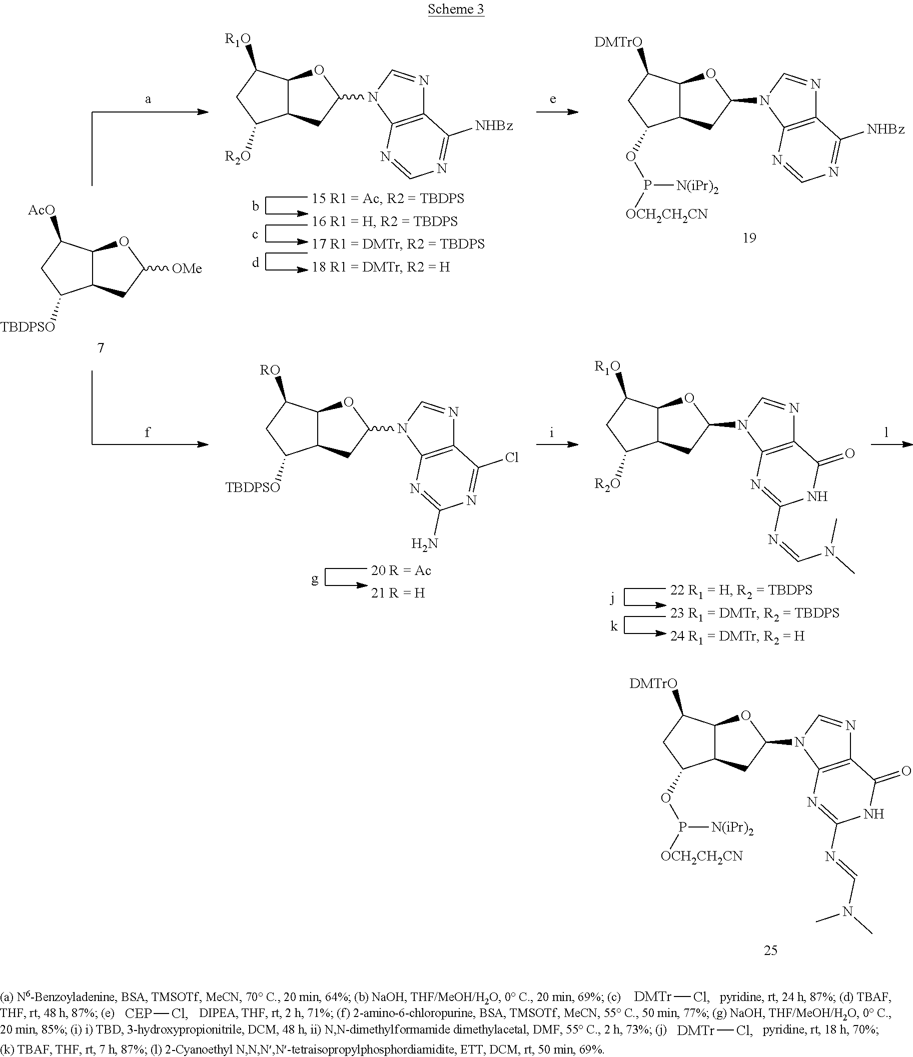

Starting from protected sugar 7 the synthesis of four preferred phosphoramidite building blocks of the present invention is developed. Treatment of a mixture of sugar 7 and in situ silylated thymine with TMSOTf results in the smooth formation of the nucleoside 35, with a favorable anomeric ratio α/β of approximately 85:15 (determined by ¹H-NMR) (Scheme 4). The chemical pathway leading to the thymidine phosphoramidite bearing the DMTr group on the 5′ position does not allow the separation of anomers by standard chromatography. Therefore, and in order to introduce the modification with polarity reversal into DNA strands, the DMTr group is introduced on the 7′ position. To this end, the silyl group of 35 is removed by a short treatment with TBAF (→36) followed by standard dimethoxytritylation (→37). Separation of the two anomers is possible after standard deacetylation, leading to the pure α-anomer 38. The thymidine building block 39 is finally obtained by phosphitylation with 2-cyanoethyl N,N,N′,N′-tetraisopropylphosphordiamidite in the presence of 5-(ethylthio)-1H-tetrazole. The intermediate 38 also offers short access to the 5-methylcytosine nucleoside, by conversion of the in situ TMS protected nucleoside 38 to the corresponding triazolide with POCl₃ and 1,2,4-triazole, followed by treatment with a mixture of ammonia and 1,4-dioxane. Direct protection with Bz₂O in DMF results in the efficient formation of nucleoside 40, the labile silyl protecting group being cleaved during the process. Final phosphitylation under conditions as described above affords the 5-methylcytidine phosphoramidite 41.

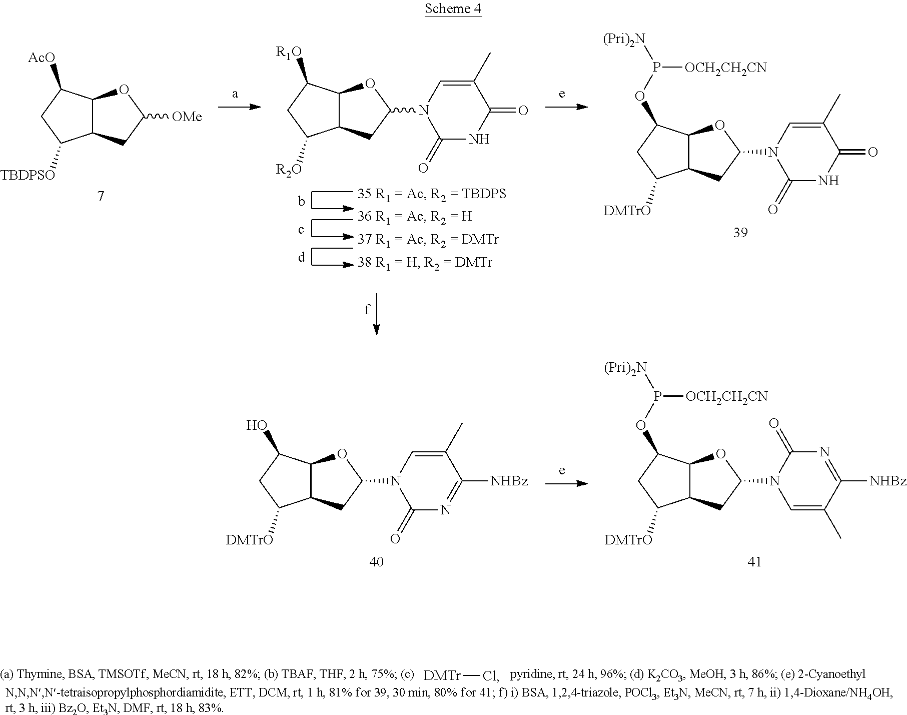

For the purine nucleobases, the introduction of the purines is performed by a short nucleosidation at slightly elevated temperature with either N⁶-benzoyladenine or 2-amino-6-chloropurine, leading to the nucleoside 15 and 20, resp. in α/β ratios of 4:1 and 7:3 (Scheme 5). To separate the anomers, acetyl groups are removed under mild conditions, yielding the pure α-anomers 42 and 48. The formation of the adenosine building block continues with the reintroduction of the acetyl protecting group (→43), removal of the TBDPS protecting group with TBAF (→44) followed by standard dimethoxytritylation (→45). Selective deprotection of the acetyl group (→46) followed by phosphitylation under conditions as described above yields the adenine building block 47.

For the guanine building block, after separation of the two anomers, the 6-chloropurine is converted to the guanine nucleobase by treatment with TBD and 3-hydroxypropionitrile yielding the guanosine nucleoside 49. Acetylation over 48 h allowed the concomitant protection of the 5′-hydroxy and 2-amino groups, yields the protected nucleoside 50. Similarly as above, the DMTr group is introduced by removal of the silyl protecting group with TBAF (→51) followed by dimethoxytritylation (→52). The two acetyl groups are removed by treatment with K₂CO₃ and the resulting polar product is directly protected with DMF to afford the guanosine nucleoside 53. Final phosphitylation yielded building block 54.

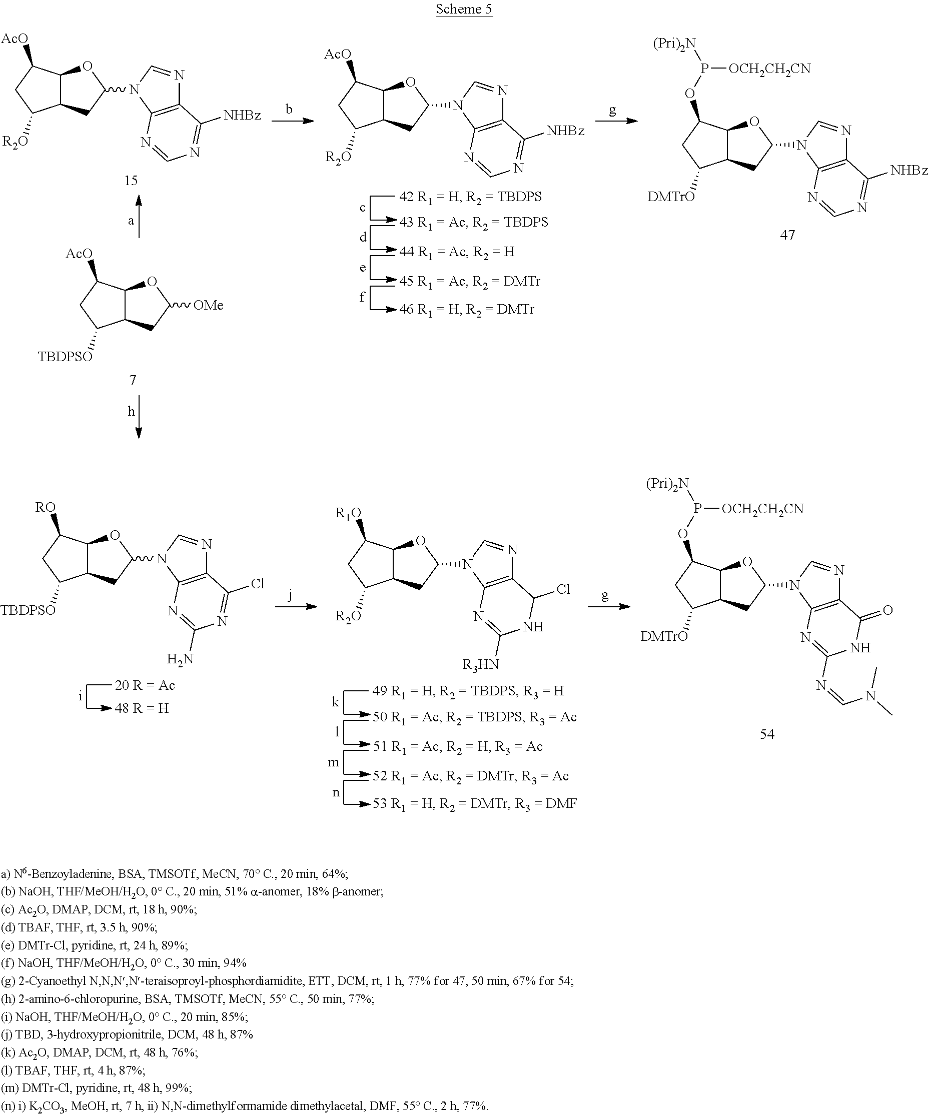

### Ethyl (E and Z, 1′R,5'S,7′R)-(7′-hydroxy-3′,3′-dimethyl-2′,4′-dioxabicyclo[3.3.0]oct-6′-ylidene)acetate (2a/b)

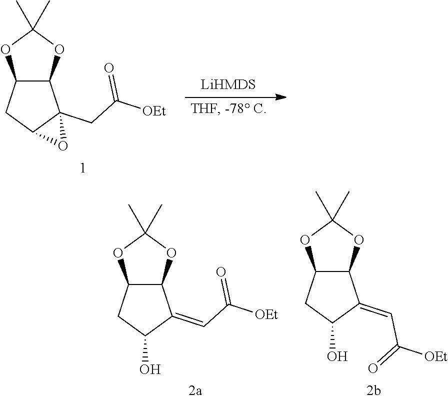

A solution of the epoxide 1 (4.46 g, 18.4 mmol) in dry THF (100 mL) is cooled down to −78° C. Then LiHMDS (1 M in THF, 22.1 mL, 22.1 mmol) is slowly added. The solution is stirred for 2 hours at −78° C. before being allowed to warm to room temperature and neutralized with the addition of 1M aqueous HCl (22.1 mL). The mixture is then diluted with EtOAc (100 mL) and THF is removed under reduced pressure. The mixture is then washed with 0.5 M NaH₂PO₄ (50 mL) and aqueous phase extracted with EtOAc (2×50 mL). The combined organic phases are dried over MgSO₄, filtered and evaporated. The crude product is purified by CC (EtOAc/hexane 3:1) to yield the two isomers 2a/b (3.30 g, 74%) as a pale yellow solid.

Data for 2a: R_{f}=0.37 (EtoAc/hexane 1:1):

¹H NMR (300 MHz, CDCl₃) δ 6.07-5.98 (m, 1H, H—C(2)), 5.59 (d, J=6.0 Hz, 1H, H—C(5′)), 4.94-4.81 (m, 1H, H—C(1′)), 4.65 (t, J=5.6 Hz, 1H, H—C(7′)), 4.18 (q, J=7.1 Hz, 2H, CH₃CH₂), 2.67 (br, 1H, OH), 2.37 (dd, J=13.5, 7.5 Hz, 1H, H—C(8′)), 1.55-1.42 (m, 1H, H—C(8′)), 1.40, 1.33 (2s, 6H, (CH₃)₂C), 1.26 (t, J=7.1 Hz, 3H, CH₂CH₃).

¹³C NMR (75 MHz, CDCl₃) δ 165.75 (C(1)), 161.61 (C(6′)), 116.53 (C(2)), 110.69 (C(3′)), 76.55 (C(5′)), 75.52 (C(1′)), 71.63 (C(7′)), 60.51 (CH₂CH₃), 37.46 (C(8′)), 26.44, 24.11 ((CH₃)₂C), 14.27 (CH₂CH₃).

ESI⁺-HRMS m/z calcd for C₁₂H₁₉O₅ ([M+H]⁺) 243.1227, found 243.1231.

Data for 2b: R_{f}=0.52 (EtoAc/hexane 1:1):

¹H NMR (300 MHz, CDCl₃) δ 6.15-6.05 (m, 1H, H—C(2)), 5.37-5.02 (m, 2H, H—C(5′), OH), 4.87 (d, J=3.4 Hz, 1H, H—C(1′)), 4.67 (t, J=4.9 Hz, 1H, H—C(7′)), 4.20 (qd, J=7.1, 0.9 Hz, 2H, CH₃CH₂), 2.55 (dd, J=14.6, 8.1 Hz, 1H, H—C(8′)), 1.94-1.77 (m, 1H, H—C(8′)), 1.39-1.25 (m, 9H, (CH₃)₂C, CH₂CH₃).

¹³C NMR (75 MHz, CDCl₃) δ 167.91 (C(1)), 167.43 (C(6′)), 120.13 (C(2)), 111.75 (C(3′)), 81.62 (C(5′)), 78.08 (C(1′)), 70.85 (C(7′)), 61.25 (CH₂CH₃), 36.53 (C(8′)), 27.38, 25.45 ((CH₃)₂C), 14.19 (CH₂CH₃).

ESI⁺-HRMS m/z calcd for C₁₂H₁₉O₅ ([M+H]⁺) 243.1227, found 243.1227.

### Ethyl (1′R,5'S,6'S,7′R)-(7′-hydroxy-3′,3′-dimethyl-2′,4′-dioxabicyclo[3.3.0]oct-6′-yl)acetate (3)

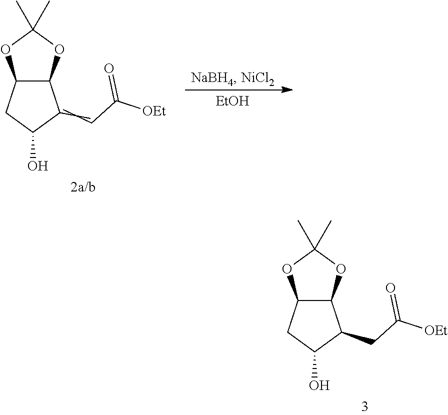 [SMILES: "EtOH" ; CCOC(=O)C=C1[C@H](O)C[C@H]2OC(C)(C)O[C@@H]12 ; CCOC(=O)C[C@@H]1[C@@H]2OC(C)(C)O[C@@H]2C[C@H]1O]

To a solution of the alcohols 2a/b (12.65 g, 52.2 mmol) and nickel chloride hexahydrate (2.48 g, 10.4 mmol) in EtOH (300 mL) is added portion wise sodium borohydride (9.88 g, 261 mmol) at 0° C. The resulting dark solution is stirred for 30 min at 0° C. and 90 min at room temperature. Then EtOH is carefully removed under reduced pressure, the resulting solid diluted with EtOAc (200 mL) and the excess of NaBH₄ quenched by addition of water (100 mL) at 0° C. followed by stirring at room temperature for 30 min. The two phases are then separated. Organic phase is washed with water (100 mL). Aqueous phases are then combined, filtered and extracted with EtOAc (2×100 mL). The combined organic phases are dried over MgSO₄, filtered and concentrated. The crude product is purified by CC (EtOAc/hexane 2:1) to yield 3 (11.4 g, 90%) as a white solid.

Data for 3: R_{f}=0.40 (EtOAc/hexane 1:1):

¹H NMR (300 MHz, CDCl₃) δ 4.65-4.52 (m, 2H, H—C(1′), H—C(5′)), 4.15 (qd, J=7.1, 1.4 Hz, 2H, CH₃CH₂), 4.05 (ddd, J=10.0, 9.99, 6.2 Hz, 1H, H—C(7′)), 2.86 (br, s, 1H, OH), 2.65 (qd, J=16.9, 7.1 Hz, 2H, H—C(2)), 2.24 (dd, J=13.7, 6.2 Hz, 1H, H—C(8′)), 1.93 (dt, J=12.7, 7.1 Hz, 1H. H—C(6′)), 1.56 (ddd, J=13.9, 10.2, 5.5 Hz, 1H, H—C(8′)), 1.38 (s, 3H, (CH₃)₂C), 1.30-1.21 (m, 6H, (CH₃)₂C, CH₂CH₃).

¹³C NMR (75 MHz, CDCl₃) δ 174.38 (C(1)), 109.06 (C(3′)), 79.65 (C(5′)), 77.19 (C(1′), 74.32 (C(7′), 60.80 (CH₂CH₃), 46.66 (C(6′)), 40.38 (C(8′)), 32.43 (C(2)), 26.00, 23.69 ((CH₃)₂C), 14.17 (CH₂CH₃).

ESI⁺-HRMS m/z calcd for C₁₂H₂₁₀₅ ([M+H]⁺) 245.1384, found 245.1388.

### Ethyl (1′R,5'S,6'S,7′R)-(7′-(tert-butyldiphenylsilyl)oxy)-3′,3′-dimethyl-2′,4′-dioxabicyclo[3.3.0]oct-6′-yl)acetate (4)

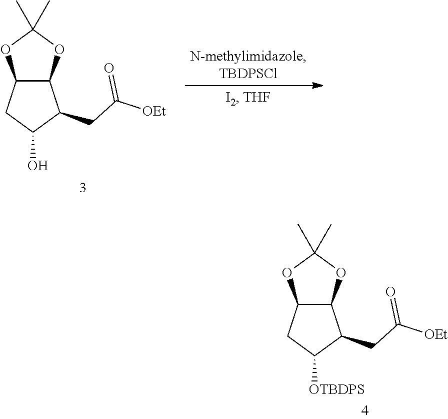

To a solution of the alcohol 3 (2.50 g, 10.2 mmol), N-methylimidazole (12.6 g, 153 mmol) and iodine (7.80 g, 30.6 mmol) in dry THF (60 mL) is added dropwise tert-butyl(chloro)diphenylsilane (3.0 mL, 11.2 mmol) at room temperature (rt). The solution is stirred for 3 hours at rt and then THF is evaporated, the mixture diluted with EtOAc (50 mL) and washed with 10% aqueous Na₂O₃S₂ (2×40 mL). Aqueous phases are then combined and extracted with EtOAc (50 mL). The combined organic phases are dried over MgSO₄, filtered and evaporated. The crude product is purified by CC (EtOAc/hexane 1:10) to yield 4 (5.01 g, quantitative yield) as a white solid.

Data for 4: R_{f}=0.87 (DCM/MeOH 10:1):

¹H NMR (300 MHz, CDCl₃) δ 7.77-7.59 (m, 4H, H-arom), 7.51-7.32 (m, 6H, H-arom), 4.61 (t, J=5.7 Hz, 1H, H—C(5′)), 4.49 (t, J=5.7 Hz, 1H, H—C(1′)), 4.15 (q, J=6.9 Hz, 2H, CH₃CH₂), 3.96 (dd, J=15.5, 9.5 Hz, 1H, H—C(7′)), 2.64-2.32 (m, 2H, H—C(2)), 2.15 (tt, J=9.0, 4.3 Hz, 1H, H—C(6′)), 1.83 (dd, J=12.7, 5.2 Hz, 1H, H—C(8′)), 1.61-1.45 (m, 1H, H—C(8′)), 1.27 (td, J=7.1, 1.9 Hz, 3H, CH₂CH₃), 1.18 (s, 6H, (CH₃)₂C), 1.09, 1.08 (2s, 9H, (CH₃)₃—C—Si)

¹³C NMR (75 MHz, CDCl₃) δ 173.07 (C(1)), 135.87, 135.85 (CH-arom), 134.08, 133.73 (C-arom), 129.80, 129.75, 127.67, 127.58 (CH-arom), 108.82 (C(3′)), 77.92 (C(5′)), 76.96 (C(1′)), 74.93 (C(7′)), 60.24 (CH₂CH₃), 47.27 (C(6′)), 40.27 (C(8′)), 31.10 (C(2)), 27.04 (CH₃)₃—C—Si), 25.86 ((CH₃)₂C), 23.83 ((CH₃)₂C), 19.23 (CH₃)₃—C—Si), 14.24 (CH₂—CH₃).

ESI⁺-HRMS m/z calcd for C₂₈H₃₉O₅Si ([M+H]⁺) 483.2561, found 483.2562.

### (1′R,5'S,6'S,7′R)-(7′-(tert-butyldiphenylsilyl)oxy)-3′,3′-dimethyl-2′,4′-dioxabicyclo[3.3.0]oct-6′-yl)acetaldehyde (5)

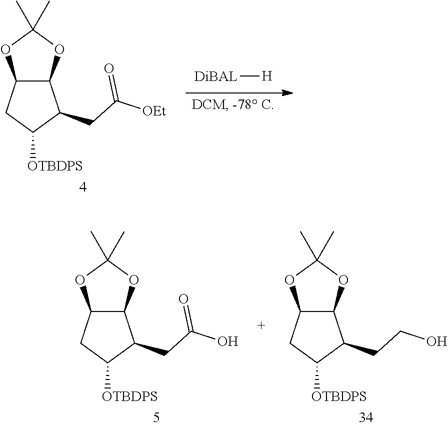

A solution of the ester 4 (8.56 g, 16.3 mmol) in dry DCM (120 mL) is cool down to −78° C. and then DiBAL-H (1 M in cyclohexane, 18 mL, 18 mmol) is slowly added. The solution is further stirred at −78° C. for 90 min before being allowed to warm to rt. Reaction is quenched by addition of 0.5 M aqueous NaH₂PO₄ (100 mL). The organic phase is separated and the aqueous phase is further extracted with DCM (2×100 mL). The combined organic phases are dried over MgSO₄, filtered and evaporated. The crude product is purified by CC (EtOAc/hexane 2:10 to 2:1) to yield aldehyde 5 (6.36 g, 89%) and alcohol 34 (0.637 g, 9%).

Data for 5: R_{f}=0.65 (EtOAc/hexane 2:1):

¹H NMR (300 MHz, CDCl₃) δ 9.72 (s, 1H, H—C(1)), 7.65 (td, J=8.0, 1.6 Hz, 4H, H-arom), 7.47-7.33 (m, 6H, H-arom), 4.57 (t, J=5.7 Hz, 1H, H—C(5′)), 4.51 (t, J=5.7 Hz, 1H, H—C(1′)), 3.99 (td, J=10.0, 5.9 Hz, 1H, H—C(7′)), 2.58-2.43 (m, 2H, H—C(2)), 2.20-2.08 (m, 1H, H—C(6′)), 1.87 (dd, J=13.5, 5.9 Hz, 1H, H—C(8′)), 1.53 (ddd, J=13.5, 10.1, 5.5 Hz, 1H, H—C(8′)), 1.16 (d, J=3.5 Hz, 6H, ((CH₃)₂C), 1.05 (s, 9H, (CH₃)₃—C—Si).

¹³C NMR (75 MHz, CDCl₃) δ 201.87 (C(1)), 135.93, 135.90 (CH-arom), 133.96, 133.73 (C-arom), 129.96, 129.89, 127.79, 127.68 (CH-arom), 108.89 (C(3′)), 77.76 (C(5′)), 77.17 (C(1′)), 74.96 (C(7′), 45.44 (C(6′)), 41.31 (C(2)), 40.16 (C(8′)), 27.08 (CH₃)₃—C—Si), 25.87 ((CH₃)₂C), 23.79 ((CH₃)₂C), 19.25 (CH₃)₃—C—Si).

ESI⁺-HRMS m/z calcd for C₂₆H₃₅O₄Si ([M+H]⁺) 439.2299, found 439.2297.

### (3aR,4R,6R,6aS)-4-((tert-butyldiphenylsilyl)oxy)-2-methoxyhexahydro-2H-cyclopenta[b]furan-6-ol (6)

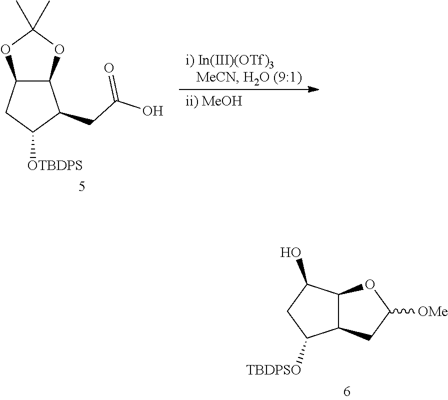

To a solution of the aldehyde 5 (13.73 g, 31.31 mmol) in MeCN (170 mL) and H₂O (19 mL) is added indium(III) trifluoromethanesulfonate (703 mg, 1.25 mmol). The solution is further stirred for 48 hours, and then solvents are removed under reduced pressure and coevaporated with toluene. The residue is dissolved in dry MeOH and stirred for 6 hours. After evaporation of solvent, the crude product is purified by CC (EtOAc/hexane 3:10) to yield a mixture of 6 (10.50 g, 81%) in an anomeric ratio α/β≈4:1 as a colorless oil.

Data for 6: R_{f}=0.53 (EtOAc/hexane 1:1):

¹H NMR (300 MHz, CDCl₃) δ 7.63 (dd, J=7.1, 0.6 Hz, 4H, H-arom), 7.46-7.34 (m, 6H, H-arom), 4.98 (d, J=4.8 Hz, 0.8H, H—C(2)), 4.91 (dd, J=5.9, 1.3 Hz, 0.2H, H—C(2)), 4.63-4.54 (m, 1H, H—C(6a)), 4.53-4.37 (m, 1H, H—C(6)), 4.09 (m, 0.2H, H—C(4)), 3.92 (br, 0.8H, H—C(4)), 3.29, 3.27 (2s, 3H, MeO), 2.79 (dd, J=17.0, 8.2 Hz, 0.8H, H—C(3a)), 2.64-2.51 (m, 0.2H, H—C(3a)), 2.29 (d, J=8.1 Hz, 1H, OH), 2.10-1.80 (m, 2.4H, H—C(3), H—C(5)), 1.65 (ddd, J=13.2, 9.1, 4.4 Hz, 0.8H, H—C(5)), 1.44-1.34 (m, 0.2H, H—C(3)), 1.22 (ddd, J=13.2, 8.1, 4.9 Hz, 0.8H, H—C(3)), 1.05 (s, 9H, (CH₃)₃—C—Si).

¹³C NMR (75 MHz, CDCl₃) δ 135.78, 135.74 (CH-arom), 133.96, 133.84 (C-arom), 129.78, 127.72 (CH-arom), 107.21, 106.50 (C(2)), 85.37, 81.76 (C(6a)), 78.11, 77.19 (C(4)), 73.03, 72.44 (C(6)), 55.30, 54.46 (MeO), 50.91, 49.67 (C(3a)), 41.13, 40.29 (C(3)), 38.16, 37.98 (C(5)), 26.96, 26.92 (CH₃)₃—C—Si), 19.07 (CH₃)₃—C—Si).

ESI⁺-HRMS m/z calcd for C₂₆H₃₅O₄Si ([M+H]⁺) 435.1962, found 435.1950.

### (3aR,4R,6R,6aS)-4-((tert-butyldiphenylsilyl)oxy)-2-methoxyhexahydro-2H-cyclopenta[b]furan-6-yl acetate (7)

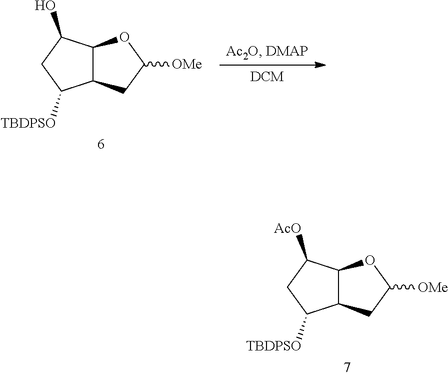

To a solution of sugar 6 (3.35 g, 8.12 mmol) and 4-dimethylaminopyridine (1.29 g, 10.6 mmol) in dry DCM (100 mL) is added acetic anhydride (3.8 mL, 41 mmol) at rt. After stirring for 2 h, the reaction is quenched by slow addition of saturated NaHCO₃(10 mL). The mixture is then diluted with saturated NaHCO₃(50 mL) and extracted with DCM (3×50 mL). The combined organic phases are dried over MgSO₄, filtered and evaporated. The crude product is purified by CC (EtOAc/hexane 1:2) to yield a mixture of 7 (3.53 g, 96%) in an anomeric ratio α/β≈4:1 as a colorless oil.

Data for 7: R_{f}=0.42 (EtOAc/hexane 1:2):

¹H NMR (400 MHz, CDCl₃) δ 7.70-7.59 (m, 4H, H-arom), 7.48-7.34 (m, 6H, H-arom), 5.41 (dt, J=11.0, 5.6 Hz, 0.8H, H—C(6)), 5.28 (ddd, J=11.7, 6.6, 5.2 Hz, 0.2H, H—C(6)), 4.99 (d, J=4.8 Hz, 0.8H, H—C(2)), 4.89-4.81 (m, 0.4H, H—C(2), H—C(6a)), 4.76-4.69 (m, 0.8H, H—C(6a)), 4.11 (d, J=5.1 Hz, 0.2H, H—C(4)), 3.90 (d, J=4.0 Hz, 0.8H, H—C(4)), 3.27, 3.24 (2s, 3H, MeO), 2.81 (dd, J=16.6, 7.6 Hz, 0.8H, H—C(3a)), 2.60 (dd, J=10.1, 7.0 Hz, 0.2H, H—C(3a)), 2.30-2.18 (m, 0.2H, H—C(5)), 2.12, 2.10 (2s, J=4.7 Hz, 3H, MeCO₂), 2.07-1.82 (m, 2.8H, H—C(5), H—C(3)), 1.24 (ddd, J=12.9, 7.6, 3.7 Hz, 1H, H—C(3)), 1.07 (s, 9H, (CH₃)₃—C—Si).

¹³C NMR (75 MHz, CDCl₃) δ 170.75, 170.66 (MeCO₂), 135.77, 135.73, 135.72 (CH-arom), 133.75, 133.65 (C-arom), 129.82, 129.74, 127.76, 127.75, 127.71 (CH-arom), 106.19, 106.15 (C(2)), 83.17, 79.80 (C(6a)), 77.49, 76.46 (C(4)), 75.64, 74.41 (C(6)), 54.34, 54.25 (MeO), 51.48, 50.17 (C(3a)), 38.05, 37.98 (C(3)), 36.96, 36.21 (C(5)), 26.95, 26.90 (CH₃)₃—C—Si), 21.09, 21.04 (MeCO₂), 19.04 (CH₃)₃—C—Si).

ESI⁺-HRMS m/z calcd for C₂₆H₃₄O₅NaSi ([M+Na]⁺) 477.2068, found 477.2063.

### (3aR,4R,6R,6aS)-4-((tert-butyldiphenylsilyl)oxy)-3a,5,6,6a-tetrahydro-4H-cyclopenta[b]furan-6-ol (8)

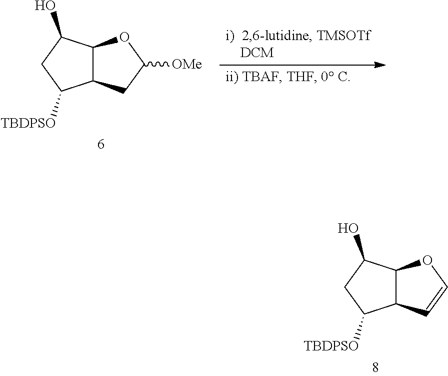

To a solution of the sugar 6 (2.08 g, 5.04 mmol) in dry DCM (35 mL) is added 2,6-lutidine (2.95 mL, 25.2 mmol) at 0° C. After stirring for 20 min at 0° C., TMSOTf (2.73 mL, 15.1 mmol) is added dropwise and then the solution is allowed to warm to rt and stirred for another 60 min. The reaction is then quenched by addition of saturated NaHCO₃(40 mL). The organic phase is separated and aqueous phase is further extracted with DCM (3×30 mL). The combined organic phases are dried over MgSO₄, filtered and evaporated.

The resulting product is dissolved in dry THF (35 mL), cooled down to 0° C., and TBAF (1M in THF, 5.6 mL, 5.6 mmol) is added. The solution is stirred for 10 min and then diluted with saturated NaHCO₃(30 mL) and extracted with DCM (4×40 mL). The combined organic phases are dried over MgSO₄, filtered and evaporated. The crude product is purified by CC (EtOAc/hexane 1:4) to yield the glycal 8 (1.76 g, 92%).

Data for 8: R_{f}=0.49 (EtOAc/hexane 1:2):

¹H NMR (300 MHz, CDCl₃) δ 7.66 (m, 4H, H-arom), 7.42 (m, 6H, H-arom), 6.22 (t, J=2.1 Hz, 1H, H—C(2)), 4.91 (dd, J=8.2, 5.3 Hz, 1H, H—C(3)), 4.70 (dt, J=11.1, 5.6 Hz, 1H, H—C(6)), 4.56 (t, J=2.8 Hz, 1H, H—C(6a)), 3.97 (d, J=4.0 Hz, 1H, H—C(4)), 3.24 (d, J=8.2 Hz, 1H, H—C(3a)), 2.30 (br, 1H, OH), 2.03 (dd, J=12.6, 5.4 Hz, 1H, H—C(5)), 1.51 (ddd, J=12.7, 11.2, 4.2 Hz, 1H, H—C(5)), 1.08 (s, 9H, (CH₃)₃—C—Si).

¹³C NMR (75 MHz, CDCl₃) δ 146.24 (C(2)), 135.72, 135.69 (CH-arom), 134.03, 133.74 (C-arom), 129.80, 129.78, 127.73 (CH-arom), 101.84 (C(3)), 84.59 (C(6a)), 76.79(C(4)), 74.10 (C(6)), 55.56 (C(3a)), 39.38 (C(5)), 26.93 (CH₃)₃—C—Si), 19.08 (CH₃)₃—C—Si).

ESI⁺-HRMS m/z calcd for C₂₃H₂₉O3Si ([M+H]⁺) 381.1880, found 381.1893.

### (((3aR,4R,6R,6aS)-6-(bis(4-methoxyphenyl)(phenyl)methoxy)-3a,5,6,6a-tetrahydro-4H-cyclopenta[b]furan-4-yl)oxy)(tert-butyl)diphenylsilane (9)

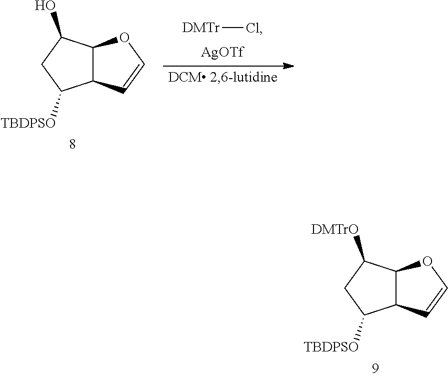

To a solution of glycal 8 (1.34 g, 3.52 mmol) and DMTr-Cl (1.43 g, 4.23 mmol) in a mixture of dry DCM (15 mL) and dry 2,6-lutidine (15 mL) is added portion wise silver triflate (1.13 g, 4.40 mmol), resulting in a deep red suspension. After stirring for 2 hours at rt, an additional portion of DMTr-Cl (239 mg, 0.705 mmol) is added. The suspension is further stirred for 2 hours and then is filtered. The organic phase is washed with saturated NaHCO₃ (100 mL) and the aqueous phases are extracted with DCM (3×30 mL). The combined organic phases are dried over MgSO₄, filtered and evaporated. The crude product is purified by CC (EtOAc/hexane 1:7, +0.5% Et₃N) to yield the protected glycal 9 (2.24, 93%) as a white foam.

Data for 9: R_{f}=0.59 (EtOAc/hexane 1:2):

¹H NMR (400 MHz, CDCl₃) δ 7.76 (d, J=7.4 Hz, 2H, H-arom), 7.69-7.60 (m, J=9.3, 5.9, 4.6 Hz, 8H, H-arom), 7.56-7.39 (m, 8H, H-arom), 7.33 (t, J=7.3 Hz, 1H, H-arom), 7.00-6.93 (m, 4H, H-arom), 6.47-6.37 (m, 1H, H—C(2)), 4.67-4.58 (m, 1H, H—C(6)), 4.58-4.50 (m, 2H, H—C(3), H—C(6a)), 3.86, 3.85 (2s, 6H, MeO), 3.82 (d, J=4.0 Hz, 1H, H—C(4)), 3.08 (d, J=8.1 Hz, 1H, H—C(3a)), 1.67 (td, J=12.4, 4.2 Hz, 1H, H—C(5)), 1.28 (dd, J=12.7, 5.4 Hz, 1H, H—C(5)), 1.11 (s, 9H, (CH₃)₃—C—Si).

¹³C NMR (75 MHz, CDCl₃) δ 158.67 (MeO—C-arom), 147.61 (C(2)), 146.26, 137.36, 137.21 (C-arom), 135.81, 135.78 (CH-arom), 134.17, 134.04 (C-arom), 130.48, 129.83, 129.81, 128.37, 127.98, 127.76, 127.73, 126.79, 113.32, 113.28 (CH-arom), 100.29 (C(3)), 86.96 (C(Ph)₃), 84.95 (C(6a)), 76.17 (C(6)), 76.07(C(4)), 55.26 (MeO-DMTr), 55.11 (C(3a)), 37.32 (C(5)), 27.04 (CH₃)₃—C—Si), 19.21 (CH₃)₃—C—Si).

ESI⁺-HRMS m/z calcd for C₄₄H₄₆O₅NaSi ([M+Na]⁺) 705.3007, found 705.3021.

### (3aS,4R,6R, 6aS)-6-(bis(4-methoxyphenyl) (phenyl)methoxy)-3a,5, 6, 6a-tetrahydro-4H-cyclopenta[b]furan-4-ol (10)

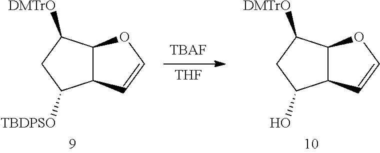

To a solution of glycal 9 (2.23 g, 3.27 mmol) in dry THF (20 mL) is added TBAF (1 M in THF, 20 mL, 20 mmol) at rt. The solution is stirred for 20 h and then is diluted with saturated NaHCO₃(100 mL) and extracted with DCM (3×80 mL). The combined organic phases are dried over MgSO₄, filtered and evaporated. The crude product is purified by CC (0.5% MeOH in DCM, +0.5% Et₃N) to yield 10 (1.45 g, quant.) as a white foam.

Data for 10: R_{f}=0.44 (EtOAc/hexane 1:1):

¹H NMR (300 MHz, CDCl₃) δ 7.53-7.46 (m, 2H, H-arom), 7.43-7.35 (m, 4H, H-arom), 7.21 (dd, J=10.7, 5.3 Hz, 2H, H-arom), 7.16-7.08 (m, 1H, H-arom), 6.80-6.71 (m, 4H, H-arom), 6.30 (t, J=2.1 Hz, 1H, H—C(2)), 4.68 (t, J=2.8 Hz, 1H, H—C(3)), 4.29-4.14 (m, 2H, H—C(6), H—C(6a)), 3.71 (s, 6H, MeO), 3.65 (d, J=3.5 Hz, 1H, H—C(4)), 2.87 (d, J=7.9 Hz, 1H, H—C(3a)), 1.59 (ddd, J=13.2, 11.6, 4.3 Hz, 1H, H—C(5)), 1.05-0.95 (m, 2H, H—C(5), OH).

¹³C NMR (75 MHz, CDCl₃) δ 158.54 (MeO—C-arom), 147.64 (C(2)), 145.82, 137.12, 137.08 (C-arom), 130.26, 128.29, 127.81, 126.71, 113.13 (CH-arom), 100.17 (C(3)), 86.75 (C(Ph)₃), 84.42 C(6a)), 75.54 (C(6)), 74.59 (C(4)), 55.22 (MeO-DMTr), 54.25 (C(3a)), 37.56 (C(5)).

ESI⁺-HRMS m/z calcd for C₃₀H₂₇O₅ ([M+H]⁺) 467.1853, found 467.1844.

### (3'S,5′R,7′R)-1-{2′,3′-Dideoxy-3′,5′-ethano-7′-hydroxy-5′-O-[(4,4′-dimethoxytriphenyl)methyl]-β-D-ribofuranosyl}thymine (11)

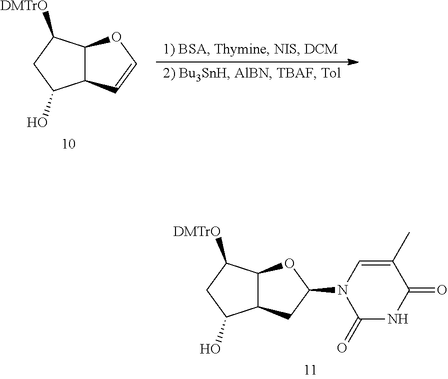

To a solution of glycal 10 (1.45 g, 3.27 mmol) in dry DCM (45 mL), at 0°, is added dropwise BSA (2.0 mL, 8.18 mmol) and then the solution is allowed to warm to rt. After stirring for 45 min, Thymine (595 mg, 4.91 mmol) is added and the reaction is further stirred for 60 min at rt. The mixture is then cooled down to 0° C. and N-iodosuccinimide (875 mg, 3.92 mmol) is added. After stirring for 3 h at 0° C. and for 4 h at rt, the reaction mixture is diluted with EtOAc (100 mL) and subsequently washed with a 10% aqueous solution of Na₂S₂O₃ (100 mL) and saturated NaHCO₃(100 mL). Aqueous phases are combined and extracted with DCM (3×50 mL). The combined organic phases are dried over MgSO₄, filtered and evaporated.

The crude product is dissolved in dry toluene (45 mL) and then Bu₃SnH (1.32 mL, 4.91 mmol) and azoisobutyronitrile (AIBN, 53 mg, 0.33 mmol) are added at rt. After heating at 70° C. for 30 min, the mixture is cool down to rt and TBAF is added (1 M in THF, 6.5 mL, 6.5 mmol). The solution is further stirred for 25 min and is diluted with saturated NaHCO₃(100 mL) and extracted with DCM (4×70 mL). The combined organic phases are dried over MgSO₄, filtered and evaporated. The crude product is purified by CC (3% MeOH in DCM, +0.5% Et₃N) to yield 11 (1.45 g, 73% over two steps) as a white foam.

Data for 11: R_{f}=0.29 (6% MeOH in DCM):

¹H NMR (400 MHz, CDCl₃) δ 9.37 (br, 1H, H—N(3)), 7.83 (d, J=1.1 Hz, 1H, H—C(6)), 7.58-7.52 (m, 2H, H-arom), 7.48-7.41 (m, 4H, H-arom), 7.28 (t, J=7.7 Hz, 2H, H-arom), 7.21 (t, J=7.2 Hz, 1H, H-arom), 6.84 (dd, J=8.9, 1.2 Hz, 4H, H-arom), 5.91 (dd, J=8.0, 5.5 Hz, 1H, H—C(1′)), 4.25 (dt, J=10.8, 6.0 Hz, 1H, H—C(5′)), 4.13-4.08 (m, 1H, H—C(4′)), 3.86 (d, J=3.4 Hz, 1H, H—C(7′), 3.79 (s, 6H, MeO), 2.70 (ddd, J=12.8, 10.2, 5.5 Hz, 1H, H—C(2′)), 2.61 (dd, J=16.9, 8.2 Hz, 1H, H—C(3′)), 1.84 (d, J=0.8 Hz, 3H, Me-C(5)), 1.80 (br, 1H, OH), 1.60 (ddd, J=14.2, 10.5, 4.2 Hz, 1H, H—C(6′)), 1.33 (dt, J=12.9, 8.0 Hz, 1H, H—C(2′)), 1.14 (dd, J=13.7, 6.1 Hz, 1H, H—C(6′)).

¹³C NMR (101 MHz, CDCl₃) δ 164.17 (C(4)), 158.64 (MeO—C-arom), 150.47 (C(2)), 145.65, 136.85, 136.71 (C-arom), 135.52 (C(6)), 130.20, 128.12, 127.91, 126.90, 113.22, 113.21 (CH-arom), 110.69 (C(5)), 87.21 (C(Ph)₃), 86.57 (C(1′)), 82.02 (C(4′)), 74.19 (C(5′)), 74.13 (C(7′)), 55.25 (MeO-DMTr), 49.40 (C(3′)), 38.51 (C(6′)), 37.64 (C(2′)), 12.58 (Me-C(5)).

ESI⁺-HRMS m/z calcd for C₃₃H₃₄O₇N₂Na ([M+Na]⁺) 593.2258, found 593.2250.

### (3′R,5′R,7′R)-1-{7′-O-[(2-Cyanoethoxy)-diisopropylaminophosphanyl]-2′,3′-dideoxy-3′,5′-ethano-5′-O-[(4,4′-dimethoxytriphenyl)methyl]-β-D-ribofuranosyl}thymine (12)

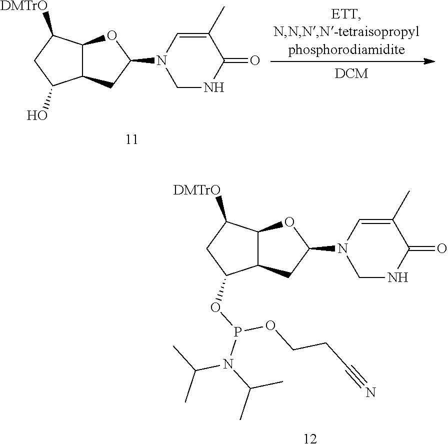

To a solution of the nucleoside 11 (232 mg, 0.406 mmol) and 5-(ethylthio)-1H-tetrazole (90 mg, 0.69 mmol) in dry DCM (10 mL) is added dropwise 2-cyanoethyl N,N,N′,N′-tetraisopropylphosphordiamidite (0.26 mL, 0.81 mmol) at rt. After stirring for 30 min, the reaction mixture is diluted with DCM (50 mL) and washed with saturated NaHCO₃ (2×30 mL) and satd NaCl (30 mL). Aqueous phases are combined and extracted with DCM (50 mL). The combined organic phases are dried over MgSO₄, filtered and evaporated. The crude product is purified by CC (1.8% MeOH in DCM, +0.5% Et₃N) to yield 12 (219 mg, mixture of two isomers, 70%) as a white foam.

Data for 11: R_{f}=0.68 (6% MeOH in DCM):

¹H NMR (300 MHz, CDCl₃) δ 8.93 (br, 1H, H—N(3)), 7.85 (d, J=1.2 Hz, 1H, H—C(6)), 7.65-7.52 (m, 2H, H-arom), 7.52-7.40 (m, 4H, H-arom), 7.40-7.21 (m, 3H, H-arom), 6.96-6.81 (m, 4H, H-arom), 6.00, 5.94 (2dd, J=8.3, 5.2 Hz, 1H, H—C(1′)), 4.29-4.17 (m, 1H, H—C(5′)), 4.12-3.89 (m, 2H, H—C(4′), H—C(7′)), 3.85, 3.84 (2s, 6H, MeO), 3.81-3.63 (m, 2H, OCH₂CH₂CN), 3.56-3.41 (m, 2H, (Me₂CH)₂N), 2.88-2.69 (m, 2H, H—C(3′), H—C(2′)), 2.61, 2.56 (dt, J=12.9 6.3 Hz, 2H, OCH₂CH₂CN), 1.92, 1.82 (2d, J=0.8 Hz, 3H, Me-C(5)), 1.75-1.56 (m, 1H, H—C(6′)), 1.52-1.36 (m, 2H, H—C(6′), H—C(2′)), 1.22-1.01 (m, 12H, (Me₂CH)₂N).

¹³C NMR (101 MHz, CDCl₃) δ 163.86 (C(4)), 158.66, 158.64 (MeO—C-arom), 150.29, 150.27 (C(2)), 145.58, 145.52, 136.76, 136.71, 136.69, 136.60 (C-arom), 135.49, 135.35 (C(6)), 130.21, 130.16, 128.17, 128.13, 127.88, 126.91, 126.89 (CH-arom), 117.49 (OCH₂CH₂CN), 113.18 (CH-arom), 110.74 (C(5)), 87.27, 87.25 (C(Ph)₃), 86.58, 86.45 (C(1′)), 81.79, 81.68 (C(4′)), 76.02, 75.50 (J_{C,P}=16.5, 15.7 Hz, C(7′)), 74.22 (C(5′)), 58.26, 58.06, 57.87 (OCH₂CH₂CN), 55.26, 55.22 (MeO-DMTr), 48.85, 48.62 (J_{C,P}=2.6, 5.0 Hz, C(3′)), 43.10, 43.04 (J_{C,P}=12.3, 12.4 Hz (Me₂CH)₂N), 37.78 (J_{C,P}=5.3 Hz C(6′)), 37.62, 37.48 (C(2′)), 37.41 (J_{C,P}=3.6 Hz C(6′)), 24.57, 24.53, 24.50, 24.46, 24.44, 24.39, 24.37 (Me₂CH)₂N), 20.35, 20.25 (J_{C,P}=7.1, 7.0 Hz, OCH₂CH₂CN), 12.58, 12.41 (7s, Me-C(5)).

³¹P NMR (122 MHz, CDCl₃) δ 147.32, 146.98.

ESI⁺-HRMS m/z calcd for C₄₂H₅₂O₈N₄P ([M+H]⁺) 771.3517, found 771.3512.

### (3'S,5′R,7′R)-M-Benzoyl-1-{2′,3′-dideoxy-3′,5′-ethano-7′-hydroxy-5′-O-[(4,4′-dimethoxytriphenyl)methyl]-β-D-ribofuranosyl}-5-methylcytosine (13)

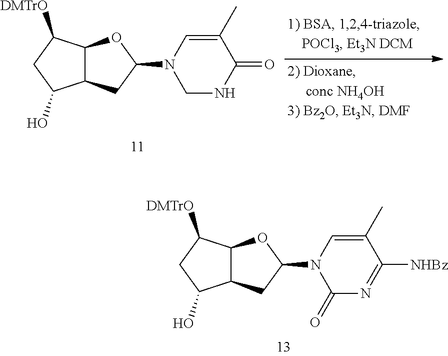

To a solution of the nucleoside 11 (302 mg, 0.530 mmol) in dry MeCN (5 mL) is added dropwise BSA (0.31 mL, 1.27 mmol) at 0°, and then the solution is stirred overnight at rt. In another flask, a suspension of 1,2,4-triazole (1.28 g, 18.55 mmol) in dry MeCN (50 mL) is cooled down to 0° C. and POCl₃ (0.40 mL, 4.2 mmol) and Et₃N (2.96 mL, 21.2 mmol) are added. The suspension is stirred for 30 min at 0° C., and then the previously prepared solution of the silylated compound 11 is added to the suspension and the mixture is further stirred for 5 h at rt. Reaction is quenched with the addition of saturated NaHCO₃(10 mL), MeCN is removed under reduced pressure and the resulting mixture diluted with saturated NaHCO₃ (35 mL) and extracted with DCM (3×40 mL). The combined organic phases are dried over MgSO₄, filtered and evaporated.

The crude product is then dissolved in a mixture of 1,4-dioxane (10 mL) and concentrated NH₄OH (10 mL). After stirring for 2 h at rt, the mixture is reduced to half of the volume in vacuo, diluted with saturated NaHCO₃(30 mL) and extracted with DCM (4×30 mL). The combined organic phases are dried over MgSO₄, filtered and evaporated.

The crude product is then dissolved in dry DMF (13 mL), Et₃N (90 μl, 0.64 mmol) followed by Bz₂O (300 mg, 1.33 mmol) are added at rt and the solution is stirred overnight. The resulting brown solution is quenched by careful addition of saturated NaHCO₃(50 mL) and extracted with DCM (4×50 mL). The combined organic phases are dried over MgSO₄, filtered and evaporated. The crude product is purified by CC (hexane/EtOAc 1:2, +0.5% Et₃N) to yield 13 (315 mg, 88%) as a white foam.

Data for 13: R_{f}=0.57 (4% MeOH in DCM):

¹H NMR (300 MHz, CDCl₃) δ 13.39 (br, 1H, NH), 8.46-8.26 (m, 2H, H-arom), 8.13 (d, J=0.5 Hz, 1H, C(6)), 7.61 (d, J=7.3 Hz, 2H, H-arom), 7.58-7.43 (m, 7H, H-arom), 7.34 (t, J=7.4 Hz, 2H, H-arom), 7.30-7.23 (m, 1H, H-arom), 6.89 (d, J=8.8 Hz, 4H, H-arom), 5.96 (dd, J=7.5, 5.8 Hz, 1H, H—C(1′)), 4.38-4.25 (m, 1H, H—C(5′)), 4.22-4.12 (m, 1H, H—C(4′)), 3.90 (d, J=3.6 Hz, 1H, H—C(7′)), 3.83 (s, 6H, MeO), 2.82 (ddd, J=13.3, 10.2, 5.7 Hz, 1H, H—C(2′)), 2.66 (dd, J=17.0, 8.1 Hz, 1H, H—C(3′)), 2.08 (s, 3H, Me-C(5)), 1.77 (br, 1H, OH), 1.71-1.57 (m, 1H, H—C(6′)), 1.49-1.36 (m, 1H, H—C(2′)), 1.21 (dd, J=13.7, 6.2 Hz, 1H, H—C(6′)).

¹³C NMR (75 MHz, CDCl₃) δ 179.56 (CONH), 160.01 (C(4)), 158.70 (MeO—C-arom), 147.96 (C(2)), 145.65 (C-arom), 137.26 (C(6)), 136.99, 136.83, 136.71 (C-arom), 132.41, 130.22, 129.89, 128.16, 128.14, 127.95, 126.94, 113.25 (CH-arom), 111.57 (C(5)), 87.34 (C(Ph)₃), 87.32 (C(1′)), 82.57 (C(4′)), 74.30 (C(5′)), 74.16 (C(7′)), 55.27 (MeO-DMTr), 49.56 (C(3′)), 38.52 (C(6′)), 38.00 (C(2′)), 13.63 (Me-C(5)).

ESI⁺-HRMS m/z calcd for C₄₀H₄₀O₇N₃ ([M+H]⁺) 674.2861, found 674.2862.

### (3′R,5′R,7′R)—N⁴-Benzoyl-1-{7′-O-[(2-cyanoethoxy)-diisopropylaminophosphanyl]-2′,3′-dideoxy-3′,5′-ethano-5′-O-[(4,4′-dimethoxytriphenyl)methyl]-β-D-ribofuranosyl}-5-methylcytosine (14)

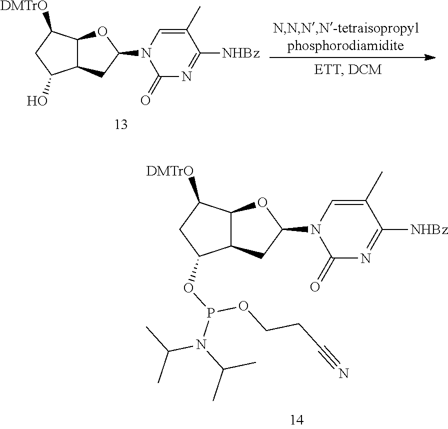

To a solution of the nucleoside 13 (276 mg, 0.409 mmol) and 5-(ethylthio)-1H-tetrazole (69 mg, 0.53 mmol) in dry DCM (10 mL) is added dropwise 2-cyanoethyl N,N,N′,N′-tetraisopropylphosphordiamidite (0.20 mL, 0.61 mmol) at rt. After stirring for 60 min, the reaction mixture is diluted with DCM (50 mL) and washed with saturated NaHCO₃(2×30 mL) and saturated NaCl (30 mL). Aqueous phases are combined and extracted with DCM (50 mL). The combined organic phases are dried over MgSO₄, filtered and evaporated. The crude product is purified by CC (EtOAc/hexane 2:3, +0.5% Et₃N) to yield 14 (268 mg, mixture of two isomers, 75%) as a white foam.

Data for 14: R_{f}=0.77 (5% MeOH in DCM):

¹H NMR (400 MHz, CDCl₃) δ 13.32 (s, 1H, NH), 8.41-8.28 (m, 2H, H-arom), 8.13-8.04 (m, 1H, C(6)), 7.61-7.51 (m, 3H, H-arom), 7.51-7.40 (m, 6H, H-arom), 7.37-7.29 (m, 2H, H-arom), 7.29-7.20 (m, 1H, H-arom), 6.92-6.82 (m, 4H, H-arom), 6.07-5.87 (m, 1H, H—C(1′)), 4.24 (dq, J=11.7, 5.8 Hz, 1H, H—C(5′)), 4.13-4.00 (m, 1H, H—C(4′)), 3.94 (ddd, J=14.5, 10.5, 2.8 Hz, 1H, H—C(7′)), 3.83, 3.82 (2s, 6H, MeO), 3.69 (m, 2H, OCH₂CH₂CN), 3.53-3.40 (m, 2H, (Me₂CH)₂N), 2.91-2.70 (m, 2H, H—C(2′), H—C(3′)), 2.57, 2.53 (2t, J=6.3 Hz, 2H, OCH₂CH₂CN), 2.08, 1.99 (2d, J=0.6 Hz, 3H, Me-C(5)), 1.72
1.56 (m, 1H, H—C(6′)), 1.54-1.36 (m, 2H, H—C(2′), H—C(6′)), 1.10 (m, 12H, (Me₂CH)₂N).

¹³C NMR (101 MHz, CDCl₃) δ 179.54 (CONH), 159.98 (C(4)), 158.69 (MeO—C-arom), 147.90 (C(2)), 145.58, 145.54 (C-arom), 137.30, 136.93 (C(6)), 136.81, 136.80, 136.73, 136.70, 136.67, 136.60 (C-arom), 132.37, 132.35, 130.22, 130.17, 129.89, 128.17, 128.15, 128.11, 127.93, 126.94 (CH-arom), 117.49 (OCH₂CH₂CN), 113.23 (CH-arom), 111.60 (C(5)), 87.36, 87.35 (C(Ph)₃), 87.33, 87.25 (C(1′)), 82.33, 82.25 (C(4′)), 76.05, 75.52 (J_{C,P}=16.4, 15.6 Hz, C(7′)), 74.32 (C(5′)), 58.18, 57.98 (J_{C,P}=19.5 Hz OCH₂CH₂CN), 55.28, 55.24 (MeO-DMTr), 48.93, 48.72 (J_{C,P}=2.7, 4.9 Hz, C(3′)), 43.11, 43.05 (J_{C,P}=12.4 Hz (Me₂CH)₂N), 38.02, 37.88 (C(2′)), 37.74, 37.40 (J_{C,P}=5.3, 3.4 Hz, C(6′)), 24.58, 24.54, 24.50, 24.47, 24.40, 24.38 (6s, Me₂CH)₂N), 20.36, 20.26 (J_{C,P}=7.1 Hz, OCH₂CH₂CN),), 13.66, 13.49 (Me-C(5)).

³¹P NMR (122 MHz, CDCl₃) δ 147.37, 147.07.

ESI⁺-HRMS m/z calcd for C₄₉H₅₇O₈N₅P ([M+H]⁺) 874.3939, found 874.3937.

### (3′R,5′R,7′R)—N⁶-Benzoyl-9-{5′-O-acetyl-7′-[(tert-butyldiphenylsilyl)oxy]-2′,3′-dideoxy-3′,5′-ethano-α,β-D-ribofuranosyl}adenine (15)

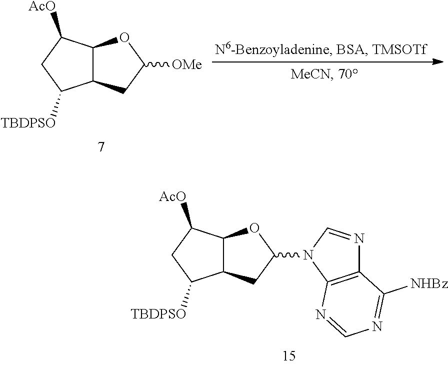

To a suspension of sugar 7 (1.86 g, 4.10 mmol) and N⁶-benzoyladenine (1.96 g, 8.20 mmol) in dry MeCN (40 mL) is added BSA (4.00 mL, 16.4 mmol) at rt. After stirring for 25 min, the suspension became a clear solution and then is heated to 70° C. TMSOTf (1.48 mL, 8.20 mmol) is added dropwise and the solution is further stirred for 20 min at 70° C. The solution is then cooled down to rt, quenched with addition of saturated NaHCO₃(100 mL) and extracted with EtOAc (4×50 mL). The combined organic phases are dried over MgSO₄, filtered and evaporated. The crude product is purified by CC (2% MeOH in DCM) to yield a mixture of 15 (1.74 g, 64%) in an anomeric ratio α/β≈4:1 as a white foam.

Data for 15: R_{f}=0.33 (EtOAc/hexane 4:1):

¹H NMR (400 MHz, CDCl₃) δ 9.33 (br, 1H, NH), 8.68 (d, J=5.4 Hz, 0.8H, H—C(2)), 8.64 (d, J=5.6 Hz, 0.2H, H—C(2)), 8.10 (d, J=1.5 Hz, 0.2H. H—C(8)), 7.99 (d, J=7.3 Hz, 2H, H-arom), 7.95 (s, 0.8H, H—C(8)), 7.63 (t, J=8.7 Hz, 4H, H-arom), 7.55 (dd, J=13.0, 6.4 Hz, 1H, H-arom), 7.50-7.34 (m, 8H, H-arom), 6.20 (dd, J=6.3, 2.5 Hz, 0.8H, H—C(1′)), 6.05 (t, J=6.5 Hz, 0.2H, H—C(1′)), 5.43-5.32 (m, 1H, H—C(5′)), 5.03-4.97 (m, 0.8H, H—C(4′)), 4.83 (t, J=6.0 Hz, 0.2H, H—C(4′)), 4.14 (br, 0.2H, H—C(7′)), 4.08 (d, J=3.7 Hz, 0.8H, H—C(7′)), 3.02 (dd, J=16.1, 6.6 Hz, 0.8H, H—C(3′)), 2.83 (dd, J=16.9, 7.7 Hz, 0.2H, H—C(3′)), 2.59-2.39 (m, 1H, H—C(2′)), 2.18-2.11 (m, 1H, H—C(6′)), 2.07 (d, J=1.6 Hz, 2.4H, MeCO₂), 2.02 (d, J=1.9 Hz, 0.6H, MeCO₂), 2.01-1.92 (m, 1H, H—C(6′)), 1.91-1.80 (m, 1H, H—C(3′)), 1.07 (s, 9H, (CH₃)₃—C—Si).

¹³C NMR (101 MHz, CDCl₃) δ 170.57, 170.49 (MeCO₂), 164.82 (CONH), 152.50 (C(2)), 151.27 (C(4)), 149.56 (C(6)), 141.37, 141.06 (C(8)), 135.72, 135.68, 135.66 (CH-arom), 133.67, 133.57, 133.24, 133.22 (C-arom), 132.73, 130.03, 129.98, 128.80, 128.78, 127.92, 127.86, 127.85 (CH-arom), 123.61 (C(5)), 87.19, 86.17 (C(1′)), 83.22, 80.96 (C(4′), 76.50, 76.04 (C(7′)), 74.38 (C(5′)), 51.07 (C(3′)), 37.29, 37.15, 36.80, 36.60 (C(2′), C(6′)), 26.89 (CH₃)₃—C—Si), 20.97, 20.90 (MeCO₂), 19.01 (CH₃)₃—C—Si).

ESI⁺-HRMS m/z calcd for C₃₇H₄₀O₅N₅Si ([M+H]⁺) 662.2793, found 662.2787.

### (3′R,5′R,7′R)—N⁶-Benzoyl-9-{7′-[(tert-butyldiphenylsilyl)oxy]-2′,3′-dideoxy-3′,5′-ethano-β-D-ribofuranosyl}adenine (16)

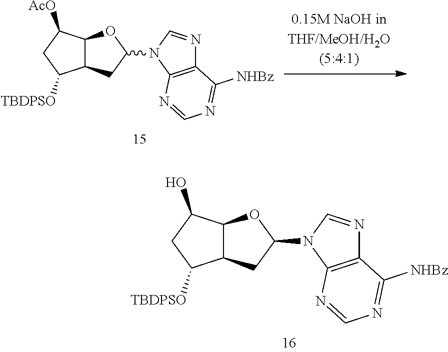

The nucleoside 15 (1.74 g, 2.64 mmol) is dissolved in 0.15 M NaOH in THF/methanol/H₂O (5:4:1, 80 mL) at 0° C. The reaction is stirred for 20 min and quenched by addition of NH₄C₁ (1.06 g). Solvents are then removed under reduced pressure and the product purified by CC (5% isopropanol in DCM) to yield 16 (287 mg, 18%) and its corresponding a anomer (836 mg, 51%) white foams.

Data for 16: R_{f}=0.44 (6% MeOH in DCM):

¹H NMR (300 MHz, CDCl₃) δ 8.70 (s, 1H, H—C(2)), 8.09-7.98 (m, 2H, H-arom), 7.97 (s, 1H, H—C(8)), 7.63 (ddd, J=7.4, 5.7, 1.5 Hz, 4H, H-arom), 7.59-7.55 (m, 1H, H-arom), 7.51 (m, 2H, H-arom), 7.44-7.33 (m, 6H, H-arom), 6.02 (dd, J=9.4, 5.5 Hz, 1H, H—C(1′)), 4.57 (dd, J=8.1, 5.0 Hz, 1H, H—C(4′)), 4.43 (dd, J=11.8, 5.3 Hz, 1H, H—C(5′)), 4.26 (br, 1H, H—C(7′)), 2.78 (q, J=8.9 Hz, 1H, H—C(3′)), 2.32-1.80 (m, 5H, H—C(2′), H—C(6′), OH), 1.06 (s, 9H, (CH₃)₃—C—Si). ¹³C NMR (101 MHz, CDCl₃) δ 164.85 (CONH), 152.56 (C(2)), 151.17 (C(4)), 149.86 (C(6)), 141.25 (C(8)), 135.68 (CH-arom), 133.87, 133.39 (C-arom), 132.78, 129.92, 128.78, 128.01, 127.78 (CH-arom), 123.51 (C(5)), 87.65 (C(1′)), 82.91 (C(4′)), 76.66 (C(7′)), 72.54 (C(5′)), 50.44 (C(3′)), 41.42 (C(6′)), 36.17 (C(2′)), 26.89 (CH₃)₃—C—Si), 19.03 (CH₃)₃—C—Si).

ESI⁺-HRMS m/z calcd for C₃₅H₃₈O₄N₅Si ([M+H]⁺) 620.2688, found 620.2671.

### (3′R,5′R,7′R)—N⁶-Benzoyl-9-{7′-[(tert-butyldiphenylsilyl)oxy]-2′,3′-dideoxy-3′,5′-ethano-5′-O-[(4,4′-dimethoxytriphenyl)methyl]-β-D-ribofuranosyl}adenine (17)

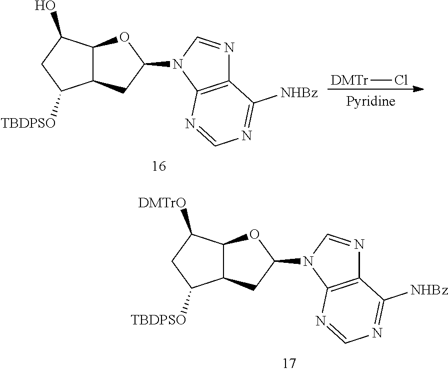

To a solution of nucleoside 16 (307 mg, 0.495 mmol) in dry pyridine (6 mL) is added DMTr-Cl (503 mg, 1.49 mmol) at rt. The solution is stirred for 1 day and then diluted with saturated NaHCO₃(50 mL) and extracted with DCM (3×70 mL). The combined organic phases are dried over MgSO₄, filtered and evaporated. The crude product is purified by CC (1.5% MeOH in DCM, +0.5% Et₃N) to yield 17 (395 mg, 87%) as a yellow foam.

Data for 17: R_{f}=0.65 (5% MeOH in DCM):

¹H NMR (300 MHz, MeOD) δ 8.64 (s, 1H, H—C(2)), 8.61 (s, 1H, H—C(8)), 8.08 (d, J=7.2 Hz, 2H, H-arom), 7.68-7.17 (m, 22H, H-arom), 6.86-6.75 (m, 4H, H-arom), 6.14 (dd, J=7.4, 6.3 Hz, 1H, H—C(1′)), 4.48-4.31 (m, 1H, H—C(5′)), 4.28-4.15 (m, 1H, H—C(4′)), 3.88 (d, J=3.8 Hz, 1H, H—C(7′)), 3.75, 3.74 (2s, 6H, MeO), 2.67 (dd, J=16.6, 6.7 Hz, 1H, H—C(3′)), 2.47 (ddd, J=13.3, 10.2, 6.1 Hz, 1H, H—C(2′)), 2.15-1.94 (m, 1H, H—C(6′)), 1.71 (ddd, J=13.0, 11.3, 4.4 Hz, 1H, H—C(2′)), 1.11 (dd, J=12.2, 4.9 Hz, 1H, H—C(6′)), 0.95 (s, 9H, (CH₃)₃—C—Si).

¹³C NMR (75 MHz, CDCl₃) δ 164.69 (CONH), 158.61, 158.60 (MeO—C-arom), 152.42 (C(2)), 151.27 (C(4)), 149.41 (C(6)), 145.81 (C-arom), 141.25 (C(8)), 137.00, 136.85 (C-arom), 135.60, 135.57 (CH-arom), 133.80, 133.69, 133.43 (C-arom), 132.70, 130.28, 130.25, 129.85, 129.81, 128.84, 128.18, 127.89, 127.71, 127.65, 126.78 (CH-arom), 123.52 (C(5)), 113.22, 113.19 (CH-arom), 87.09 (C(Ph)₃), 86.41 (C(1′)), 83.52 (C(4′)), 76.05 (C(7′)), 74.78 (C(5′)), 55.20 (MeO-DMTr), 50.43 (C(3′)), 38.10 (C(2′), C(6′)), 26.84 (CH₃)₃—C—Si), 19.00 (CH₃)₃—C—Si).

ESI⁺-HRMS m/z calcd for C₅₆H₅₆O₆N₅Si ([M+H]⁺) 922.3994, found 922.3953.

### (3'S,5′R,7′R)—N⁶-Benzoyl-9-{2′,3′-dideoxy-3′,5′-ethano-7′-hydroxy-5′-O-[(4,4′-dimethoxytriphenyl)methyl]-β-D-ribofuranosyl}adenine (18)

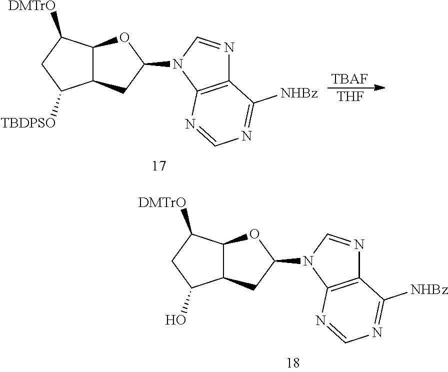

To a solution of nucleoside 17 (376 mg, 0.408 mmol) in dry THF (9 mL) is added TBAF (1 M in THF, 1.22 mL, 1.22 mmol) at rt. The solution is stirred for 2 days and is then diluted with saturated NaHCO₃(25 mL) and extracted with DCM (4×25 mL). The combined organic phases are dried over MgSO₄, filtered and evaporated. The crude product is purified by CC (4% MeOH in DCM, +0.5% Et₃N) to yield 18 (242 mg, 87%) as a white foam.

Data for 18: R_{f}=0.33 (5% MeOH in DCM):

¹H NMR (300 MHz, CD₃CN) 6 9.35 (br, 1H, NH), 8.67 (s, 1H, C(2)), 8.46 (s, 1H, C(8)), 8.01 (d, J=7.4 Hz, 2H, H-arom), 7.54 (m, 5H, H-arom), 7.35 (m, 4H, H-arom), 7.30-7.17 (m, 3H, H-arom), 6.84 (d, J=8.9 Hz, 4H, H-arom), 6.09 (dd, J=7.8, 6.2 Hz, 1H, H—C(1′)), 4.12 (dt, J=11.2, 5.8 Hz, 1H, C(5′)), 3.87-3.79 (m, 2H, C(4′), C(7′)), 3.75 (s, 6H, MeO), 2.83-2.64 (m, 2H, C(2′), OH), 2.58-2.46 (m, 1H, C(3′)), 2.21 (dd, J=13.9, 7.1 Hz, 1H, C(2′)), 1.92-1.82 (m, 1H, C(6′)), 1.29-1.17 (m, 1H, C(6′)).

¹³C NMR (75 MHz, CDCl₃) δ 165.03 (CONH), 158.57 (MeO—C-arom), 152.40 (C(2)), 151.23 (C(4)), 149.52 (C(6)), 145.68 (C-arom), 141.49 (C(8)), 136.86, 136.84, 133.77 (C-arom), 132.77, 130.22, 128.81, 128.16, 128.02, 127.89, 126.84 (CH-arom), 123.40 (C(5)), 113.19 (CH-arom), 87.06 (C(Ph)₃), 86.74 (C(1′)), 83.58 (C(4′)), 74.62 (C(5′)), 74.38 (C(8′)), 55.25 (MeO-DMTr), 49.77 (C(3′)), 38.55, 38.32 (C(6′), C(2′)).

ESI⁺-HRMS m/z calcd for C₄₀H₃₈O₆N₅ ([M+H]⁺) 684.2817, found 684.2830.

### (3′R,5′R,7′R)—N⁶-Benzoyl-9-{7′-O-[(2-cyanoethoxy)-diisopropylaminophosphanyl]-2′,3′-dideoxy-3′,5′-ethano-5′-O-[(4,4′-dimethoxytriphenyl)methyl]-β-D-ribofuranosyl}adenine (19)

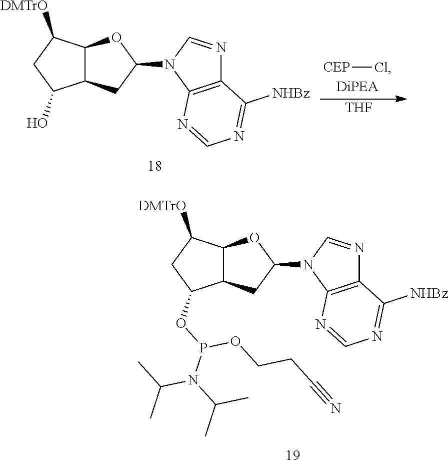

To a solution of the nucleoside 18 (173 mg, 0.253 mmol) and N,N-diisopropylethylamine (0.18 mL, 1.0 mmol) in dry THF (8 mL) is added N,N-diisopropylchlorophosphoramidite (0.11 mL, 0.50 mmol) at rt. The solution is stirred for 2 hours and then is diluted with saturated NaHCO₃(40 mL) and extracted with DCM (4×40 mL). The combined organic phases are dried over MgSO₄, filtered and evaporated. The crude product is purified by CC (EtOAc, +0.5% Et₃N) to yield 19 (177 mg, mixture of two isomers, 71%) as a white foam.

Data for 19: R_{f}=0.38, 0.44 (EtOAc):

¹H NMR (400 MHz, CDCl₃) δ 9.05 (br, 1H, NH), 8.70, 8.70 (2s, 1H, H—C(2)), 8.47, 8.46(2s, 1H, H—C(8)), 7.97 (d, J=7.5 Hz, 2H, H-arom), 7.57-7.50 (m, 1H, H-arom), 7.49-7.41 (m, 4H, H-arom), 7.39-7.31 (m, 4H, H-arom), 7.24-7.17 (m, 5.4 Hz, 2H, H-arom), 7.13 (dt, J=12.5, 6.2 Hz, 1H, H-arom), 6.83-6.70 (m, 4H, H-arom), 6.14-5.97 (m, 1H, H—C(1′)), 4.14 (ddd, J=11.1, 7.8, 3.4 Hz, 1H, H—C(5′)), 3.91-3.74 (m, 2H, H-(4′), H—C(7′)), 3.71, 3.70 (2s, 6H, MeO), 3.65-3.50 (m, 2H, OCH₂CH₂CN), 3.37 (ddq, J=13.9, 10.2, 6.8 Hz, 2H, (Me₂CH)₂N), 2.90-2.76 (m, 1H, H—C(2′)), 2.75-2.60 (m, 1H, H—C(3′)), 2.47, 2.42 (2t, J=6.3 Hz, 2H, OCH₂CH₂CN), 2.11 (dt, J=12.7, 6.1 Hz, 1H, H—C(2′)), 1.73 (ddt, J=13.6, 10.4, 5.1 Hz, 1H, H—C(6′)), 1.39 (ddd, J=50.2, 13.4, 6.2 Hz, 1H, H—C(6′)), 1.10-0.89 (m, 12H, (Me₂CH)₂N).

¹³C NMR (101 MHz, CDCl₃) δ 164.66 (CONH), 158.57 (MeO—C-arom), 152.46 (C(2)), 151.32, 151.26 (C(4)), 149.45, 149.43 (C(6)), 145.60, 145.59 (C-arom), 141.52, 141.47 (C(8)), 136.88, 136.83, 136.81, 133.78 (C-arom), 132.75, 132.73, 130.22, 130.21, 130.19, 130.17, 128.87, 128.17, 127.87, 126.82, 126.80 (CH-arom), 123.59 (C(5)), 117.53, 117.50 (OCH₂CH₂CN), 113.17 (CH-arom), 87.10, 87.07 (C(Ph)₃), 86.72, 86.68 (C(1′)), 83.36, 83.25 (C(4′)), 76.55, 75.81 (J_{C,P}=16.9, 15.7 Hz, C(7′)), 74.63, 74.60 (C(5′)), 58.24, 57.86 (J_{C,P}=19.1, 19.2 Hz OCH₂CH₂CN), 55.25, 55.21 (MeO-DMTr), 49.29, 49.08 (J_{C,P}=2.6, 4.7 Hz, C(3′)), 43.12, 43.00 (J_{C,P}=2.4, 2.3 Hz (Me₂CH)₂N), 38.27, 38.23 (C(2′)), 37.41, 37.22 (J_{C,P}=5.3, 3.5 Hz, C(6′)) 24.56, 24.53, 24.49, 24.47, 24.43, 24.41, 24.36, 24.33 (8s, Me₂CH)₂N), 20.36, 20.25 (J_{C,P}=7.2, 7.0 Hz, OCH₂CH₂CN).

³¹P NMR (122 MHz, CDCl₃) δ 147.64, 146.87.

ESI⁺-HRMS m/z calcd for C₄₉H₅₅O₇N₇ ([M+H]⁺) 884.3895, found 884.3898.

### (3′R,5′R,7′R)-2-Amino-6-chloro-9-{5′-O-acetyl-7′-[(tert-butyldiphenylsilyl)oxy]-2′,3′-dideoxy-3′,5′-ethano-α,β-D-ribofuranosyl}purine (20)

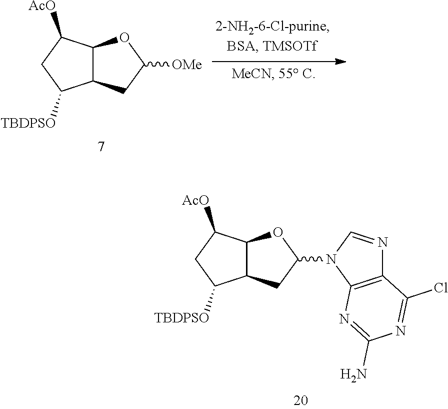

To a suspension of sugar 7 (1.75 g, 3.85 mmol) and 2-amino-6-chloropurine (1.05 g, 6.17 mmol) in dry MeCN (20 mL) is added BSA (3.80 mL, 15.4 mmol) at rt. The suspension is heated to 55° C. and stirred for 30 min. Then TMSOTf (1.05 mL, 5.78 mmol) is added dropwise and the solution is further stirred for 50 min at 55° C. The solution is cooled down to rt, quenched with addition of saturated NaHCO₃(10 mL), diluted with EtOAc (50 mL) and filtered through a short pad of SiO₂. The SiO₂ is washed with additional EtOAc. The mixture is then washed with saturated NaHCO₃(2×80 mL), aqueous phases are combined and extracted with EtOAc (3×50 mL). The combined organic phases are dried over MgSO₄, filtered and evaporated. The crude product is purified by CC (2.5% MeOH in DCM) to yield a mixture of 20 (1.77 g, 77%) in an anomeric ratio α/β≈7:3 as a white foam.

Data for 20: R_{f}=0.54 (EtOAc/hexane 5:1):

¹H NMR (300 MHz, CDCl₃) δ 7.86 (s, 0.3H, H—C(8)), 7.69 (s, 0.7H, H—C(8)), 7.68-7.60 (m, 4H, H-arom), 7.47-7.34 (m, 6H, H-arom), 6.04 (dd, J=6.9, 3.0 Hz, 0.7H, H—C(1′)), 5.87 (dd, J=8.0, 6.2 Hz, 0.3H, H—C(1′)), 5.37 (dt, J=14.2, 4.6 Hz, 1H, H—C(5′)), 5.16 (br, 2H, NH₂), 4.91 (dd, J=6.5, 5.1 Hz, 0.7H, H—C(4′)), 4.79 (dd, J=6.9, 5.2 Hz, 0.3H, H—C(4′)), 4.13 (br, 0.3H, H—C(7′)), 4.06 (d, J=4.0 Hz, 0.7H, H—C(7′)), 2.95 (dd, J=16.3, 6.6 Hz, 0.7H, H—C(3′)), 2.81 (dd, J=17.0, 7.4 Hz, 0.3H, H—C(3′)), 2.49-2.30 (m, 1H, H—C(2′)), 2.14 (dd, J=13.1, 6.7 Hz, 1H, H—C(6′)), 2.08 (s, 2.1H, MeCO₂), 2.02 (s, 0.9H, MeCO₂), 2.02-1.91 (m, 1H, H—C(6′)), 1.80 (td, J=13.4, 6.8 Hz, 1H, H—C(2′)), 1.07, 1.06 (2s, 9H, (CH₃)₃—C—Si).

¹³C NMR (75 MHz, CDCl₃) δ 170.55, 170.44 (MeCO₂), 158.98, 158.91 (C(2)), 153.18, 152.95 (C(4)), 151.40, 151.34 (C(6)), 140.38, 140.14 (C(8)), 135.73, 135.70 (CH-arom), 133.78, 133.62, 133.24, 133.17 (C-arom), 130.03, 130.00, 127.88, 127.86 (CH-arom), 125.65, 125.57 (C(5)), 86.59, 85.74 (C(1′)), 82.93, 80.99 (C(4′)), 76.57, 76.14 (C(7′)), 74.34, 74.32 (C(5′)), 51.15, 51.10 (C(3′)), 37.19, 36.99 (C(6′)), 36.70, 36.25 (C(2′)), 26.87 (CH₃)₃—C—Si), 20.95, 20.86 (MeCO₂), 19.00 (CH₃)₃—C—Si).

ESI⁺-HRMS m/z calcd for C₃₀H₃₅O₄N₅ClSi ([M+H]⁺) 592.2141, found 592.2158.

### (3′R,5′R,7′R)-2-Amino-6-chloro-9-{7-[(tert-butyldiphenylsilyl)oxy]-2′,3′-dideoxy-3′,5′-ethano-β-D-ribofuranosyl}purine (22b)

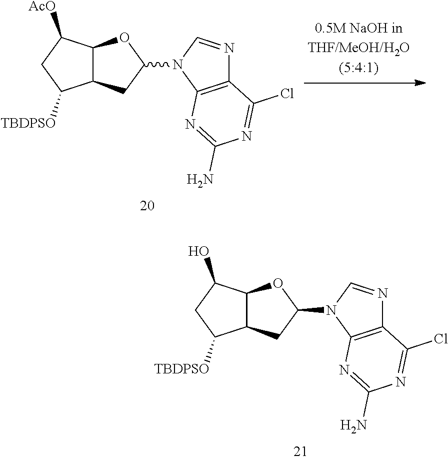

The nucleoside 20 (1.78 g, 3.01 mmol) is dissolved in 0.5 M NaOH in THF/methanol/H₂O (5:4:1, 15 mL) at 0° C. The reaction is stirred for 20 min at 0° C. and quenched by addition of NH₄Cl (484 mg). The suspension is then diluted with saturated NaHCO₃(100 mL) and extracted with DCM (4×75 mL). The combined organic phases are dried over MgSO₄, filtered and evaporated. The crude product is purified by CC (3% MeOH in DCM) to yield 21 (428 mg, 25%) and its corresponding a anomer (992 mg, 60%) as white foams.

Data for 21: R_{f}=0.43 (5% MeOH in DCM):

¹H NMR (300 MHz, CDCl₃) δ 7.71 (s, 1H, H—C(8)), 7.68-7.60 (m, 4H, H-arom), 7.44 7.33 (m, 6H, H-arom), 5.85 (dd, J=9.3, 5.8 Hz, 1H, H—C(1′)), 5.33 (br, 2H, NH₂), 4.62 (dd, J=8.4, 4.9 Hz, 1H, H—C(4′)), 4.44 (dd, J=10.7, 5.3 Hz, 1H, H—C(5′)), 4.40-4.15 (m, 2H, H—C(7′), OH), 2.79 (q, J=8.7 Hz, 1H, H—C(3′)), 2.22 (dd, J=15.2, 9.3 Hz, 1H, H—C(6′)), 2.11-2.02 (m, 1H, H—C(6′)), 2.02-1.85 (m, 2H, H—C(2′)), 1.06 (s, 9H, (CH₃)₃—C—Si).

¹³C NMR (75 MHz, CDCl₃) δ 158.73 (C(2)), 152.78 (C(4)), 151.94 (C(6)), 140.70 (C(8)), 135.70 (CH-arom), 133.91, 133.48 (C-arom), 129.90, 127.78 (CH-arom), 125.97 (C(5)), 87.96 (C(1′)), 82.88 (C(5′)), 76.85 (C(7′)), 72.36 (C(5′)), 50.41 (C(3′)), 41.96 (C(6′)), 35.73 (C(2′)), 26.90 (CH₃)₃—C—Si), 19.02 (CH₃)₃—C—Si).

ESI⁺-HRMS m/z calcd for C₂₈H₃₃O₃N₅ClSi ([M+H]⁺) 550.2036, found 550.2015.

### (3′R,5′R,7′R)—N²—(N,N-Dimethylformamidino)-9-{7-[(tert-butyldiphenylsilyl)oxy]-2′,3′-dideoxy-3′,5′-ethano-β-D-ribofuranosyl}guanine (22)

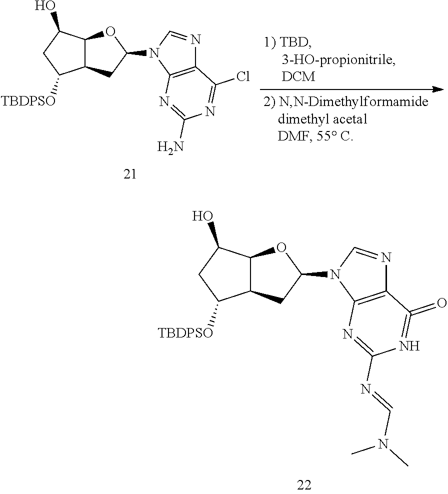

To a solution of 21 (380 mg, 0.645 mmol) and 3-hydroxypropionitrile (0.22 mL, 3.23 mmol) in dry DCM (15 mL) is added 1,5,7-triazabicyclo[4.4.0]dec-5-ene (400 mg, 2.87 mmol) at 0° C. The solution is stirred for 3 hours at 0° C. and then for 2 days at rt. Reaction is stopped by addition of silica. After evaporation of solvent, the SiO₂ powder is filtered, washed with MeOH and solvent evaporated to yield a brown foam.

The crude product is dissolved in dry DMF (5 mL) and N,N-dimethylformamide dimethyl acetal (0.43 mL, 3.2 mmol) is added. The solution is stirred for 2 hours at 55° C. and then the solvents are removed under reduced pressure. The crude product is purified by CC (6% MeOH in DCM) to yield 23 (274 mg, 73%) as a yellowish foam.

Data for 22: R_{f}=0.45 (12% MeOH in DCM):

¹H NMR (300 MHz, CDCl₃) δ 9.52 (s, 1H, NH), 8.46 (s, 1H, NCHN(CH₃)₂), 7.63 (dd, J=7.7, 1.5 Hz, 4H, H-arom), 7.50 (s, 1H, H—C(8)), 7.44-7.30 (m, 6H, H-arom), 5.83 (dd, J=9.3, 6.0 Hz, 1H, H—C(1′)), 4.61 (dd, J=8.7, 5.0 Hz, 1H, H—C(4′)), 4.43-4.32 (m, 1H, H—C(5′)), 4.29 (dd, J=7.0, 4.8 Hz, 1H, H—C(7′)), 3.95 (d, J=5.1 Hz, 1H, OH), 2.98 (s, 6H, NCHN(CH₃)₂), 2.79 (dd, J=18.0, 7.0 Hz, 1H, H—C(3′)), 2.20 (dt, J=12.8, 5.4 Hz, 1H, H—C(6′)), 2.09-1.88 (m, 3H, H—C(6′), H—C(2′)), 1.05 (s, 9H, (CH₃)₃—C—Si)).

¹³C NMR (75 MHz, CDCl₃) δ 158.73 (C(2)), 157.79 (C(6)), 156.91 (NCHN(CH₃)₂), 149.84 (C(4)), 137.00 (C(8)), 135.70, 135.67 (CH-arom), 133.78, 133.60 (C-arom), 129.93, 129.86, 127.78, 127.72 (CH-arom), 121.61 (C(5)), 88.04 (C(1′)), 82.21 (C(4′)), 77.49 (C(7′)), 71.94 (C(5′)), 50.13 (C(3′)), 42.23 (C(6′)), 41.20 (NCHN(CH₃)₂), 35.50 (C(2′)), 34.97 (NCHN(CH₃)₂), 26.87 (CH₃)₃—C—Si), 19.02 (CH₃)₃—C—Si).

ESI⁺-HRMS m/z calcd for C₃₁H₃₈O₄N₆Si ([M+H]⁺) 586.2718, found 586.2703.

### (3′R,5′R,7′R)—N²—(N,N-Dimethylformamidino)-9-{7-[(tert-butyldiphenylsilyl)oxy]-2′,3′-dideoxy-3′,5′-ethano-5′-O-[(4,4′-dimethoxytriphenyl)methyl]-β-D-ribofuranosyl}guanine (23)

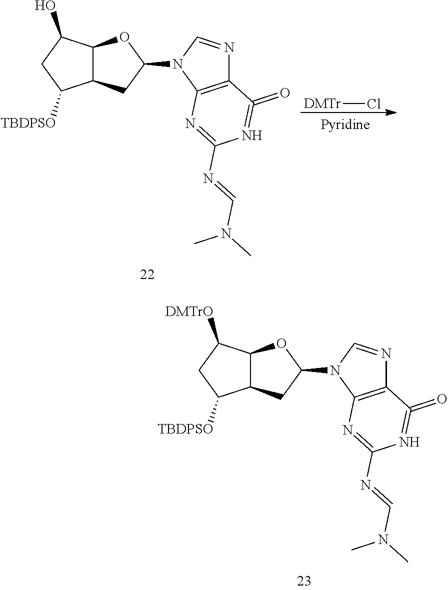

To a solution of 22 (139 mg, 0.237 mmol) in dry pyridine (2 mL) is added DMTr-Cl (240 mg, 0.708 mmol) in six portions over 3 hours at rt. After stirring overnight, the orange solution is diluted with saturated NaHCO₃(20 mL) and extracted with DCM (3×20 mL). The combined organic phases are dried over MgSO₄, filtered and evaporated. The crude product is purified by CC (4% MeOH in DCM, +0.5% Et₃N) to yield 23 (148 mg, 70%) as a yellowish foams.

Data for 23: R_{f}=0.52 (10% MeOH in DCM):

¹H NMR (400 MHz, CDCl₃) δ 9.49 (s, 1H, NH), 8.38 (s, 1H, NCHN(CH₃)₂), 7.80 (s, 1H, C(8)), 7.50-7.43 (m, 2H, H-arom), 7.42-7.27 (m, 10H, H-arom), 7.26-7.15 (m, 6H, H-arom), 7.14-7.08 (m, 1H, H-arom), 6.77-6.68 (m, 4H, H-arom), 5.78 (dd, J=8.2, 5.9 Hz, 1H, H—C(1′)), 4.25 (dt, J=11.0, 5.6 Hz, 1H, H—C(5′)), 4.14-4.03 (m, 1H, H—C(4′)), 3.70-3.64 (m, 7H, MeO, H—C(7′)), 3.00 (s, 3H, NCHN(CH₃)₂), 2.97 (s, 3H, NCHN(CH₃)₂), 2.43 (dd, J=16.7, 7.5 Hz, 1H, H—C(3′)), 2.24 (ddd, J=13.3, 10.1, 5.8 Hz, 1H, H—C(2′)), 1.62 (td, J=13.1, 4.3 Hz, 1H, H—C(6′)), 1.43 (dt, J=13.5, 8.0 Hz, 1H, H—C(2′)), 0.99 (dd, J=13.3, 6.2 Hz, 1H), 0.86 (s, 9H, (CH₃)₃—C—Si)).

¹³C NMR (101 MHz, CDCl₃) δ 158.51, 158.49 (MeO—C-arom), 158.04 (C(2)), 157.91 (C(6)), 156.60 (NCHN(CH₃)₂), 149.76 (C(4)), 145.83, 137.12, 136.94 (C-arom), 136.01 (C(8)), 135.60, 135.59 (CH-arom), 133.81, 133.47 (C-arom), 130.32, 130.26, 129.77, 128.24, 127.82, 127.65, 127.62, 126.67 (CH-arom), 120.65 (C(5)), 113.13, 113.09 (CH-arom), 86.82 (C(Ph)₃), 85.01 (C(1′)), 82.26 (C(4′)), 76.14 (C(7′)), 74.61 (C(5′)), 55.19 (MeO-DMTr), 50.18 (C(3′)), 41.29 (NCHN(CH₃)₂), 38.01 (C(6′)), 37.76 (C(2′)), 35.14 (NCHN(CH₃)₂) 26.81 87 (CH₃)₃—C—Si), 19.01 (CH₃)₃—C—Si).

ESI⁺-HRMS m/z calcd for C₅₂H₅₇O₆N₆Si ([M+H]⁺) 889.4103, found 889.4128.

### (3'S,5′R,7′R)—N²—(N,N-Dimethylformamidino)-9{2′,3′-dideoxy-3′,5′-ethano-7′-hydroxy-5′-O-[(4,4′-dimethoxytriphenyl)methyl]-β-D-ribofuranosyl}guanine (24)

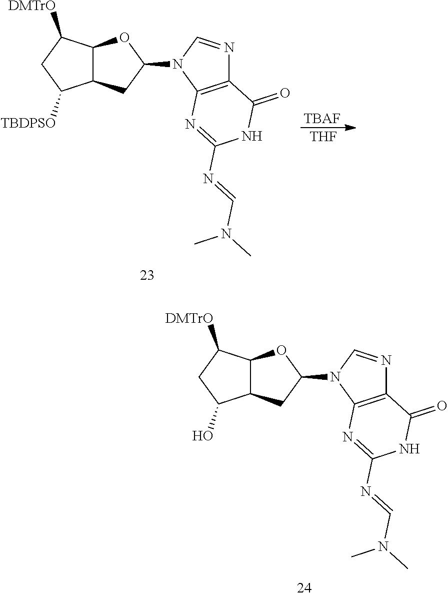

To a solution of 23 (243 mg, 0.273 mmol) in dry THF (2 mL) is added TBAF (1 M in THF, 1.65 mL, 1.63 mmol) at rt. The solution is stirred for 7 hours and then is diluted with saturated NaHCO₃(30 mL) and extracted with DCM (4×30 mL). The combined organic phases are dried over MgSO₄, filtered and evaporated. The crude product is purified by CC (7% MeOH in DCM, +0.5% Et₃N) to yield 24 (155 mg, 87%) as a white foam still containing traces of TBAF.

Data for 24: R_{f}=0.44 (10% MeOH in DCM):

¹H NMR (400 MHz, CDCl₃) δ 9.55 (s, 1H, NH), 8.45 (s, 1H, NCHN(CH₃)₂), 8.00 (s, 1H, H—C(8)), 7.60-7.50 (m, 2H, H-arom), 7.49-7.39 (m, 4H, H-arom), 7.31-7.23 (m, 2H, H-arom), 7.21-7.12 (m, 1H, H-arom), 6.81 (d, J=8.5 Hz, 4H, H-arom), 5.93 (dd, J=7.5, 6.1 Hz, 1H, H—C(1′)), 4.26 (dt, J=11.1, 5.8 Hz, 1H, H—C(5′)), 4.07-3.98 (m, 1H, H—C(4′)), 3.91 (d, J=4.3 Hz, 1H, H—C(7′)), 3.77 (s, 6H, MeO), 3.14 (s, 3H, NCHN(CH₃)₂), 3.04 (s, 3H, NCHN(CH₃)₂), 2.73 (ddd, J=13.3, 10.1, 6.0 Hz, 1H, H—C(2′)), 2.63-2.48 (m, 1H, H—C(3′)), 2.12 (br, 1H, OH), 1.95-1.82 (m, 2H, H—C(6′), H—C(2′)), 1.14 (dd, J=13.4, 6.1 Hz, 1H, H—C(6′)).

¹³C NMR (101 MHz, CDCl₃) δ 158.52 (MeO—C-arom), 158.12 (C(2)), 157.88 (C(6)), 156.65 (NCHN(CH₃)₂), 149.78 (C(4)), 145.69, 137.02, 136.99 (C-arom), 136.07 (C(8)), 130.26, 128.26, 127.82, 126.74 (CH-arom), 120.53 (C(5)), 113.12 (CH-arom), 86.81 (C(Ph)₃), 85.35 (C(1′)), 82.64 (C(4′)), 74.61 (C(7′)), 74.48 (C(5′)), 55.23 (MeO-DMTr), 49.63 (C(3′)), 41.37 (NCHN(CH₃)₂), 38.55 (C(6′)), 38.23 (C(2′)), 35.14 (NCHN(CH₃)₂).

ESI⁺-HRMS m/z calcd for C₃₆H₃₉O₆N₆ ([M+H]⁺) 651.2926, found 651.2912.

### (3′R,5′R,7′R)—N²—(N,N-Dimethylformamidino)-9{7′-O-[(2-cyanoethoxy)-diisopropylaminophosphanyl]-2′,3′-dideoxy-3′,5′-ethano-5′-O-[(4,4′-dimethoxytriphenyl)methyl]-β-D-ribofuranosyl}guanine (25)

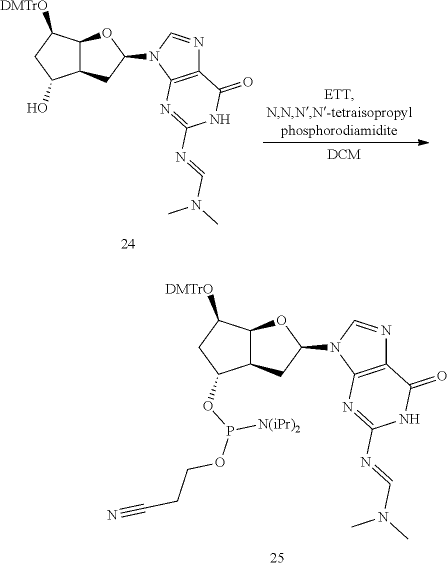

To a solution of the nucleoside 24 (143 mg, 0.220 mmol) and 5-(ethylthio)-1H-tetrazole (43 mg, 0.33 mmol) in dry DCM (10 mL) is added dropwise 2-cyanoethyl N,N,N′,N′-tetraisopropylphosphordiamidite (0.12 mL, 0.38 mmol) at rt. After stirring for 50 min, the reaction mixture is diluted with saturated NaHCO₃(20 mL) and extracted with DCM (3×20 mL). The combined organic phases are dried over MgSO₄, filtered and evaporated. The crude product is purified by CC (3.5% MeOH in DCM, +0.5% Et₃N) to yield 25 (130 mg, mixture of two isomers, 69%) as a white foam.

Data for 25: R_{f}=0.60 (10% MeOH in DCM):

¹H NMR (300 MHz, CDCl₃) δ 9.54, 9.47 (2s, 1H, NH), 8.54, 8.52 (2s, 1H, NCHN(CH₃)₂), 8.02. 8.00 (2s, 1H, H—C(8)), 7.58-7.49 (m, 2H, H-arom), 7.46-7.36 (m, 4H, H-arom), 7.25 (dd, J=11.0, 3.5 Hz, 2H, H-arom), 7.21-7.13 (m, 1H, H-arom), 6.80 (dd, J=8.8, 2.2 Hz, 4H, H-arom), 6.00-5.82 (m, 1H, H—C(1′)), 4.16 (dd, J=10.7, 5.4 Hz, 1H, H—C(5′)), 4.00-3.82 (m, 2H, H—C(4′), H—C(7′)), 3.77, 3.77 (2s, 6H, MeO), 3.62 (dt, J=12.2, 6.1 Hz, 2H, OCH₂CH₂CN), 3.51-3.33 (m, 2H, (Me₂CH)₂N), 3.15, 3.14 (2s, 3H, NCHN(CH₃)₂), 3.07 (s, 3H, NCHN(CH₃)₂), 2.85-2.61 (m, 2H, C(2′), C(3′)), 2.59-2.44 (m, 2H, OCH₂CH₂CN), 2.00-1.79 (m, 2H, H—(C2′), H—C(6′)), 1.53-1.26 (m, 1H, H—C(6′)), 1.10, 1.01 (2t, J=6.4 Hz, 12H, (Me₂CH)₂N).

¹³C NMR (101 MHz, CDCl₃) δ 158.50 (MeO—C-arom), 158.04, 158.00 (C(2)), 157.93 (C(6)), 156.61, 156.60 (NCHN(CH₃)₂), 149.73, 149.72 (C(4)), 145.62, 145.62, 136.97, 136.94 (C-arom), 136.14 (C(8)), 130.27, 130.24, 130.22, 128.26, 127.81, 126.73 (CH-arom), 120.81, 120.76 (C(5)), 117.67, 117.56 (OCH₂CH₂CN), 113.10 (CH-arom), 86.88, 86.85 (C(Ph)₃), 85.58, 85.37 (C(1′)), 82.41, 82.07 (C(4′)), 77.08, 76.01 (J_{C,P}=37.0, 15.1 Hz, C(7′)), 74.52, 74.46 (C(5′)), 58.19, 57.74 (J_{C,P}=18.9, 19.0 Hz OCH₂CH₂CN), 55.25, 55.21 (MeO-DMTr), 49.10, 48.83 (J_{C,P}=2.2, 4.8 Hz, C(3′)), 43.12, 43.00 ((Me₂CH)₂N), 41.34, 41.33 (NCHN(CH₃)₂), 38.48, 38.41 (C(2′)), 37.23, 36.92 (J_{C,P}=5.7, 3.3 Hz C(6′)), 35.17 ((Me₂CH)₂N), 24.56, 24.53, 24.48, 24.47, 24.43, 25.36, 24.35 (7s, Me₂CH)₂N), 20.39, 20.28 (J_{C,P}=7.1, 6.9 Hz, OCH₂CH₂CN).

³¹P NMR (122 MHz, CDCl₃) δ 147.69, 146.37.

ESI⁺-HRMS m/z calcd for C₄₅H₅₆O₇N₈P ([M+H]⁺) 851.4004, found 851.4018.

### (3′S,5′R,7′R)-1-{7′-[(tert-butyldiphenylsilyl)oxy]-2′,3′-dideoxy-3′,5′-ethano-β-D-ribofuranosyl}uracil (26)

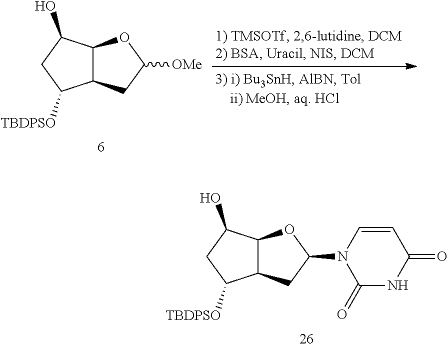

To a solution of the sugar 6 (669 mg, 1.62 mmol) in dry DCM (13 mL) is added 2,6-lutidine (0.94 mL, 8.10 mmol) at 0° C. After stirring for 20 min at 0° C., TMSOTf (0.89 mL, 4.86 mmol) is added dropwise and then the solution is allowed to warm to rt and is stirred for an additional 3 h. The reaction is then quenched by addition of saturated NaHCO₃(20 mL). The organic phase is separated and aqueous phase is further extracted with DCM (2×20 mL). The combined organic phases are dried over MgSO₄, filtered and evaporated.

The crude product is dissolved in dry DCM (12 mL) and then uracil (545 mg, 4.86 mmol) and BSA (1.8 mL, 7.29 mmol) are added at rt. After stirring for 60 min at rt, the resulting fine suspension is cooled down to 0° C. and N-iodosuccinimide (578 mg, 2.52 mmol) is added. After stirring for 30 min at 0° C. and for 4 h at rt, the reaction mixture is diluted with EtOAc (50 mL) and subsequently washed with a 10% aqueous solution of Na₂S₂O₃ (30 mL) and saturated NaHCO₃(30 mL). Aqueous phases are combined and extracted with DCM (2×20 mL). The combined organic phases are dried over MgSO₄, filtered and evaporated.

The crude product is dissolved in dry toluene (15 mL) and then Bu₃SnH (0.65 mL, 2.43 mmol) and azoisobutyronitrile (AIBN, 13 mg, 0.081 mmol) are added at rt. After heating at 95° C. for 2 h, the mixture is cooled down to rt and MeOH (7 mL) and HCl (1 M in water, 1.6 mL, 1.6 mmol) are added. The solution is further stirred for 15 min and is then diluted with saturated NaHCO₃(50 mL) and extracted with DCM (3×50 mL). The combined organic phases are dried over MgSO₄, filtered and evaporated. The crude product is purified by CC (EtOAc/hexane 4:1) to yield 26 (490 mg, 61% over three steps) as a white foam.

Data for 26: R_{f}=0.15 (EtOAc/hexane 2:1):

¹H NMR (300 MHz, CDCl₃) δ 9.95 (br, 1H, H—N(3)), 7.69 (d, J=6.4 Hz, 4H, H-arom), 7.54-7.39 (m, 7H, H—C(6), H-arom), 5.98 (dd, J=9.3, 5.6 Hz, 1H, H—C(1′)), 5.71 (d, J=8.1 Hz, 1H, H—C(5)), 4.51 (dd, J=13.7, 6.3 Hz, 2H, H—C(4′), H—C(5′)), 4.14 (br, 1H, H—C(7′)), 3.25 (br, 1H, OH), 2.74 (dd, J=17.1, 8.7 Hz, 1H, H—C(3′)), 2.26-1.87 (m, 3H, H—C(2′), H—C(6′)), 1.49-1.19 (m, 1H, H—C(2′)), 1.12 (s, 9H, (CH₃)₃—C—Si).

¹³C NMR (75 MHz, CDCl₃) δ 163.65 (C(4)), 150.46 (C(2)), 139.85 (C(6)), 135.69, 135.66 (CH-arom), 133.71, 133.42 (C-arom), 129.98, 129.93, 127.85, 127.81 (CH-arom), 102.84 (C(5)), 86.17 (C(1′)), 81.83 (C(4′)), 76.94 (C(7′)), 72.45 (C(5′)), 50.09 (C(3′)), 40.93 (C(6′)), 35.83 (C(2′)), 26.91 (CH₃)₃—C—Si), 19.03 (CH₃)₃—C—Si).

ESI⁺-HRMS m/z calcd for C₂₇H₃₂O₅N₂NaSi ([M+Na]⁺) 515.1973, found 515.1963.

### (3′S,5′R,7′R)-1-{7′-[(tert-butyldiphenylsilyl)oxy]-2′,3′-dideoxy-3′,5′-ethano-5′-O-[(4,4′-dimethoxytriphenyl)methyl]-β-D-ribofuranosyl}uracil (27)

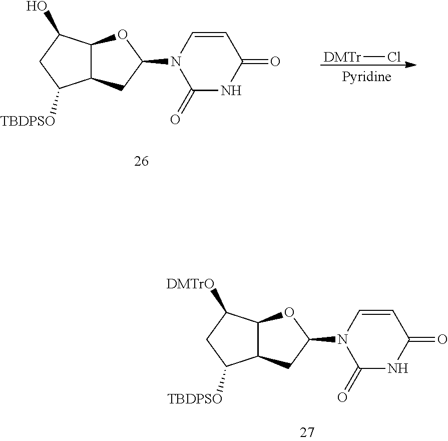

To a solution of nucleoside 26 (438 mg, 0.889 mmol) in dry pyridine (7 mL) is added DMTr-Cl (1.20 g, 3.55 mmol) at rt. The solution is stirred for 1 day at rt and then diluted with saturated NaHCO₃(30 mL) and extracted with DCM (3×40 mL). The combined organic phases are dried over MgSO₄, filtered and evaporated. The crude product is purified by CC (1.5% MeOH in DCM, +0.5% Et₃N) to yield 27 (601 mg, 80%) as a yellow foam.

Data for 27: R_{f}=0.48 (EtOAc/hexane 2:1):

¹H NMR (300 MHz, CDCl₃) δ 9.26 (br, 1H, H—N(3)), 7.84 (d, J=8.1 Hz, 1H, H—C(6)), 7.40-7.08 (m, 19H, H-arom), 6.69 (dd, J=8.8, 4.9 Hz, 4H, H-arom), 5.70 (dd, J=7.8, 5.8 Hz, 1H, H—C(1′)), 5.49 (dd, J=8.1, 1.5 Hz, 1H, H—C(5)), 4.24-4.11 (m, 1H, H—C(5′)), 4.05 3.95 (m, 1H, H—C(4′)), 3.65 (d, J=1.7 Hz, 6H, MeO), 3.62 (d, J=3.0 Hz, 1H, H—C(7′)), 2.41 (dd, J=17.2, 8.5 Hz, 1H, H—C(3′)), 2.24 (ddd, J=13.5, 10.2, 5.7 Hz, 1H, H—C(2′)), 1.39 1.24 (m, 1H, H—C(6′)), 1.04 (dd, J=13.1, 5.7 Hz, 1H, H—C(6′)), 0.89 (dt, J=13.8, 8.3 Hz, 1H, H—C(2′)), 0.81 (s, 9H, (CH₃)₃—C—Si).

¹³C NMR (75 MHz, CDCl₃) δ 163.58 (C(4)), 158.66 (MeO—C-arom), 150.38 (C(2)), 145.61 (C-arom), 139.92 (C(6)), 136.71, 136.56(C-arom), 135.61, 135.55 (CH-arom), 133.55, 133.41 (C-arom), 130.30, 129.92, 129.84, 128.16, 127.90, 127.74, 127.67, 126.90, 113.19, 113.15 (CH-arom), 102.12 (C(5)), 87.41 (C(Ph)₃), 86.80 (C(1′)), 82.32 (C4′)), 75.54 (C(7′)), 74.41 (C(5′)), 55.23 (MeO-DMTr), 50.05 (C(3′)), 38.49 (C(6′)), 37.53 (C(2′)), 26.81 (CH₃)₃—C—Si), 18.99 (CH₃)₃—C—Si).

ESI⁺-HRMS m/z calcd for C₄₈H₅₀O₇N₂NaSi ([M+Na]⁺) 817.3279, found 817.3286.

### (3'S,5′R,7′R)-1-{7′-[(tert-butyldiphenylsilyl)oxy]-2′,3′-dideoxy-3′,5′-ethano-5′-O-[(4,4′-dimethoxytriphenyl)methyl]-β-D-ribofuranosyl}cytosine (28)

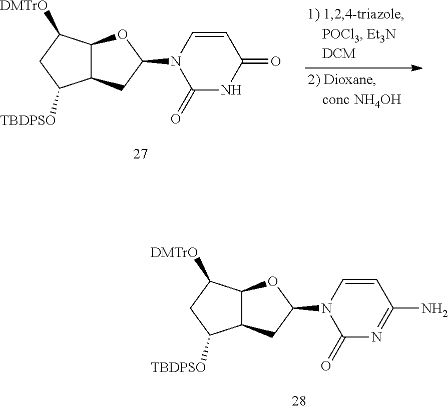

To a suspension of 1,2,4-triazole (1.83 g, 26.5 mmol) in dry MeCN (70 mL), at 0° C., are added POCl₃ (0.57 mL, 6.05 mmol) followed by Et₃N (4.2 mL, 30.2 mmol). The suspension is stirred for 30 min at 0° C. and then a solution of the nucleoside 27 (601 mg, 0.756 mmol) in dry MeCN (4 mL) is added at 0° C. After for 4 h of stirring at rt, the reaction is quenched with addition saturated NaHCO₃(20 mL), MeCN removed under reduced pressure and the resulting mixture diluted with saturated NaHCO₃(30 mL) and extracted with DCM (3×60 mL). The combined organic phases are dried over MgSO₄, filtered and evaporated.

The crude product is then dissolved in a mixture of 1,4-dioxane (18 mL) and concentrated NH₄OH (18 mL). After stirring for 3 h at rt, the mixture is reduced to half of the volume in vacuo, diluted with saturated NaHCO₃(30 mL) and extracted with DCM (3×30 mL). The combined organic phases are dried over MgSO₄, filtered and evaporated. The crude product is purified by CC (5% MeOH in DCM, +0.5% Et₃N) to yield 28 (520 mg, 87%) as a white foam.

Data for 28: R_{f}=0.41 (10% MeOH in DCM):

¹H NMR (300 MHz, CDCl₃) δ 7.96 (d, J=7.4 Hz, 1H, H—C(6)), 7.45 (d, J=7.4 Hz, 2H, H-arom), 7.38-7.08 (m, 17H, H-arom), 6.73 (dd, J=8.7, 4.7 Hz, 4H, H-arom), 5.73 (t, J=8.6 Hz, 2H, H—C(5), H—C(1′)), 4.32-4.16 (m, 1H, H—C(5′)), 4.03 (t, J=5.6 Hz, 1H, H—C(4′)), 3.66 (d, J=0.9 Hz, 6H, MeO), 3.61 (d, J=2.9 Hz, 1H, H—C(7′)), 2.50-2.33 (m, 2H, H—C(2′), H—C(3′)), 1.47-1.28 (m, 1H, H—C(6′)), 1.03 (dd, J=12.9, 5.6 Hz, 1H, H—C(6′)), 0.92-0.75 (m, 10H, H—C(2′), (CH₃)₃—C—Si).

¹³C NMR (75 MHz, CDCl₃) δ 165.78 (C(4)), 158.59 (MeO—C-arom), 155.94 (C(2)), 145.88 (C-arom), 140.68 (C(6)), 136.93, 136.78 (C-arom), 135.59, 135.53 (CH-arom), 133.60, 133.54 (C-arom), 130.31, 129.86, 129.77, 128.15, 127.88, 127.71, 127.64, 126.79, 113.18, 113.14 (CH-arom), 94.53 (C(5)), 87.55 (C(Ph)₃), 87.22 (C(1′)), 82.23 (C(4′)), 75.76 (C(7′)), 74.68 (C(5′)), 55.21 (MeO-DMTr), 50.18 (C(3′)), 38.25 (C(6′)), 38.08 (C(2′)), 26.83 (CH₃)₃—C—Si), 19.00 (CH₃)₃—C—Si).

ESI⁺-HRMS m/z calcd for C₄₈H₅₂O₆N₃Si ([M+H]⁺) 794.3620, found 794.3649.

### (3′S,5′R,7′R)— N⁴-Benzoyl-1-{7-[(tert-butyldiphenylsilyl)oxy]-2′,3′-dideoxy-3′,5′-ethano-5′-O-[(4,4′-dimethoxytriphenyl)methyl]-β-D-ribofuranosyl}cytosine (29)

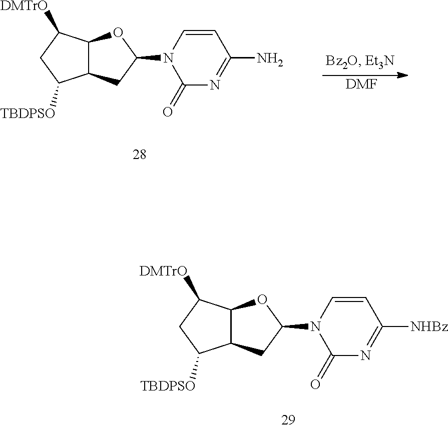

To a solution of nucleoside 28 (519 mg, 0.653 mmol) in dry DMF (15 mL) are added Et₃N (110 μL, 0.784 mmol) followed by Bz₂O (370 mg, 1633 mmol) at rt and the solution is stirred overnight. Then the solution is quenched by careful addition of saturated NaHCO₃(60 mL) and extracted with DCM (3×70 mL). The combined organic phases are dried over MgSO₄, filtered and evaporated. The crude product is purified by CC (hexane/EtOAc 2:3, +0.5% Et₃N) to yield 29 (580 mg, 99%) as a white foam.

Data for 29: R_{f}=0.51 (EtOAc):

¹H NMR (300 MHz, CDCl₃) δ 8.61 (d, J=7.4 Hz, 1H, H—C(6)), 7.81 (d, J=7.5 Hz, 2H, H-arom), 7.49-7.13 (m, 24H, H-arom, H—C(5)), 6.77 (dd, J=8.5, 4.4 Hz, 4H, H-arom), 5.73 (t, J=6.4 Hz, 1H, H—C(1′)), 4.39-4.20 (m, 1H, H—C(5′)), 4.05 (t, J=6.1 Hz, 1H, H—C(4′)), 3.70 (s, 6H, MeO), 3.63 (d, J=2.3 Hz, 1H, H—C(7′)), 2.72-2.55 (m, 1H, H—C(2′)), 2.48 (dd, J=16.0, 8.4 Hz, 1H, H—C(3′)), 1.42-1.29 (m, 1H, H—C(6′)), 1.19-1.11 (m, 1H, H—C(6′)), 1.07-0.96 (m, 1H, H—C(2′)), 0.85 (s, 9H, (CH₃)₃—C—Si).

¹³C NMR (75 MHz, CDCl₃) δ 166.64 (CONH), 162.25 (C(4)), 158.70 (MeO—C-arom), 154.84 (C(2)), 145.71 (C-arom), 144.84 (C(6)), 136.74, 136.67 (C-arom), 135.59, 135.51 (CH-arom), 133.52, 133.42, 133.24 (C-arom), 133.11, 130.30, 129.92, 129.85, 129.02, 128.12, 127.97, 127.76, 127.68, 127.61, 126.94, 113.25, 113.22(CH-arom), 96.22 (C(5)), 89.07 (C(Ph)₃), 87.53 (C(1′)), 83.46 (C(4′)), 75.59 (C(7′)), 74.71 (C(5′)), 55.24 (MeO— DMTr), 50.35 (C(3′)), 38.61 (C(6′)), 38.15 (C(2′)), 26.82 (CH₃)₃—C—Si), 19.00 (CH₃)₃—C—Si).

ESI⁺-HRMS m/z calcd for C₅₅H₅₆O₇N₃Si ([M+H]⁺) 898.3882, found 898.3898.

### (3′S,5′R,7′R)— N⁴-Benzoyl-1-{-2′,3′-dideoxy-3′,5′-ethano-7′-hydroxy-5′-O-[(4,4′-dimethoxytriphenyl)methyl]-β-D-ribofuranosyl}cytosine (30)

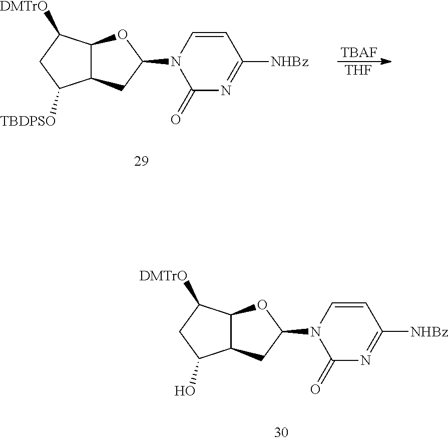

To a solution of 29 (580 mg, 0.648 mmol) in dry THF (14 mL) is added TBAF (1 M in THF, 3.25 mL, 3.25 mmol) at rt. The solution is stirred for 1 day and then is diluted with saturated NaHCO₃(50 mL) and extracted with DCM (3×40 mL). The combined organic phases are dried over MgSO₄, filtered and evaporated. The crude product is purified by CC (3% MeOH in DCM, +0.5% Et₃N) to yield 30 (366 mg, 85%) as a white foam.

Data for 30: R_{f}=0.31 (5% MeOH in DCM):

¹H NMR (300 MHz, CDCl₃) δ 8.90 (br, 1H, NH), 8.73 (d, J=7.5 Hz, 1H, H—C(6)), 7.82 (d, J=7.3 Hz, 2H, H-arom), 7.55-7.31 (m, 10H, H-arom, H—C(5)), 7.28-7.09 (m, 3H, H-arom), 6.76 (dd, J=8.8, 1.7 Hz, 4H, H-arom), 5.73 (t, J=6.3 Hz, 1H, H—C(1′)), 4.28-4.13 (m, 1H, H—C(5′)), 3.83 (t, J=6.0 Hz, 1H, H—C(4′)), 3.75 (d, J=3.6 Hz, 1H, H—C(7′)), 3.70 (s, 6H, MeO), 2.86 (d, J=14.7 Hz, 1H, H—C-(2′)), 2.54 (dd, J=17.4, 7.4 Hz, 1H, H—C(3′)), 1.68-1.55 (m, 1H, H—C(6′)), 1.45-1.13 (m, 3H, H—C(2′), H—C(6′), OH).

¹³C NMR (75 MHz, CDCl₃) δ 166.63 (CONH), 162.34 (C(4)), 158.65 (MeO—C-arom), 155.00 (C(2)), 145.62 (C-arom), 145.11 (C(6)), 136.72, 136.64, 133.16 (C-arom), 130.25, 129.02, 128.12, 127.93, 127.61, 126.95, 113.20 (CH-arom), 96.24 (C(5)), 89.20 (C(Ph)₃), 87.48 (C(1′)), 83.40 (C(4′)), 74.50, (C(5′)) 73.90 (C(7′)), 55.25 (MeO-DMTr), 50.05 (C(3′)), 38.90 (C(6′)), 38.40 (C(2′)).

ESI⁺-HRMS m/z calcd for C₃₉H₃₈O₇N₃ ([M+H]⁺) 660.2704, found 660.2707.

### (3′S,5′R,7′R)— N⁴-Benzoyl-1-{7′-O-[(2-cyanoethoxy)-diisopropylaminophosphanyl]-2′,3′-dideoxy-3′,5′-ethano-5′-O-[(4,4′-dimethoxytriphenyl)methyl]-β-D-ribofuranosyl}cytosine (31)

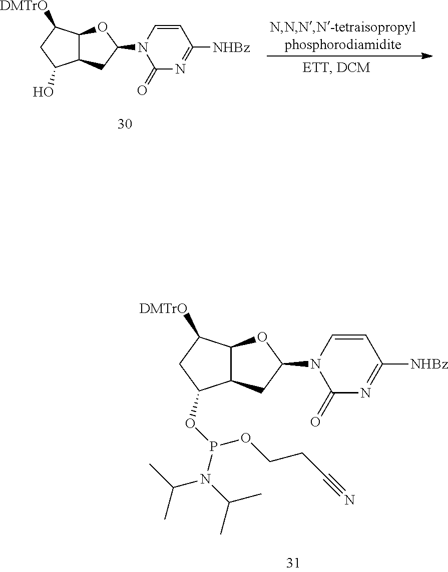

To a solution of the nucleoside 30 (67 mg, 0.101 mmol) and 5-(ethylthio)-1H-tetrazole (22 mg, 0.17 mmol) in dry DCM (3 mL) is added dropwise 2-cyanoethyl N,N,N′,N′-tetraisopropylphosphordiamidite (65 μL, 0.20 mmol) at rt. After stirring for 40 min, the reaction mixture is diluted with DCM (20 mL) and washed with saturated NaHCO₃(2×15 mL) and saturated NaCl (15 mL). Aqueous phases are combined and extracted with DCM (20 mL). The combined organic phases are dried over MgSO₄, filtered and evaporated. The crude product is purified by CC (EtOAc, +0.5% Et₃N) to yield 31 (75 mg, mixture of two isomers, 86%) as a white foam.

Data for 31: R_{f}=0.67 (4% MeOH in DCM):

¹H NMR (300 MHz, CDCl₃) δ 8.88 (s, 1H, NH), 8.79 (d, J=7.5 Hz, 1H, H—C(6)), 7.93 (d, J=7.5 Hz, 2H, H-arom), 7.67-7.40 (m, 10H, H-arom, H—C(5)), 7.39-7.22 (m, 3H, H-arom), 6.93-6.79 (m, 4H, H-arom), 5.97-5.77 (m, 1H, H—C(1′)), 4.22 (dt, J=14.5, 5.6 Hz, 1H, H-(5′)), 3.98-3.84 (m, 2H, H—C(4′), H—C(7′)), 3.82 (s, 6H, MeO), 3.66 (ddd, J=16.8, 13.5, 6.7 Hz, 2H, OCH₂CH₂CN), 3.53-3.37 (m, 2H, (Me₂CH)₂N), 3.14-2.93 (m, 1H, H—C(2′)), 2.84-2.66 (m, 1H, H—C(3′)), 2.53 (dt, J=12.4, 6.3 Hz, 2H, OCH₂CH₂CN), 1.83-1.56 (m, 2H, H—C(6′)), 1.46 (td, J=14.1, 7.0 Hz, 1H, H—C(2′)), 1.18-0.97 (m, 12H, (Me₂CH)₂N).

¹³C NMR (75 MHz, CDCl₃) δ 166.70 (CONH), 162.32, 162.28 (C(4)), 158.68 (MeO—C-arom), 154.93 (C(2)), 145.53 (C-arom), 144.95, 144.89 (C(6)), 136.69, 136.63, 136.56, 136.52, 133.24 (C-arom), 133.10, 130.24, 130.20, 129.01, 128.10, 127.94, 127.60, 126.96 (CH-arom), 117.53 (OCH₂CH₂CN), 113.20 (CH-arom), 96.24 (C(5)), 89.15, 89.10 (C(Ph)₃), 87.55, 87.54 (C(1′)), 83.11, 83.04 (C(4′)), 75.93, 75.37 (J_{C,P}=16.7, 15.5 Hz, C(7′)), 74.48 (C(5′)), 58.25, 57.99 (J_{C,P}=17.9, 18.1 Hz OCH₂CH₂CN), 55.27, 55.24 (MeO-DMTr), 49.27, 49.03 (J_{C,P}=3.1, 4.8 Hz, C(3′)), 43.15, 42.98 ((Me₂CH)₂N), 38.89, 38.80 (C(2′)), 37.44, 37.24 (J_{C,P}=5.2, 3.2 Hz, C(6′)), 24.58, 24.54, 24.48, 24.45, 24.35 (5s, Me₂CH)₂N), 20.33, 20.24 (J_{C,P}=5.8, 5.7 Hz, OCH₂CH₂CN).

³¹P NMR (121 MHz, CDCl₃) δ 147.19, 146.94.

ESI⁺-HRMS m/z calcd for C₄₈H₅₅O₈N₅P ([M+H]⁺) 860.3783, found 860.3791.

### (3′S, 5′R,7′R)-1-{2′,3′-dideoxy-3′,5′-ethano-7′-O-(4-nitrobenzoyl)-5′-O-[(4,4′-dimethoxytriphenyl)methyl]-β-D-ribofuranosyl}thymine (32)

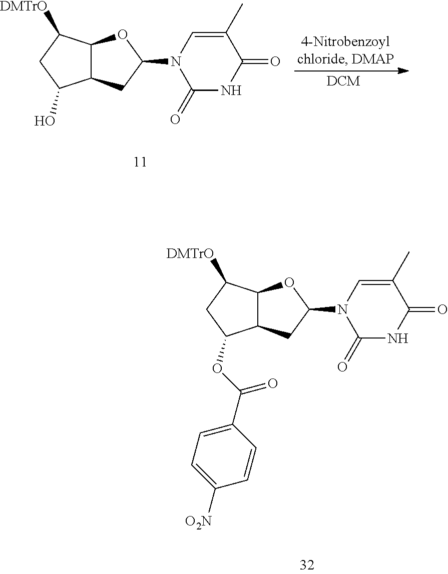

To a solution of nucleoside 11 (100 mg, 0.175 mmol) and 4-dimethylaminopyridine (26 mg, 0.21 mmol) in dry DCM (8 mL) is added 4-nitrobenzoyl chloride (59 mg, 0.315 mmol) at rt. After stirring for 6 h, the reaction is quenched by addition of saturated NaHCO₃(5 mL). The mixture is then diluted with saturated NaHCO₃(15 mL) and extracted with DCM (3×15 mL). The combined organic phases are dried over MgSO₄, filtered and evaporated. The crude product is purified by CC (2.5% MeOH in DCM, +0.5% Et₃N) to yield 32 (98 mg, 78%) as a white foam, containing traces of Et₃N.

Data for 32: R_{f}=0.42 (5% MeOH in DCM):

¹H NMR (300 MHz, CDCl₃) δ 8.26 (t, J=7.3 Hz, 3H, H-arom, HN(3)), 8.00 (d, J=8.9 Hz, 2H, H-arom), 7.72 (d, J=1.0 Hz, 1H, H—C(6)), 7.55 (d, J=6.9 Hz, 2H, H-arom), 7.44 (dd, J=8.8, 6.6 Hz, 4H, H-arom), 7.35-7.18 (m, 3H, H-arom), 6.83 (dd, J=9.0, 2.6 Hz, 4H, H-arom), 6.01 (dd, J=8.2, 5.2 Hz, 1H, H—C(1′)), 4.96 (d, J=3.3 Hz, 1H, H—C(7′)), 4.33
4.24 (m, 1H, H—C(4′)), 4.24-4.13 (m, 1H, H—C(5′)), 3.78 (d, J=0.9 Hz, 6H, MeO), 2.92-2.72 (m, 2H, H—C(3′), H—C(2′)), 1.81 (d, J=0.6 Hz, 3H, Me-C(5)), 1.79-1.62 (m, 2H, H—C(6′)), 1.22 (d, J=5.9 Hz, 1H, H—C(2′)).

¹³C NMR (75 MHz, CDCl₃) δ 164.05, 163.84 (C(4), CO₂R), 158.81 (MeO—C-arom), 150.64, 150.52 (02N—C-arom, C(2)), 145.29, 136.43, 136.34 (C-arom), 135.18 (C(6)), 130.62, 130.20, 130.17, 128.16, 128.01, 127.15, 123.58, 113.30, 113.27 (C-arom), 111.17 (C(5)), 87.53 (C(Ph)₃), 86.29 (C(1′)), 81.59 (C(4′)), 78.65 (C(7′)), 74.16 (C(5′)), 55.26 (MeO-DMTr), 47.07 (C(3′)), 37.35 (C(2′)), 35.71 (C(6′)), 12.51 (Me-C(5)).

ESI⁺-HRMS m/z calcd for C₄₀H₃₇O₁₀N₃Na ([M+Na]⁺) 742.2371, found 742.2375.

### ((3′S,5′R,7′R)-1-{2′,3′-dideoxy-3′,5′-ethano-7′-O-(4-nitrobenzoyl)-β-D-ribofuranosyl}thymine (33)

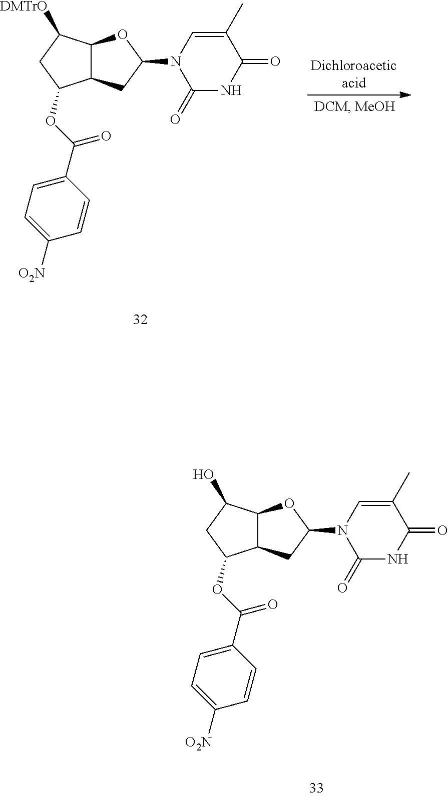

To a solution of 32 (60 mg, 0.083 mmol) in a mixture of dry DCM (1 mL) and MeOH (0.4 mL), is added dropwise dichloroacetic acid (0.2 mL) at rt. After stirring for 3 h, the mixture is then diluted with saturated NaHCO₃(15 mL) and extracted with DCM (3×10 mL). The combined organic phases are dried over MgSO₄, filtered and evaporated. The crude product is purified by CC (5% MeOH in DCM) to yield 33 (29 mg, 84%) as a white foam. Crystals suitable for X-ray analysis are obtained by recrystallization in a mixture of H₂O/MeOH.

Data for 33: R_{f}=0.18 (5% MeOH in DCM):

¹H NMR (400 MHz, DMSO) δ 11.33 (s, 1H, H—N(3)), 8.34 (d, J=8.8 Hz, 2H, H-arom), 8.27-8.13 (m, 2H, H-arom), 7.78 (s, 1H, H—C(6)), 5.96 (dd, J=9.3, 5.6 Hz, 1H, H—C(1′)), 5.18 (t, J=3.8 Hz, 1H, H—C(7′)), 5.12 (d, J=6.0 Hz, 1H, OH), 4.33 (dd, J=7.3, 4.7 Hz, 1H, H—C(4′)), 4.27 (td, J=10.5, 5.5 Hz, 1H, H—C(5′)), 2.90 (dd, J=17.2, 8.5 Hz, 1H, H—C(3′)), 2.58-2.46 (m, 1H, H—C(2′)), 2.30 (ddd, J=13.8, 8.8, 5.3 Hz, 1H, H—C(6′)), 2.03 (dd, J=9.6, 4.2 Hz, 1H, H—C(6′)), 1.92-1.76 (m, 4H, H—C(2′), Me-C(5)).

¹³C NMR (101 MHz, DMSO) δ 164.33, 164.23 (C(4), CO₂R), 150.91, 150.75 (O₂N—C-arom, C(2)), 136.79 (C-arom), 135.69 (C(6)), 131.20, 124.32 (CH-arom), 109.89 (C(5)), 85.31 (C(1′)), 81.48 (C(4′)), 80.07 (C(7′)), 71.72 (C(5′)), 47.18 (C(3′)), 37.77 (C(6′)), 35.48 (C(2′)), 12.66 12.58 (Me-C(5)).

ESI⁺-HRMS m/z calcd for C₁₉H₂₀O₈N₃ ([M+H]⁺) 418.1245, found 418.1242.

### (3′R,5′R,7′R)-1-{5′-O-Acetyl-7′-[(tert-butyldiphenylsilyl)oxy]-2′,3′-dideoxy-3′,5′-ethano-α,β-D-ribofuranosyl}thymine (35)

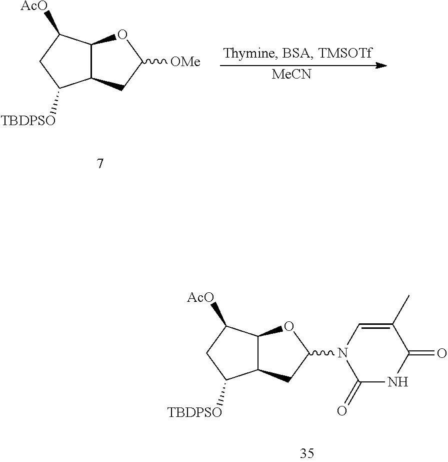

To a solution of the sugar 7 (933 mg, 2.05 mmol) and thymine (372 mg, 3.08 mmol) in dry MeCN (12 mL) is added dropwise BSA (1.5 mL, 6.15 mmol) at rt. After stirring for 50 min at rt, the solution is cooled down to 0° C. and TMSOTf (0.45 mL, 2.5 mmol) is added dropwise. After further stirring for 3 h at 0° C. and for 15 h at rt, the reaction mixture is diluted with saturated NaHCO₃(100 mL) and extracted with DCM (4×40 mL). The combined organic phases are dried over MgSO₄, filtered and evaporated. The crude product is purified by CC (2.5% isopropanol in DCM) to yield a mixture of 35 (924 mg, 82%) in an anomeric ratio α/β≈85:15 as a white foam.

Data for 35: R_{f}=0.56 (7% MeOH in DCM):

¹H NMR (300 MHz, CDCl₃) δ 9.14 (br, 1H, H—N(3)), 7.53 (dd, J=7.7, 1.6 Hz, 4H, H-arom), 7.39-7.23 (m, 6H, H-arom), 7.09 (d, J=1.0 Hz, 0.15H, H—C(6)), 6.87 (d, J=1.0 Hz, 0.85H, H—C(6)), 5.83 (t, J=6.2 Hz, 0.85H, H—C(1′)), 5.80-5.70 (m, 0.15H, H—C(1′)), 5.36-5.04 (m, 1H, H—C(5′)), 4.89 (dd, J=6.3, 5.2 Hz, 1H, H—C(4′)), 4.62 (dd, J=7.1, 5.6 Hz, 0.15H, H—C(4′)), 4.01-3.85 (m, 1H, H—C(7′)), 2.76-2.55 (m, 1H, H—C(3′)), 2.09-1.91 (m, 4H, H—C(6′), MeCO₂), 1.90-1.58 (m, 6H, H—C(6′), H—C(2′), Me-C(5)), 0.96 (s, 9H, (CH₃)₃—C—Si).

¹³C NMR (75 MHz, CDCl₃) δ 170.70 (MeCO₂), 163.87 (C(4)), 150.29 (C(2)), 135.69, 135.67 (CH-arom), 134.99 (C(6)), 133.58, 133.18 (C-arom), 130.03, 127.87 (CH-arom), 111.05 (C(5)), 87.56 (C(1′)), 82.85 (C(4′)), 76.50 (C(7′)), 74.76 (C(5′)), 50.72 (C(3′)), 37.79 (C(6′)), 36.94 (C(2′)), 26.88 ((CH₃)₃—C—Si), 20.95 (MeCO₂), 19.01 ((CH₃)₃—C—Si), 12.63 (Me-C(5)).

ESI⁺-HRMS m/z calcd for C₃₀H₃₇O₆N₂Si ([M+H]⁺) 549.2415, found 549.2401.

### (3′S,5′R,7′R)-1-{5′-O-Acetyl-2′,3′-dideoxy-3′,5′-ethano-7′-hydroxy-α,β-D-ribofuranosyl}thymine (36)

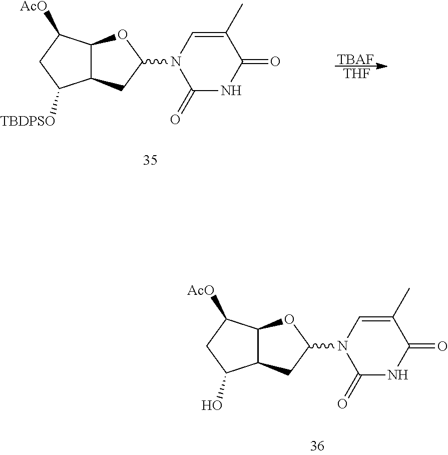

To a solution of the nucleoside 35 (924 mg, 1.68 mmol) in dry THF (10 mL) is added TBAF (1M in THF, 3.4 mL, 3.4 mmol) at rt. After stirring for 2 h at rt, the reaction mixture is diluted with saturated NaHCO₃(80 mL) and extracted with EtOAc (3×80 mL) and DCM (2×80 mL). The combined organic phases are dried over MgSO₄, filtered and evaporated. The crude product is purified by CC (5% MeOH in DCM) to yield an anomeric mixture of 36 (391 mg, 75%).

Data for 36: R_{f}=0.24 (7% MeOH in DCM):

¹H NMR (400 MHz, CDCl₃) δ 9.66 (br, 0.15H, H—N(3)), 9.63 (br, 0.85H, H—N(3)), 7.27 (d, J=1.0 Hz, 0.15H, H—C(6)), 7.06 (d, J=1.0 Hz, 0.85H, H—C(6)), 6.00 (t, J=6.1 Hz, 0.85H, H—C(1′)), 5.91 (dd, J=8.8, 5.5 Hz, 0.15H, H—C(1′)), 5.26-5.10 (m, 1H, H—C(5′)), 4.92 (dd, J=6.5, 5.3 Hz, 0.85H, H—C(4′)), 4.65 (dd, J=6.9, 5.7 Hz, 0.15H, H—C(4′)), 4.19-4.03 (m, 1H, H—C(7′)), 2.91-2.72 (m, 2H, H—C(3′), OH), 2.64 (ddd, J=13.3, 9.8, 5.5 Hz, 0.15H, H—C(2′)), 2.25-2.15 (m, 1.70H, H—C(2′)), 2.05 (s, 0.45H, MeCO₂), 2.04 (s, 2.55H, MeCO₂), 2.03-1.89 (m, 2H, H—C(6′)), 1.88 (d, J=0.7 Hz, 0.45H, Me-C(5)), 1.85 (d, J=0.6 Hz, 2.55H, Me-C(5)), 1.42-1.28 (m, 0.15H, H—C(2′)).

¹³C NMR (101 MHz, CDCl₃) δ 170.87 (MeCO₂), 164.26 (C(4)), 150.66 (C(2)), 135.54 (C(6)), 111.22 (C(5)), 87.97 (C(1′)), 82.97 (C(4′)), 75.08 (C(7′)), 74.52 (C(5′)), 50.07 (C(3′)), 37.81 (C(2′)), 37.23 (C(6′)), 21.02 (MeCO₂), 12.67 (Me-C(5)).

ESI⁺-HRMS m/z calcd for C₁₄H₁₉O₆N₂ ([M+H]⁺) 311.1238, found 311.1234.

### (3′S,5′R,7′R)-1-{5′-O-Acetyl-2′,3′-dideoxy-3′,5′-ethano-7′-O-[(4,4′-dimethoxytriphenyl)methyl]-α,β-D-ribofuranosyl}thymine (37)

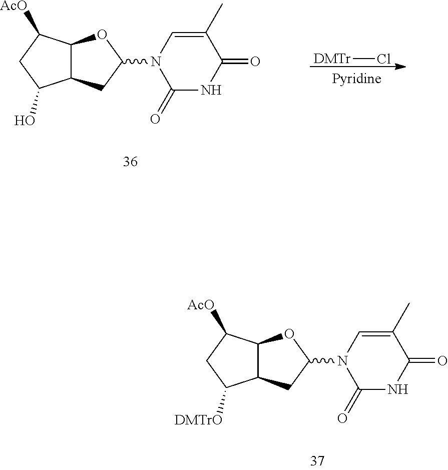

To a solution of the nucleoside 36 (364 mg, 1.17 mmol) in dry pyridine (7 mL) is added DMTr-Cl (1.19 g, 3.51 mmol) at rt. The solution is stirred for 1 day and then is diluted with saturated NaHCO₃(50 mL) and extracted with DCM (3×50 mL). The combined organic phases are dried over MgSO₄, filtered and evaporated. The crude product is purified by CC (EtOAc/hexane 2:1, +0.5% Et₃N) to yield an anomeric mixture of 37 (690 mg, 96%) as a yellow foam.

Data for 37: R_{f}=0.70 (8% MeOH in DCM):

¹H NMR (300 MHz, CDCl₃) δ 9.17 (br, 0.85H, H—N(3)), 8.56 (br, 0.15H, H—N(3)), 7.38
7.32 (m, 2H, H-arom), 7.29-7.15 (m, 7H, H-arom), 6.82 (d, J=1.1 Hz, 1H, H—C(6)), 6.76 (d, J=8.9 Hz, 4H, H-arom), 5.86 (t, J=6.0 Hz, 0.85H, H—C(1′)), 5.71 (dd, J=8.9, 5.4 Hz, 0.15H, H—C(1′)), 5.25 (dd, J=10.2, 5.6 Hz, 0.15H, H—C(5′)), 5.21-5.11 (m, 0.85H, H—(C_{5′})), 4.78 (dd, J=6.7, 4.8 Hz, 0.85H, H—C(4′)), 4.49 (dd, J=7.1, 5.3 Hz, 0.15H, H—C(4′)), 3.84 (br, 1H, H—C(7′)), 3,72, 3.71 (2s, 6H, MeO), 2.34-2.23 (m, 1H, H—C(3′)), 2.01, 1.99 (2s, 3H, MeCO₂), 1.82 (d, J=0.5 Hz, Me-C(5)), 1.80-1.56 (m, 4H, H—C(2′), H—C(6′)).

¹³C NMR (75 MHz, CDCl₃) δ 170.69 (MeCO₂), 163.91 (C(4)), 158.82 (MeO—C-arom), 150.33 (C(2)), 145.34, 136.64, 136.58 (C-arom), 135.00 (C(6)), 130.25, 128.39, 128.07, 127.15, 113.41 (CH-arom), 111.04 (C(5)), 87.70 (C(Ph)₃), 87.31 (C(1′)), 83.15 (C(4′)), 77.16 (C(7′)), 74.96 (C(5′)), 55.37 (MeO-DMTr), 49.12 (C(3′)), 37.55 (C(2′)), 36.82 (C(6′)), 21.07 (MeCO₂), 12.66 (Me-C(5)).

ESI⁺-HRMS m/z calcd for C₃₅H₃₆O₈N₂ ([M+H]⁺) 612.2466, found 612.2453.

### (3′S,5′R,7′R)-1-{2′,3′-Dideoxy-3′,5′-ethano-7′-O-[(4,4′-dimethoxytriphenyl)methyl]-α-D-ribofuranosyl}thymine (38)

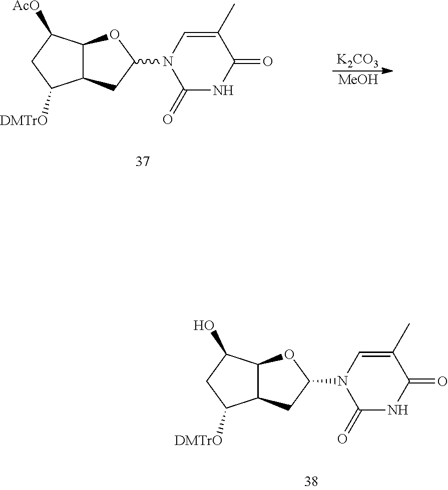

To a solution of the nucleoside 37 (690 mg, 1.12 mmol) in dry MeOH (10 mL) is added K₂CO₃ (467 mg, 3.36 mmol) at rt. The solution is stirred for 3 h and then diluted with satd NaCl (60 mL) and extracted with DCM (3×60 mL). The combined organic phases are dried over MgSO₄, filtered and evaporated. The crude product is purified by CC (3% isopropanol in Et₂O, +0.5% Et₃N) to yield the α-anomer 38 (550 mg, 86%) as a white solid.

Data for 38: R_{f}=0.39 (5% MeOH in DCM):

¹H NMR (400 MHz, CDCl₃) δ 9.37 (br, s, 1H, H—N(3)), 7.39-7.31 (m, 2H, H-arom), 7.25 (d, J=8.3 Hz, 4H, H-arom), 7.20 (t, J=7.7 Hz, 2H, H-arom), 7.16-7.08 (m, 1H, H-arom), 6.78 (d, J=1.1 Hz, 1H, H—C(6)), 6.74 (d, J=8.8 Hz, 4H, H-arom), 5.91 (dd, J=6.5, 4.9 Hz, 1H, H—C(1′)), 4.57 (dd, J=7.2, 4.4 Hz, 1H, H—C(4′)), 4.35-4.18 (m, 1H, H—C(5′)), 3.86 (d, J=4.7 Hz, 1H, H—C(7′)), 3.69 (s, 6H, MeO), 2.53 (br, 1H, OH), 2.22 (dd, J=15.3, 6.3 Hz, 1H, H—C(3′)), 1.85-1.69 (m, 5H, Me-C(5), H—C(2′), H—C(6′)), 1.66-1.49 (m, 2H, H—C(2′), H—C(6′)).

¹³C NMR (101 MHz, CDCl₃) δ 163.98 (C(4)), 158.67 (MeO—C-arom), 150.47 (C(2)), 145.48, 136.80, 136.75 (C-arom), 134.94 (C(6)), 130.19, 130.18, 128.35, 127.97, 127.01, 113.31 (CH-arom), 111.04 (C(5)), 87.82 (C(Ph)₃), 87.05 (C(1′)), 85.74 (C(4′)), 78.26 (C(7′)), 73.33 (C(5′)), 55.31 (MeO-DMTr), 48.81 (C(3′)), 40.21 (C(6′)), 37.68 (C(2′)), 12.65 (Me-C(5)).

ESI⁺-HRMS m/z calcd for C₃₃H₃₅O₇N₂ ([M+H]⁺) 571.2439, found 571.2421.

### (3′S,5′R,7′R)-1{5′-O-[(2-Cyanoethoxy)-diisopropylaminophosphanyl]2′,3′-dideoxy-3′,5′-ethano-7′-O-[(4,4′-dimethoxytriphenyl)methyl]-α-D-ribofuranosyl}thymine (39)

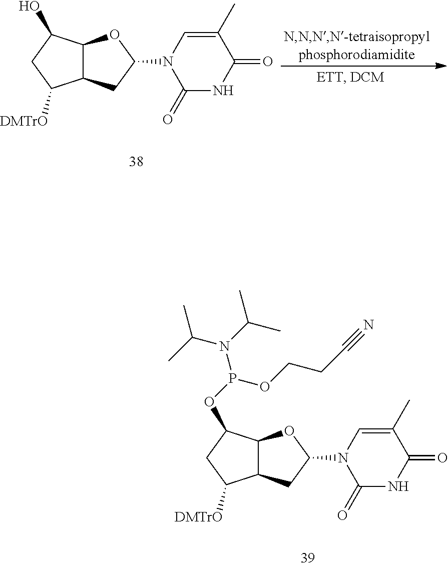

To a solution of the nucleoside 38 (200 mg, 0.350 mmol) and 5-(ethylthio)-1H-tetrazole (59 mg, 0.46 mmol) in dry DCM (7 mL) is added dropwise 2-cyanoethyl N,N,N′,N′-tetraisopropylphosphordiamidite (0.17 mL, 0.53 mmol) at rt. After stirring for 1 h, the reaction mixture is diluted with DCM (50 mL) and washed with saturated NaHCO₃(2×25 mL) and saturated NaCl (25 mL). Aqueous phases are combined and extracted with DCM (30 mL). The combined organic phases are dried over MgSO₄, filtered and evaporated. The crude product is purified by CC (2% MeOH in DCM, +0.5% Et₃N) to yield 39 (220 mg, mixture of two isomers, 81%) as a white solid.

Data for 39: R_{f}=0.44 (4% MeOH in DCM):

¹H NMR (300 MHz, CDCl₃) δ 9.03 (br, 1H, H—N(3)), 7.36 (d, J=8.1 Hz, 2H, H-arom), 7.30-7.07 (m, 7H, H-arom), 6.84 (s, 1H, H—C(6)), 6.80-6.69 (m, 4H, H-arom), 5.95, 5.88 (2dd, J=6.6, 4.8 Hz, 1H, H—C(1′)), 4.70, 4.61 (2dd, J=7.3, 4.3 Hz, 1H, H—C(4′)), 4.41-4.20 (m, 1H, H—C(5′)), 3.94-3.82 (m, 1H, H—C(7′)), 3.81-3.62 (m, 8H, MeO, OCH₂CH₂CN), 3.59-3.40 (m, 2H, (Me₂CH)₂N), 2.61-2.46 (m, 2H, OCH₂CH₂CN), 2.28 (ddd, J=14.1, 13.2, 7.3 Hz, 1H, H—C(3′)), 1.91-1.73 (m, 5H, Me-C(5), H—C(6′), H—C(2′)), 1.72-1.46 (m, 2H, H—C(6′), H—C(2′)), 1.16-1.00 (m, 12H, (Me₂CH)₂N).

¹³C NMR (75 MHz, CDCl₃) δ 164.01, 163.98 (C(4)), 158.70 (MeO—C-arom), 150.39, 150.17 (C(2)), 145.52, 136.84, 136.78 (C-arom), 135.44, 135.39 (C(6)), 130.21, 128.36, 128.32, 128.00, 127.03 (CH-arom), 118.02, 117.76 (OCH₂CH₂CN), 113.32 (CH-arom), 110.91, 110.59 (C(5)), 88.31, 88.06 (C(Ph)₃), 87.11, 87.06 (C(1′)), 85.44, 85.39 (J_{C,P}=4.6, 3.1 Hz, C(4′)), 78.25, 78.13 (C(7′)), 74.70, 74.34 (J_{C,P}=13.5, 18.5 Hz, C(5′)), 58.73, 58.47 (J_{C,P}=18.9, 20.1 Hz, (OCH₂CH₂CN)), 55.35, 55.32 (MeO-DMTr), 48.80, 48.64 (C(3′)), 43.22, 43.06 (J_{C,P}=12.4, 11.0 Hz (Me₂CH)₂N), 39.68, 39.63 (C(6′)), 38.06, 37.93 (C(2′)), 24.81, 24.74, 24.71, 24.68, 24.65, 24.59 (6s, Me₂CH)₂N), 20.37, 20.35 (J_{C,P}=7.1, 6.8 Hz, OCH₂CH₂CN), 12.66 (Me-C(5)).

³¹P NMR (122 MHz, CDCl₃) δ 148.18, 147.80.

ESI⁺-HRMS m/z calcd for C₄₂H₅₂O₈N₄P ([M+H]⁺) 771.3517, found 771.3517.

### (3′S,5′R,7′R)—N⁴-Benzoyl-1-{2′,3′-dideoxy-3′,5′-ethano-7′-O-[(4,4′-dimethoxytriphenyl)methyl]-α-D-ribofuranosyl}-5-methylcytosine (40)

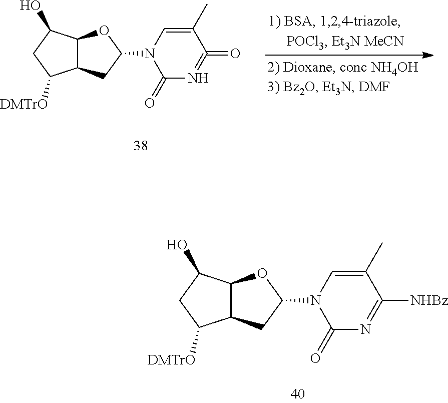

To a solution of the nucleoside 38 (268 mg, 0.470 mmol) in dry MeCN (5 mL) is added dropwise BSA (0.28 mL, 1.13 mmol) at 0°, and then the solution is stirred overnight at rt. In another flask, a suspension of 1,2,4-triazole (1.14 g, 16.5 mmol) in dry MeCN (50 mL) is cooled down to 0° C. and POCl₃ (0.35 mL, 3.8 mmol) followed Et₃N (2.62 mL, 18.8 mmol) are added. The suspension is stirred for 30 min at 0° C., and then the previously prepared solution of the silylated compound 38 is added to the suspension and the mixture is further stirred for 7 h at rt. Reaction is quenched with the addition of saturated NaHCO₃(10 mL), MeCN removed under reduced pressure and the resulting mixture diluted with saturated NaHCO₃(30 mL) and extracted with DCM (3×30 mL). The combined organic phases are dried over MgSO₄, filtered and evaporated.

The crude product is then dissolved in a mixture of 1,4-dioxane (10 mL) and concentrated NH₄OH (10 mL). After stirring for 3 h at rt, the mixture is reduced to half of its volume in vacuo, diluted with saturated NaHCO₃(25 mL) and extracted with DCM (4×30 mL). The combined organic phases are dried over MgSO₄, filtered and evaporated.

The crude product is then dissolved in dry DMF (10 mL). Et₃N (80 μL, 0.56 mmol) followed by Bz₂O (266 mg, 1.18 mmol) are added at rt and the solution is stirred overnight. The resulting brownish solution is quenched by careful addition of saturated NaHCO₃(40 mL) and extracted with DCM (4×40 mL). The combined organic phases are dried over MgSO₄, filtered and evaporated. The crude product is purified by CC (EtOAc/hexane 1:1, +0.5% Et₃N) to yield 40 (263 mg, 83%) as a white foam.

Data for 40: R_{f}=0.53 (EtOAc/hexane 3:1):

¹H NMR (300 MHz, CDCl₃) δ 13.11 (br, 1H, NH), 8.30-8.10 (m, 2H, H-arom), 7.47-7.29 (m, 5H, H-arom), 7.28-7.06 (m, 7H, H-arom), 7.00 (d, J=0.8 Hz, 1H, H—C(6)), 6.74 (d, J=8.6 Hz, 4H, H-arom), 5.89 (dd, J=6.3, 4.6 Hz, 1H, H—C(1′)), 4.61 (dd, J=7.2, 4.5 Hz, 1H, H—C(4′)), 4.33-4.20 (m, 1H, H—C(5′)), 3.87 (br, 1H, H—C(7′)), 3.69 (s, 6H, MeO), 2.32-2.13 (m, 2H, H—C(3′), OH), 1.99 (s, 3H, Me-C(5)), 1.87-1.73 (m, 2H, H—C(2′), H—C(6′)), 1.66-1.47 (m, 2H, H—C(2′), H—C(6′)).

¹³C NMR (75 MHz, CDCl₃) δ 179.61 (CONH), 159.76 (C(4)), 158.74 (MeO—C-arom), 147.87 (C(2)), 145.47 (C-arom), 137.17 (C(6)), 136.77, 136.68, 136.03 (C-arom), 132.55, 130.21, 129.98, 128.34, 128.21, 128.03, 127.07, 113.35 (CH-arom), 111.81 (C(5)), 88.74 (C(Ph)₃), 87.13 (C(1′)), 86.12 (C(4′)), 78.17 (C(7′)), 73.31 (C(5′)), 55.35 (MeO-DMTr), 48.63 (C(3′)), 40.35 (C(6′)), 38.06 (C(2′)), 13.78 (Me-C(5)).

ESI⁺-HRMS m/z calcd for C₄₀H₄₀O₇N₃ ([M+H]⁺) 674.2861, found 674.2877.

### (3′S,5′R, 7′R)—N4-Benzoyl-1-{5′-O-[(2-cyanoethoxy)-diisopropylaminophosphanyl]2′,3′-dideoxy-3′,5′-ethano-7′-O-[(4,4′-dimethoxytriphenyl)methyl]-α-D-ribofuranosyl}-5-methylcytosine (41)

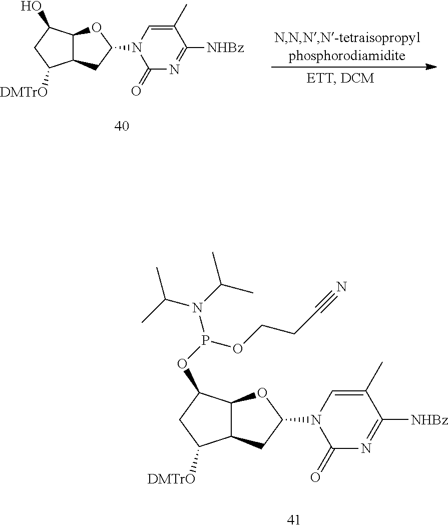

To a solution of the nucleoside 40 (250 mg, 0.371 mmol) and 5-(ethylthio)-1H-tetrazole (73 mg, 0.56 mmol) in dry DCM (8 mL) is added dropwise 2-cyanoethyl N,N,N′,N′-tetraisopropylphosphordiamidite (0.20 mL, 0.63 mmol) at rt. After stirring for 30 min, the reaction mixture is diluted with DCM (30 mL) and washed with saturated NaHCO₃(2×20 mL) and saturated NaCl (20 mL). Aqueous phases are combined and extracted with DCM (20 mL). The combined organic phases are dried over MgSO₄, filtered and evaporated. The crude product is purified by CC (EtOAc/hexane 1:1, +0.5% Et₃N) to yield 41 (260 mg, mixture of two isomers, 80%) as a white foam.

Data for 41: R_{f}=0.57 (EtOAc/hexane 1:1):

¹H NMR (300 MHz, CDCl₃) δ 13.26 (br, 1H, NH), 8.32 (d, J=7.2 Hz, 2H, H-arom), 7.58-7.39 (m, 5H, H-arom), 7.38-7.14 (m, 8H, H-arom, H—C(6)), 6.88-6.77 (m, 4H, H-arom), 6.01, 5.96 (2dd, J=6.3, 4.6 Hz, 1H, H—C(1′)), 4.82, 4.74 (2dd, J=7.3, 4.3 Hz, 1H, H—C(4′)), 4.42 (td, J=10.6, 6.0 Hz, 1H, H—C(5′)), 3.97 (br, 1H, H—C(7′)), 3.91-3.68 (m, 8H, MeO, OCH₂CH₂CN), 3.59 (dtd, J=16.7, 6.7, 3.4 Hz, 2H, (Me₂CH)₂N)), 2.62 (dt, J=15.5, 6.4 Hz, 2H, OCH₂CH₂CN), 2.49-2.23 (m, 1H, H—C(3′)), 2.11, 2.09 (2d, J=0.5 Hz, 3H, Me-C(5)), 2.00-1.82 (m, 2H, H—C(6′), H—C(2′)), 1.82-1.55 (m, 2H, H—C(6′), H—C(2′)), 1.17 (dd, J=16.3, 6.8 Hz, 12H, (Me₂CH)₂N).

¹³C NMR (101 MHz, CDCl₃) δ 179.60 (CONH), 159.97 (C(4)), 158.76 (MeO—C-arom), 147.81, 147.70 (C(2)), 145.54 (C-arom), 137.34, 136.83 (C(6)), 136.77, 136.72, 136.65, 136.55 (C-arom), 132.45, 130.22, 130.20, 129.96, 128.34, 128.31, 128.18, 128.00, 127.04 (CH-arom), 117.89, 117.71 (OCH₂CH₂CN), 113.35 (CH-arom), 111.60, 111.36 (C(5)), 89.24, 89.01 (C(Ph)₃), 87.16, 87.12 (C(1′)), 85.78, 85.62 (J_{C,P}=4.3, 3.2 Hz, C(4′)), 78.20, 77.98 (C(7′)), 74.68, 74.37 (J_{C,P}=13.4, 18.2 Hz, C(5′)), 58.70, 58.44 (J_{C,P}=18.5, 20.0 Hz, (OCH₂CH₂CN)), 55.36, 55.33 (MeO-DMTr), 48.65, 48.44 (C(3′)), 43.27, 43.14 (J_{C,P}=12.4, 12.3 Hz (Me₂CH)₂N), 39.87, 39.64 (J_{C,P}=3.4, 3.7 Hz (C(6′)), 38.30, 38.22 (C(2′)), 24.80, 24.72, 24.70, 24.67, 24.63 (Me₂CH)₂N), 20.39, 20.37 (J_{C,P}=7.2, 6.8 Hz, OCH₂CH₂CN), 13.72 (Me-C(5)).

³¹P NMR (121 MHz, CDCl₃) δ 148.18, 147.96.

ESI⁺-HRMS m/z calcd for C₄₉H₅₇O₈N₅P ([M+H]⁺) 874.3939, found 874.3946.

### (3′R,5′R,7′R)—N⁶-Benzoyl-9-{7′-[(tert-butyldiphenylsilyl)oxy]-2′,3′-dideoxy-3′,5′-ethano-α-D-ribofuranosyl}adenine (42)

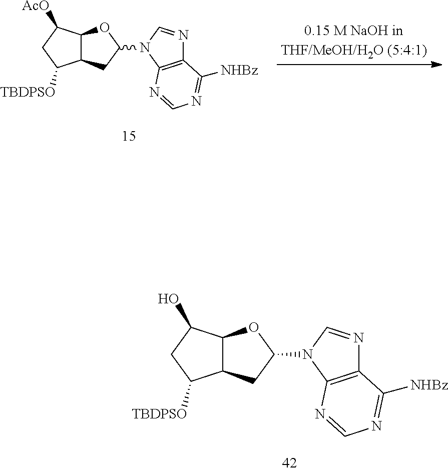

The nucleoside 15 (1.74 g, 2.64 mmol) is dissolved in 0.15 M NaOH in THF/methanol/H₂O (5:4:1, 80 mL) at 0° C. The reaction is stirred for 20 min and quenched by addition of NH₄Cl (1.06 g). Solvents are then removed under reduced pressure and the product purified by CC (5% isopropanol in DCM) to yield 42 (α-anomer, 836 mg, 51%) and 16 (β-anomer, 287 mg, 18%) as white foams.

Data for 42: R_{f}=0.35 (5% MeOH in DCM):

¹H NMR (300 MHz, CDCl₃) δ 9.34 (s, 1H, NH), 8.71 (s, 1H, H—C(2)), 8.02 (d, J=7.4 Hz, 2H, H-arom)), 7.92 (s, 1H, H—C(8)), 7.68-7.58 (m, 4H, H-arom), 7.58-7.31 (m, 9H, H-arom), 6.23 (dd, J=6.7, 2.4 Hz, 1H, H—C(1′)), 4.74 (dd, J=6.6, 4.9 Hz, 1H, H—C(4′)), 4.49 (dt, J=12.5, 6.3 Hz, 1H, H—C(5′)), 4.10 (br, 1H, H—C(7′)), 3.07 (d, J=6.7 Hz, 1H, OH), 2.92 (dd, J=15.4, 7.3 Hz, 1H, H—C(3′)), 2.52-2.35 (m, 1H, H—C(2′)), 2.10-1.97 (m, 1H, H—C(6′)), 1.94-1.77 (m, 2H, H—C(2′), H—C(6′)), 1.06 (s, 9H, (CH₃)₃—C—Si).

¹³C NMR (75 MHz, CDCl₃) δ 164.98 (CONH), 152.65 (C(2)), 151.31 (C(4)), 149.69 (C(6)), 140.93 (C(8)), 135.74 (CH-arom), 133.82, 133.68, 133.39 (C-arom), 132.77, 130.02, 129.98, 128.76, 128.06, 127.87, 127.85 (CH-arom), 123.38 (C(5)), 87.16 (C(1′)), 85.35 (C(4′)), 77.40 (C(7′)), 72.79 (C(5′)), 50.63 (C(3′)), 40.86 (C(6′)), 37.25 (C(2′)), 26.94 ((CH₃)₃—C—Si), 19.05 ((CH₃)₃—C—Si).

ESI⁺-HRMS m/z calcd for C₃₅H₃₈O₄N₅Si ([M+H]⁺) 620.2688, found 620.2671.

### (3′R,5′R,7′R)—N⁶-Benzoyl-9-{5′-O-acetyl-7′-[(tert-butyldiphenylsilyl)oxy]-2′,3′-dideoxy-3′,5′-ethano-α-D-ribofuranosyl}adenine (43)

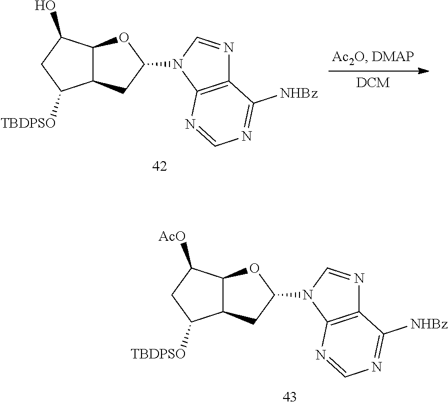

To a solution of the nucleoside 42 (1.09 g, 1.75 mmol) and 4-dimethylaminopyridine (321 mg, 2.63 mmol) in dry DCM (50 mL) is added acetic anhydride (0.83 mL, 8.8 mmol) at rt. After stirring overnight, the reaction is quenched by addition of saturated NaHCO₃(50 mL). The phases are separated and aqueous phase further extracted with DCM (2×80 mL). The combined organic phases are dried over MgSO₄, filtered and evaporated. The crude product is purified by CC (2.5% MeOH in DCM) to yield 43 (1.04 g, 90%) as a white foam.

Data for 43: R_{f}=0.33 (EtOAc/hexane 4:1):

¹H NMR (300 MHz, CDCl₃) δ 8.99 (br, 1H, NH), 8.73 (s, 1H, H—C(2)), 8.09-7.99 (m, 2H, H-arom), 7.98 (s, 1H, H—C(8)), 7.70-7.58 (m, 5H, H-arom), 7.57-7.48 (m, 2H, H-arom), 7.47-7.34 (m, 6H, H-arom), 6.22 (dd, J=6.8, 3.2 Hz, 1H, H—C(1′)), 5.45-5.35 (m, 1H, H—C(5′)), 5.01 (dd, J=6.7, 5.0 Hz, 1H, H—C(4′)), 4.09 (d, J=4.1 Hz, 1H, H—C(7′)), 3.02 (dt, J=9.5, 6.5 Hz, 1H, H—C(3′)), 2.55 (ddd, J=13.5, 10.0, 3.2 Hz, 1H, H—C(2′)), 2.15 (dd, J=13.2, 6.2 Hz, 1H, H—C(6′)), 2.09 (s, 3H, MeCO₂), 2.01 (dt, J=8.0, 3.5 Hz, 1H, H—C(2′)), 1.88 (dt, J=13.6, 5.3 Hz, 1H, H—C(6′)), 1.08 (s, 9H, (CH₃)₃—C—Si).

¹³C NMR (101 MHz, CDCl₃) δ 170.61 (MeCO₂), 164.75 (CONH), 152.67 (C(2)), 151.37 (C(4)), 149.64 (C(6)), 141.41 (C(8)), 135.85 (CH-arom), 133.71, 133.38 (C-arom), 132.91, 130.15, 130.10, 128.99, 128.02, 127.99, 127.97 (CH-arom), 123.64 (C(5)), 87.37 (C(1′)), 83.37 (C(4′)), 76.63 (C(7′)), 74.51 (C(5′)), 51.19 (C(3′)), 37.44 (C(2′)), 37.32 (C(6′)), 27.01 ((CH₃)₃—C—Si), 21.08 (MeCO₂), 19.14 ((CH₃)₃—C—Si).

ESI⁺-HRMS m/z calcd for C₃₇H₄₀O₅N₅Si ([M+H]⁺) 662.2793, found 662.2787.

### (3′S,5′R,7′R)—N⁶-Benzoyl-9-{5′-O-acetyl-2′,3′-dideoxy-3′,5′-ethano-7′-hydroxy-α-D-ribofuranosyl}adenine (44)

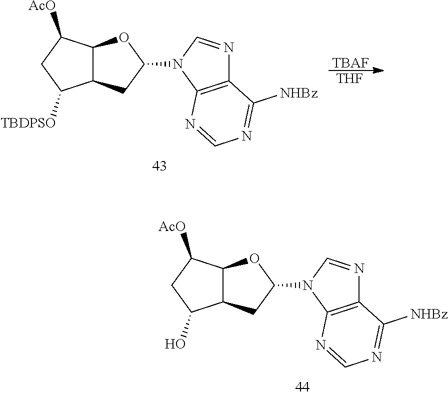

To a solution of the nucleoside 43 (990 mg, 1.50 mmol) in dry THF (50 mL) is added TBAF (1 M in THF, 3.0 mL, 3.0 mmol) at rt. After stirring for 3.5 hours at rt, the solution is diluted with EtOAc (30 mL) and THF is removed under reduced pressure. The mixture is then diluted with saturated NaHCO₃(50 mL) and extracted with DCM (4×50 mL). The combined organic phases are dried over MgSO₄, filtered and evaporated. The crude product is purified by CC (6% MeOH in DCM) to yield 44 (570 mg, 90%) as a white foam, containing traces of TBAF.

Data for 44: R_{f}=0.33 (10% MeOH in DCM):

¹H NMR (400 MHz, CDCl₃) δ 9.60 (br, 1H, NH), 8.67 (s, 1H, H—C(2)), 8.09 (s, 1H, H—C(8)), 7.96 (d, J=7.4 Hz, 2H, H-arom), 7.51 (t, J=7.4 Hz, 1H, H-arom), 7.42 (t, J=7.5 Hz, 2H, H-arom), 6.33 (dd, J=6.7, 3.1 Hz, 1H, H—C(1′)), 5.25 (ddd, J=9.7, 6.4, 5.3 Hz, 1H, H—C(5′)), 4.92 (dd, J=6.4, 5.4 Hz, 1H, H—C(1′)), 4.14 (br, 2H, H—C(7′), OH), 3.06 (dd, J=16.0, 6.6 Hz, 1H, H—C(3′)), 2.87 (ddd, J=13.2, 9.9, 3.0 Hz, 1H, H—C(2′)), 2.26-2.17 (m, 1H, H—C2′)), 2.10-1.98 (m, 5H, H—C(6′), MeCO₂).

¹³C NMR (75 MHz, CDCl₃) δ 170.64 (MeCO₂), 165.27 (CONH), 152.49 (C(2)), 151.26 (C(4)), 149.58 (C(6)), 141.64 (C(8)), 133.60 (C-arom), 132.82, 128.76, 128.06 (CH-arom), 123.30 (C(5)), 87.30 (C(1′)), 83.17 (C(4′)), 74.67 (C(7′)), 74.20 (C(5′)), 50.41 (C(3′)), 37.43 (C(2′)), 36.92 (C(6′)), 20.96 (MeCO₂).

ESI⁺-HRMS m/z calcd for C₂₁H₂₂O₅N₅ ([M+H]⁺) 424.1615, found 424.1623.

### (3′S,5′R,7′R)—N⁶-Benzoyl-9-{5′-O-acetyl-2′,3′-dideoxy-3′,5′-ethano-7′-O-[(4,4′-dimethoxytriphenyl)methyl]-α-D-ribofuranosyl}adenine (45)

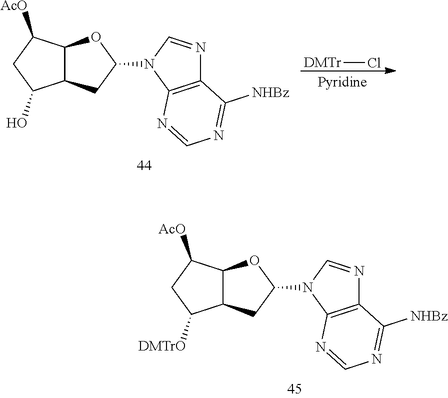

To a solution of nucleoside 44 (570 mg, 1.35 mmol) in dry pyridine (16 mL) is added DMTr-Cl (1.37 g, 4.04 mmol) at rt. The solution is stirred for 1 day and then is diluted with saturated NaHCO₃(100 mL) and extracted with DCM (3×80 mL). The combined organic phases are dried over MgSO₄, filtered and evaporated. The crude product is purified by CC (2% MeOH in DCM, +0.5% Et₃N) to yield 45 (876 mg, 89%) as a yellow foam.

Data for 45: R_{f}=0.81 (5% MeOH in DCM):

¹H NMR (300 MHz, CDCl₃) δ 9.42 (d, J=14.6 Hz, 1H, NH), 8.73 (s, 1H, H—C(2)), 8.03 (d, J=7.6 Hz, 2H, H-arom), 7.93 (s, 1H, H—C(8)), 7.66-7.55 (m, 1H, H-arom), 7.55-7.45 (m, 4H, H-arom), 7.45-7.22 (m, 7H, H-arom), 6.87 (d, J=8.7 Hz, 4H, H-arom), 6.25 (dd, J=6.6, 2.4 Hz, 1H, H—C(1′)), 5.47-5.33 (m, 1H, H—C(5′)), 4.89 (dd, J=6.7, 4.9 Hz, 1H, H—C(4′)), 4.02 (d, J=2.5 Hz, 1H, H—C(7′)), 3.79 (s, 6H, MeO), 2.58 (dd, J=16.0, 6.9 Hz, 1H, H—C(3′)), 2.38 (ddd, J=12.7, 10.0, 2.4 Hz, 1H, H—C(2′)), 2.11 (s, 3H, MeCO₂), 2.09 1.87 (m, 3H, H—C(2′), H—C(6′)).

¹³C NMR (75 MHz, CDCl₃) δ 170.40 (MeCO₂), 164.84 (CONH), 158.66 (MeO—C-arom), 152.45 (C(2)), 151.22 (C(4)), 149.51 (C(6)), 145.23 (C-arom), 141.23 (C(8)), 136.51, 133.65 (C-arom), 132.68, 130.12, 128.75, 128.33, 127.95, 127.90, 127.03 (CH-arom), 123.55 (C(5)), 113.27 (CH-arom), 87.19 (C(Ph)₃), 87.12 (C(1′)), 83.25 (C(4′)), 77.16 (C(7′)), 74.41 (C(5′)), 55.23 (MeO-DMTr), 49.23 (C(3′)), 37.61 (C(2′)), 36.22 (C(6′)), 20.98 (MeCO₂).

ESI⁺-HRMS m/z calcd for C₄₂H₄₀O₇N₅ ([M+H]⁺) 726.2922, found 726.2905.

### (3′S,5′R,7′R)—N⁶-Benzoyl-9-{2′,3′-dideoxy-3′,5′-ethano-7′-O-[(4,4′-dimethoxytriphenyl)methyl]-α-D-ribofuranosyl}adenine (46)

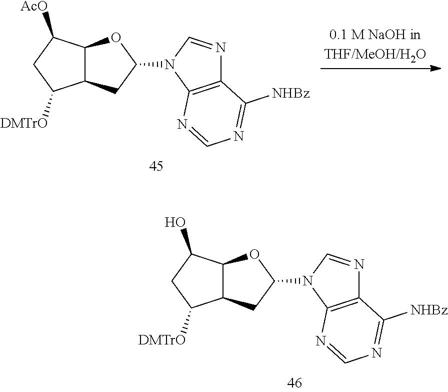

The nucleoside 45 (870 mg, 1.20 mmol) is dissolved in 0.1 M NaOH in THF/methanol/H₂O (5:4:1, 50 mL) at 0° C. The reaction is stirred for 30 min at 0° C. and then quenched by addition of NH₄Cl (321 mg). The solution is diluted with saturated NaHCO₃ (100 mL) and extracted with DCM (4×80 mL). The combined organic phases are dried over MgSO₄, filtered and evaporated. The crude product is purified by CC (3% MeOH in DCM, +0.5% Et₃N) to yield 46 (777 mg, 94%) as a white foam.

Data for 46: R_{f}=0.26 (5% MeOH in DCM):

¹H NMR (300 MHz, CDCl₃) δ 9.39 (s, 1H, NH), 8.61 (s, 1H, H—C(2)), 7.93 (d, J=7.4 Hz, 2H, H-arom), 7.75 (s, 1H, H—C(8)), 7.46 (t, J=7.3 Hz, 1H, H-arom), 7.40-7.31 (m, 4H, H-arom), 7.29-7.16 (m, 6H, H-arom), 7.11 (t, J=7.2 Hz, 1H, H-arom), 6.73 (d, J=8.7 Hz, 4H, H-arom), 6.12 (dd, J=6.5, 1.9 Hz, 1H, H—C(1′)), 4.53 (dd, J=7.5, 4.5 Hz, 1H, H—C(4′)), 4.32 (br, 1H, H—C(5′)), 3.90 (t, J=4.5 Hz, 1H, H—C(7′)), 3.66, 3.65 (2s, 6H, MeO), 3.31 (br, 1H, OH), 2.36 (dd, J=16.5, 8.1 Hz, 1H, H—C(3′)), 2.04 (ddd, J=12.0, 9.9, 2.0 Hz, 1H, H—C(2′)), 1.92-1.69 (m, 3H, H—C(2′), H—C(6′)).

¹³C NMR (75 MHz, CDCl₃) δ 164.92 (CONH), 158.64 (MeO—C-arom), 152.60 (C(2)), 151.28 (C(4)), 149.61 (C(6)), 145.44 (C-arom), 140.71 (C(8)), 136.77, 133.65 (C-arom), 132.72, 130.15, 130.12, 128.73, 128.39, 128.04, 127.96, 127.02 (CH-arom), 123.27 (C(5)), 113.28 (CH-arom), 87.11 (C(1′)), 87.01 (C(Ph)₃), 85.60 (C(4′)), 78.16 (C(7′)), 72.72 (C(5′)), 55.28 (MeO-DMTr), 48.89 (C(3′)), 39.93 (C(6′)), 37.55 (C(2′)).

ESI⁺-HRMS m/z calcd for C₄₀H₃₈O₆N₅ ([M+H]⁺) 684.2817, found 684.2800.

### (3′S,5′R,7′R)—N⁶-Benzoyl-9-{5′-O-[(2-cyanoethoxy)-diisopropylaminophosphanyl]-2′,3′-dideoxy-3′,5′-ethano-7′-O-[(4,4′-dimethoxytriphenyl)methyl]-α-D-ribofuranosyl}adenine (47)

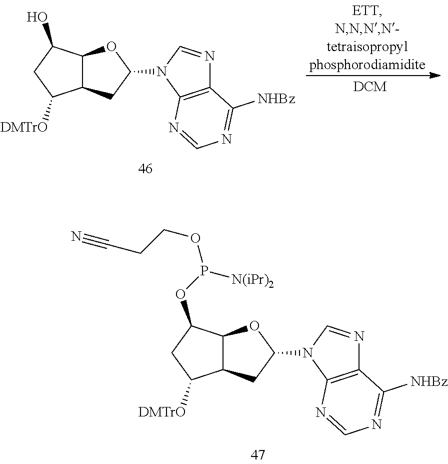

To a solution of the nucleoside 46 (199 mg, 0.290 mmol) and 5-(ethylthio)-1H-tetrazole (57 mg, 0.44 mmol) in dry DCM (7 mL) is added dropwise 2-cyanoethyl N,N,N′,N′-tetraisopropylphosphordiamidite (0.16 mL, 0.49 mmol) at rt. After stirring for 60 min, the reaction mixture is diluted with saturated NaHCO₃(20 mL) and extracted with DCM (3×20 mL). The combined organic phases are dried over MgSO₄, filtered and evaporated. The crude product is purified by CC (EtOAc, +0.5% Et₃N) to yield 47 (197 mg, mixture of two isomers, 77%) as a white foam.

Data for 47: R_{f}=0.75 (5% MeOH in DCM):

¹H NMR (300 MHz, CDCl₃) δ 8.98 (br, 1H, NH), 8.68, 8.67 (2s, 1H, C(2)), 7.94 (d, J=7.6 Hz, 2H, H-arom), 7.90, 7.84 (2s, 1H, C(8)), 7.56-7.49 (m, 1H, H-arom), 7.48-7.34 (m, 4H, H-arom), 7.30-7.10 (m, 7H, H-arom), 6.80-6.69 (m, 4H, Harom), 6.21, 6.15 (2dd, J=6.8, 2.2 Hz, 1H, H—C(1′)), 4.69, 4.59 (2dd, J=7.3, 4.5 Hz, 1H, H—C(4′)), 4.44 (tt, J=12.3, 6.3 Hz, 1H, H—C(5′)), 3.90 (dd, J=9.0, 3.8 Hz, 1H, H—C(5′)), 3.82-3.63 (m, 8H, MeO, OCH₂CH₂CN), 3.59-3.43 (m, 2H, (Me₂CH)₂N), 2.61-2.49 (m, 2H, OCH₂CH₂CN), 2.47-2.07 (m, 2H, H—C(3′), H—C(2′)), 1.98-1.66 (m, 3H, H—C(2′), H—C(6′)), 1.15-1.03 (m, 12H, (Me₂CH)₂N).

¹³C NMR (101 MHz, CDCl₃) δ 164.67 (CONH), 158.77 (MeO—C-arom), 152.58 (C(2)), 151.34, 151.29 (C(4)), 149.46 (C(6)), 145.55, 145.54 (C-arom), 141.58, 141.50 (C(8)), 136.87, 136.85, 136.84, 133.85 (C-arom), 132.85, 130.26, 130.23, 130.20, 128.97, 128.47, 128.43, 128.02, 127.96, 127.08 (CH-arom), 123.62, 123.58 (C(5)), 117.91, 117.70 (OCH₂CH₂CN), 113.37 (CH-arom), 87.80, 87.67 (C(1′)), 87.20, 87.14 (C(Ph)₃), 85.29, 85.22 ((J_{C,P}=4.2, 3.1 Hz, C(4′)), 78.16, 77.96 (C(7′)), 74.28, 73.98 (J_{C,P}=14.8, 18.4 Hz, C(5′)), 58.80, 58.61 (J_{C,P}=16.2, 17.3 Hz OCH₂CH₂CN), 55.37, 55.35 (MeO-DMTr), 49.02, 48.91 (C(3′)), 43.29, 43.16 (J_{C,P}=8.9, 9.0 Hz, ((Me₂CH)₂N), 39.09 (C(6′)), 37.99, 37.95 (C(2′)), 24.82, 24.77, 24.74, 24.70, 24.64 ((Me₂CH)₂N), 20.43, 20.42 (J_{C,P}=1.4, 1.9 Hz, OCH₂CH₂CN).

³¹P NMR (121 MHz, CDCl₃) δ 148.14, 148.11.

ESI⁺-HRMS m/z calcd for C₄₅H₅₆O₇N₈P ([M+H]⁺) 884.3895, found 884.3904.

### (3′R,5′R,7′R)-2-Amino-6-chloro-9-{7′-[(tert-butyldiphenylsilyl)oxy]-2′,3′-dideoxy-3′,5′-ethano-α-D-ribofuranosyl}purine (48)

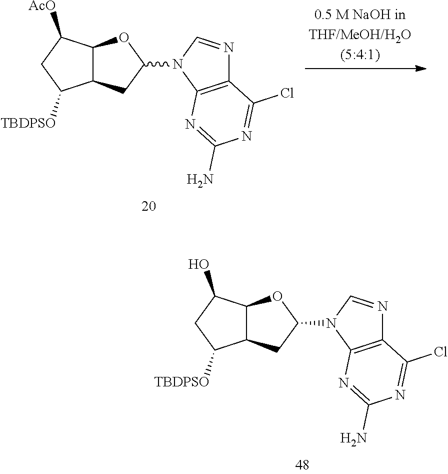

The nucleoside 20 (1.78 g, 3.01 mmol) is dissolved in 0.5 M NaOH in THF/methanol/H₂O (5:4:1, 15 mL) at 0° C. The reaction is stirred for 20 min at 0° C. and is quenched by addition of NH₄C₁ (484 mg). The suspension is then diluted with saturated NaHCO₃(100 mL) and extracted with DCM (4×75 mL). The combined organic phases are dried over MgSO₄, filtered and evaporated. The crude product is purified by CC (3% MeOH in DCM) to yield 48 (α-anomer, 992 mg, 60%) and 21 (β-anomer, 428 mg, 25%) as white foams.

Data for 48: R_{f}=0.34 (5% MeOH in DCM):

¹H NMR (400 MHz, CDCl₃) δ 7.71-7.60 (m, 5H, H-arom, H—(C(8)), 7.49-7.34 (m, 6H, H-arom), 6.08 (dd, J=6.9, 2.6 Hz, 1H, H—C(1′)), 5.26 (s, 2H, NH₂), 4.70 (dd, J=7.5, 4.8 Hz, 1H, H—C(4′)), 4.47 (dt, J=10.0, 5.1 Hz, 1H, H—C(5′)), 4.11 (t, J=3.3 Hz, 1H, H—C(7′)), 2.87 (dd, J=16.5, 7.7 Hz, 1H, H—C(3′)), 2.57 (br, 1H, OH), 2.27 (ddd, J=14.0, 9.9, 2.6 Hz, 1H, H—C(2′)), 2.10-2.01 (m, 1H, H—C(6′)), 1.92-1.76 (m, 2H, H—C(2′), H—C(6′)), 1.06 (s, 9H, (CH₃)₃—C—Si).

¹³C NMR (75 MHz, CDCl₃) δ 159.09 (C(2)), 153.05 (C(4)), 151.46 (C(6)), 139.91 (C(8)), 135.71 (CH-arom), 133.96, 133.27 (C-arom), 130.00, 129.96, 127.86, 127.83 (CH-arom), 125.52 (C(5)), 86.46 (C(1′)), 84.92 (C(4′)), 77.40 (C(7′)), 72.63 (C(5′)), 50.55 (C(3′)), 40.92 (C(6′)), 36.78 (C(2′)), 26.88 ((CH₃)₃—C—Si), 19.01 ((CH₃)₃—C—Si).

ESI⁺-HRMS m/z calcd for C₂₈H₃₃O₃N₅ClSi ([M+H]⁺) 550.2036, found 550.2019.

### (3′R,5′R,7′R)-9-{7′-[(tert-Butyldiphenylsilyl)oxy]-2′,3′-dideoxy-3′,5′-ethano-α-D-ribofuranosyl}guanine (49)

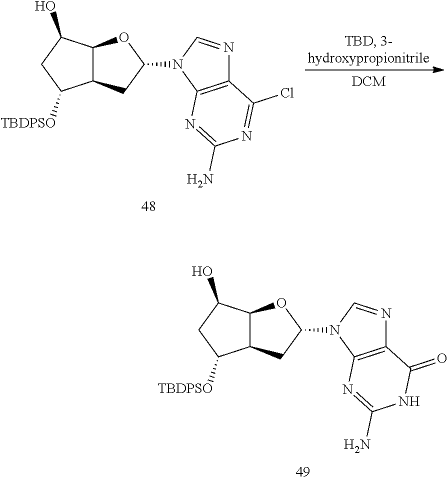

To a solution of the nucleoside 48 (610 mg, 1.03 mmol) in dry DCM (15 mL) are added 3-hydroxypropionitrile (0.28 mL, 4.12 mmol) followed by 1,5,7-triazabicyclo[4.4.0]dec-5-ene (287 mg, 2.06 mmol) at rt. After 4 hours of stirring at rt, a second portion of 3-hydroxypropionitrile (0.28 mL, 3.23 mmol) followed by 1,5,7-triazabicyclo[4.4.0]dec-5-ene (287 mg, 2.06 mmol) are added. The reaction is further stirred for 2 days and then is directly purified by CC (10% MeOH in DCM) to yield 49 (500 mg, 87%) as white foam.

Data for 49: R_{f}=0.30 (10% MeOH in DCM):

¹H NMR (400 MHz, MeOD) δ 7.73-7.61 (m, 5H, H-arom, H—C(8)), 7.53-7.32 (m, 6H, H-arom), 6.06 (dd, J=6.9, 3.7 Hz, 1H, H—C(1′)), 4.74 (dd, J=7.0, 4.6 Hz, 1H, H—C(4′)), 4.46-4.36 (m, 1H, H—C(5′)), 4.11 (br, 1H, H—C(7′)), 2.91 (dd, J=16.2, 6.6 Hz, 1H, H—C(3′)), 2.31 (ddd, J=13.8, 10.0, 3.7 Hz, 1H, H—C(2′)), 1.98-1.78 (m, 3H, H—C(2′), H—C(3′)), 1.07 (s, 9H, (CH₃)₃—C—Si).

¹³C NMR (101 MHz, MeOD) δ 159.30 (C(2)), 155.14 (C(6)), 152.38 (C(4)), 137.28 (C(8)), 136.93, 136.88 (CH-arom), 135.13, 134.78 (C-arom), 131.07, 131.06, 128.91, 128.89 (CH-arom), 117.98 (C(5)), 87.72 (C(1′)), 86.25 (C(4′)), 79.21, (C(7′)) 73.87 (C(5′)), 52.13 (C(3′)), 41.44 (C(6′)), 38.35 (C(2′)), 27.42 ((CH₃)₃—C—Si), 19.82 ((CH₃)₃—C—Si)).

ESI⁺-HRMS m/z calcd for C₂₈H₃₄O₄N₅Si ([M+H]⁺) 532.2386, found 532.2367.

### (3′R,5′R,7′R)—N2-Acetyl-9-{5′-O-acetyl-7′-[(tert-butyldiphenylsilyl)oxy]-2′,3′-dideoxy-3′,5′-ethano-α-D-ribofuranosyl}guanine (50)

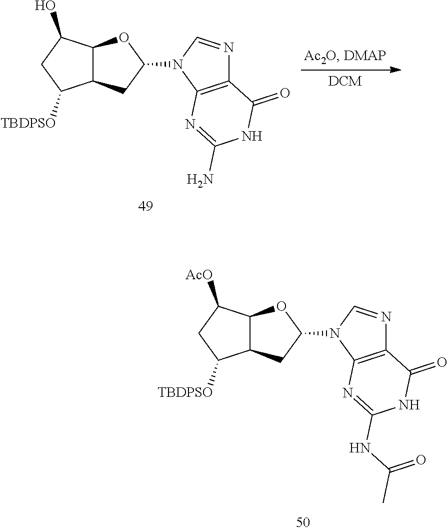

To a solution of nucleoside 49 (500 mg, 0.940 mmol) and 4-dimethylaminopyridine (276 mg, 2.4 mmol) in dry DCM (15 mL) is added acetic anhydride (1.0 mL, 10.3 mmol) at rt. After stirring for 2 days, reaction is quenched by addition of saturated NaHCO₃(30 mL). The mixture is then extracted with DCM (3×30 mL). The combined organic phases are dried over MgSO₄, filtered and evaporated. The crude product is purified by CC (3.5% MeOH in DCM) to yield 50 (441 mg, 76%) as white foam.

Data for 50: R_{f}=0.62 (10% MeOH in DCM):

¹H NMR (300 MHz, CDCl₃) δ 12.11 (br, 1H, NH—C(4)), 9.94 (br, 1H, H—N(1)), 7.62 (d, J=6.7 Hz, 5H, H-arom, H—C(8)), 7.46-7.31 (m, 6H, H-arom), 6.03 (dd, J=6.7, 2.7 Hz, 1H, H—C(1′)), 5.31 (dt, J=10.3, 5.2 Hz, 1H, H—(C_{5′})), 4.99-4.81 (m, 1H, H—C(4′)), 4.02 (d, J=3.8 Hz, 1H, H—C(7′)), 2.88 (dd, J=16.0, 6.6 Hz, 1H, H—C(3′)), 2.44-2.20 (m, 4H, MeCONH, H—C(2′)), 2.12-1.73 (m, 6H, MeCO₂, H—C(6′), H—C(2′)), 1.04 (s, 9H, (CH₃)₃—C—Si).

¹³C NMR (75 MHz, CDCl₃) δ 172.73 (MeCONH), 170.46 (MeCO₂), 155.87 (C(6)), 148.09 (C(4)), 147.47 (C(2)), 137.13 (C(8)), 135.74 (CH-arom), 133.62, 133.29 (C-arom), 130.13, 130.09, 127.96, 127.93 (CH-arom), 121.54 (C(5)), 86.47 (C(1′)), 82.81 (C(4′)), 76.60 (C(7′)), 74.37 (C(5′)), 51.23 (C(3′)), 37.04, 37.01, (C(2′), C(6′)) 26.92 ((CH₃)₃—C—Si), 24.46 (MeCONH), 21.00 (MeCO₂), 19.05 ((CH₃)₃—C—Si).

ESI⁺-HRMS m/z calcd for C₃₂H₃₈O₆N₅Si ([M+H]⁺) 616.2586, found 616.2580.

### (3′S,5′R,7′R)— N²-Acetyl-9-{5′-O-acetyl-2′,3′-dideoxy-3′,5′-ethano-7′-hydroxy-α-D-ribofuranosyl}guanine (51)

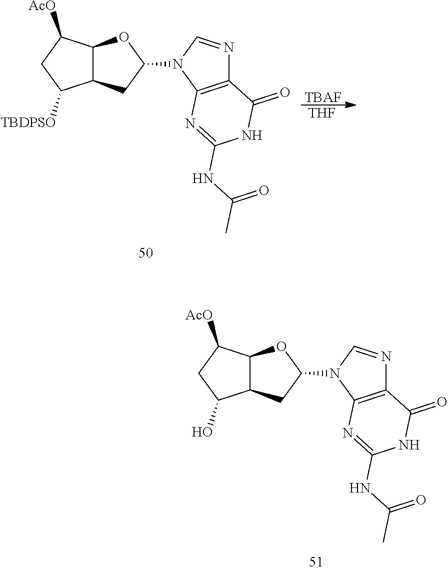

To a solution of nucleoside 50 (440 mg, 0.714 mmol) in dry THF (5 mL) is added TBAF (1M in THF, 1.1 mL, 1.1 mmol) at rt. The solution is stirred for 4 hours at rt and then is directly purified by CC (13% MeOH in DCM) to yield 51 (235 mg, 87%) as a white foam. Crystals suitable for X-ray analysis are obtained by recrystallization from a mixture of H₂O/MeOH.

Data for 51: R_{f}=0.25 (13% MeOH in DCM):

¹H NMR (300 MHz, MeOD) δ 8.03 (s, 1H, H—C(8)), 6.28 (dd, J=7.0, 3.8 Hz, 1H, H—C(1′)), 5.21 (ddd, J=9.2, 6.8, 5.1 Hz, 1H, H—C(5′)), 4.98 (dd, J=6.7, 5.0 Hz, 1H, H-(4′)), 4.13-4.05 (m, 1H, H—C(7′)), 3.17-3.05 (m, 1H, H—C(3′)), 2.86 (ddd, J=13.8, 10.0, 3.8 Hz, 1H, H—C(2′)), 2.39-2.27 (m, 1H, H—C(2′)), 2.24 (s, 3H, MeCONH), 2.16-2.00 (m, 5H, MeCO₂, H—C(6′)).

¹³C NMR (101 MHz, MeOD) δ 174.95 (MeCONH), 172.32 (MeCO₂), 157.50 (C(6)), 149.96 (C(4)), 149.38 (C(2)), 139.66 (C(8)), 121.76 (C(5)), 88.23 (C(1′)), 84.23 (C(4′)), 75.83 (C(5′), C(7′)), 51.65 (C(3′)), 38.04, 37.93 (C(2′), C(6′)), 23.83 (MeCONH), 20.71 (MeCO₂).

ESI⁺-HRMS m/z calcd for C₁₆H₂₀O₆N₅ ([M+H]⁺) 378.1408, found 378.1419.

### (3′S,5′R,7′R)— N²-Acetyl-9-{5′-O-acetyl-2′,3′-dideoxy-3′,5′-ethano-7′-O-[(4,4′-dimethoxytriphenyl)methyl]-α-D-ribofuranosyl}guanine (52)

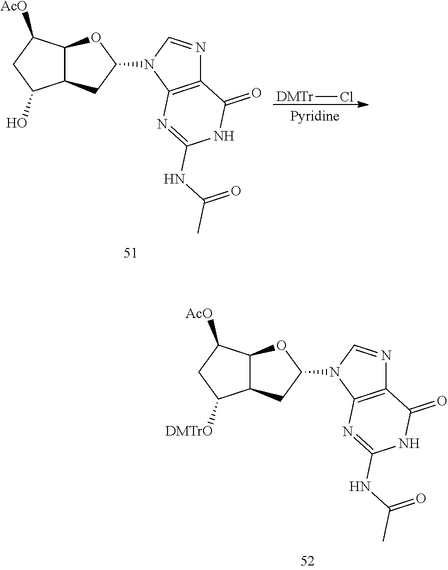

To a solution of the nucleoside 51 (186 mg, 0.492 mmol) in dry pyridine (10 mL) is added DMTr-Cl (501 mg, 1.48 mmol) at rt. The solution is stirred for 2 days and then is diluted with saturated NaHCO₃(40 mL) and extracted with DCM (3×30 mL). The combined organic phases are dried over MgSO₄, filtered and evaporated. The crude product is purified by CC (3% MeOH in DCM, +0.5% Et₃N) to yield 52 (333 mg, 99%) as a yellow foam.

Data for 52: R_{f}=0.56 (10% MeOH in DCM):

¹H NMR (300 MHz, CDCl₃) δ 12.05 (br, 1H, NH—C(4)), 9.90 (br, 1H, H—N(1)), 7.40 (s, 1H, H—C(8)), 7.38-7.31 (m, 2H, H-arom), 7.28-7.08 (m, 7H, H-arom), 6.75 (dd, J=9.0, 2.7 Hz, 4H, H-arom), 5.95-5.85 (m, 1H, H—C(1′)), 5.30-5.10 (m, 1H, H—C(5′)), 4.70-4.58 (m, 1H, H—C(4′)), 3.81 (br, 1H, H—C(7′)), 3.68, 3.68 (2s, 6H, MeO), 2.25-2.07 (m, 5H, MeCONH, H—C(3′), H—C(2′)), 1.96-1.79 (m, 5H, MeCO₂, H—C(2′), H—C(6′)), 1.74-1.59 (m, 1H, H—C(6′)).

¹³C NMR (75 MHz, CDCl₃) δ 172.65 (MeCONH), 170.42 (MeCO₂), 158.73, 158.70 (MeO—C-arom), 155.86 (C(6)), 147.96 (C(4)), 147.43 (C(2)), 145.31 (C-arom), 137.17 (C(8)), 136.69, 136.44 (C-arom), 130.32, 130.21, 128.29, 128.05, 127.09 (CH-arom), 121.53 (C(5)), 113.38, 113.35 (CH-arom), 87.25 (C(Ph)₃), 86.73 (C(1′)), 82.77 (C(4′)), 77.19 (C(7′)), 74.37 (C(5′)), 55.38 (MeO-DMTr), 49.28 (C(3′)), 37.25 (C(2′)), 36.06 (C(6′)), 24.40 (MeCONH), 21.01 (MeCO₂).

ESI⁺-HRMS m/z calcd for C₃₇H₃₈O₈N₅ ([M+H]⁺) 680.2715, found 680.2718.

### (3′S,5′R,7′R)—N2-(N,N-Dimethylformamidino)-9-{2′,3′-dideoxy-3′,5′-ethano-7′-O-[(4,4′-dimethoxytriphenyl)methyl]-α-D-ribofuranosyl}guanine (53)

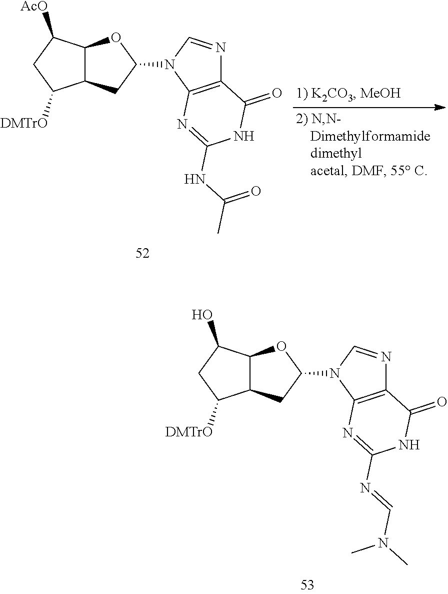

To a solution of the nucleoside 52 (333 mg, 0.490 mmol) in dry MeOH (10 mL) is added K₂CO₃ (305 mg, 2.20 mmol) at rt. The suspension is stirred for 7 h at rt, then NH₄Cl (78 mg, 1.46 mmol) is added and the resulting mixture is filtered through a short pad of SiO₂. The SiO₂ is washed with additional MeOH and then solvent is evaporated.

The crude product is dissolved in dry DMF (10 mL) and N,N-dimethylformamide dimethyl acetal (0.33 mL, 2.5 mmol) is added. The solution is stirred for 2 hours at 55° C. and then the solvents are removed under reduced pressure. The crude product is purified by CC (7% MeOH in DCM, +0.5% Et₃N) to yield 53 (245 mg, 77%) as white foam containing traces of Et₃N.

Data for 53: R_{f}=0.32 (12% MeOH in DCM):

¹H NMR (300 MHz, CDCl₃) δ 9.75 (br, 1H, H—N(1)), 8.25 (s, 1H, NCHN(CH₃)₂), 7.37 (d, J=7.3 Hz, 2H, H-arom), 7.29-7.08 (m, 8H, H-arom, H—C(8)), 6.74 (d, J=8.1 Hz, 4H, H-arom), 6.03 (dd, J=6.7, 2.8 Hz, 1H, H—C(1′)), 4.57 (dd, J=7.5, 4.6 Hz, 1H, H—C(4′)), 4.37-4.26 (m, 1H, H—C(5′)), 3.89 (t, J=3.9 Hz, 1H, H—C(7′)), 3.67, 3.67 (2s, 6H, MeO), 3.24 (br, 1H, OH), 2.94 (s, 3H, NCHN(CH₃)₂), 2.87 (s, 3H, NCHN(CH₃)₂), 2.35 (dd, J=15.9, 7.6 Hz, 1H, H—C(3′)), 1.94-1.68 (m, 4H, H—C(2′), H—C(6′)).

¹³C NMR (75 MHz, CDCl₃) δ 158.61 (MeO—C-arom), 158.28 (C(2)), 157.92 (NCHN(CH₃)₂), 156.69 (C(6)), 149.90 (C(4)), 145.52, 136.86, 136.77 (C-arom), 135.50 (C(8)), 130.15, 128.32, 127.92, 126.95 (CH-arom), 120.27 (C(5)), 113.24 (CH-arom), 86.92 (C(Ph)₃), 85.57 (C(1′)), 85.12 (C(4′)), 78.31 (C(7′)), 72.69 (C(5′)), 55.28 (MeO-DMTr), 49.28 (C(3′)), 41.38 (NCHN(CH₃)₂), 39.77 (C(6′)), 37.58 (C(2′)), 35.04 (NCHN(CH₃)₂).

ESI⁺-HRMS m/z calcd for C₃₆H₃₉O₆N₆ ([M+H]⁺) 651.2926, found 651.2921.

### (3′S,5′R,7′R)—N²—(N,N-Dimethylformamidino)-9-{5′-O-[(2-cyanoethoxy)-diisopropylaminophosphanyl]-2′,3′-dideoxy-3′,5′-ethano-7′-O-[(4,4′-dimethoxytriphenyl)methyl]-α-D-ribofuranosyl}guanine (54)

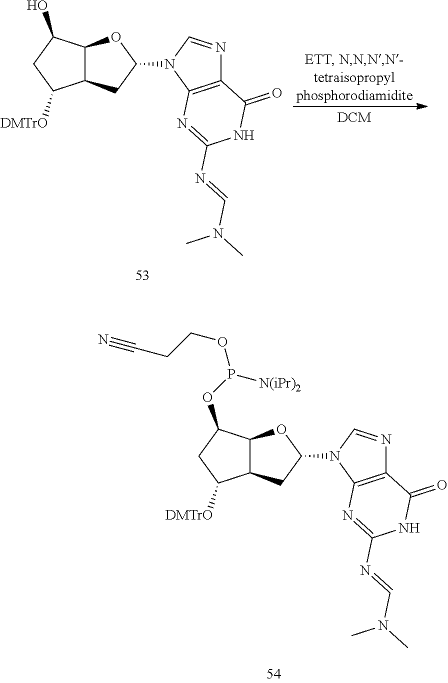

To a solution of the nucleoside 53 (245 mg, 0.377 mmol) and 5-(ethylthio)-1H-tetrazole (74 mg, 0.57 mmol) in dry DCM (15 mL) is added dropwise 2-cyanoethyl N,N,N′,N′-tetraisopropylphosphordiamidite (0.20 mL, 0.64 mmol) at rt. After stirring for 50 min, the reaction mixture is diluted with saturated NaHCO₃(25 mL) and extracted with DCM (3×25 mL). The combined organic phases are dried over MgSO₄, filtered and evaporated. The crude product is purified by CC (3% MeOH in DCM, +0.5% Et₃N) to yield 54 (212 mg, mixture of two isomers, 67%) as a white foam.

Data for 54: R_{f}=0.42 (7% MeOH in DCM):

¹H NMR (300 MHz, CDCl₃) δ 9.35 (br, 1H, H—N(1)), 8.51, 8.49 (2s, 1H, NCHN(CH₃)₂), 7.41-7.10 (m, 10H, H-arom, H—C(8)), 6.83-6.70 (m, 4H, H-arom), 6.15-6.00 (m, 1H, H—C(1′)), 4.64-4.36 (m, 2H, H—C(4′), H—C(5′)), 3.90-3.82 (m, 1H, H—C(7′)), 3.80-3.62 (m, 8H, MeO, OCH₂CH₂CN), 3.59-3.43 (m, 2H, (Me₂CH)₂N), 3.04, 3.02 (2s, 6H, NCHN(CH₃)₂), 2.67-2.48 (m, 2H, OCH₂CH₂CN), 2.32 (ddd, J=24.1, 15.1, 6.7 Hz, 1H, H—C(3′)), 2.02-1.63 (m, 4H, H—C(2′), H—C(6′)), 1.14-1.03 (m, 12H, (Me₂CH)₂N).

¹³C NMR (101 MHz, CDCl₃) δ 158.76 (MeO—C-arom), 158.17, 158.12 (C(2)), 158.03 (NCHN(CH₃)₂), 156.66, 156.59 (C(6)), 149.85, 149.79 (C(4)), 145.51, 145.49, 136.84, 136.77, 136.73, 136.71 (C-arom), 135.76, 135.59 (C(8)), 130.24, 130.20, 128.41, 128.33, 128.02, 127.10, 127.08 (CH-arom), 120.74, 120.70 (C(5)), 117.98, 117.72 (OCH₂CH₂CN), 113.34 (CH-arom), 87.16, 87.10 (C(Ph)₃), 86.00, 85.72 (C(1′)), 84.13, 84.10 (J_{C,P}=3.6, 2.5 Hz, C(4′)), 78.02, 77.67 (C(7′)), 74.15, 73.74 (J_{C,P}=15.3, 18.7 Hz, C(5′)), 58.90, 58.67 (J_{C,P}=18.7, 19.7 Hz OCH₂CH₂CN), 55.38, 55.36 (MeO-DMTr), 49.20, 49.09 (C(3′)), 43.20, 43.15 (J_{C,P}=12.4, 12.6 Hz, ((Me₂CH)₂N), 41.42, 41.38 (NCHN(CH₃)₂), 38.68, 38.65 (C(6′)), 37.97, 37.84 (C(2′)), 35.25 (NCHN(CH₃)₂), 24.83, 24.75, 24.68, 24.60, 24.53 ((Me₂CH)₂N), 20.35, 20.28 (OCH₂CH₂CN).

³¹P NMR (121 MHz, CDCl₃) δ 148.21, 148.01.

ESI⁺-HRMS m/z calcd for C₄₅H₅₆O₇N₈P ([M+H]⁺) 851.4004, found 851.4013.

### (3aR,4R,6R,6aS)-4((Tert-butyldiphenylsilyl)oxy)-2-methoxyhexahydro-2H-cyclopenta[b]furan-6-yl (4-nitrobenzoate) (55)

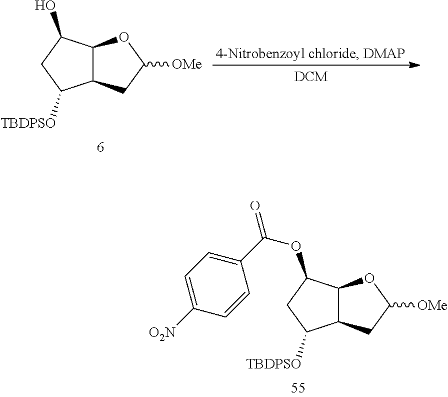

To a solution of the sugar 6 (195 mg, 0.437 mmol) and 4-dimethylaminopyridine (70 mg, 0.568 mmol) in dry DCM (10 mL) is added 4-nitrobenzoyl chloride (158 mg, 0.850 mmol) at rt. After stirring overnight, reaction is quenched by slow addition of saturated NaHCO₃(3 mL). The mixture is then diluted with saturated NaHCO₃(15 mL) and extracted with DCM (3×15 mL). The combined organic phases are dried over MgSO₄, filtered and evaporated. The crude product is purified by CC (EtOAc/hexane 1:5) to yield a mixture of 55 (260 mg, 98%) in an anomeric ratio α/β≈4:1 as a white solid.

Data for 55: R_{f}=0.62 (EtOAc/hexane 1:2):

¹H NMR (300 MHz, CDCl₃) δ 8.33-8.17 (m, 4H, H-arom), 7.72-7.61 (m, 4H, H-arom), 7.51-7.32 (m, 6H, H-arom), 5.65-5.47 (m, 1H, H—C(6)), 4.97 (dd, J=9.2, 5.6 Hz, 1H, H—C(2)), 4.87 (t, J=5.8 Hz, 1H, H—C(6a)), 4.18 (d, J=5.0 Hz, 0.2H, H—C(4)), 3.98 (d, J=3.5 Hz, 0.8H, H—C(4)), 3.21 (d, J=15.1 Hz, 3H, MeO), 2.88 (dd, J=16.6, 7.9 Hz, 0.8H, H—C(3a)), 2.75-2.62 (m, 0.2H, H—C(3a)), 2.49-2.34 (m, 0.2H, H—C(5)), 2.24-1.83 (m, 2.8H, H-(5), H—C(3)), 1.28 (ddd, J=13.0, 7.9, 4.9 Hz, 1H, H—C(3)), 1.09 (s, 9H, (CH₃)₃—C—Si).

¹³C NMR (75 MHz, CDCl₃) δ 164.46, 164.41 (CO₂R), 150.63 (02N—C-arom), 135.87, 135.82 (CH-arom), 134.07, 133.75, 133.69 (CH-arom), 130.98, 130.89, 129.98, 129.96, 129.91, 127.89, 127.87, 127.85, 123.59 (CH-arom), 106.49, 106.39 (C(2)), 83.21, 79.87 (C(6a)), 76.54 (C(4)), 76.09 (C(6)), 54.55, 54.47 (MeO), 51.69, 50.30 (C(3a), 38.07 (C(3)), 37.17, 36.65 (C(5)), 27.04, 26.99 90 ((CH₃)₃—C—Si), 19.14 ((CH₃)₃—C—Si).

ESI⁺-HRMS m/z calcd for C₃₁H₃₅O₇NaSi ([M+Na]⁺) 584.2075, found 584.2085.

### (3′R,5′R,7′R)-1-{7′-[(tert-Butyldiphenylsilyl)oxy]-2′,3′-dideoxy-3′,5′-ethano-5′-O-(4-nitrobenzoate)-α,β-D-ribofuranosyl}thymine (56)

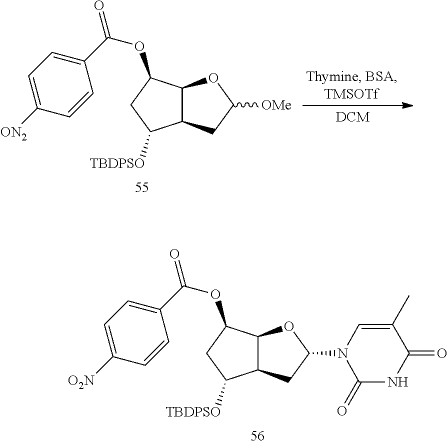

To a solution of the sugar 55 (260 mg, 0.463 mmol) and thymine (84 mg, 0.695 mmol) in dry MeCN (3 mL) is added dropwise BSA (0.34 mL, 1.4 mmol) at rt. After stirring for 30 min at rt, the solution is cooled down to 0° C. and TMSOTf (0.10 mL, 1.3 mmol) is added dropwise. After further stirring for 2 h at 0° C. and for 18 h at rt, the reaction mixture is diluted with satd NaHCO₃(30 mL) and extracted with DCM (4×40 mL). The combined organic phases are dried over MgSO₄, filtered and evaporated. The crude product is purified by CC (2% MeOH in DCM) to yield a mixture of 56 (240 mg, 79%) in an anomeric ratio α/β≈88:12 as white foam.

Data for 56: R_{f}=0.56 (DCM+3% MeOH):

¹H NMR (300 MHz, CDCl₃) δ 9.38 (br, 1H, (s, 1H, H—N(3)), 8.32-8.23 (m, 2H, H-arom), 8.22-8.11 (m, 2H, H-arom), 7.65 (dd, J=7.7, 1.5 Hz, 4H, H-arom), 7.50-7.36 (m, 6H, H-arom), 6.95 (d, J=0.9 Hz, 1H, H—C(6)), 5.96 (t, J=6.3 Hz, 1H, H—C(1′)), 5.55 (dt, J=9.9, 6.0 Hz, 1H, H—C(5′)), 5.13 (dd, J=6.4, 5.4 Hz, 1H, H—C(4′)), 4.20-4.05 (m, 1H, H—C(7′)), 2.94-2.78 (m, 1H, H—C(3′)), 2.22 (dd, J=13.3, 6.4 Hz, 1H, H—C(6′)), 2.09-1.73 (m, 6H, H—C(6′), H—C(2′), Me-C(5)), 1.09 (s, 9H, (CH₃)₃—C—Si).

¹³C NMR (75 MHz, CDCl₃) δ 164.32, 163.79 (C(4), CO₂R), 150.65, 150.39 (02N—C-arom, C(2)), 135.70, 135.68 (CH-arom), 135.13 (C-arom), 134.83 (C(6)), 133.46, 133.10 (C-arom), 130.91, 130.73, 130.11, 127.93, 123.60 (CH-arom), 111.30 (C(5)), 87.26 (C(1′)), 82.44 (C(4′)), 76.40 (C(7′)), 76.07 (C(5′)), 50.76 (C(3′)), 37.94 (C(6′)), 36.68 (C(2′)), 26.89 ((CH₃)₃—C—Si), 19.03 ((CH₃)₃—C—Si), 12.62 (Me-C(5)).

ESI⁺-HRMS m/z calcd for C₃₅H₃₇O₈N₃NaSi ([M+Na]⁺) 678.2242, found 678.2254.

### (3′R,5′R,7′R)-1-{2′,3′-Dideoxy-3′,5′-ethano-7′-hydroxy-5′-O-(4-nitrobenzoyl)-α,β-D-ribofuranosyl}thymine (57)

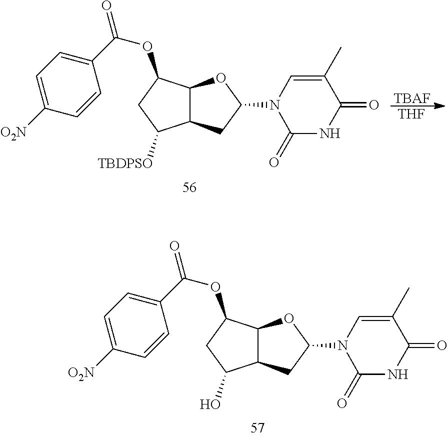

To a solution of the nucleoside 56 (220 mg, 0.335 mmol) in dry THF (2 mL) is added TBAF (1M in THF, 0.84 mL, 0.84 mmol) at rt. After stirring for 4 h at rt, the reaction mixture is diluted with saturated NaHCO₃(20 mL) and extracted with EtOAc (3×20 mL) and DCM (2×80 mL). The combined organic phases are dried over MgSO₄, filtered and evaporated. The crude product is purified by CC (5% MeOH in DCM) to yield an anomeric mixture of 57 (101 mg, 72%). Crystals suitable for X-ray analysis are obtained by recrystallization in EtOAc.

Data for 57: R_{f}=0.50 (DCM+7% MeOH):

¹H NMR (300 MHz, CDCl₃) δ 8.96 (br, 1H, H—N(3)), 8.34-8.17 (m, 4H, H-arom), 7.07 (d, J=1.1 Hz, 1H, H—C(6)), 6.11 (t, J=6.3 Hz, 1H, H—C(1′)), 5.57-5.45 (m, 1H, H—C(5′)), 5.15 (dd, J=6.6, 5.4 Hz, 1H, H—C(4′)), 4.38-4.23 (m, 1H, H—C(7′)), 2.96 (dd, J=13.5, 6.9 Hz, 1H, H—C(3′)), 2.26 (ddd, J=13.1, 10.3, 5.4 Hz, 4H, H—C(2′), H—C(6′)), 1.91 (d, J=0.9 Hz, 3H, Me-C(5)).

ESI⁺-HRMS m/z calcd for C₁₉H₁₉O₈N₃Na ([M+Na]⁺) 440.1064, found 440.1072.

Process of Alpha Anomeric Oligonucleotide Synthesis, Deprotection and Purification

An oligonucleotide comprising at least two alpha anomeric bicyclo-DNA (abc-DNA) residues connected by a phosphodiester bond can be synthesized on a synthesizer, for example, a Pharmaci-Gene-Assembler-Plus DNA synthesizer according to methods well known in the art and described herein below. The steps of synthesis of an abc-DNA oligonucleotide of the invention are shown below:

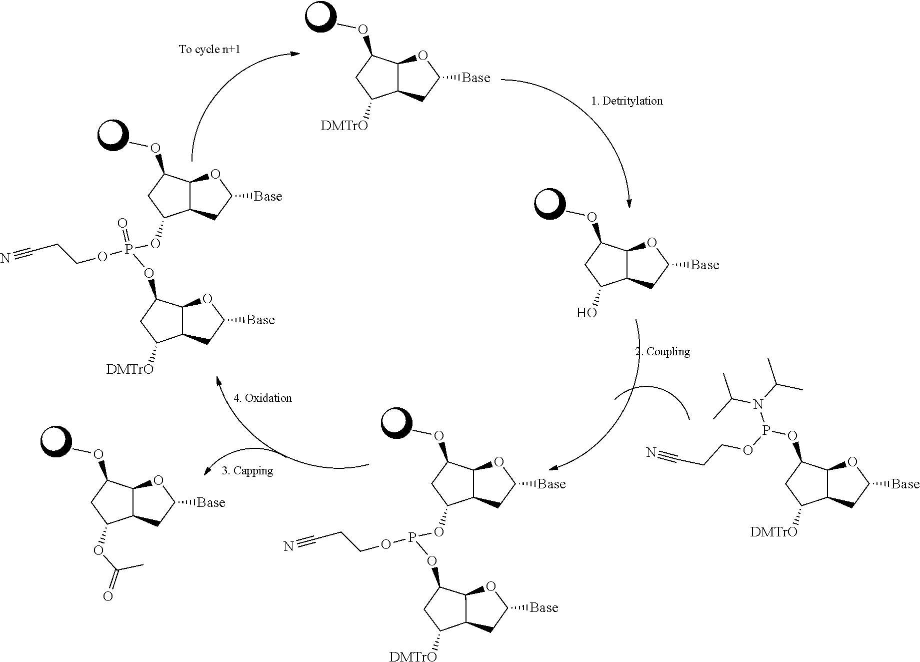

Oligonucleotide syntheses are performed on a Pharmaci-Gene-Assembler-Plus DNA synthesizer on a 1.3 μmol scale, following the protocols recommended by the manufacturer of the Gene Assembler. Natural DNA phosphoramidites (dT, dC4bz, dG2DMF, dA6Bz) and solid support (Glen Unysupport 500) are purchased from Glen Research. Natural DNA phosphoramidites are prepared as 0.1 M solution in MeCN and are coupled using a 4 min step. abc-DNA phosphoramidites are prepared as 0.1 M solutions in 1,2-dichloroethane and are coupled using an extended 12 min step using 5-(ethylthio)-1H-tetrazole (0.25 M in MeCN) is used as coupling agent. Detritylation of modified nucleoside is performed with a solution of 5% dichloroacetic acid in dichloroethane. Oxidation is performed with a solution of 0.01 M iodine in MeCN/water/collidine (32:3:15) and with a reaction time of 1 min. Sulfurization is performed with a solution of 0.2 M phenylacetyl disulfide in MeCN/pyridine (1:1) and with a reaction time of 3.5 min. Capping is performed with standard conditions. Cleavage from solid support and deprotection of oligonucleotides is achieved by treatment with concentrated ammonia at 55° C. for 16 h. After centrifugation, the supernatant are collected, the beads further washed with H₂O (0.5 mL×2) and the resulting solutions are evaporated to dryness. Crude oligonucleotides are purified by ion-exchange HPLC (Dionex—DNAPac PA200). Buffer solutions of 25 mM Trizma in H₂O, pH 8.0, is used as the mobile phase “A” and 25 mM Trizma, 1.25 M NaCl in H₂O, pH 8.0, was used as the mobile phase “B”. For the phosphorothioate strand, a buffer solution of 10 mM NaOH in H₂O, pH 12.0, was used as the mobile phase “A” and 10 mM NaOH, 2.50 M NaCl in H₂O, pH 12.0, was used as the mobile phase “B”. The purified oligonucleotides are then desalted with Sep-pak C-18 cartridges. Concentrations are determined by measuring the absorbance at 260 nm with a Nanodrop spectrophotometer, using the extinction coefficient of the corresponding natural DNA oligonucleotides. Characterizations of oligonucleotides are performed by ESL mass spectrometry or by LC-MS.

Pharmaceutical Compositions

In certain embodiments, the present invention provides for a pharmaceutical composition comprising the oligonucleotide of the present invention. The oligonucleotide sample can be suitably formulated and introduced into the environment of the cell by any means that allows for a sufficient portion of the sample to enter the cell to induce an effect, for example, exon skipping. In certain embodiments, the oligonucleotide is pre-loaded onto albumin and administered as an oligonucleotide-albumin complex. Many formulations for oligonucleotides are known in the art and can be used so long as the oligonucleotide gains entry to the target cell so that it can act. For example, the oligonucleotide agent of the instant invention can be formulated in buffer solutions such as phosphate buffered saline solutions, liposomes, micellar structures, and capsids. Formulations of oligonucleotide agent with cationic lipids can be used to facilitate transfection of the oligonucleotide agent into cells. For example, cationic lipids, such as lipofectin (U.S. Pat. No. 5,705,188), cationic glycerol derivatives, and polycationic molecules, such as polylysine (published PCT International Application WO 97/30731), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Such compositions typically include the nucleic acid molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral, intranasal, transdermal (topical), transmucosal, intrathecal, intracerebroventricular, intraperitoneal and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL.™. (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The invention also provides for dry powder delivery methods.

The compounds can also be prepared in the form of suppositories (e.g., with conventional suppository bases such as cocoa butter and other glycerides) or retention enemas for rectal delivery.

The compounds can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

The compounds can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a nucleic acid molecule (i.e., an effective dosage) depends on the nucleic acid selected. For instance, single dose amounts in the range of approximately 1 pg to 1000 mg may be administered; in some embodiments, 10, 30, 100, or 1000 pg, or 10, 30, 100, or 1000 ng, or 10, 30, 100, or 1000 μg, or 10, 30, 100, or 1000 mg may be administered. In some embodiments, 1-5 g of the compositions can be administered. The compositions can be administered from one or more times per day to one or more times per week; including once every other day or one or more times per month. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of an oligonucleotide of the invention can include a single treatment or, preferably, can include a series of treatments.

In certain embodiments, the dosage of an oligonucleotide according to the invention is in the range of 5 mg/kg/week to 500 mg/kg/week, for example 5 mg/kg/week, 10 mg/kg/week, 15 mg/kg/week, 20 mg/kg/week, 25 mg/kg/week, 30 mg/kg/week, 35 mg/kg/week, 40 mg/kg/week, 45 mg/kg/week, 50 mg/kg/week, 55 mg/kg/week, 60 mg/kg/week, 65 mg/kg/week, 70 mg/kg/week, 75 mg/kg/week, 80 mg/kg/week, 85 mg/kg/week, 90 mg/kg/week, 95 mg/kg/week, 100 mg/kg/week, 150 mg/kg/week, 200 mg/kg/week, 250 mg/kg/week, 300 mg/kg/week, 350 mg/kg/week, 400 mg/kg/week, 450 mg/kg/week and 500 mg/kg/week. In certain embodiments, the dosage of an oligonucleotide according to the invention is in the range of 10 mg/kg/week to 200 mg/kg/week, 20 mg/kg/week to 150 mg/kg/week or 25 mg/kg/week to 100 mg/kg/week. In certain embodiments the oligonucleotide is administered 1× per week for a duration of 2 weeks to 6 months, for example, 2 weeks, 3 weeks, 4, weeks, 5 weeks, 6 weeks, 7 weeks, 8 weeks, 9 weeks, 10 weeks, 11 weeks, 12 weeks, 13 weeks, 26 weeks, 6 months, 8 months, 10 months or 1 year or more. In certain embodiments, the oligonucleotide is administered 2×per week. In other embodiments, the oligonucleotide is administered every other week. In certain embodiments, the oligonucleotide is administered intravenously.

It can be appreciated that the method of introducing oligonucleotide agents into the environment of the cell will depend on the type of cell and the makeup of its environment. For example, when the cells are found within a liquid, one preferable formulation is with a lipid formulation such as in lipofectamine and the oligonucleotide agents can be added directly to the liquid environment of the cells. Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate oligonucleotide agents in a buffer or saline solution and directly inject the formulated oligonucleotide agents into cells, as in studies with oocytes. The direct injection of oligonucleotides may also be done.

Suitable amounts of an oligonucleotide agent must be introduced and these amounts can be empirically determined using standard methods. Typically, effective concentrations of individual oligonucleotide agent species in the environment of a cell will be about 50 nanomolar or less, 10 nanomolar or less, or compositions in which concentrations of about 1 nanomolar or less can be used. In another embodiment, methods utilizing a concentration of about 200 picomolar or less, and even a concentration of about 50 picomolar or less, about 20 picomolar or less, about 10 picomolar or less, or about 5 picomolar or less can be used in many circumstances.

The method can be carried out by addition of the oligonucleotide agent compositions to any extracellular matrix in which cells can live provided that the oligonucleotide agent composition is formulated so that a sufficient amount of the oligonucleotide agent can enter the cell to exert its effect. For example, the method is amenable for use with cells present in a liquid such as a liquid culture or cell growth media, in tissue explants, or in whole organisms, including animals, such as mammals and especially humans.

The oligonucleotide agent can be formulated as a pharmaceutical composition which comprises a pharmacologically effective amount of an oligonucleotide agent and pharmaceutically acceptable carrier. A pharmacologically or therapeutically effective amount refers to that amount of an oligonucleotide agent effective to produce the intended pharmacological, therapeutic or preventive result. The phrases “pharmacologically effective amount” and “therapeutically effective amount” or simply “effective amount” refer to that amount of an oligonucleotide effective to produce the intended pharmacological, therapeutic or preventive result. For example, if a given clinical treatment is considered effective when there is at least a 20% reduction in a measurable parameter associated with a disease or disorder, a therapeutically effective amount of a drug for the treatment of that disease or disorder is the amount necessary to effect at least a 20% reduction in that parameter.

Suitably formulated pharmaceutical compositions of this invention can be administered by any means known in the art such as by parenteral routes, including intravenous, intramuscular, intraperitoneal, subcutaneous, transdermal, airway (aerosol), rectal, vaginal and topical (including buccal and sublingual) administration. In some embodiments, the pharmaceutical compositions are administered by intravenous or intraparenteral infusion or injection.

In general, a suitable dosage unit of oligonucleotide will be in the range of 0.001 to 0.25 milligrams per kilogram body weight of the recipient per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. In certain embodiments, the dosage is in the range of 0.1 mg/kg body weight per day to 5 mg/kg body weight per day, for example, 0.1, 0.2, 0.3, 0.4, 0.5, 1, 1.5, 2, 2.5, 3, 3.5, 4, 4.5 and 5 mg/kg body weight. Pharmaceutical composition comprising the oligonucleotide can be administered once daily. However, the therapeutic agent may also be dosed in dosage units containing two, three, four, five, six or more sub-doses administered at appropriate intervals throughout the day. In that case, the oligonucleotide contained in each sub-dose must be correspondingly smaller in order to achieve the total daily dosage unit. The dosage unit can also be compounded for a single dose over several days, e.g., using a conventional sustained release formulation which provides sustained and consistent release of the oligonucleotide over a several day period. Sustained release formulations are well known in the art. In this embodiment, the dosage unit contains a corresponding multiple of the daily dose. Regardless of the formulation, the pharmaceutical composition must contain oligonucleotide in a quantity sufficient to be active, for example, to cause exon skipping or inhibit expression of a target gene in the animal or human being treated. The composition can be compounded in such a way that the sum of the multiple units of oligonucleotide together contain a sufficient dose.

Data can be obtained from cell culture assays and animal studies to formulate a suitable dosage range for humans. The dosage of compositions of the invention lies within a range of circulating concentrations that include the ED₅₀ (as determined by known methods) with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound that includes the IC₅₀ (i.e., the concentration of the test compound which achieves a half-maximal inhibition of symptoms) as determined in cell culture. Such information can be used to more accurately determine useful doses in humans. Levels of oligonucleotide in plasma may be measured by standard methods, for example, by high performance liquid chromatography.

The pharmaceutical compositions can be included in a kit, container, pack, or dispenser together with instructions for administration.

Methods of Treatment

The present invention provides for both prophylactic and therapeutic methods of treating a subject at risk of (or susceptible to) a disease or disorder caused, in whole or in part, by the expression of a target RNA and/or the presence of such target RNA.

“Treatment”, or “treating” as used herein, is defined as the application or administration of a therapeutic agent (e.g., an oligonucleotide agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent (e.g., an oligonucleotide agent or vector or transgene encoding same). Subjects at risk for the disease can be identified by, for example, any or a combination of diagnostic or prognostic assays as described herein. Administration of a prophylactic agent can occur prior to the detection of, e.g., viral particles in a subject, or the manifestation of symptoms characteristic of the disease or disorder, such that the disease or disorder is prevented or, alternatively, delayed in its progression.

Another aspect of the invention pertains to methods of treating subjects therapeutically, i.e., alter onset of symptoms of the disease or disorder. These methods can be performed in vitro (e.g., by culturing the cell with the oligonucleotide agent) or, alternatively, in vivo (e.g., by administering the oligonucleotide agent to a subject).

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”). Pharmacogenomics allows a clinician or physician to target prophylactic or therapeutic treatments to patients who will most benefit from the treatment and to avoid treatment of patients who will experience toxic drug-related side effects.

Therapeutic agents can be tested in an appropriate animal model. For example, an oligonucleotide agent (or expression vector or transgene encoding same) as described herein can be used in an animal model to determine the efficacy, toxicity, or side effects of treatment with the agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

Moreover, the therapeutic effect of an abc-DNA lipid group conjugated oligonucleotide is determined by assessing muscle function, grip strength, respiratory function, heart function by MRI, muscle physiology. Complement activation and blood coagulation are also determined to investigate the negative side effects of the oligonucleotide.

Diseases

The oligonucleotides of the invention are useful for modulating gene expression by interfering with transcription, translation, splicing and/or degradation and/or by inhibition the function of non-coding RNA, for treatment or prevention of a disease based on aberrant levels of an mRNA or non-coding RNA. A subject is said to be treated for a disease, if following administration of the cells of the invention, one or more symptoms of the disease are decreased or eliminated.

The abc-DNA lipid group conjugated oligonucleotides of the invention can modulate the level or activity of a target RNA. The level or activity of a target RNA can be determined by any suitable method now known in the art or that is later developed. It can be appreciated that the method used to measure a target RNA and/or the expression of a target RNA can depend upon the nature of the target RNA. For example, if the target RNA encodes a protein, the term “expression” can refer to a protein or the RNA/transcript derived from the target RNA. In such instances, the expression of a target RNA can be determined by measuring the amount of RNA corresponding to the target RNA or by measuring the amount of the protein product. Protein can be measured in protein assays such as by staining or immunoblotting or, if the protein catalyzes a reaction that can be measured, by measuring reaction rates. All such methods are known in the art and can be used. Where target RNA levels are to be measured, any art-recognized methods for detecting RNA levels can be used (e.g., RT-PCR, Northern Blotting, etc.). Any of the above measurements can be made on cells, cell extracts, tissues, tissue extracts or any other suitable source material.

The abc-DNA lipid conjugated oligonucleotides of the invention are used to modulate expression of a microRNA or other non-coding RNA that modulates mRNA expression.

MicroRNAs are small noncoding RNAs that direct post-transcriptional regulation of gene expression, and are approximately 20-25 nucleotides in length. They regulate the expression of multiple target genes through sequence-specific hybridization to the 3′ untranslated region of messenger RNAs. These microRNAs can block the translation or they can cause direct degradation of their target messenger RNAs.

abc-DNA lipid group conjugated oligonucleotides of the invention that bind to an miRNA of interest are synthesized. These oligonucleotides are designed to bind to the miRNA, and prevent binding of the miRNA to its target mRNA. abc-DNA lipid group conjugated oligonucleotides are used to modulate miRNA binding in vitro and in vivo as described in the examples above.

Long non-coding RNAs (lncRNAs) are a large and diverse class of transcribed RNA molecules with a length of more than 200 nucleotides that do not encode proteins that do not encode proteins (or lack >100 amino acid open reading frame). lncRNAs are important regulators of gene expression, and lncRNAs are thought to have a wide range of functions in cellular and developmental processes. lncRNAs may carry out both gene inhibition and gene activation through a range of diverse mechanisms. Validated functions of lncRNAs suggest that they are master regulators of gene expression and often exert their influences via epigenetic mechanisms by modulating chromatin structure.

abc-DNA lipid group conjugated oligonucleotides of the invention complementary to a target lncRNA of interest are synthesized. In the nucleus, they hybridize with targeted lncRNAs to form heteroduplexes.

The invention provides for treatment or prevention of a disease including but not limited to Duchenne Muscular Dystrophy, Spinal Muscular Atrophy (exon 7 inclusion in the SMN2 gene), Myotonic Dystrophy (target CUGexp-DMPK transcript with CAGₙ), Huntington's disease (allele selective and non-selective approaches targeting the CAG triplet expansion), Amyotrophic lateral sclerosis (genetically heterogeneous disorder with several causative genes), and Pompé disease (target splice mutation c.-32 IVS1-13T>G, which is found in over half of all Caucasian patients.

Sequences

The invention provides for any abc-DNA oligonucleotide, with predominantly phosphate internucleosidic bonds, one or two linkers and a lipid group. The sequence can be designed to any target. The sequence of exemplary abc-DNA oligonucleotides of the invention are provided below.

In certain embodiments, the oligonucleotides have a length of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides or more, for example 21-50 nucleotides, for example, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 nucleotides. In one embodiment, the oligonucleotides have a length of 14 nucleotides, 15 nucleotides, 16 nucleotides, 17 nucleotides, 18 nucleotides or 19 nucleotides. In one embodiment, the oligonucleotides have a length of 15 nucleotides. In one embodiment, the oligonucleotides have a length of 16 nucleotides. In one embodiment, the oligonucleotides have a length of 17 nucleotides. In one embodiment, the oligonucleotides have a length of 18 nucleotides. In one embodiment, the oligonucleotides have a length of 19 nucleotides.

DMD Targeting Oligonucleotides

Duchenne muscular dystrophy (DMD) affects 1 in 3500 newborn males, while Becker muscular dystrophy (BMD) affects 1 in 20,000. Both DMD and BMD are caused by mutations in the DMD gene, which is located on the X chromosome and codes for dystrophin. DMD patients suffer from progressive muscle weakness, are wheelchair bound before the age of 13, and often die before the third decade of their life. BMD is generally milder and patients often remain ambulant for over 40 years and have longer life expectancies compared to DMD patients.

Dystrophin is an essential component of the dystrophin-glycoprotein complex (DGC). Amongst other things, DGC maintains the membrane stability of muscle fibers. Frame-shifting mutations in the DMD gene result in dystrophin deficiency in muscle cells, which is accompanied by reduced levels of other DGC proteins and results in the severe phenotype found in DMD patients. Mutations in the DMD gene that keep the reading frame intact, generate shorter but partly functional dystrophins, and are associated with the less severe BMD. In Duchnenne Muscular Dystrophy (DMD) patients, frame-shifting mutations in the DMD gene cause an out-of-frame mRNA to be produced, resulting in a truncated, non-functional dystrophin protein. This in-frame mature mRNA encodes an in-frame dystrophin protein that is still partly functional and results in a milder Becker's Muscular Dystrophy (BMD) phenotype.

In certain embodiments the oligonucleotides of the invention are complementary to portions of the DMD gene, for example, Exon 51, Exon 53 and Exon 45.

### Exon 51

The sequence of exon 51 of the DMD gene (SEO ID NO: 4011 is shown below:

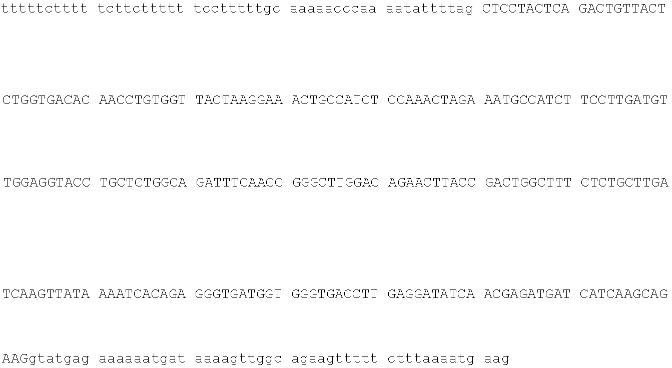

The corresponding transcript sequence of the highlighted portion is:

(SEQ ID NO: 402)
5′ CC AAA CTA GAA ATG CCA TCT TCC TTG ATG T 3′.

Oligonucleotides complementary to Exon 51 of the DMD gene, useful according to the invention include but are not limited to:

5′ GG TTT GAT CTT TAC GGT AGA AGG AAC TAC A 7′ (SEQ ID NO: 403) and the oligonucleotides provided in Table 3:

TABLE 3
Exon 51

| Sequence (5′ to 7′) | SEQ ID NO: |
|---|---|
| GGTTTGATCTTTACGGTA | 1 |
| GTTTGATCTTTACGGTAG | 2 |
| TTTGATCTTTACGGTAGA | 3 |
| TTGATCTTTACGGTAGAA | 4 |
| TGATCTTTACGGTAGAAG | 5 |
| GATCTTTACGGTAGAAGG | 6 |
| ATCTTTACGGTAGAAGGA | 7 |
| TCTTTACGGTAGAAGGAA | 8 |
| CTTTACGGTAGAAGGAAC | 9 |
| TTTACGGTAGAAGGAACT | 10 |
| TTACGGTAGAAGGAACTA | 11 |
| TACGGTAGAAGGAACTAC | 12 |
| ACGGTAGAAGGAACTACA | 13 |
| GGTTTGATCTTTACGGT | 14 |
| GTTTGATCTTTACGGTA | 15 |
| TTTGATCTTTACGGTAG | 16 |
| TTGATCTTTACGGTAGA | 17 |
| TGATCTTTACGGTAGAA | 18 |
| GATCTTTACGGTAGAAG | 19 |
| ATCTTTACGGTAGAAGG | 20 |
| TCTTTACGGTAGAAGGA | 21 |
| CTTTACGGTAGAAGGAA | 22 |
| TTTACGGTAGAAGGAAC | 23 |
| TTACGGTAGAAGGAACT | 24 |
| TACGGTAGAAGGAACTA | 25 |
| ACGGTAGAAGGAACTAC | 26 |
| CGGTAGAAGGAACTACA | 27 |
| GGTTTGATCTTTACGG | 28 |
| GTTTGATCTTTACGGT | 29 |
| TTTGATCTTTACGGTA | 30 |
| TTGATCTTTACGGTAG | 31 |
| TGATCTTTACGGTAGA | 32 |
| GATCTTTACGGTAGAA | 33 |
| ATCTTTACGGTAGAAG | 34 |
| TCTTTACGGTAGAAGG | 35 |
| CTTTACGGTAGAAGGA | 36 |
| TTTACGGTAGAAGGAA | 37 |
| TTACGGTAGAAGGAAC | 38 |
| TACGGTAGAAGGAACT | 39 |
| ACGGTAGAAGGAACTA | 40 |
| CGGTAGAAGGAACTAC | 41 |
| GGTAGAAGGAACTACA | 42 |
| GGTTTGATCTTTACG | 43 |
| GTTTGATCTTTACGG | 44 |
| TTTGATCTTTACGGT | 45 |
| TTGATCTTTACGGTA | 46 |
| TGATCTTTACGGTAG | 47 |
| GATCTTTACGGTAGA | 48 |
| ATCTTTACGGTAGAA | 49 |
| TCTTTACGGTAGAAG | 50 |
| CTTTACGGTAGAAGG | 51 |
| TTTACGGTAGAAGGA | 52 |
| TTACGGTAGAAGGAA | 53 |
| TACGGTAGAAGGAAC | 54 |
| ACGGTAGAAGGAACT | 55 |
| CGGTAGAAGGAACTA | 56 |
| GGTAGAAGGAACTAC | 57 |
| GTAGAAGGAACTACA | 58 |
| GGTTTGATCTTTAC | 59 |
| GTTTGATCTTTACG | 60 |
| TTTGATCTTTACGG | 61 |
| TTGATCTTTACGGT | 62 |
| TGATCTTTACGGTA | 63 |
| GATCTTTACGGTAG | 64 |
| ATCTTTACGGTAGA | 65 |
| TCTTTACGGTAGAA | 66 |
| CTTTACGGTAGAAG | 67 |
| TTTACGGTAGAAGG | 68 |
| TTACGGTAGAAGGA | 69 |
| TACGGTAGAAGGAA | 70 |
| ACGGTAGAAGGAAC | 71 |
| CGGTAGAAGGAACT | 72 |
| GGTAGAAGGAACTA | 73 |
| GTAGAAGGAACTAC | 74 |
| TAGAAGGAACTACA | 75 |

In one embodiment, said oligonucleotide comprises a sequence selected from the group consisting of SEQ ID NOs: 1 to 75. In one embodiment, said oligonucleotide comprises a sequence selected from the group consisting of SEQ ID NOs: 1 to 75, wherein the oligonucleotide has a length of 14 to 20 nucleotides. In one embodiment, said oligonucleotide comprises a sequence selected from the group consisting of SEQ ID NOs: 1 to 75, wherein the oligonucleotide has a length of 14 to 19 nucleotides. In one embodiment, said oligonucleotide has a length of 14 to 19 nucleotides. In one embodiment, said oligonucleotide has a length of 14 nucleotides. In one embodiment, said oligonucleotide has a length of 15 nucleotides. In one embodiment, said oligonucleotide has a length of 16 nucleotides. In one embodiment, said oligonucleotide has a length of 17 nucleotides. In one embodiment, said oligonucleotide has a length of 18 nucleotides. In one embodiment, said oligonucleotide has a length of 19 nucleotides. In one embodiment, said oligonucleotide has a length of 20 nucleotides.

In one embodiment, said oligonucleotide comprises a sequence selected from the group consisting of SEQ ID NOs: 1 to 75, wherein said oligonucleotide has a length of 19 nucleotides. In such embodiments, said oligonucleotide is a 19-mer. In one embodiment, said oligonucleotide comprises the sequence 5′ CTTTACGGTAGAAGGAACT 7′ (SEQ ID NO: 404; 19 mer). In one embodiment, said oligonucleotide consists of the sequence of SEQ ID NO: 404.

In one embodiment, said oligonucleotide comprises a sequence selected from the group consisting of SEQ ID NOs: 1 to 75, wherein said oligonucleotide has a length of 18 nucleotides. In such embodiments, said oligonucleotide is a 18-mer. In one embodiment, said oligonucleotide comprises a sequence selected from the group consisting of SEQ ID NOs: 1 to 13. In one embodiment, said oligonucleotide consists of a sequence selected from the group consisting of SEQ ID NOs: 1 to 13. In one embodiment, said oligonucleotide comprises the sequence of SEQ ID NO: 4 or of SEQ ID NO: 5. In one embodiment, said oligonucleotide comprises the sequence of SEQ ID NO: 4. In one embodiment, said oligonucleotide comprises the sequence of SEQ ID NO: 5. In one embodiment, said oligonucleotide consists of the sequence of SEQ ID NO: 4 or of SEQ ID NO: 5. In one embodiment, said oligonucleotide consists of the sequence of SEQ ID NO: 4. In one embodiment, said oligonucleotide consists of the sequence of SEQ ID NO: 5.

In one embodiment, said oligonucleotide comprises a sequence selected from the group consisting of SEQ ID NOs: 1 to 75, wherein said oligonucleotide has a length of 17 nucleotides. In such embodiments, said oligonucleotide is a 17-mer. In one embodiment, said oligonucleotide comprises a sequence selected from the group consisting of SEQ ID NOs: 14 to 27. In one embodiment, said oligonucleotide consists of a sequence selected from the group consisting of SEQ ID NOs: 14 to 27. In one embodiment, said oligonucleotide comprises the sequence of SEQ ID NO: 22, SEQ ID NO: 23 or of SEQ ID NO: 24. In one embodiment, said oligonucleotide comprises the sequence of SEQ ID NO: 22. In one embodiment, said oligonucleotide comprises the sequence of SEQ ID NO: 23. In one embodiment, said oligonucleotide comprises the sequence of SEQ ID NO: 24. In one embodiment, said oligonucleotide consists of the sequence of SEQ ID NO: 22, SEQ ID NO: 23 or of SEQ ID NO: 24. In one embodiment, said oligonucleotide consists of the sequence of SEQ ID NO: 22. In one embodiment, said oligonucleotide consists of the sequence of SEQ ID NO: 23. In one embodiment, said oligonucleotide consists of the sequence of SEQ ID NO:

24.

In one embodiment, said oligonucleotide comprises a sequence selected from the group consisting of SEQ ID NOs: 1 to 75, wherein said oligonucleotide has a length of 16 nucleotides. In such embodiments, said oligonucleotide is a 16-mer. In one embodiment, said oligonucleotide comprises a sequence selected from the group consisting of SEQ ID NOs: 28 to 42. In one embodiment, said oligonucleotide consists of a sequence selected from the group consisting of SEQ ID NOs: 28 to 42. In one embodiment, said oligonucleotide comprises the sequence of SEQ ID NO: 36, SEQ ID NO: 37, SEQ ID NO: 38 or of SEQ ID NO: 39. In one embodiment, said oligonucleotide comprises the sequence of SEQ ID NO: 36. In one embodiment, said oligonucleotide comprises the sequence of SEQ ID NO: 37. In one embodiment, said oligonucleotide comprises the sequence of SEQ ID NO: 38. In one embodiment, said oligonucleotide comprises the sequence of SEQ ID NO: 39. In one embodiment, said oligonucleotide consists of the sequence of SEQ ID NO: 36, SEQ ID NO: 37, SEQ ID NO: 38 or of SEQ ID NO: 39. In one embodiment, said oligonucleotide consists of the sequence of SEQ ID NO: 36. In one embodiment, said oligonucleotide consists of the sequence of SEQ ID NO: 37. In one embodiment, said oligonucleotide consists of the sequence of SEQ ID NO: 38. In one embodiment, said oligonucleotide consists of the sequence of SEQ ID NO: 39.

In one embodiment, said oligonucleotide comprises a sequence selected from the group consisting of SEQ ID NOs: 1 to 75, wherein said oligonucleotide has a length of 15 nucleotides. In such embodiments, said oligonucleotide is a 15-mer. In one embodiment, said oligonucleotide comprises a sequence selected from the group consisting of SEQ ID NOs: 43 to 58. In one embodiment, said oligonucleotide consists of a sequence selected from the group consisting of SEQ ID NOs: 43 to 58. In one embodiment, said oligonucleotide comprises the sequence of SEQ ID NO: 51, SEQ ID NO: 52, SEQ ID NO: 53, SEQ ID NO: 54 or of SEQ ID NO: 55. In one embodiment, said oligonucleotide comprises the sequence of SEQ ID NO: 51. In one embodiment, said oligonucleotide comprises the sequence of SEQ ID NO: 52. In one embodiment, said oligonucleotide comprises the sequence of SEQ ID NO: 53. In one embodiment, said oligonucleotide comprises the sequence of SEQ ID NO: 54. In one embodiment, said oligonucleotide comprises the sequence of SEQ ID NO: 55. In one embodiment, said oligonucleotide consists of the sequence of SEQ ID NO: 51, SEQ ID NO: 52, SEQ ID NO: 53, SEQ ID NO: 54 or of SEQ ID NO: 55. In one embodiment, said oligonucleotide consists of the sequence of SEQ ID NO: 51. In one embodiment, said oligonucleotide consists of the sequence of SEQ ID NO: 52. In one embodiment, said oligonucleotide consists of the sequence of SEQ ID NO: 53. In one embodiment, said oligonucleotide consists of the sequence of SEQ ID NO: 54. In one embodiment, said oligonucleotide consists of the sequence of SEQ ID NO: 55.

In one embodiment, said oligonucleotide comprises a sequence selected from the group consisting of SEQ ID NOs: 1 to 75, wherein said oligonucleotide has a length of 14 nucleotides. In such embodiments, said oligonucleotide is a 14-mer. In one embodiment, said oligonucleotide comprises a sequence selected from the group consisting of SEQ ID NOs: 59 to 75. In one embodiment, said oligonucleotide consists of a sequence selected from the group consisting of SEQ ID NOs: 59 to 75. In one embodiment, said oligonucleotide comprises the sequence of SEQ ID NO: 67, SEQ ID NO: 68, SEQ ID NO: 69 or of SEQ ID NO: 70. In one embodiment, said oligonucleotide comprises the sequence of SEQ ID NO: 67. In one embodiment, said oligonucleotide comprises the sequence of SEQ ID NO: 68. In one embodiment, said oligonucleotide comprises the sequence of SEQ ID NO: 69. In one embodiment, said oligonucleotide comprises the sequence of SEQ ID NO: 70. In one embodiment, said oligonucleotide consists of the sequence of SEQ ID NO: 67, SEQ ID NO: 68, SEQ ID NO: 69 or of SEQ ID NO: 70. In one embodiment, said oligonucleotide consists of the sequence of SEQ ID NO: 67. In one embodiment, said oligonucleotide consists of the sequence of SEQ ID NO: 68. In one embodiment, said oligonucleotide consists of the sequence of SEQ ID NO: 69. In one embodiment, said oligonucleotide consists of the sequence of SEQ ID NO: 70.

In one embodiment, said oligonucleotide comprises a sequence selected from the group consisting of SEQ ID NOs: 4, 5, 22 to 24, 36 to 39, 51 to 55, 67 to 70 and SEQ ID NO: 404. In one embodiment, said oligonucleotide comprises a sequence selected from the group consisting of SEQ ID NOs: 4, 5, 22 to 24, 36 to 39, 51 to 55, 67 to 70 and SEQ ID NO: 404, wherein all of the residues are abc-DNA residues. In one embodiment, said oligonucleotide consists of the sequence selected from the group consisting of SEQ ID NOs: 4, 5, 22 to 24, 36 to 39, 51 to 55, 67 to 70 and SEQ ID NO: 404. In one embodiment, said oligonucleotide consists of the sequence selected from the group consisting of SEQ ID NOs: 4, 5, 22 to 24, 36 to 39, 51 to 55, 67 to 70 and SEQ ID NO: 404, wherein all of the residues are abc-DNA residues. In one embodiment, said oligonucleotide comprises a sequence selected from the group consisting of SEQ ID NOs: 4, 5, 22 to 24, 36 to 39, 51 to 55 and SEQ ID NO: 404. In one embodiment, said oligonucleotide comprises a sequence selected from the group consisting of SEQ ID NOs: 4, 5, 22 to 24, 36 to 39, 51 to 55 and SEQ ID NO: 404, wherein all of the residues are abc-DNA residues. In one embodiment, said oligonucleotide consists of the sequence selected from the group consisting of SEQ ID NOs: 4, 5, 22 to 24, 36 to 39, 51 to 55 and SEQ ID NO: 404. In one embodiment, said oligonucleotide consists of the sequence selected from the group consisting of SEQ ID NOs: 4, 5, 22 to 24, 36 to 39, 51 to 55 and SEQ ID NO: 404, wherein all of the residues are abc-DNA residues. In one embodiment, said oligonucleotide comprises a sequence selected from the group consisting of SEQ ID NO: 417 and SEQ ID NO: 418. In one embodiment, said oligonucleotide comprises the sequence of SEQ ID NO: 417. In one embodiment, said oligonucleotide comprises the sequence of SEQ ID NO: 418. In one embodiment, said oligonucleotide consists of a sequence selected from the group consisting of SEQ ID NO: 417 and SEQ ID NO: 418. In one embodiment, said oligonucleotide consists of the sequence of SEQ ID NO: 417. In one embodiment, said oligonucleotide consists of the sequence of SEQ ID NO: 418.

### Exon 53

The sequence of Exon 53 of the DMD gene (SEQ ID NO: 405) is shown below: Exon 53

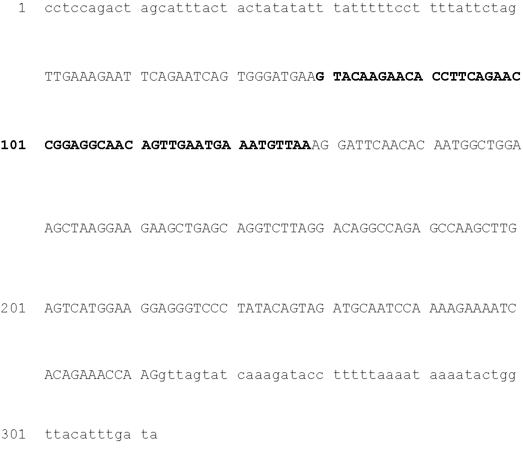

The corresponding transcript sequence of the highlighted portion is:

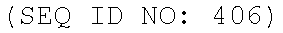

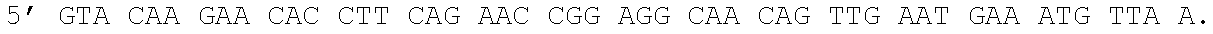

Oligonucleotides complementary to Exon 53 of the DMD gene, useful according to the invention include but are not limited to:

5′ CAT GTT CTT GTG GAA GTC TTG GCC TCC GTT GTC AAC TTA CTT TAC AAT 7′ (SEQ ID NO: 407) and the oligonucleotides provided in Table 4.

TABLE 4
Exon 53

| Sequence (5′ to 7′) | SEQ ID NO: |
|---|---|
| CATGTTCTTGTGGAAGTC | 76 |
| ATGTTCTTGTGGAAGTCT | 77 |
| TGTTCTTGTGGAAGTCTT | 78 |
| GTTCTTGTGGAAGTCTTG | 79 |
| TTCTTGTGGAAGTCTTGG | 80 |
| TCTTGTGGAAGTCTTGGC | 81 |
| CTTGTGGAAGTCTTGGCC | 82 |
| TTGTGGAAGTCTTGGCCT | 83 |
| TGTGGAAGTCTTGGCCTC | 84 |
| GTGGAAGTCTTGGCCTCC | 85 |
| TGGAAGTCTTGGCCTCCG | 86 |
| GGAAGTCTTGGCCTCCGT | 87 |
| GAAGTCTTGGCCTCCGTT | 88 |
| AAGTCTTGGCCTCCGTTG | 89 |
| AGTCTTGGCCTCCGTTGT | 90 |
| GTCTTGGCCTCCGTTGTC | 91 |
| TCTTGGCCTCCGTTGTCA | 92 |
| CTTGGCCTCCGTTGTCAA | 93 |
| TTGGCCTCCGTTGTCAAC | 94 |
| TGGCCTCCGTTGTCAACT | 95 |
| GGCCTCCGTTGTCAACTT | 96 |
| GCCTCCGTTGTCAACTTA | 97 |
| CCTCCGTTGTCAACTTAC | 98 |
| CTCCGTTGTCAACTTACT | 99 |
| TCCGTTGTCAACTTACTT | 100 |
| CCGTTGTCAACTTACTTT | 101 |
| CGTTGTCAACTTACTTTA | 102 |
| GTTGTCAACTTACTTTAC | 103 |
| TTGTCAACTTACTTTACA | 104 |
| TGTCAACTTACTTTACAA | 105 |
| GTCAACTTACTTTACAAT | 106 |
| CATGTTCTTGTGGAAGT | 107 |
| ATGTTCTTGTGGAAGTC | 108 |
| TGTTCTTGTGGAAGTCT | 109 |
| GTTCTTGTGGAAGTCTT | 110 |
| TTCTTGTGGAAGTCTTG | 111 |
| TCTTGTGGAAGTCTTGG | 112 |
| CTTGTGGAAGTCTTGGC | 113 |
| TTGTGGAAGTCTTGGCC | 114 |
| TGTGGAAGTCTTGGCCT | 115 |
| GTGGAAGTCTTGGCCTC | 116 |
| TGGAAGTCTTGGCCTCC | 117 |
| GGAAGTCTTGGCCTCCG | 118 |
| GAAGTCTTGGCCTCCGT | 119 |
| AAGTCTTGGCCTCCGTT | 120 |
| AGTCTTGGCCTCCGTTG | 121 |
| GTCTTGGCCTCCGTTGT | 122 |
| TCTTGGCCTCCGTTGTC | 123 |
| CTTGGCCTCCGTTGTCA | 124 |
| TTGGCCTCCGTTGTCAA | 125 |
| TGGCCTCCGTTGTCAAC | 126 |
| GGCCTCCGTTGTCAACT | 127 |
| GCCTCCGTTGTCAACTT | 128 |
| CCTCCGTTGTCAACTTA | 129 |
| CTCCGTTGTCAACTTAC | 130 |
| TCCGTTGTCAACTTACT | 131 |
| CCGTTGTCAACTTACTT | 132 |
| CGTTGTCAACTTACTTT | 133 |
| GTTGTCAACTTACTTTA | 134 |
| TTGTCAACTTACTTTAC | 135 |
| TGTCAACTTACTTTACA | 136 |
| GTCAACTTACTTTACAA | 137 |
| TCAACTTACTTTACAAT | 138 |
| CATGTTCTTGTGGAAG | 139 |
| ATGTTCTTGTGGAAGT | 140 |
| TGTTCTTGTGGAAGTC | 141 |
| GTTCTTGTGGAAGTCT | 142 |
| TTCTTGTGGAAGTCTT | 143 |
| TCTTGTGGAAGTCTTG | 144 |
| CTTGTGGAAGTCTTGG | 145 |
| TTGTGGAAGTCTTGGC | 146 |
| TGTGGAAGTCTTGGCC | 147 |
| GTGGAAGTCTTGGCCT | 148 |
| TGGAAGTCTTGGCCTC | 149 |
| GGAAGTCTTGGCCTCC | 150 |
| GAAGTCTTGGCCTCCG | 151 |
| AAGTCTTGGCCTCCGT | 152 |
| AGTCTTGGCCTCCGTT | 153 |
| GTCTTGGCCTCCGTTG | 154 |
| TCTTGGCCTCCGTTGT | 155 |
| CTTGGCCTCCGTTGTC | 156 |
| TTGGCCTCCGTTGTCA | 157 |
| TGGCCTCCGTTGTCAA | 158 |
| GGCCTCCGTTGTCAAC | 159 |
| GCCTCCGTTGTCAACT | 160 |
| CCTCCGTTGTCAACTT | 161 |
| CTCCGTTGTCAACTTA | 162 |
| TCCGTTGTCAACTTAC | 163 |
| CCGTTGTCAACTTACT | 164 |
| CGTTGTCAACTTACTT | 165 |
| GTTGTCAACTTACTTT | 166 |
| TTGTCAACTTACTTTA | 167 |
| TGTCAACTTACTTTAC | 168 |
| GTCAACTTACTTTACA | 169 |
| TCAACTTACTTTACAA | 170 |
| CAACTTACTTTACAAT | 171 |
| CATGTTCTTGTGGAA | 172 |
| ATGTTCTTGTGGAAG | 173 |
| TGTTCTTGTGGAAGT | 174 |
| GTTCTTGTGGAAGTC | 175 |
| TTCTTGTGGAAGTCT | 176 |
| TCTTGTGGAAGTCTT | 177 |
| CTTGTGGAAGTCTTG | 178 |
| TTGTGGAAGTCTTGG | 179 |
| TGTGGAAGTCTTGGC | 180 |
| GTGGAAGTCTTGGCC | 181 |
| TGGAAGTCTTGGCCT | 182 |
| GGAAGTCTTGGCCTC | 183 |
| GAAGTCTTGGCCTCC | 184 |
| AAGTCTTGGCCTCCG | 185 |
| AGTCTTGGCCTCCGT | 186 |
| GTCTTGGCCTCCGTT | 187 |
| TCTTGGCCTCCGTTG | 188 |
| CTTGGCCTCCGTTGT | 189 |
| TTGGCCTCCGTTGTC | 190 |
| TGGCCTCCGTTGTCA | 191 |
| GGCCTCCGTTGTCAA | 192 |
| GCCTCCGTTGTCAAC | 193 |
| CCTCCGTTGTCAACT | 194 |
| CTCCGTTGTCAACTT | 195 |
| TCCGTTGTCAACTTA | 196 |
| CCGTTGTCAACTTAC | 197 |
| CGTTGTCAACTTACT | 198 |
| GTTGTCAACTTACTT | 199 |
| TTGTCAACTTACTTT | 200 |
| TGTCAACTTACTTTA | 201 |
| GTCAACTTACTTTAC | 202 |
| TCAACTTACTTTACA | 203 |
| CAACTTACTTTACAA | 204 |
| AACTTACTTTACAAT | 205 |
| CATGTTCTTGTGGA | 206 |
| ATGTTCTTGTGGAA | 207 |
| TGTTCTTGTGGAAG | 208 |
| GTTCTTGTGGAAGT | 209 |
| TTCTTGTGGAAGTC | 210 |
| TCTTGTGGAAGTCT | 211 |
| CTTGTGGAAGTCTT | 212 |
| TTGTGGAAGTCTTG | 213 |
| TGTGGAAGTCTTGG | 214 |
| GTGGAAGTCTTGGC | 215 |
| TGGAAGTCTTGGCC | 216 |
| GGAAGTCTTGGCCT | 217 |
| GAAGTCTTGGCCTC | 218 |
| AAGTCTTGGCCTCC | 219 |
| AGTCTTGGCCTCCG | 220 |
| GTCTTGGCCTCCGT | 221 |
| TCTTGGCCTCCGTT | 222 |
| CTTGGCCTCCGTTG | 223 |
| TTGGCCTCCGTTGT | 224 |
| TGGCCTCCGTTGTC | 225 |
| GGCCTCCGTTGTCA | 226 |
| GCCTCCGTTGTCAA | 227 |
| CCTCCGTTGTCAAC | 228 |
| CTCCGTTGTCAACT | 229 |
| TCCGTTGTCAACTT | 230 |
| CCGTTGTCAACTTA | 231 |
| CGTTGTCAACTTAC | 232 |
| GTTGTCAACTTACT | 233 |
| TTGTCAACTTACTT | 234 |
| TGTCAACTTACTTT | 235 |
| GTCAACTTACTTTA | 236 |
| TCAACTTACTTTAC | 237 |
| CAACTTACTTTACA | 238 |
| AACTTACTTTACAA | 239 |
| ACTTACTTTACAAT | 240 |

In one embodiment, said oligonucleotide comprises a sequence selected from the group consisting of SEQ ID NOs: 76 to 240. In one embodiment, said oligonucleotide comprises a sequence selected from the group consisting of SEQ ID NOs: 76 to 240, wherein the oligonucleotide has a length of 14 to 20 nucleotides. In one embodiment, said oligonucleotide comprises a sequence selected from the group consisting of SEQ ID NOs: 76 to 240, wherein the oligonucleotide has a length of 14 to 19 nucleotides. In one embodiment, said oligonucleotide has a length of 14 nucleotides. In one embodiment, said oligonucleotide has a length of 15 nucleotides. In one embodiment, said oligonucleotide has a length of 16 nucleotides. In one embodiment, said oligonucleotide has a length of 17 nucleotides. In one embodiment, said oligonucleotide has a length of 18 nucleotides. In one embodiment, said oligonucleotide has a length of 19 nucleotides.

In one embodiment, said oligonucleotide comprises a sequence selected from the group consisting of SEQ ID NOs: 76 to 240, wherein said oligonucleotide has a length of 18 nucleotides. In such embodiments, said oligonucleotide is a 18-mer. In one embodiment, said oligonucleotide comprises a sequence selected from the group consisting of SEQ ID NOs: 76 to 106. In one embodiment, said oligonucleotide consists of a sequence selected from the group consisting of SEQ ID NOs: 76 to 106.

In one embodiment, said oligonucleotide comprises a sequence selected from the group consisting of SEQ ID NOs: 76 to 240, wherein said oligonucleotide has a length of 17 nucleotides. In such embodiments, said oligonucleotide is a 17-mer. In one embodiment, said oligonucleotide comprises a sequence selected from the group consisting of SEQ ID NOs: 107 to 138. In one embodiment, said oligonucleotide consists of a sequence selected from the group consisting of SEQ ID NOs: 107 to 138.

In one embodiment, said oligonucleotide comprises a sequence selected from the group consisting of SEQ ID NOs: 76 to 240, wherein said oligonucleotide has a length of 16 nucleotides. In such embodiments, said oligonucleotide is a 16-mer. In one embodiment, said oligonucleotide comprises a sequence selected from the group consisting of SEQ ID NOs: 139 to 171. In one embodiment, said oligonucleotide consists of a sequence selected from the group consisting of SEQ ID NOs: 139 to 171.

In one embodiment, said oligonucleotide comprises a sequence selected from the group consisting of SEQ ID NOs: 76 to 240, wherein said oligonucleotide has a length of 15 nucleotides. In such embodiments, said oligonucleotide is a 15-mer. In one embodiment, said oligonucleotide comprises a sequence selected from the group consisting of SEQ ID NOs: 172 to 205. In one embodiment, said oligonucleotide consists of a sequence selected from the group consisting of SEQ ID NOs: 172 to 205.

In one embodiment, said oligonucleotide comprises a sequence selected from the group consisting of SEQ ID NOs: 76 to 240, wherein said oligonucleotide has a length of 14 nucleotides. In such embodiments, said oligonucleotide is a 14-mer. In one embodiment, said oligonucleotide comprises a sequence selected from the group consisting of SEQ ID NOs: 206 to 240. In one embodiment, said oligonucleotide consists of a sequence selected from the group consisting of SEQ ID NOs: 206 to 240.

### Exon 45

The sequence of Exon 45 of the DMD gene (SEQ ID NO: 408) is shown below:

Exon 45

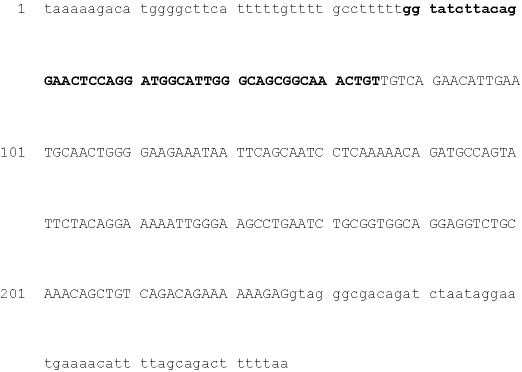

The corresponding transcript sequence of the highlighted portion is:

(SEQ ID NO: 409)

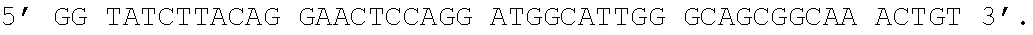

Oligonucleotides complementary to Exon 45 of the DMD gene, useful according to the invention include but are not limited to: 5′ CC ATAGAATGTC CTTGAGGTCC TACCGTAACC CGTCGCCGTT TGACA 7′ (SEQ ID NO: 410) and any one of the sequences presented in Table 5.

TABLE 5
Exon 45

| Sequence (5′ to 7′) | SEQ ID NO: |
|---|---|
| CCATAGAATGTCCTTGAG | 241 |
| CATAGAATGTCCTTGAGG | 242 |
| ATAGAATGTCCTTGAGGT | 243 |
| TAGAATGTCCTTGAGGTC | 244 |
| AGAATGTCCTTGAGGTCC | 245 |
| GAATGTCCTTGAGGTCCT | 246 |
| AATGTCCTTGAGGTCCTA | 247 |
| ATGTCCTTGAGGTCCTAC | 248 |
| TGTCCTTGAGGTCCTACC | 249 |
| GTCCTTGAGGTCCTACCG | 250 |
| TCCTTGAGGTCCTACCGT | 251 |
| CCTTGAGGTCCTACCGTA | 252 |
| CTTGAGGTCCTACCGTAA | 253 |
| TTGAGGTCCTACCGTAAC | 254 |
| TGAGGTCCTACCGTAACC | 255 |
| GAGGTCCTACCGTAACCC | 256 |
| AGGTCCTACCGTAACCCG | 257 |
| GGTCCTACCGTAACCCGT | 258 |
| GTCCTACCGTAACCCGTC | 259 |
| TCCTACCGTAACCCGTCG | 260 |
| CCTACCGTAACCCGTCGC | 261 |
| CTACCGTAACCCGTCGCC | 262 |
| TACCGTAACCCGTCGCCG | 263 |
| ACCGTAACCCGTCGCCGT | 264 |
| CCGTAACCCGTCGCCGTT | 265 |
| CGTAACCCGTCGCCGTTT | 266 |
| GTAACCCGTCGCCGTTTG | 267 |
| TAACCCGTCGCCGTTTGA | 268 |
| AACCCGTCGCCGTTTGAC | 269 |
| ACCCGTCGCCGTTTGACA | 270 |
| CCATAGAATGTCCTTGA | 271 |
| CATAGAATGTCCTTGAG | 272 |
| ATAGAATGTCCTTGAGG | 273 |
| TAGAATGTCCTTGAGGT | 274 |
| AGAATGTCCTTGAGGTC | 275 |
| GAATGTCCTTGAGGTCC | 276 |
| AATGTCCTTGAGGTCCT | 277 |
| ATGTCCTTGAGGTCCTA | 278 |
| TGTCCTTGAGGTCCTAC | 279 |
| GTCCTTGAGGTCCTACC | 280 |
| TCCTTGAGGTCCTACCG | 281 |
| CCTTGAGGTCCTACCGT | 282 |
| CTTGAGGTCCTACCGTA | 283 |
| TTGAGGTCCTACCGTAA | 284 |
| TGAGGTCCTACCGTAAC | 285 |
| GAGGTCCTACCGTAACC | 286 |
| AGGTCCTACCGTAACCC | 287 |
| GGTCCTACCGTAACCCG | 288 |
| GTCCTACCGTAACCCGT | 289 |
| TCCTACCGTAACCCGTC | 290 |
| CCTACCGTAACCCGTCG | 291 |
| CTACCGTAACCCGTCGC | 292 |
| TACCGTAACCCGTCGCC | 293 |
| ACCGTAACCCGTCGCCG | 294 |
| CCGTAACCCGTCGCCGT | 295 |
| CGTAACCCGTCGCCGTT | 296 |
| GTAACCCGTCGCCGTTT | 297 |
| TAACCCGTCGCCGTTTG | 298 |
| AACCCGTCGCCGTTTGA | 299 |
| ACCCGTCGCCGTTTGAC | 300 |
| CCCGTCGCCGTTTGACA | 301 |
| CCATAGAATGTCCTTG | 302 |
| CATAGAATGTCCTTGA | 303 |
| ATAGAATGTCCTTGAG | 304 |
| TAGAATGTCCTTGAGG | 305 |
| AGAATGTCCTTGAGGT | 306 |
| GAATGTCCTTGAGGTC | 307 |
| AATGTCCTTGAGGTCC | 308 |
| ATGTCCTTGAGGTCCT | 309 |
| TGTCCTTGAGGTCCTA | 310 |
| GTCCTTGAGGTCCTAC | 311 |
| TCCTTGAGGTCCTACC | 312 |
| CCTTGAGGTCCTACCG | 313 |
| CTTGAGGTCCTACCGT | 314 |
| TTGAGGTCCTACCGTA | 315 |
| TGAGGTCCTACCGTAA | 316 |
| GAGGTCCTACCGTAAC | 317 |
| AGGTCCTACCGTAACC | 318 |
| GGTCCTACCGTAACCC | 319 |
| GTCCTACCGTAACCCG | 320 |
| TCCTACCGTAACCCGT | 321 |
| CCTACCGTAACCCGTC | 322 |
| CTACCGTAACCCGTCG | 323 |
| TACCGTAACCCGTCGC | 324 |
| ACCGTAACCCGTCGCC | 325 |
| CCGTAACCCGTCGCCG | 326 |
| CGTAACCCGTCGCCGT | 327 |
| GTAACCCGTCGCCGTT | 328 |
| TAACCCGTCGCCGTTT | 329 |
| AACCCGTCGCCGTTTG | 330 |
| ACCCGTCGCCGTTTGA | 331 |
| CCCGTCGCCGTTTGAC | 332 |
| CCGTCGCCGTTTGACA | 333 |
| CCATAGAATGTCCTT | 334 |
| CATAGAATGTCCTTG | 335 |
| ATAGAATGTCCTTGA | 336 |
| TAGAATGTCCTTGAG | 337 |
| AGAATGTCCTTGAGG | 338 |
| GAATGTCCTTGAGGT | 339 |
| AATGTCCTTGAGGTC | 340 |
| ATGTCCTTGAGGTCC | 341 |
| TGTCCTTGAGGTCCT | 342 |
| GTCCTTGAGGTCCTA | 343 |
| TCCTTGAGGTCCTAC | 344 |
| CCTTGAGGTCCTACC | 345 |
| CTTGAGGTCCTACCG | 346 |
| TTGAGGTCCTACCGT | 347 |
| TGAGGTCCTACCGTA | 348 |
| GAGGTCCTACCGTAA | 349 |
| AGGTCCTACCGTAAC | 350 |
| GGTCCTACCGTAACC | 351 |
| GTCCTACCGTAACCC | 352 |
| TCCTACCGTAACCCG | 353 |
| CCTACCGTAACCCGT | 354 |
| CTACCGTAACCCGTC | 355 |
| TACCGTAACCCGTCG | 356 |
| ACCGTAACCCGTCGC | 357 |
| CCGTAACCCGTCGCC | 358 |
| CGTAACCCGTCGCCG | 359 |
| GTAACCCGTCGCCGT | 360 |
| TAACCCGTCGCCGTT | 361 |
| AACCCGTCGCCGTTT | 362 |
| ACCCGTCGCCGTTTG | 363 |
| CCCGTCGCCGTTTGA | 364 |
| CCGTCGCCGTTTGAC | 365 |
| CGTCGCCGTTTGACA | 366 |
| CCATAGAATGTCCT | 367 |
| CATAGAATGTCCTT | 368 |
| ATAGAATGTCCTTG | 369 |
| TAGAATGTCCTTGA | 370 |
| AGAATGTCCTTGAG | 371 |
| GAATGTCCTTGAGG | 372 |
| AATGTCCTTGAGGT | 373 |
| ATGTCCTTGAGGTC | 374 |
| TGTCCTTGAGGTCC | 375 |
| GTCCTTGAGGTCCT | 376 |
| TCCTTGAGGTCCTA | 377 |
| CCTTGAGGTCCTAC | 378 |
| CTTGAGGTCCTACC | 379 |
| TTGAGGTCCTACCG | 380 |
| TGAGGTCCTACCGT | 381 |
| GAGGTCCTACCGTA | 382 |
| AGGTCCTACCGTAA | 383 |
| GGTCCTACCGTAAC | 384 |
| GTCCTACCGTAACC | 385 |
| TCCTACCGTAACCC | 386 |
| CCTACCGTAACCCG | 387 |
| CTACCGTAACCCGT | 388 |
| TACCGTAACCCGTC | 389 |
| ACCGTAACCCGTCG | 390 |
| CCGTAACCCGTCGC | 391 |
| CGTAACCCGTCGCC | 392 |
| GTAACCCGTCGCCG | 393 |
| TAACCCGTCGCCGT | 394 |
| AACCCGTCGCCGTT | 395 |
| ACCCGTCGCCGTTT | 396 |
| CCCGTCGCCGTTTG | 397 |
| CCGTCGCCGTTTGA | 398 |
| CGTCGCCGTTTGAC | 399 |
| GTCGCCGTTTGACA | 400 |

In one embodiment, said oligonucleotide comprises a sequence selected from the group consisting of SEQ ID NOs: 241 to 400. In one embodiment, said oligonucleotide comprises a sequence selected from the group consisting of SEQ ID NOs: 241 to 270, wherein the oligonucleotide has a length of 14 to 20 nucleotides. In one embodiment, said oligonucleotide comprises a sequence selected from the group consisting of SEQ ID NOs: 241 to 400, wherein the oligonucleotide has a length of 14 to 19 nucleotides. In one embodiment, said oligonucleotide has a length of 14 nucleotides. In one embodiment, said oligonucleotide has a length of 15 nucleotides. In one embodiment, said oligonucleotide has a length of 16 nucleotides. In one embodiment, said oligonucleotide has a length of 17 nucleotides. In one embodiment, said oligonucleotide has a length of 18 nucleotides. In one embodiment, said oligonucleotide has a length of 19 nucleotides.

In one embodiment, said oligonucleotide comprises a sequence selected from the group consisting of SEQ ID NOs: 241 to 400, wherein said oligonucleotide has a length of 18 nucleotides. In such embodiments, said oligonucleotide is a 18-mer. In one embodiment, said oligonucleotide comprises a sequence selected from the group consisting of SEQ ID NOs: 241 to 270. In one embodiment, said oligonucleotide consists of a sequence selected from the group consisting of SEQ ID NOs: 241 to 270.

In one embodiment, said oligonucleotide comprises a sequence selected from the group consisting of SEQ ID NOs: 241 to 400, wherein said oligonucleotide has a length of 17 nucleotides. In such embodiments, said oligonucleotide is a 17-mer. In one embodiment, said oligonucleotide comprises a sequence selected from the group consisting of SEQ ID NOs: 271 to 301. In one embodiment, said oligonucleotide consists of a sequence selected from the group consisting of SEQ ID NOs: 271 to 301.

In one embodiment, said oligonucleotide comprises a sequence selected from the group consisting of SEQ ID NOs: 241 to 400, wherein said oligonucleotide has a length of 16 nucleotides. In such embodiments, said oligonucleotide is a 16-mer. In one embodiment, said oligonucleotide comprises a sequence selected from the group consisting of SEQ ID NOs: 302 to 333. In one embodiment, said oligonucleotide consists of a sequence selected from the group consisting of SEQ ID NOs: 302 to 333.

In one embodiment, said oligonucleotide comprises a sequence selected from the group consisting of SEQ ID NOs: 241 to 400, wherein said oligonucleotide has a length of 15 nucleotides. In such embodiments, said oligonucleotide is a 15-mer. In one embodiment, said oligonucleotide comprises a sequence selected from the group consisting of SEQ ID NOs: 334 to 366. In one embodiment, said oligonucleotide consists of a sequence selected from the group consisting of SEQ ID NOs: 334 to 366.

In one embodiment, said oligonucleotide comprises a sequence selected from the group consisting of SEQ ID NOs: 241 to 400, wherein said oligonucleotide has a length of 14 nucleotides. In such embodiments, said oligonucleotide is a 14-mer. In one embodiment, said oligonucleotide comprises a sequence selected from the group consisting of SEQ ID NOs: 367 to 240. In one embodiment, said oligonucleotide consists of a sequence selected from the group consisting of SEQ ID NOs: 367 to 240.

The invention also provides for oligonucleotides that are complementary to the intronic splicing silencer N1 (ISS-N1) in Spinal Muscular Atrophy, for example TCACTTTCATAATGCTGG (SEQ ID NO: 411).

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control.

The materials, methods, and examples are illustrative only and not intended to be limiting to the various embodiments of the invention described herein.

### EXAMPLES

### Example 1

### Affinity of Alpha Anomeric Oligonucleotides Toward Complementary Parallel RNA

The affinity toward complementary parallel RNA was assessed for several alpha anomeric oligonucleotides by UV-melting experiments (Table 1).

The temperatures of melting range from 53.3° C. to 77.0° C., demonstrating the good affinity of alpha anomeric oligonucleotides for their RNA complements.

TABLE 1
Tₘ data from UV-melting curves (260 nm) of alpha anomeric
oligonucleotides in duplex with complementary parallel RNA.

| | | | Tₘ(° C.) vs |
|---|---|---|---|
| Entry | SEQ ID NO: | Sequence ^{a} | parallel RNA |
| ON1 ^{b,c} | 412 | 5′-(tccattcggctccaa*palm)-7′ | 76.2 |
| ON2 ^{b,c} | 413 | 5′-(t*c*c*a*t*t*c*g*g*c*t*c*c*a*a)-7′ | 77.0 |
| ON3 ^{d} | 414 | 5′-(gatctttacggtagaagg)-7′ | 72.5 |
| ON4 ^{d} | 415 | 5′-(atctttacggtagaagga)-7′ | 70.2 |
| ON5 ^{d} | 416 | 5′-(tctttacggtagaaggaa)-7′ | 69.1 |
| ON6 ^{d} | 417 | 5′-(ctttacggtagaaggaac)-7′ | 68.7 |
| ON7 ^{d} | 418 | 5′-(tttacggtagaaggaact)-7′ | 69.3 |
| ON8 ^{d} | 419 | 5′-(ttacggtagaaggaacta)-7′ | 70.8 |
| ON9 ^{d} | 420 | 5′-(tacggtagaaggaactac)-7′ | 71.0 |
| ON10 ^{d} | 421 | 5′-(aactagttcaatatttta)-7′ | 53.3 |
| ON11 ^{d} | 422 | 5′-(ctagttcaatattttagt)-7′ | 54.7 |
| ON12 ^{d} | 423 | 5′-(agttcaatattttagtgt)-7′ | 57.4 |
| ON13 ^{d} | 424 | 5′-(ttcaatattttagtgtct)-7′ | 54.7 |
| ON14 ^{d} | 425 | 5′-(caatattttagtgtctcc)-7′ | 60.4 |

^{a} a, g, t, c corresponds to abc-DNA modified adenine, guanine, thymine and methylcytosine respectively, * denotes a phosphorothioate linkage, palm correspond to a palmitic acid conjugated via an alkyl linker to the oligonucleotide
^{b} total strand conc. 2 μM in 10 mM NaH₂PO₄, 150 mM NaCl, pH 7.0
^{c} Tₘ of unmodified duplexes, DNA/RNA: 67.4° C.
^{d} total strand conc. 2 μM in 10 mM NaH₂PO₄, 75 mM NaCl, pH 7.0

### Example 2

### Stability of Alpha Anomeric Oligonucleotides in Acidic Condition

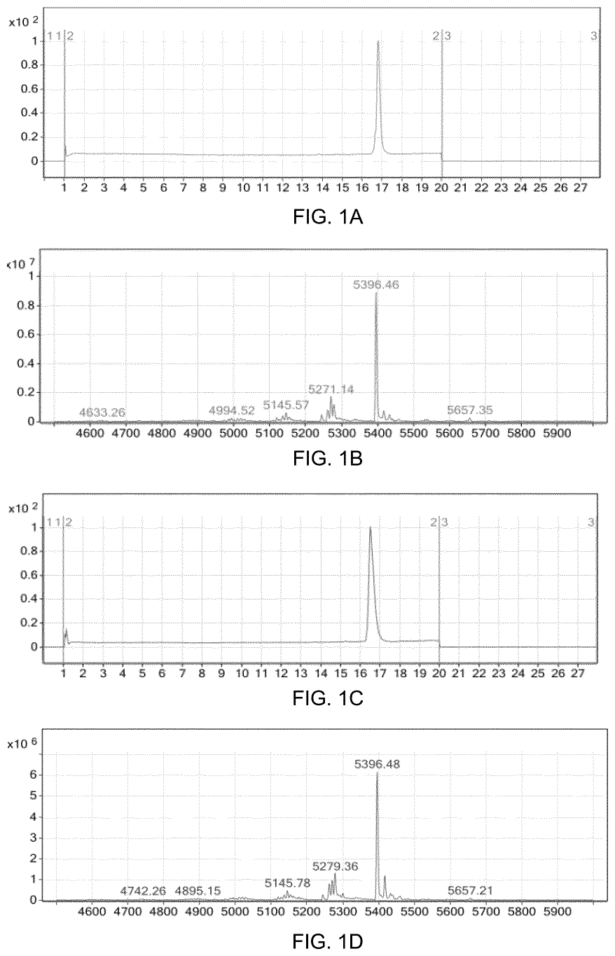

The acidic stability of ON1 was assessed by diluting ON1 to 10 μM with an acetate buffer solution (0.1M, pH=4.5) and incubating the resulting solution for 24 hours at 37° C. The untreated ON1 was used as reference. The integrity of ON1 was measured by LC-MS. No differences can be observed between the chromatogram and the fragmentation pattern of untreated ON1 (FIG. 1A, FIG. 1B) and the chromatogram and fragmentation pattern of ON1 treated for 24 hours in acidic conditions (FIG. 1C, FIG. 1D). The experiment demonstrates the stability of alpha anomeric oligonucleotides in acidic conditions that can be encountered, for example, in lysosome compartments of cells.

### Example 3

### Thermal Stability of Alpha Anomeric Oligonucleotides

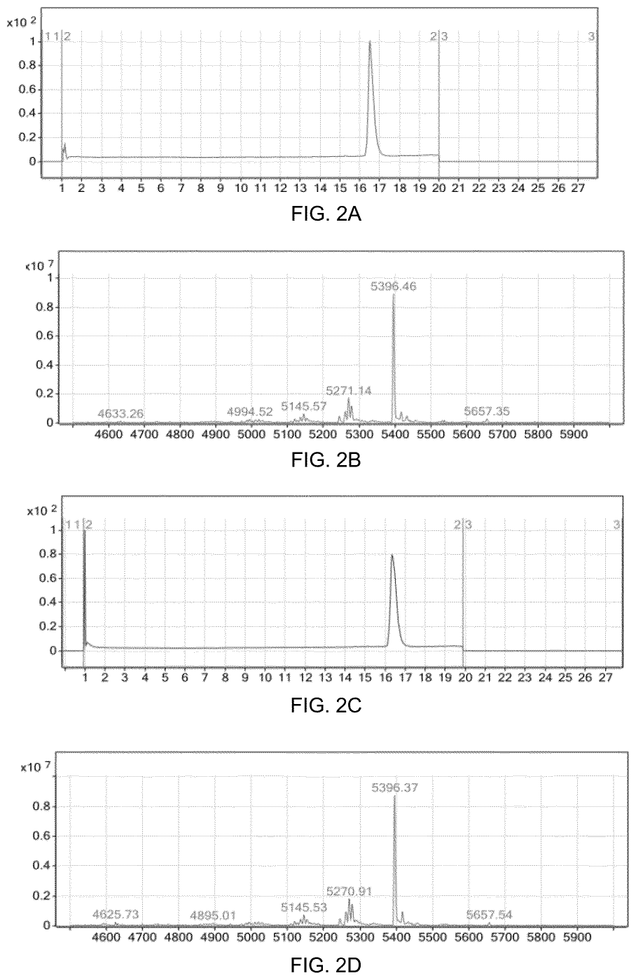

The thermal stability of ON1 was assessed by diluting ON1 to 10 μM with a PBS solution (137 mM NaCl, 2.7 mM KCl, 10 mM Na₂HPO₄,1.8 mM KH₂PO₄, pH=7.4) and incubating the resulting solution for 60 min. at 95° C. The untreated ON1 was used as reference. The integrity of ON1 was measured by LC-MS. No difference can be observed between the chromatogram and the fragmentation pattern of untreated ON1 (FIG. 2A, FIG. 2B) and the chromatogram and fragmentation pattern of ON1 heated at 95° C. (FIG. 2C, FIG. 2D). The experiment demonstrates the chemical stability of alpha anomeric oligonucleotides in aqueous solutions.

### Example 4

### Biostability of Alpha Anomeric Oligonucleotides

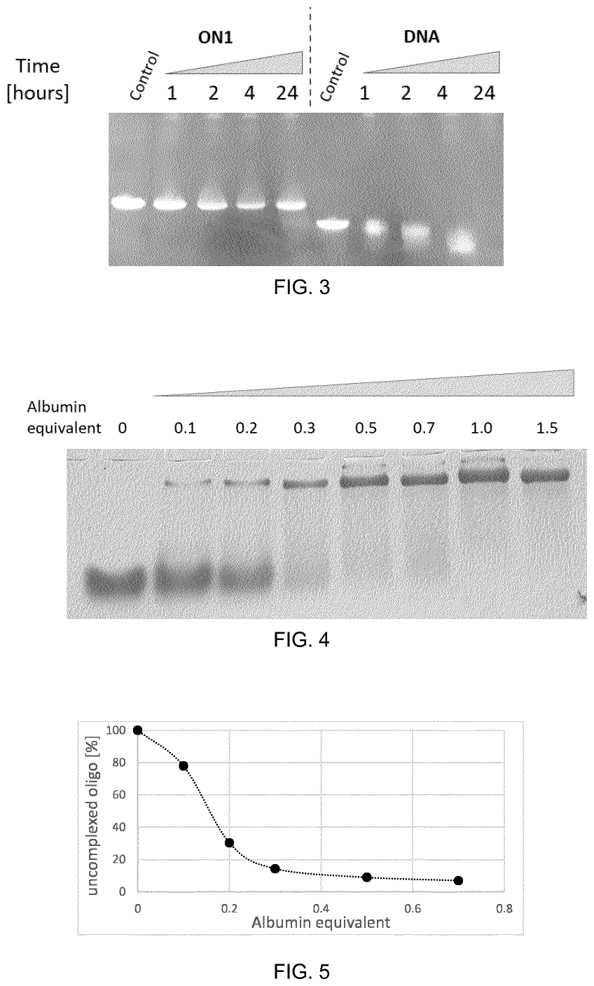

ON1 and its corresponding natural oligonucleotide were diluted to 10 μM in a 1:1 mixture of H₂O and mice serum (Sigma). The reactions were performed at a 20 μL scale and were incubated at 37° C. Control reactions were performed by incubating the oligonucleotides at 10 μM in H₂O at 37° C. for 24 hours. The reactions were stopped at specific times (1 h, 2 h, 4 h and 24 h) by addition of formamide (20 μW. The resulting mixtures were stored at −20° C. before being heat denaturated for 5 min at 90° C. and then analyzed by 20% denaturing PAGE (FIG. 3). Visualization was performed with a stains-all solution according to standard protocol. The result of the experiment shows complete digestion of natural DNA strand already after 4 hours, where ON1 remained completely stable even after 24 hours.

### Example 5

### Binding to Albumin of Alpha Anomeric Oligonucleotides Conjugated to Lipid Groups

The binding of ON1 to albumin was assessed by a mobility shift assay (FIG. 4). The test solutions were prepared by incubating ON1 at 40 μM for one hour at 37° C., in PBS solutions (137 mM NaCl, 2.7 mM KCl, 10 mM Na₂HPO₄, 1.8 mM KH₂PO₄, pH=7.4) containing 0, 0.1, 0.2, 0.3, 0.5, 0.7, 1.0 and 1.5 albumin equivalent (Albumin from mouse serum, lyophilized powder, ≥96% (Sigma-Aldrich)). 10 μL of each sample were analyzed by 10% native-PAGE (40V, 170 min, running at 7° C.). Visualization was performed with a stains-all solution according to standard protocol. The lower bands indicate the presence of uncomplexed ON1 and the upper bands indicate the presence of ON1 in complex with albumin. The experiment demonstrates that ON1 can efficiently bind to albumin at an albumin concentration≥0.3 equivalent.

The quantification of albumin binding of ON1 was assessed by ultrafiltration experiments (FIG. 5). Briefly, the test solutions were prepared by incubating ON1 at 55 μM for one hour at 37° C., in PBS solutions (137 mM NaCl, 2.7 mM KCl, 10 mM Na₂HPO₄, 1.8 mM KH₂PO₄, pH=7.4) containing 0, 0.1, 0.2, 0.3, 0.4, 0.4, 0.6 and 0.7 albumin equivalent (Albumin from mouse serum, lyophilized powder, ≥96% (Sigma-Aldrich)). Solutions were then filtered with Spin Column (Amicon Ultra-0.5 Centrifugal Filter Unit (Sigma-Aldrich)). The percentage of uncomplexed ON1 was calculated by measuring the absorbance of ON1 in the filtrates with a Nanodrop spectrophotometer and taking the solution with 0 equivalent albumin as reference. The result of the experiment shows that, at 0.3 equivalent albumin, only 14% of the oligonucleotide remains uncomplexed in solution.

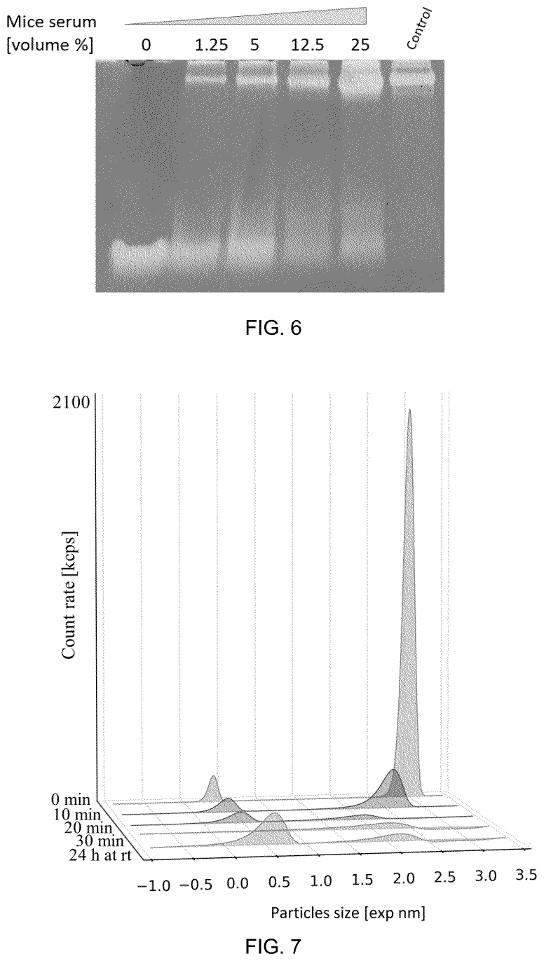

The binding of ON1 to albumin in mice serum (Sigma-Aldrich) was assessed by a mobility shift assay (FIG. 6). The test solutions were prepared by incubating ON1 at 40 μM for one hour at 37° C., in PBS solutions (137 mM NaCl, 2.7 mM KCl, 10 mM Na₂HPO₄, 1.8 mM KH₂PO₄, pH=7.4) containing 25% glycerol and 0%, 1.25%, 5.0%, 12.5% and 25.0% volume of mice serum. The control solution was prepared by incubating ON1 at 40 μM for one hour at 37° C., in PBS solutions containing 25% glycerol and 80 μM mice albumin. 10 μL of each sample were analyzed by 15% native-PAGE (60V, 260 min). Visualization was performed with a stains-all solution according to standard protocol. The lower bands indicate the presence of uncomplexed ON1 and the upper bands indicate the presence of ON1 in complex with albumin. The result of the experiment demonstrates that ON1 can efficiently bind to albumin in serum.

### Example 6

### Presence and Dissolution of Aggregates

The formation and dissolution of aggregates was analyzed with a Zetasizer Nano ZS (FIG. 7). ON1 was dissolved in a PBS solution (137 mM NaCl, 2.7 mM KCl, 10 mM Na₂HPO₄, 1.8 mM KH₂PO₄, pH=7.4) at a concentration of 7.5 mg/mL. The initial presence of nanoparticles was recorded (0 min). The solution was then heated to 95° C. and the presence of nanoparticles was recorded after 10 min, 20 min and 30 min of heating. The solution was then allowed to stand at rt for 24 hours and then the presence of nanoparticles was again recorded. Initially, a strong signal can be measured for particles with a size around 1000 nm. Heating the solution will lead to the disappearance of the signal. The result of the experiment demonstrates that an oligonucleotide conjugated to a lipophilic moiety will form aggregates in an aqueous solution. However, heating the solution for at least 20 min at 95° C. will assure the dispersion of the aggregates. The aggregates will not reappear after standing at rt for 24 hours.

### Example 7

### Determination of Exon Skipping Efficiency

Exon skipping involves the use of antisense oligonucleotides to cause one or more exons to be excluded from the mature mRNA. Through the use of exon skipping, one may cause one or more exons to be excluded from the mature mRNA, resulting in a mature mRNA that is in-frame. The skipping of an exon can be induced by the binding of antisense oligonucleotides targeting either one or both of the splice sites, or internal exon sequences. Since an exon will only be included in the mRNA when both the splice sites are recognized by the spliceosome complex, splice sites are obvious targets for antisense oligonucleotides.

To determine if an abc-DNA lipid group conjugated oligonucleotide of the invention causes exon skipping of the pre-mRNA of a gene of interest, cells are incubated with the oligonucleotide conjugate targeting a given exon(s) for a period of time. In certain embodiments, cells are transfected with lipofectamine. Exon skipping is detected through the use of reverse transcription polymerase chain reaction (RT-PCR) or DNA sequencing. Total RNA is extracted from the cells and RT-PCR is performed across the targeted exon and the size of the RT-PCR product is assessed via gel electrophoresis. If exon skipping has occurred, the product will not contain the targeted exon, and the size of this product will be of a predictably shorter size, compared to a product containing the targeted exon. Similarly, one may sequence the mature mRNA across the targeted exon to determine whether the targeted exon's sequence is absent from the mature mRNA.

To further determine the effect of an abc-DNA lipid group conjugated oligonucleotide of the invention dystrophin restoration is validated by western blot of a sample taken from a muscle biopsy, and the % dystrophin positive muscle fibers are determined by microscopy.

By the targeted skipping of a specific exon, a DMD phenotype can be converted into the milder BMD phenotype. Exon skipping is detected by incubating a differentiated human myoblast cell, a muscle cell derived for a DMD patient or a healthy patient with an antisense abc-DNA lipid group conjugated oligonucleotide that binds to the pre-mRNA of the DMD gene, as described in this Example. Alternatively, and as also described in this Example, cells are derived from a MDX mouse, a mouse model for DMD. In addition to comparing the level of exon skipping in a normal cell to the level of exon skipping in a DMD cell, the level of exon skipping in a cell derived from a DMD patient or MDX mouse is compared to the level of exon skipping in the absence of the abc-DNA lipid group conjugated oligonucleotide. In certain embodiments, the activity of the abc-DNA lipid group conjugated oligonucleotide of interest is compared to the level of exon skipping following administration of eteplirsen or drisapersen.

In the present example, the concentration of the antisense oligonucleotide was estimated by measuring the absorbance of a diluted aliquot at 260 nm. Specified amounts of the antisense oligonucleotides (AON) were then tested for their ability to induce exon skipping in an in vitro assay as described below.

Briefly, experiments were conducted in mice control immortalized myoblast cultures (C2C12) or in human control immortalized myoblast cultures (KM155). The cells were propagated and differentiated into myotubes using standard culturing techniques. The cells were transfected with the AONs by using, as a transfection reagent, Lipofectamine for mouse cell culture and oligofectamine for human cell culture. Complementary AON with a 2′-OMe-phosphorodithioate (2OMePS) backbone and a scrambled (non-functional) 2OMePS AON were used as positive and negative controls, respectively.

After 24 hours total RNA was extracted and molecular analysis was conducted. Reverse transcriptase amplification (RT-PCR), using a two-step (nested) PCR reaction, was undertaken to study the targeted regions of the dystrophin pre-mRNA or induced exonic re-arrangements.

For analyzing the AONs aiming to induce skipping of exon 23, the RT-PCR was conducted on the region spanning exon 23. After cDNA synthesis, first round PCR was performed using specific primers in mouse exons 21 and 26 (region 21-26) and the second round PCR was performed using specific primers in mouse exons 22 and 24 (region 22-24)

For analyzing the AONs aiming to induce skipping of exon 51, the RT-PCR was conducted on the region spanning exon 51. After cDNA synthesis, first round PCR was performed using specific primers in human exons 48 and 53 (region 48-53) and the second round PCR was performed using specific primers in human exons 49 and 52 (region 49-52).

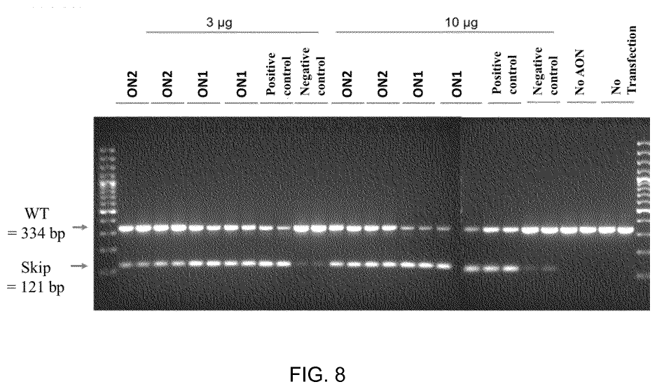

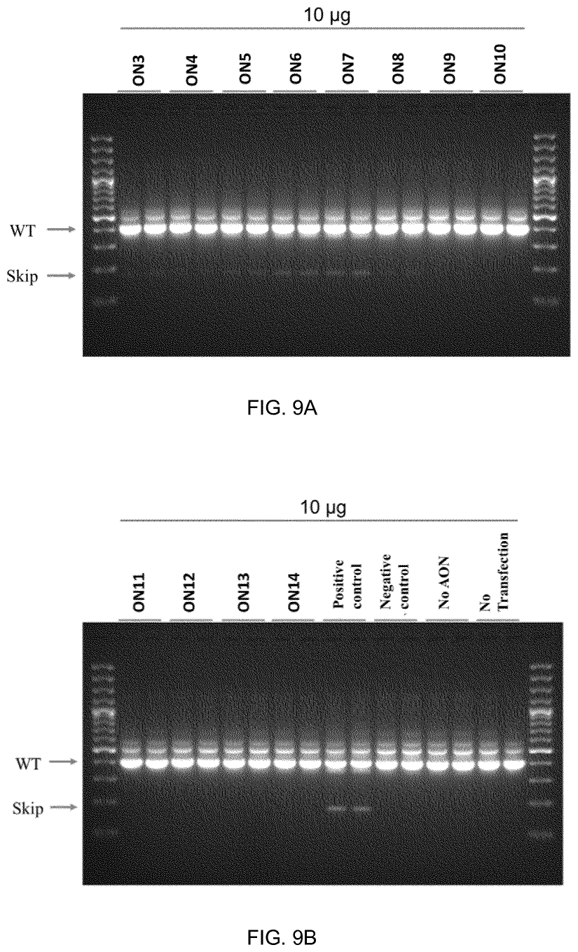

Expected product sizes for the non-skipped and skipped products were calculated. The intensity of the reaction products were estimated on an agarose gel, including a size standard. Hereby a potential bias has to be considered, since shorter or exon skipped products tend to be amplificated more efficiently than larger products, leading to an overestimation of skip efficiency. Bands indicating exon skipping product can be measured in mouse cells (FIG. 8) or in human cells (FIG. 9A, FIG. 9B). The result of the experiment demonstrates the capability of alpha anomeric oligonucleotides to modulate gene expression in vitro. The exon skipping capability of the inventive conjugate has been confirmed in vivo in the mdx mouse model of muscular dystrophy. Hereby, mdx23 mice received 12 weekly intravenous injections (50 mg/kg/week) of the inventive abc-DNA lipid group conjugated oligonucleotide having a sequence comprising SEQ ID NO: 412. Following treatment, tissues of diaphragm and gastrocnemicus were isolated and exon skipping determined.

### Example 8

### Exon Skipping in an hDMDde152/Mdx Mouse Model

Exon skipping efficacy is determined in the hDMDde152/mdx mouse model of muscular dystrophy. Mice receive intravenous injections of an abc-DNA lipid group conjugated oligonucleotide having a sequence comprising SEQ ID NO: 418, a corresponding phosphorodiamidate morpholino oligomer (PMO) or saline. Mice receive twelve weekly injections (50 mg/kg/week) of the oligonucleotide. Following treatment, the following tissues are isolated: heart, diaphragm, tibialis anterior, gastrocnemicus, quadricep, tricep, brain, liver and kidney, and exon skipping is determined. In certain embodiments, mice are treated with etiplersen or drisapersen in place of the abc-DNA lipid group conjugated oligonucleotide. Exon skipping is determined by RT-PCR, Western blot, immunofluorescence and/or digital PCR.

1. An oligonucleotide-lipid group conjugate wherein the oligonucleotide comprises at least two alpha anomeric bicyclo-DNA (abc-DNA) residues connected by a phosphodiester bond and wherein said lipid group is covalently attached to the oligonucleotide.

2. The oligonucleotide conjugate of claim 1, wherein said lipid group is covalently attached to the oligonucleotide via a linker.

3. The oligonucleotide conjugate of claim 1, wherein the oligonucleotide comprises 12 to 24 residues.

4. (canceled)

5. The oligonucleotide conjugate of claim 1, wherein said abc-DNA residue has the formula (V) 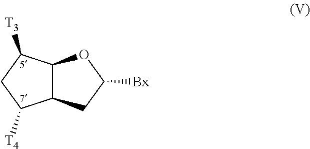
wherein independently for each of the at least two abc-DNA residue of formula (IV)
one of T₃ or T₄ is a nucleosidic linkage group; the other of T₃ and T₄ is OR₁, OR₂, a 5′ terminal group, a 7′ terminal group or a nucleosidic linkage group, wherein
R₁ is H or a hydroxyl protecting group, and
R₂ is a phosphorus moiety; and
Bx is a nucleobase.

6. The oligonucleotide conjugate of claim 1, wherein all of the residues are abc-DNA residues.

7. (canceled)

8. The oligonucleotide conjugate of claim 1, wherein all of the residues are abc-DNA residues and are connected via phosphodiester bonds.

9. The oligonucleotide conjugate of claim 1, wherein said lipid group is covalently attached to a terminal residue of the oligonucleotide.

10. The oligonucleotide conjugate of claim 1, wherein the oligonucleotide comprises residues connected via a phosphorus containing nucleosidic linkage group selected from the group consisting of: a phosphodiester linkage group, a phosphotriester linkage group, a phosphorothioate linkage group, a phosphorodithioate linkage group, a phosphonate linkage group, a phosphonothioate linkage group, a phosphinate linkage group, a phosphorthioamidate linkage and a phosphoramidate linkage group.

11. The oligonucleotide conjugate of claim 2, wherein the linker is a hydrocarbon linker or a polyethylene glycol (PEG) linker.

12. The oligonucleotide conjugate of claim 2, wherein the linker is selected from the group consisting of: an amino-alkyl-phosphorothioate linker, an amino-PEG-phosphorothioate linker, an alpha-carboxylate-amino-alkyl phosphorothioate linker, and an alpha-carboxylate-amino-PEG-phosphorothioate linker.

13. (canceled)

14. The oligonucleotide conjugate of claim 1, wherein the lipid group is a fatty acid derived group.

15. The oligonucleotide conjugate of claim 14, wherein the fatty acid is saturated or unsaturated.

16. The oligonucleotide conjugate of claim 14, wherein the fatty acid has a length from 4 to 28 carbon atoms.

17. The oligonucleotide conjugate of claim 14, wherein the fatty acid derived group comprises a carboxylic acid group.

18. The oligonucleotide conjugate of claim 14, wherein the fatty acid is selected from the fatty acids presented in Table 1 or Table 2.

19. The oligonucleotide conjugate of claim 14, wherein the fatty acid is hexadecanoic acid.

20. The oligonucleotide conjugate of claim 1, wherein the lipid group is attached to the oligonucleotide via a thiophosphate group.

21. The oligonucleotide conjugate of claim 1, wherein the oligonucleotide conjugate binds to the pre-mRNA corresponding to a portion of exon 51 of the Duchenne Muscular Dystrophy (DMD) gene.

22. The oligonucleotide conjugate of claim 21, wherein the oligonucleotide conjugate comprises a sequence selected from the group consisting of SEQ ID NOs: 4, 5, 22 to 24, 36 to 39, 51 to 55, 404 and 414 to 425.

23. The oligonucleotide conjugate of claim 1, wherein the oligonucleotide comprises any one of the sequences provided in Table 3.

24. The oligonucleotide conjugate of claim 1, wherein the oligonucleotide conjugate binds to the pre-mRNA corresponding to a portion of exon 53 of the DMD gene.

25. The oligonucleotide conjugate of claim 24 wherein the oligonucleotide conjugate comprises any one of the sequences provided in Table 4.

26. The oligonucleotide conjugate of claim 1, wherein the oligonucleotide conjugate binds to the pre-mRNA corresponding to a portion of exon 45 of the DMD gene.

27. The oligonucleotide conjugate of claim 26 wherein the oligonucleotide conjugate comprises any one of the sequences provided in Table 5.

28. A pharmaceutical composition comprising the oligonucleotide-lipid group conjugate of claim 1 in combination with a suitable carrier.

29. A method for altering expression of a gene by permitting hybridization of an oligonucleotide conjugate according to claim 1, to an RNA expressed from said gene, said oligonucleotide comprising a sequence that is complementary to a portion of said RNA.

30. A method for inducing the skipping of exon 51 of the human dystrophin pre-mRNA in a subject with Duchenne Muscular Dystrophy (DMD) or Becker Muscular Dystrophy (BMD), or in a cell derived from the subject, the method comprising providing an oligonucleotide conjugate of claim 1, which comprises a sequence selected from the group consisting of SEQ ID NOs: 4, 5, 22 to 24, 36 to 39, 51 to 55, 404 and 414 to 425, wherein the oligonucleotide conjugate induces skipping of the exon in the subject or the cell, and wherein mRNA produced from skipping exon 51 of the dystrophin pre-mRNA encodes a functional dystrophin protein or a dystrophin protein of a Becker subject.

31. A method of treating Duchenne Muscular Dystrophy (DMD) or Becker Muscular Dystrophy (BMD) in a subject or in a cell derived from the subject by inducing the skipping of exon 51 of the human dystrophin pre-mRNA, the method comprising providing to the subject or the cell a composition comprising an oligonucleotide conjugate of claim 1, comprising a sequence selected from the group consisting of SEQ ID NOs: 4, 5, 22 to 24, 36 to 39, 51 to 55, 404 and 414 to 425, wherein the oligonucleotide conjugate induces skipping of the exon in the subject or the cell, and wherein mRNA produced from skipping exon 51 of the dystrophin pre-mRNA encodes a functional dystrophin protein or a dystrophin protein of a Becker subject.


SEQUENCE LISTING (425 sequences)

SEQ ID NO: 1 - DNA, 18 bp, Artificial Sequence
  Synthetic Construct
ggtttgatctttacggta

SEQ ID NO: 2 - DNA, 18 bp, Artificial Sequence
  Synthetic Construct
gtttgatctttacggtag

SEQ ID NO: 3 - DNA, 18 bp, Artificial Sequence
  Synthetic Construct
tttgatctttacggtaga

SEQ ID NO: 4 - DNA, 18 bp, Artificial Sequence
  Synthetic Construct
ttgatctttacggtagaa

SEQ ID NO: 5 - DNA, 18 bp, Artificial Sequence
  Synthetic Construct
tgatctttacggtagaag

SEQ ID NO: 6 - DNA, 18 bp, Artificial Sequence
  Synthetic Construct
gatctttacggtagaagg

SEQ ID NO: 7 - DNA, 18 bp, Artificial Sequence
  Synthetic Construct
atctttacggtagaagga

SEQ ID NO: 8 - DNA, 18 bp, Artificial Sequence
  Synthetic Construct
tctttacggtagaaggaa

SEQ ID NO: 9 - DNA, 18 bp, Artificial Sequence
  Synthetic Construct
ctttacggtagaaggaac

SEQ ID NO: 10 - DNA, 18 bp, Artificial Sequence
  Synthetic Construct
tttacggtagaaggaact

SEQ ID NO: 11 - DNA, 18 bp, Artificial Sequence
  Synthetic Construct
ttacggtagaaggaacta

SEQ ID NO: 12 - DNA, 18 bp, Artificial Sequence
  Synthetic Construct
tacggtagaaggaactac

SEQ ID NO: 13 - DNA, 18 bp, Artificial Sequence
  Synthetic Construct
acggtagaaggaactaca

SEQ ID NO: 14 - DNA, 17 bp, Artificial Sequence
  Synthetic Construct
ggtttgatctttacggt

SEQ ID NO: 15 - DNA, 17 bp, Artificial Sequence
  Synthetic Construct
gtttgatctttacggta

SEQ ID NO: 16 - DNA, 17 bp, Artificial Sequence
  Synthetic Construct
tttgatctttacggtag

SEQ ID NO: 17 - DNA, 17 bp, Artificial Sequence
  Synthetic Construct
ttgatctttacggtaga

SEQ ID NO: 18 - DNA, 17 bp, Artificial Sequence
  Synthetic Construct
tgatctttacggtagaa

SEQ ID NO: 19 - DNA, 17 bp, Artificial Sequence
  Synthetic Construct
gatctttacggtagaag

SEQ ID NO: 20 - DNA, 17 bp, Artificial Sequence
  Synthetic Construct
atctttacggtagaagg

SEQ ID NO: 21 - DNA, 17 bp, Artificial Sequence
  Synthetic Construct
tctttacggtagaagga

SEQ ID NO: 22 - DNA, 17 bp, Artificial Sequence
  Synthetic Construct
ctttacggtagaaggaa

SEQ ID NO: 23 - DNA, 17 bp, Artificial Sequence
  Synthetic Construct
tttacggtagaaggaac

SEQ ID NO: 24 - DNA, 17 bp, Artificial Sequence
  Synthetic Construct
ttacggtagaaggaact

SEQ ID NO: 25 - DNA, 17 bp, Artificial Sequence
  Synthetic Construct
tacggtagaaggaacta

SEQ ID NO: 26 - DNA, 17 bp, Artificial Sequence
  Synthetic Construct
acggtagaaggaactac

SEQ ID NO: 27 - DNA, 17 bp, Artificial Sequence
  Synthetic Construct
cggtagaaggaactaca

SEQ ID NO: 28 - DNA, 16 bp, Artificial Sequence
  Synthetic Construct
ggtttgatctttacgg

SEQ ID NO: 29 - DNA, 16 bp, Artificial Sequence
  Synthetic Construct
gtttgatctttacggt

SEQ ID NO: 30 - DNA, 16 bp, Artificial Sequence
  Synthetic Construct
tttgatctttacggta

SEQ ID NO: 31 - DNA, 16 bp, Artificial Sequence
  Synthetic Construct
ttgatctttacggtag

SEQ ID NO: 32 - DNA, 16 bp, Artificial Sequence
  Synthetic Construct
tgatctttacggtaga

SEQ ID NO: 33 - DNA, 16 bp, Artificial Sequence
  Synthetic Construct
gatctttacggtagaa

SEQ ID NO: 34 - DNA, 16 bp, Artificial Sequence
  Synthetic Construct
atctttacggtagaag

SEQ ID NO: 35 - DNA, 16 bp, Artificial Sequence
  Synthetic Construct
tctttacggtagaagg

SEQ ID NO: 36 - DNA, 16 bp, Artificial Sequence
  Synthetic Construct
ctttacggtagaagga

SEQ ID NO: 37 - DNA, 16 bp, Artificial Sequence
  Synthetic Construct
tttacggtagaaggaa

SEQ ID NO: 38 - DNA, 16 bp, Artificial Sequence
  Synthetic Construct
ttacggtagaaggaac

SEQ ID NO: 39 - DNA, 16 bp, Artificial Sequence
  Synthetic Construct
tacggtagaaggaact

SEQ ID NO: 40 - DNA, 16 bp, Artificial Sequence
  Synthetic Construct
acggtagaaggaacta

SEQ ID NO: 41 - DNA, 16 bp, Artificial Sequence
  Synthetic Construct
cggtagaaggaactac

SEQ ID NO: 42 - DNA, 16 bp, Artificial Sequence
  Synthetic Construct
ggtagaaggaactaca

SEQ ID NO: 43 - DNA, 15 bp, Artificial Sequence
  Synthetic Construct
ggtttgatctttacg

SEQ ID NO: 44 - DNA, 15 bp, Artificial Sequence
  Synthetic Construct
gtttgatctttacgg

SEQ ID NO: 45 - DNA, 15 bp, Artificial Sequence
  Synthetic Construct
tttgatctttacggt

SEQ ID NO: 46 - DNA, 15 bp, Artificial Sequence
  Synthetic Construct
ttgatctttacggta

SEQ ID NO: 47 - DNA, 15 bp, Artificial Sequence
  Synthetic Construct
tgatctttacggtag

SEQ ID NO: 48 - DNA, 15 bp, Artificial Sequence
  Synthetic Construct
gatctttacggtaga

SEQ ID NO: 49 - DNA, 15 bp, Artificial Sequence
  Synthetic Construct
atctttacggtagaa

SEQ ID NO: 50 - DNA, 15 bp, Artificial Sequence
  Synthetic Construct
tctttacggtagaag

SEQ ID NO: 51 - DNA, 15 bp, Artificial Sequence
  Synthetic Construct
ctttacggtagaagg

SEQ ID NO: 52 - DNA, 15 bp, Artificial Sequence
  Synthetic Construct
tttacggtagaagga

SEQ ID NO: 53 - DNA, 15 bp, Artificial Sequence
  Synthetic Construct
ttacggtagaaggaa

SEQ ID NO: 54 - DNA, 15 bp, Artificial Sequence
  Synthetic Construct
tacggtagaaggaac

SEQ ID NO: 55 - DNA, 15 bp, Artificial Sequence
  Synthetic Construct
acggtagaaggaact

SEQ ID NO: 56 - DNA, 15 bp, Artificial Sequence
  Synthetic Construct
cggtagaaggaacta

SEQ ID NO: 57 - DNA, 15 bp, Artificial Sequence
  Synthetic Construct
ggtagaaggaactac

SEQ ID NO: 58 - DNA, 15 bp, Artificial Sequence
  Synthetic Construct
gtagaaggaactaca

SEQ ID NO: 59 - DNA, 14 bp, Artificial Sequence
  Synthetic Construct
ggtttgatctttac

SEQ ID NO: 60 - DNA, 14 bp, Artificial Sequence
  Synthetic Construct
gtttgatctttacg

SEQ ID NO: 61 - DNA, 14 bp, Artificial Sequence
  Synthetic Construct
tttgatctttacgg

SEQ ID NO: 62 - DNA, 14 bp, Artificial Sequence
  Synthetic Construct
ttgatctttacggt

SEQ ID NO: 63 - DNA, 14 bp, Artificial Sequence
  Synthetic Construct
tgatctttacggta

SEQ ID NO: 64 - DNA, 14 bp, Artificial Sequence
  Synthetic Construct
gatctttacggtag

SEQ ID NO: 65 - DNA, 14 bp, Artificial Sequence
  Synthetic Construct
atctttacggtaga

SEQ ID NO: 66 - DNA, 14 bp, Artificial Sequence
  Synthetic Construct
tctttacggtagaa

SEQ ID NO: 67 - DNA, 14 bp, Artificial Sequence
  Synthetic Construct
ctttacggtagaag

SEQ ID NO: 68 - DNA, 14 bp, Artificial Sequence
  Synthetic Construct
tttacggtagaagg

SEQ ID NO: 69 - DNA, 14 bp, Artificial Sequence
  Synthetic Construct
ttacggtagaagga

SEQ ID NO: 70 - DNA, 14 bp, Artificial Sequence
  Synthetic Construct
tacggtagaaggaa

SEQ ID NO: 71 - DNA, 14 bp, Artificial Sequence
  Synthetic Construct
acggtagaaggaac

SEQ ID NO: 72 - DNA, 14 bp, Artificial Sequence
  Synthetic Construct
cggtagaaggaact

SEQ ID NO: 73 - DNA, 14 bp, Artificial Sequence
  Synthetic Construct
ggtagaaggaacta

SEQ ID NO: 74 - DNA, 14 bp, Artificial Sequence
  Synthetic Construct
gtagaaggaactac

SEQ ID NO: 75 - DNA, 14 bp, Artificial Sequence
  Synthetic Construct
tagaaggaactaca

SEQ ID NO: 76 - DNA, 18 bp, Artificial Sequence
  Synthetic Construct
catgttcttgtggaagtc

SEQ ID NO: 77 - DNA, 18 bp, Artificial Sequence
  Synthetic Construct
atgttcttgtggaagtct

SEQ ID NO: 78 - DNA, 18 bp, Artificial Sequence
  Synthetic Construct
tgttcttgtggaagtctt

SEQ ID NO: 79 - DNA, 18 bp, Artificial Sequence
  Synthetic Construct
gttcttgtggaagtcttg

SEQ ID NO: 80 - DNA, 18 bp, Artificial Sequence
  Synthetic Construct
ttcttgtggaagtcttgg

SEQ ID NO: 81 - DNA, 18 bp, Artificial Sequence
  Synthetic Construct
tcttgtggaagtcttggc

SEQ ID NO: 82 - DNA, 18 bp, Artificial Sequence
  Synthetic Construct
cttgtggaagtcttggcc

SEQ ID NO: 83 - DNA, 18 bp, Artificial Sequence
  Synthetic Construct
ttgtggaagtcttggcct

SEQ ID NO: 84 - DNA, 18 bp, Artificial Sequence
  Synthetic Construct
tgtggaagtcttggcctc

SEQ ID NO: 85 - DNA, 18 bp, Artificial Sequence
  Synthetic Construct
gtggaagtcttggcctcc

SEQ ID NO: 86 - DNA, 18 bp, Artificial Sequence
  Synthetic Construct
tggaagtcttggcctccg

SEQ ID NO: 87 - DNA, 18 bp, Artificial Sequence
  Synthetic Construct
ggaagtcttggcctccgt

SEQ ID NO: 88 - DNA, 18 bp, Artificial Sequence
  Synthetic Construct
gaagtcttggcctccgtt

SEQ ID NO: 89 - DNA, 18 bp, Artificial Sequence
  Synthetic Construct
aagtcttggcctccgttg

SEQ ID NO: 90 - DNA, 18 bp, Artificial Sequence
  Synthetic Construct
agtcttggcctccgttgt

SEQ ID NO: 91 - DNA, 18 bp, Artificial Sequence
  Synthetic Construct
gtcttggcctccgttgtc

SEQ ID NO: 92 - DNA, 18 bp, Artificial Sequence
  Synthetic Construct
tcttggcctccgttgtca

SEQ ID NO: 93 - DNA, 18 bp, Artificial Sequence
  Synthetic Construct
cttggcctccgttgtcaa

SEQ ID NO: 94 - DNA, 18 bp, Artificial Sequence
  Synthetic Construct
ttggcctccgttgtcaac

SEQ ID NO: 95 - DNA, 18 bp, Artificial Sequence
  Synthetic Construct
tggcctccgttgtcaact

SEQ ID NO: 96 - DNA, 18 bp, Artificial Sequence
  Synthetic Construct
ggcctccgttgtcaactt

SEQ ID NO: 97 - DNA, 18 bp, Artificial Sequence
  Synthetic Construct
gcctccgttgtcaactta

SEQ ID NO: 98 - DNA, 18 bp, Artificial Sequence
  Synthetic Construct
cctccgttgtcaacttac

SEQ ID NO: 99 - DNA, 18 bp, Artificial Sequence
  Synthetic Construct
ctccgttgtcaacttact

SEQ ID NO: 100 - DNA, 18 bp, Artificial Sequence
  Synthetic Construct
tccgttgtcaacttactt

SEQ ID NO: 101 - DNA, 18 bp, Artificial Sequence
  Synthetic Construct
ccgttgtcaacttacttt

SEQ ID NO: 102 - DNA, 18 bp, Artificial Sequence
  Synthetic Construct
cgttgtcaacttacttta

SEQ ID NO: 103 - DNA, 18 bp, Artificial Sequence
  Synthetic Construct
gttgtcaacttactttac

SEQ ID NO: 104 - DNA, 18 bp, Artificial Sequence
  Synthetic Construct
ttgtcaacttactttaca

SEQ ID NO: 105 - DNA, 18 bp, Artificial Sequence
  Synthetic Construct
tgtcaacttactttacaa

SEQ ID NO: 106 - DNA, 18 bp, Artificial Sequence
  Synthetic Construct
gtcaacttactttacaat

SEQ ID NO: 107 - DNA, 17 bp, Artificial Sequence
  Synthetic Construct
catgttcttgtggaagt

SEQ ID NO: 108 - DNA, 17 bp, Artificial Sequence
  Synthetic Construct
atgttcttgtggaagtc

SEQ ID NO: 109 - DNA, 17 bp, Artificial Sequence
  Synthetic Construct
tgttcttgtggaagtct

SEQ ID NO: 110 - DNA, 17 bp, Artificial Sequence
  Synthetic Construct
gttcttgtggaagtctt

SEQ ID NO: 111 - DNA, 17 bp, Artificial Sequence
  Synthetic Construct
ttcttgtggaagtcttg

SEQ ID NO: 112 - DNA, 17 bp, Artificial Sequence
  Synthetic Construct
tcttgtggaagtcttgg

SEQ ID NO: 113 - DNA, 17 bp, Artificial Sequence
  Synthetic Construct
cttgtggaagtcttggc

SEQ ID NO: 114 - DNA, 17 bp, Artificial Sequence
  Synthetic Construct
ttgtggaagtcttggcc

SEQ ID NO: 115 - DNA, 17 bp, Artificial Sequence
  Synthetic Construct
tgtggaagtcttggcct

SEQ ID NO: 116 - DNA, 17 bp, Artificial Sequence
  Synthetic Construct
gtggaagtcttggcctc

SEQ ID NO: 117 - DNA, 17 bp, Artificial Sequence
  Synthetic Construct
tggaagtcttggcctcc

SEQ ID NO: 118 - DNA, 17 bp, Artificial Sequence
  Synthetic Construct
ggaagtcttggcctccg

SEQ ID NO: 119 - DNA, 17 bp, Artificial Sequence
  Synthetic Construct
gaagtcttggcctccgt

SEQ ID NO: 120 - DNA, 17 bp, Artificial Sequence
  Synthetic Construct
aagtcttggcctccgtt

SEQ ID NO: 121 - DNA, 17 bp, Artificial Sequence
  Synthetic Construct
agtcttggcctccgttg

SEQ ID NO: 122 - DNA, 17 bp, Artificial Sequence
  Synthetic Construct
gtcttggcctccgttgt

SEQ ID NO: 123 - DNA, 17 bp, Artificial Sequence
  Synthetic Construct
tcttggcctccgttgtc

SEQ ID NO: 124 - DNA, 17 bp, Artificial Sequence
  Synthetic Construct
cttggcctccgttgtca

SEQ ID NO: 125 - DNA, 17 bp, Artificial Sequence
  Synthetic Construct
ttggcctccgttgtcaa

SEQ ID NO: 126 - DNA, 17 bp, Artificial Sequence
  Synthetic Construct
tggcctccgttgtcaac

SEQ ID NO: 127 - DNA, 17 bp, Artificial Sequence
  Synthetic Construct
ggcctccgttgtcaact

SEQ ID NO: 128 - DNA, 17 bp, Artificial Sequence
  Synthetic Construct
gcctccgttgtcaactt

SEQ ID NO: 129 - DNA, 17 bp, Artificial Sequence
  Synthetic Construct
cctccgttgtcaactta

SEQ ID NO: 130 - DNA, 17 bp, Artificial Sequence
  Synthetic Construct
ctccgttgtcaacttac

SEQ ID NO: 131 - DNA, 17 bp, Artificial Sequence
  Synthetic Construct
tccgttgtcaacttact

SEQ ID NO: 132 - DNA, 17 bp, Artificial Sequence
  Synthetic Construct
ccgttgtcaacttactt

SEQ ID NO: 133 - DNA, 17 bp, Artificial Sequence
  Synthetic Construct
cgttgtcaacttacttt

SEQ ID NO: 134 - DNA, 17 bp, Artificial Sequence
  Synthetic Construct
gttgtcaacttacttta

SEQ ID NO: 135 - DNA, 17 bp, Artificial Sequence
  Synthetic Construct
ttgtcaacttactttac

SEQ ID NO: 136 - DNA, 17 bp, Artificial Sequence
  Synthetic Construct
tgtcaacttactttaca

SEQ ID NO: 137 - DNA, 17 bp, Artificial Sequence
  Synthetic Construct
gtcaacttactttacaa

SEQ ID NO: 138 - DNA, 17 bp, Artificial Sequence
  Synthetic Construct
tcaacttactttacaat

SEQ ID NO: 139 - DNA, 16 bp, Artificial Sequence
  Synthetic Construct
catgttcttgtggaag

SEQ ID NO: 140 - DNA, 16 bp, Artificial Sequence
  Synthetic Construct
atgttcttgtggaagt

SEQ ID NO: 141 - DNA, 16 bp, Artificial Sequence
  Synthetic Construct
tgttcttgtggaagtc

SEQ ID NO: 142 - DNA, 16 bp, Artificial Sequence
  Synthetic Construct
gttcttgtggaagtct

SEQ ID NO: 143 - DNA, 16 bp, Artificial Sequence
  Synthetic Construct
ttcttgtggaagtctt

SEQ ID NO: 144 - DNA, 16 bp, Artificial Sequence
  Synthetic Construct
tcttgtggaagtcttg

SEQ ID NO: 145 - DNA, 16 bp, Artificial Sequence
  Synthetic Construct
cttgtggaagtcttgg

SEQ ID NO: 146 - DNA, 16 bp, Artificial Sequence
  Synthetic Construct
ttgtggaagtcttggc

SEQ ID NO: 147 - DNA, 16 bp, Artificial Sequence
  Synthetic Construct
tgtggaagtcttggcc

SEQ ID NO: 148 - DNA, 16 bp, Artificial Sequence
  Synthetic Construct
gtggaagtcttggcct

SEQ ID NO: 149 - DNA, 16 bp, Artificial Sequence
  Synthetic Construct
tggaagtcttggcctc

SEQ ID NO: 150 - DNA, 16 bp, Artificial Sequence
  Synthetic Construct
ggaagtcttggcctcc

SEQ ID NO: 151 - DNA, 16 bp, Artificial Sequence
  Synthetic Construct
gaagtcttggcctccg

SEQ ID NO: 152 - DNA, 16 bp, Artificial Sequence
  Synthetic Construct
aagtcttggcctccgt

SEQ ID NO: 153 - DNA, 16 bp, Artificial Sequence
  Synthetic Construct
agtcttggcctccgtt

SEQ ID NO: 154 - DNA, 16 bp, Artificial Sequence
  Synthetic Construct
gtcttggcctccgttg

SEQ ID NO: 155 - DNA, 16 bp, Artificial Sequence
  Synthetic Construct
tcttggcctccgttgt

SEQ ID NO: 156 - DNA, 16 bp, Artificial Sequence
  Synthetic Construct
cttggcctccgttgtc

SEQ ID NO: 157 - DNA, 16 bp, Artificial Sequence
  Synthetic Construct
ttggcctccgttgtca

SEQ ID NO: 158 - DNA, 16 bp, Artificial Sequence
  Synthetic Construct
tggcctccgttgtcaa

SEQ ID NO: 159 - DNA, 16 bp, Artificial Sequence
  Synthetic Construct
ggcctccgttgtcaac

SEQ ID NO: 160 - DNA, 16 bp, Artificial Sequence
  Synthetic Construct
gcctccgttgtcaact

SEQ ID NO: 161 - DNA, 16 bp, Artificial Sequence
  Synthetic Construct
cctccgttgtcaactt

SEQ ID NO: 162 - DNA, 16 bp, Artificial Sequence
  Synthetic Construct
ctccgttgtcaactta

SEQ ID NO: 163 - DNA, 16 bp, Artificial Sequence
  Synthetic Construct
tccgttgtcaacttac

SEQ ID NO: 164 - DNA, 16 bp, Artificial Sequence
  Synthetic Construct
ccgttgtcaacttact

SEQ ID NO: 165 - DNA, 16 bp, Artificial Sequence
  Synthetic Construct
cgttgtcaacttactt

SEQ ID NO: 166 - DNA, 16 bp, Artificial Sequence
  Synthetic Construct
gttgtcaacttacttt

SEQ ID NO: 167 - DNA, 16 bp, Artificial Sequence
  Synthetic Construct
ttgtcaacttacttta

SEQ ID NO: 168 - DNA, 16 bp, Artificial Sequence
  Synthetic Construct
tgtcaacttactttac

SEQ ID NO: 169 - DNA, 16 bp, Artificial Sequence
  Synthetic Construct
gtcaacttactttaca

SEQ ID NO: 170 - DNA, 16 bp, Artificial Sequence
  Synthetic Construct
tcaacttactttacaa

SEQ ID NO: 171 - DNA, 16 bp, Artificial Sequence
  Synthetic Construct
caacttactttacaat

SEQ ID NO: 172 - DNA, 15 bp, Artificial Sequence
  Synthetic Construct
catgttcttgtggaa

SEQ ID NO: 173 - DNA, 15 bp, Artificial Sequence
  Synthetic Construct
atgttcttgtggaag

SEQ ID NO: 174 - DNA, 15 bp, Artificial Sequence
  Synthetic Construct
tgttcttgtggaagt

SEQ ID NO: 175 - DNA, 15 bp, Artificial Sequence
  Synthetic Construct
gttcttgtggaagtc

SEQ ID NO: 176 - DNA, 15 bp, Artificial Sequence
  Synthetic Construct
ttcttgtggaagtct

SEQ ID NO: 177 - DNA, 15 bp, Artificial Sequence
  Synthetic Construct
tcttgtggaagtctt

SEQ ID NO: 178 - DNA, 15 bp, Artificial Sequence
  Synthetic Construct
cttgtggaagtcttg

SEQ ID NO: 179 - DNA, 15 bp, Artificial Sequence
  Synthetic Construct
ttgtggaagtcttgg

SEQ ID NO: 180 - DNA, 15 bp, Artificial Sequence
  Synthetic Construct
tgtggaagtcttggc

SEQ ID NO: 181 - DNA, 15 bp, Artificial Sequence
  Synthetic Construct
gtggaagtcttggcc

SEQ ID NO: 182 - DNA, 15 bp, Artificial Sequence
  Synthetic Construct
tggaagtcttggcct

SEQ ID NO: 183 - DNA, 15 bp, Artificial Sequence
  Synthetic Construct
ggaagtcttggcctc

SEQ ID NO: 184 - DNA, 15 bp, Artificial Sequence
  Synthetic Construct
gaagtcttggcctcc

SEQ ID NO: 185 - DNA, 15 bp, Artificial Sequence
  Synthetic Construct
aagtcttggcctccg

SEQ ID NO: 186 - DNA, 15 bp, Artificial Sequence
  Synthetic Construct
agtcttggcctccgt

SEQ ID NO: 187 - DNA, 15 bp, Artificial Sequence
  Synthetic Construct
gtcttggcctccgtt

SEQ ID NO: 188 - DNA, 15 bp, Artificial Sequence
  Synthetic Construct
tcttggcctccgttg

SEQ ID NO: 189 - DNA, 15 bp, Artificial Sequence
  Synthetic Construct
cttggcctccgttgt

SEQ ID NO: 190 - DNA, 15 bp, Artificial Sequence
  Synthetic Construct
ttggcctccgttgtc

SEQ ID NO: 191 - DNA, 15 bp, Artificial Sequence
  Synthetic Construct
tggcctccgttgtca

SEQ ID NO: 192 - DNA, 15 bp, Artificial Sequence
  Synthetic Construct
ggcctccgttgtcaa

SEQ ID NO: 193 - DNA, 15 bp, Artificial Sequence
  Synthetic Construct
gcctccgttgtcaac

SEQ ID NO: 194 - DNA, 15 bp, Artificial Sequence
  Synthetic Construct
cctccgttgtcaact

SEQ ID NO: 195 - DNA, 15 bp, Artificial Sequence
  Synthetic Construct
ctccgttgtcaactt

SEQ ID NO: 196 - DNA, 15 bp, Artificial Sequence
  Synthetic Construct
tccgttgtcaactta

SEQ ID NO: 197 - DNA, 15 bp, Artificial Sequence
  Synthetic Construct
ccgttgtcaacttac

SEQ ID NO: 198 - DNA, 15 bp, Artificial Sequence
  Synthetic Construct
cgttgtcaacttact

SEQ ID NO: 199 - DNA, 15 bp, Artificial Sequence
  Synthetic Construct
gttgtcaacttactt

SEQ ID NO: 200 - DNA, 15 bp, Artificial Sequence
  Synthetic Construct
ttgtcaacttacttt

SEQ ID NO: 201 - DNA, 15 bp, Artificial Sequence
  Synthetic Construct
tgtcaacttacttta

SEQ ID NO: 202 - DNA, 15 bp, Artificial Sequence
  Synthetic Construct
gtcaacttactttac

SEQ ID NO: 203 - DNA, 15 bp, Artificial Sequence
  Synthetic Construct
tcaacttactttaca

SEQ ID NO: 204 - DNA, 15 bp, Artificial Sequence
  Synthetic Construct
caacttactttacaa

SEQ ID NO: 205 - DNA, 15 bp, Artificial Sequence
  Synthetic Construct
aacttactttacaat

SEQ ID NO: 206 - DNA, 14 bp, Artificial Sequence
  Synthetic Construct
catgttcttgtgga

SEQ ID NO: 207 - DNA, 14 bp, Artificial Sequence
  Synthetic Construct
atgttcttgtggaa

SEQ ID NO: 208 - DNA, 14 bp, Artificial Sequence
  Synthetic Construct
tgttcttgtggaag

SEQ ID NO: 209 - DNA, 14 bp, Artificial Sequence
  Synthetic Construct
gttcttgtggaagt

SEQ ID NO: 210 - DNA, 14 bp, Artificial Sequence
  Synthetic Construct
ttcttgtggaagtc

SEQ ID NO: 211 - DNA, 14 bp, Artificial Sequence
  Synthetic Construct
tcttgtggaagtct

SEQ ID NO: 212 - DNA, 14 bp, Artificial Sequence
  Synthetic Construct
cttgtggaagtctt

SEQ ID NO: 213 - DNA, 14 bp, Artificial Sequence
  Synthetic Construct
ttgtggaagtcttg

SEQ ID NO: 214 - DNA, 14 bp, Artificial Sequence
  Synthetic Construct
tgtggaagtcttgg

SEQ ID NO: 215 - DNA, 14 bp, Artificial Sequence
  Synthetic Construct
gtggaagtcttggc

SEQ ID NO: 216 - DNA, 14 bp, Artificial Sequence
  Synthetic Construct
tggaagtcttggcc

SEQ ID NO: 217 - DNA, 14 bp, Artificial Sequence
  Synthetic Construct
ggaagtcttggcct

SEQ ID NO: 218 - DNA, 14 bp, Artificial Sequence
  Synthetic Construct
gaagtcttggcctc

SEQ ID NO: 219 - DNA, 14 bp, Artificial Sequence
  Synthetic Construct
aagtcttggcctcc

SEQ ID NO: 220 - DNA, 14 bp, Artificial Sequence
  Synthetic Construct
agtcttggcctccg

SEQ ID NO: 221 - DNA, 14 bp, Artificial Sequence
  Synthetic Construct
gtcttggcctccgt

SEQ ID NO: 222 - DNA, 14 bp, Artificial Sequence
  Synthetic Construct
tcttggcctccgtt

SEQ ID NO: 223 - DNA, 14 bp, Artificial Sequence
  Synthetic Construct
cttggcctccgttg

SEQ ID NO: 224 - DNA, 14 bp, Artificial Sequence
  Synthetic Construct
ttggcctccgttgt

SEQ ID NO: 225 - DNA, 14 bp, Artificial Sequence
  Synthetic Construct
tggcctccgttgtc

SEQ ID NO: 226 - DNA, 14 bp, Artificial Sequence
  Synthetic Construct
ggcctccgttgtca

SEQ ID NO: 227 - DNA, 14 bp, Artificial Sequence
  Synthetic Construct
gcctccgttgtcaa

SEQ ID NO: 228 - DNA, 14 bp, Artificial Sequence
  Synthetic Construct
cctccgttgtcaac

SEQ ID NO: 229 - DNA, 14 bp, Artificial Sequence
  Synthetic Construct
ctccgttgtcaact

SEQ ID NO: 230 - DNA, 14 bp, Artificial Sequence
  Synthetic Construct
tccgttgtcaactt

SEQ ID NO: 231 - DNA, 14 bp, Artificial Sequence
  Synthetic Construct
ccgttgtcaactta

SEQ ID NO: 232 - DNA, 14 bp, Artificial Sequence
  Synthetic Construct
cgttgtcaacttac

SEQ ID NO: 233 - DNA, 14 bp, Artificial Sequence
  Synthetic Construct
gttgtcaacttact

SEQ ID NO: 234 - DNA, 14 bp, Artificial Sequence
  Synthetic Construct
ttgtcaacttactt

SEQ ID NO: 235 - DNA, 14 bp, Artificial Sequence
  Synthetic Construct
tgtcaacttacttt

SEQ ID NO: 236 - DNA, 14 bp, Artificial Sequence
  Synthetic Construct
gtcaacttacttta

SEQ ID NO: 237 - DNA, 14 bp, Artificial Sequence
  Synthetic Construct
tcaacttactttac

SEQ ID NO: 238 - DNA, 14 bp, Artificial Sequence
  Synthetic Construct
caacttactttaca

SEQ ID NO: 239 - DNA, 14 bp, Artificial Sequence
  Synthetic Construct
aacttactttacaa

SEQ ID NO: 240 - DNA, 14 bp, Artificial Sequence
  Synthetic Construct
acttactttacaat

SEQ ID NO: 241 - DNA, 18 bp, Artificial Sequence
  Synthetic Construct
ccatagaatgtccttgag

SEQ ID NO: 242 - DNA, 18 bp, Artificial Sequence
  Synthetic Construct
catagaatgtccttgagg

SEQ ID NO: 243 - DNA, 18 bp, Artificial Sequence
  Synthetic Construct
atagaatgtccttgaggt

SEQ ID NO: 244 - DNA, 18 bp, Artificial Sequence
  Synthetic Construct
tagaatgtccttgaggtc

SEQ ID NO: 245 - DNA, 18 bp, Artificial Sequence
  Synthetic Construct
agaatgtccttgaggtcc

SEQ ID NO: 246 - DNA, 18 bp, Artificial Sequence
  Synthetic Construct
gaatgtccttgaggtcct

SEQ ID NO: 247 - DNA, 18 bp, Artificial Sequence
  Synthetic Construct
aatgtccttgaggtccta

SEQ ID NO: 248 - DNA, 18 bp, Artificial Sequence
  Synthetic Construct
atgtccttgaggtcctac

SEQ ID NO: 249 - DNA, 18 bp, Artificial Sequence
  Synthetic Construct
tgtccttgaggtcctacc

SEQ ID NO: 250 - DNA, 18 bp, Artificial Sequence
  Synthetic Construct
gtccttgaggtcctaccg

SEQ ID NO: 251 - DNA, 18 bp, Artificial Sequence
  Synthetic Construct
tccttgaggtcctaccgt

SEQ ID NO: 252 - DNA, 18 bp, Artificial Sequence
  Synthetic Construct
ccttgaggtcctaccgta

SEQ ID NO: 253 - DNA, 18 bp, Artificial Sequence
  Synthetic Construct
cttgaggtcctaccgtaa

SEQ ID NO: 254 - DNA, 18 bp, Artificial Sequence
  Synthetic Construct
ttgaggtcctaccgtaac

SEQ ID NO: 255 - DNA, 18 bp, Artificial Sequence
  Synthetic Construct
tgaggtcctaccgtaacc

SEQ ID NO: 256 - DNA, 18 bp, Artificial Sequence
  Synthetic Construct
gaggtcctaccgtaaccc

SEQ ID NO: 257 - DNA, 18 bp, Artificial Sequence
  Synthetic Construct
aggtcctaccgtaacccg

SEQ ID NO: 258 - DNA, 18 bp, Artificial Sequence
  Synthetic Construct
ggtcctaccgtaacccgt

SEQ ID NO: 259 - DNA, 18 bp, Artificial Sequence
  Synthetic Construct
gtcctaccgtaacccgtc

SEQ ID NO: 260 - DNA, 18 bp, Artificial Sequence
  Synthetic Construct
tcctaccgtaacccgtcg

SEQ ID NO: 261 - DNA, 18 bp, Artificial Sequence
  Synthetic Construct
cctaccgtaacccgtcgc

SEQ ID NO: 262 - DNA, 18 bp, Artificial Sequence
  Synthetic Construct
ctaccgtaacccgtcgcc

SEQ ID NO: 263 - DNA, 18 bp, Artificial Sequence
  Synthetic Construct
taccgtaacccgtcgccg

SEQ ID NO: 264 - DNA, 18 bp, Artificial Sequence
  Synthetic Construct
accgtaacccgtcgccgt

SEQ ID NO: 265 - DNA, 18 bp, Artificial Sequence
  Synthetic Construct
ccgtaacccgtcgccgtt

SEQ ID NO: 266 - DNA, 18 bp, Artificial Sequence
  Synthetic Construct
cgtaacccgtcgccgttt

SEQ ID NO: 267 - DNA, 18 bp, Artificial Sequence
  Synthetic Construct
gtaacccgtcgccgtttg

SEQ ID NO: 268 - DNA, 18 bp, Artificial Sequence
  Synthetic Construct
taacccgtcgccgtttga

SEQ ID NO: 269 - DNA, 18 bp, Artificial Sequence
  Synthetic Construct
aacccgtcgccgtttgac

SEQ ID NO: 270 - DNA, 18 bp, Artificial Sequence
  Synthetic Construct
acccgtcgccgtttgaca

SEQ ID NO: 271 - DNA, 17 bp, Artificial Sequence
  Synthetic Construct
ccatagaatgtccttga

SEQ ID NO: 272 - DNA, 17 bp, Artificial Sequence
  Synthetic Construct
catagaatgtccttgag

SEQ ID NO: 273 - DNA, 17 bp, Artificial Sequence
  Synthetic Construct
atagaatgtccttgagg

SEQ ID NO: 274 - DNA, 17 bp, Artificial Sequence
  Synthetic Construct
tagaatgtccttgaggt

SEQ ID NO: 275 - DNA, 17 bp, Artificial Sequence
  Synthetic Construct
agaatgtccttgaggtc

SEQ ID NO: 276 - DNA, 17 bp, Artificial Sequence
  Synthetic Construct
gaatgtccttgaggtcc

SEQ ID NO: 277 - DNA, 18 bp, Artificial Sequence
  Synthetic Construct
taatgtccttgaggtcct

SEQ ID NO: 278 - DNA, 17 bp, Artificial Sequence
  Synthetic Construct
atgtccttgaggtccta

SEQ ID NO: 279 - DNA, 17 bp, Artificial Sequence
  Synthetic Construct
tgtccttgaggtcctac

SEQ ID NO: 280 - DNA, 17 bp, Artificial Sequence
  Synthetic Construct
gtccttgaggtcctacc

SEQ ID NO: 281 - DNA, 17 bp, Artificial Sequence
  Synthetic Construct
tccttgaggtcctaccg

SEQ ID NO: 282 - DNA, 17 bp, Artificial Sequence
  Synthetic Construct
ccttgaggtcctaccgt

SEQ ID NO: 283 - DNA, 17 bp, Artificial Sequence
  Synthetic Construct
cttgaggtcctaccgta

SEQ ID NO: 284 - DNA, 17 bp, Artificial Sequence
  Synthetic Construct
ttgaggtcctaccgtaa

SEQ ID NO: 285 - DNA, 17 bp, Artificial Sequence
  Synthetic Construct
tgaggtcctaccgtaac

SEQ ID NO: 286 - DNA, 17 bp, Artificial Sequence
  Synthetic Construct
gaggtcctaccgtaacc

SEQ ID NO: 287 - DNA, 17 bp, Artificial Sequence
  Synthetic Construct
aggtcctaccgtaaccc

SEQ ID NO: 288 - DNA, 17 bp, Artificial Sequence
  Synthetic Construct
ggtcctaccgtaacccg

SEQ ID NO: 289 - DNA, 17 bp, Artificial Sequence
  Synthetic Construct
gtcctaccgtaacccgt

SEQ ID NO: 290 - DNA, 17 bp, Artificial Sequence
  Synthetic Construct
tcctaccgtaacccgtc

SEQ ID NO: 291 - DNA, 17 bp, Artificial Sequence
  Synthetic Construct
cctaccgtaacccgtcg

SEQ ID NO: 292 - DNA, 17 bp, Artificial Sequence
  Synthetic Construct
ctaccgtaacccgtcgc

SEQ ID NO: 293 - DNA, 17 bp, Artificial Sequence
  Synthetic Construct
taccgtaacccgtcgcc

SEQ ID NO: 294 - DNA, 17 bp, Artificial Sequence
  Synthetic Construct
accgtaacccgtcgccg

SEQ ID NO: 295 - DNA, 17 bp, Artificial Sequence
  Synthetic Construct
ccgtaacccgtcgccgt

SEQ ID NO: 296 - DNA, 17 bp, Artificial Sequence
  Synthetic Construct
cgtaacccgtcgccgtt

SEQ ID NO: 297 - DNA, 17 bp, Artificial Sequence
  Synthetic Construct
gtaacccgtcgccgttt

SEQ ID NO: 298 - DNA, 17 bp, Artificial Sequence
  Synthetic Construct
taacccgtcgccgtttg

SEQ ID NO: 299 - DNA, 17 bp, Artificial Sequence
  Synthetic Construct
aacccgtcgccgtttga

SEQ ID NO: 300 - DNA, 17 bp, Artificial Sequence
  Synthetic Construct
acccgtcgccgtttgac

SEQ ID NO: 301 - DNA, 17 bp, Artificial Sequence
  Synthetic Construct
cccgtcgccgtttgaca

SEQ ID NO: 302 - DNA, 16 bp, Artificial Sequence
  Synthetic Construct
ccatagaatgtccttg

SEQ ID NO: 303 - DNA, 16 bp, Artificial Sequence
  Synthetic Construct
catagaatgtccttga

SEQ ID NO: 304 - DNA, 16 bp, Artificial Sequence
  Synthetic Construct
atagaatgtccttgag

SEQ ID NO: 305 - DNA, 16 bp, Artificial Sequence
  Synthetic Construct
tagaatgtccttgagg

SEQ ID NO: 306 - DNA, 16 bp, Artificial Sequence
  Synthetic Construct
agaatgtccttgaggt

SEQ ID NO: 307 - DNA, 16 bp, Artificial Sequence
  Synthetic Construct
gaatgtccttgaggtc

SEQ ID NO: 308 - DNA, 16 bp, Artificial Sequence
  Synthetic Construct
aatgtccttgaggtcc

SEQ ID NO: 309 - DNA, 16 bp, Artificial Sequence
  Synthetic Construct
atgtccttgaggtcct

SEQ ID NO: 310 - DNA, 16 bp, Artificial Sequence
  Synthetic Construct
tgtccttgaggtccta

SEQ ID NO: 311 - DNA, 16 bp, Artificial Sequence
  Synthetic Construct
gtccttgaggtcctac

SEQ ID NO: 312 - DNA, 16 bp, Artificial Sequence
  Synthetic Construct
tccttgaggtcctacc

SEQ ID NO: 313 - DNA, 16 bp, Artificial Sequence
  Synthetic Construct
ccttgaggtcctaccg

SEQ ID NO: 314 - DNA, 16 bp, Artificial Sequence
  Synthetic Construct
cttgaggtcctaccgt

SEQ ID NO: 315 - DNA, 16 bp, Artificial Sequence
  Synthetic Construct
ttgaggtcctaccgta

SEQ ID NO: 316 - DNA, 16 bp, Artificial Sequence
  Synthetic Construct
tgaggtcctaccgtaa

SEQ ID NO: 317 - DNA, 16 bp, Artificial Sequence
  Synthetic Construct
gaggtcctaccgtaac

SEQ ID NO: 318 - DNA, 16 bp, Artificial Sequence
  Synthetic Construct
aggtcctaccgtaacc

SEQ ID NO: 319 - DNA, 16 bp, Artificial Sequence
  Synthetic Construct
ggtcctaccgtaaccc

SEQ ID NO: 320 - DNA, 16 bp, Artificial Sequence
  Synthetic Construct
gtcctaccgtaacccg

SEQ ID NO: 321 - DNA, 16 bp, Artificial Sequence
  Synthetic Construct
tcctaccgtaacccgt

SEQ ID NO: 322 - DNA, 16 bp, Artificial Sequence
  Synthetic Construct
cctaccgtaacccgtc

SEQ ID NO: 323 - DNA, 16 bp, Artificial Sequence
  Synthetic Construct
ctaccgtaacccgtcg

SEQ ID NO: 324 - DNA, 16 bp, Artificial Sequence
  Synthetic Construct
taccgtaacccgtcgc

SEQ ID NO: 325 - DNA, 16 bp, Artificial Sequence
  Synthetic Construct
accgtaacccgtcgcc

SEQ ID NO: 326 - DNA, 16 bp, Artificial Sequence
  Synthetic Construct
ccgtaacccgtcgccg

SEQ ID NO: 327 - DNA, 16 bp, Artificial Sequence
  Synthetic Construct
cgtaacccgtcgccgt

SEQ ID NO: 328 - DNA, 16 bp, Artificial Sequence
  Synthetic Construct
gtaacccgtcgccgtt

SEQ ID NO: 329 - DNA, 16 bp, Artificial Sequence
  Synthetic Construct
taacccgtcgccgttt

SEQ ID NO: 330 - DNA, 16 bp, Artificial Sequence
  Synthetic Construct
aacccgtcgccgtttg

SEQ ID NO: 331 - DNA, 16 bp, Artificial Sequence
  Synthetic Construct
acccgtcgccgtttga

SEQ ID NO: 332 - DNA, 16 bp, Artificial Sequence
  Synthetic Construct
cccgtcgccgtttgac

SEQ ID NO: 333 - DNA, 16 bp, Artificial Sequence
  Synthetic Construct
ccgtcgccgtttgaca

SEQ ID NO: 334 - DNA, 15 bp, Artificial Sequence
  Synthetic Construct
ccatagaatgtcctt

SEQ ID NO: 335 - DNA, 15 bp, Artificial Sequence
  Synthetic Construct
catagaatgtccttg

SEQ ID NO: 336 - DNA, 15 bp, Artificial Sequence
  Synthetic Construct
atagaatgtccttga

SEQ ID NO: 337 - DNA, 15 bp, Artificial Sequence
  Synthetic Construct
tagaatgtccttgag

SEQ ID NO: 338 - DNA, 15 bp, Artificial Sequence
  Synthetic Construct
agaatgtccttgagg

SEQ ID NO: 339 - DNA, 15 bp, Artificial Sequence
  Synthetic Construct
gaatgtccttgaggt

SEQ ID NO: 340 - DNA, 15 bp, Artificial Sequence
  Synthetic Construct
aatgtccttgaggtc

SEQ ID NO: 341 - DNA, 15 bp, Artificial Sequence
  Synthetic Construct
atgtccttgaggtcc

SEQ ID NO: 342 - DNA, 15 bp, Artificial Sequence
  Synthetic Construct
tgtccttgaggtcct

SEQ ID NO: 343 - DNA, 15 bp, Artificial Sequence
  Synthetic Construct
gtccttgaggtccta

SEQ ID NO: 344 - DNA, 15 bp, Artificial Sequence
  Synthetic Construct
tccttgaggtcctac

SEQ ID NO: 345 - DNA, 15 bp, Artificial Sequence
  Synthetic Construct
ccttgaggtcctacc

SEQ ID NO: 346 - DNA, 15 bp, Artificial Sequence
  Synthetic Construct
cttgaggtcctaccg

SEQ ID NO: 347 - DNA, 15 bp, Artificial Sequence
  Synthetic Construct
ttgaggtcctaccgt

SEQ ID NO: 348 - DNA, 15 bp, Artificial Sequence
  Synthetic Construct
tgaggtcctaccgta

SEQ ID NO: 349 - DNA, 15 bp, Artificial Sequence
  Synthetic Construct
gaggtcctaccgtaa

SEQ ID NO: 350 - DNA, 15 bp, Artificial Sequence
  Synthetic Construct
aggtcctaccgtaac

SEQ ID NO: 351 - DNA, 15 bp, Artificial Sequence
  Synthetic Construct
ggtcctaccgtaacc

SEQ ID NO: 352 - DNA, 15 bp, Artificial Sequence
  Synthetic Construct
gtcctaccgtaaccc

SEQ ID NO: 353 - DNA, 15 bp, Artificial Sequence
  Synthetic Construct
tcctaccgtaacccg

SEQ ID NO: 354 - DNA, 15 bp, Artificial Sequence
  Synthetic Construct
cctaccgtaacccgt

SEQ ID NO: 355 - DNA, 15 bp, Artificial Sequence
  Synthetic Construct
ctaccgtaacccgtc

SEQ ID NO: 356 - DNA, 15 bp, Artificial Sequence
  Synthetic Construct
taccgtaacccgtcg

SEQ ID NO: 357 - DNA, 15 bp, Artificial Sequence
  Synthetic Construct
accgtaacccgtcgc

SEQ ID NO: 358 - DNA, 15 bp, Artificial Sequence
  Synthetic Construct
ccgtaacccgtcgcc

SEQ ID NO: 359 - DNA, 15 bp, Artificial Sequence
  Synthetic Construct
cgtaacccgtcgccg

SEQ ID NO: 360 - DNA, 15 bp, Artificial Sequence
  Synthetic Construct
gtaacccgtcgccgt

SEQ ID NO: 361 - DNA, 15 bp, Artificial Sequence
  Synthetic Construct
taacccgtcgccgtt

SEQ ID NO: 362 - DNA, 15 bp, Artificial Sequence
  Synthetic Construct
aacccgtcgccgttt

SEQ ID NO: 363 - DNA, 15 bp, Artificial Sequence
  Synthetic Construct
acccgtcgccgtttg

SEQ ID NO: 364 - DNA, 15 bp, Artificial Sequence
  Synthetic Construct
cccgtcgccgtttga

SEQ ID NO: 365 - DNA, 15 bp, Artificial Sequence
  Synthetic Construct
ccgtcgccgtttgac

SEQ ID NO: 366 - DNA, 15 bp, Artificial Sequence
  Synthetic Construct
cgtcgccgtttgaca

SEQ ID NO: 367 - DNA, 14 bp, Artificial Sequence
  Synthetic Construct
ccatagaatgtcct

SEQ ID NO: 368 - DNA, 14 bp, Artificial Sequence
  Synthetic Construct
catagaatgtcctt

SEQ ID NO: 369 - DNA, 14 bp, Artificial Sequence
  Synthetic Construct
atagaatgtccttg

SEQ ID NO: 370 - DNA, 14 bp, Artificial Sequence
  Synthetic Construct
tagaatgtccttga

SEQ ID NO: 371 - DNA, 14 bp, Artificial Sequence
  Synthetic Construct
agaatgtccttgag

SEQ ID NO: 372 - DNA, 14 bp, Artificial Sequence
  Synthetic Construct
gaatgtccttgagg

SEQ ID NO: 373 - DNA, 14 bp, Artificial Sequence
  Synthetic Construct
aatgtccttgaggt

SEQ ID NO: 374 - DNA, 14 bp, Artificial Sequence
  Synthetic Construct
atgtccttgaggtc

SEQ ID NO: 375 - DNA, 14 bp, Artificial Sequence
  Synthetic Construct
tgtccttgaggtcc

SEQ ID NO: 376 - DNA, 14 bp, Artificial Sequence
  Synthetic Construct
gtccttgaggtcct

SEQ ID NO: 377 - DNA, 14 bp, Artificial Sequence
  Synthetic Construct
tccttgaggtccta

SEQ ID NO: 378 - DNA, 14 bp, Artificial Sequence
  Synthetic Construct
ccttgaggtcctac

SEQ ID NO: 379 - DNA, 14 bp, Artificial Sequence
  Synthetic Construct
cttgaggtcctacc

SEQ ID NO: 380 - DNA, 14 bp, Artificial Sequence
  Synthetic Construct
ttgaggtcctaccg

SEQ ID NO: 381 - DNA, 14 bp, Artificial Sequence
  Synthetic Construct
tgaggtcctaccgt

SEQ ID NO: 382 - DNA, 14 bp, Artificial Sequence
  Synthetic Construct
gaggtcctaccgta

SEQ ID NO: 383 - DNA, 14 bp, Artificial Sequence
  Synthetic Construct
aggtcctaccgtaa

SEQ ID NO: 384 - DNA, 14 bp, Artificial Sequence
  Synthetic Construct
ggtcctaccgtaac

SEQ ID NO: 385 - DNA, 14 bp, Artificial Sequence
  Synthetic Construct
gtcctaccgtaacc

SEQ ID NO: 386 - DNA, 14 bp, Artificial Sequence
  Synthetic Construct
tcctaccgtaaccc

SEQ ID NO: 387 - DNA, 14 bp, Artificial Sequence
  Synthetic Construct
cctaccgtaacccg

SEQ ID NO: 388 - DNA, 14 bp, Artificial Sequence
  Synthetic Construct
ctaccgtaacccgt

SEQ ID NO: 389 - DNA, 14 bp, Artificial Sequence
  Synthetic Construct
taccgtaacccgtc

SEQ ID NO: 390 - DNA, 14 bp, Artificial Sequence
  Synthetic Construct
accgtaacccgtcg

SEQ ID NO: 391 - DNA, 14 bp, Artificial Sequence
  Synthetic Construct
ccgtaacccgtcgc

SEQ ID NO: 392 - DNA, 14 bp, Artificial Sequence
  Synthetic Construct
cgtaacccgtcgcc

SEQ ID NO: 393 - DNA, 14 bp, Artificial Sequence
  Synthetic Construct
gtaacccgtcgccg

SEQ ID NO: 394 - DNA, 14 bp, Artificial Sequence
  Synthetic Construct
taacccgtcgccgt

SEQ ID NO: 395 - DNA, 14 bp, Artificial Sequence
  Synthetic Construct
aacccgtcgccgtt

SEQ ID NO: 396 - DNA, 14 bp, Artificial Sequence
  Synthetic Construct
acccgtcgccgttt

SEQ ID NO: 397 - DNA, 14 bp, Artificial Sequence
  Synthetic Construct
cccgtcgccgtttg

SEQ ID NO: 398 - DNA, 14 bp, Artificial Sequence
  Synthetic Construct
ccgtcgccgtttga

SEQ ID NO: 399 - DNA, 14 bp, Artificial Sequence
  Synthetic Construct
cgtcgccgtttgac

SEQ ID NO: 400 - DNA, 14 bp, Artificial Sequence
  Synthetic Construct
gtcgccgtttgaca

SEQ ID NO: 401 - DNA, 333 bp, Homo sapiens
tttttctttttcttcttttttcctttttgcaaaaacccaaaatattttagctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggtatgagaaaaaatgataaaagttggcagaagtttttctttaaaatgaag

SEQ ID NO: 402 - DNA, 30 bp, Artificial Sequence
  Synthetic Construct
ccaaactagaaatgccatcttccttgatgt

SEQ ID NO: 403 - DNA, 30 bp, Artificial Sequence
  Synthetic Construct
ggtttgatctttacggtagaaggaactaca

SEQ ID NO: 404 - DNA, 19 bp, Artificial Sequence
  Synthetic Construct
ctttacggtagaaggaact

SEQ ID NO: 405 - DNA, 312 bp, Homo sapiens
cctccagactagcatttactactatatatttatttttccttttattctagttgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaaggttagtatcaaagatacctttttaaaataaaatactggttacatttgata

SEQ ID NO: 406 - DNA, 49 bp, Artificial Sequence
  Synthetic Construct
gtacaagaacaccttcagaaccggaggcaacagttgaatgaaatgttaa

SEQ ID NO: 407 - DNA, 48 bp, Artificial Sequence
  Synthetic Construct
catgttcttgtggaagtcttggcctccgttgtcaacttactttacaat

SEQ ID NO: 408 - DNA, 276 bp, Homo sapiens
taaaaagacatggggcttcatttttgttttgcctttttggtatcttacaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggtagggcgacagatctaataggaatgaaaacattttagcagactttttaa

SEQ ID NO: 409 - DNA, 47 bp, Artificial Sequence
  Synthetic Construct
ggtatcttacaggaactccaggatggcattgggcagcggcaaactgt

SEQ ID NO: 410 - DNA, 47 bp, Artificial Sequence
  Synthetic Construct
ccatagaatgtccttgaggtcctaccgtaacccgtcgccgtttgaca

SEQ ID NO: 411 - DNA, 18 bp, Artificial Sequence
  Synthetic Construct
tcactttcataatgctgg

SEQ ID NO: 412 - DNA, 15 bp, Artificial Sequence
  Synthetic Construct
  misc_feature (1)..(15): a, g, t, c corresponds to abc-DNA modified adenine, guanine, thymine and methylcytosine, respectively.
tccattcggctccaa

SEQ ID NO: 413 - DNA, 15 bp, Artificial Sequence
  Synthetic Construct
  misc_feature (1)..(15): a, g, t, c corresponds to abc-DNA modified adenine, guanine, thymine and methylcytosine, respectively.
  misc_feature (1)..(15): Phosphorothioate internucleotide linkage group at the 7′ end of the nucleotide.
tccattcggctccaa

SEQ ID NO: 414 - DNA, 18 bp, Artificial Sequence
  Synthetic Construct
  misc_feature (1)..(18): a, g, t, c corresponds to abc-DNA modified adenine, guanine, thymine and methylcytosine, respectively.
gatctttacggtagaagg

SEQ ID NO: 415 - DNA, 18 bp, Artificial Sequence
  Synthetic Construct
  misc_feature (1)..(18): a, g, t, c corresponds to abc-DNA modified adenine, guanine, thymine and methylcytosine, respectively.
atctttacggtagaagga

SEQ ID NO: 416 - DNA, 18 bp, Artificial Sequence
  Synthetic Construct
  misc_feature (1)..(18): a, g, t, c corresponds to abc-DNA modified adenine, guanine, thymine and methylcytosine, respectively.
tctttacggtagaaggaa

SEQ ID NO: 417 - DNA, 18 bp, Artificial Sequence
  Synthetic Construct
  misc_feature (1)..(18): a, g, t, c corresponds to abc-DNA modified adenine, guanine, thymine and methylcytosine, respectively.
ctttacggtagaaggaac

SEQ ID NO: 418 - DNA, 18 bp, Artificial Sequence
  Synthetic Construct
  misc_feature (1)..(18): a, g, t, c corresponds to abc-DNA modified adenine, guanine, thymine and methylcytosine, respectively.
tttacggtagaaggaact

SEQ ID NO: 419 - DNA, 18 bp, Artificial Sequence
  Synthetic Construct
  misc_feature (1)..(18): a, g, t, c corresponds to abc-DNA modified adenine, guanine, thymine and methylcytosine, respectively.
ttacggtagaaggaacta

SEQ ID NO: 420 - DNA, 18 bp, Artificial Sequence
  Synthetic Construct
  misc_feature (1)..(18): a, g, t, c corresponds to abc-DNA modified adenine, guanine, thymine and methylcytosine, respectively.
tacggtagaaggaactac

SEQ ID NO: 421 - DNA, 18 bp, Artificial Sequence
  Synthetic Construct
  misc_feature (1)..(18): a, g, t, c corresponds to abc-DNA modified adenine, guanine, thymine and methylcytosine, respectively.
aactagttcaatatttta

SEQ ID NO: 422 - DNA, 18 bp, Artificial Sequence
  Synthetic Construct
  misc_feature (1)..(18): a, g, t, c corresponds to abc-DNA modified adenine, guanine, thymine and methylcytosine, respectively.
ctagttcaatattttagt

SEQ ID NO: 423 - DNA, 18 bp, Artificial Sequence
  Synthetic Construct
  misc_feature (1)..(18): a, g, t, c corresponds to abc-DNA modified adenine, guanine, thymine and methylcytosine, respectively.
agttcaatattttagtgt

SEQ ID NO: 424 - DNA, 18 bp, Artificial Sequence
  Synthetic Construct
  misc_feature (1)..(18): a, g, t, c corresponds to abc-DNA modified adenine, guanine, thymine and methylcytosine, respectively.
ttcaatattttagtgtct

SEQ ID NO: 425 - DNA, 18 bp, Artificial Sequence
  Synthetic Construct
  misc_feature (1)..(18): a, g, t, c corresponds to abc-DNA modified adenine, guanine, thymine and methylcytosine, respectively.
caatattttagtgtctcc